(19) United States
(12) Patent Application Publication
(10) Pub. No.: US 2021/0353610 A1
(43) Pub. Date: Nov. 18, 2021

(54) SMALL MOLECULE MENIN INHIBITORS

(71) Applicants: THE REGENTS OF THE UNIVERSITY OF MICHIGAN, Ann Arbor, MI (US); AGIOS PHARMACEUTICALS, INC., Cambridge, MA (US)

(72) Inventors: Shaomeng WANG, Ann Arbor, MI (US); Shilin XU, Ann Arbor, MI (US); Angelo AGUILAR, Ann Arbor, MI (US); Liyue HUANG, Ann Arbor, MI (US); Tianfeng XU, Ann Arbor, MI (US); Meng ZHANG, Ann Arbor, MI (US); Renqi XU, Ann Arbor, MI (US); Fuming XU, Ann Arbor, MI (US); Haibin ZHOU, Ann Arbor, MI (US); Tao LIU, Cambridge, MA (US)

(21) Appl. No.: 17/277,954

(22) PCT Filed: Sep. 30, 2019
(86) PCT No.: PCT/US2019/053904

§ 371 Date: Mar. 19, 2021

Related U.S. Application Data
(60) Provisional application No. 62/740,561, filed on Oct. 3, 2018.
(60) Provisional application No. 62/740,567, filed on Oct. 3, 2018.

(51) Int. Cl. A61K 31/454; A61K 45/06; A61P 35/00; C07C 271/24; C07D 401/14; C07D 413/14; C07D 405/14; C07D 401/06; A61K 31/277; A61K 31/4523

(52) U.S. Cl. CPC A61K 31/454; A61K 45/06; A61P 35/00; C07C 271/24; A61K 31/4523; C07D 413/14; C07D 405/14; C07D 401/06; A61K 31/277; C07D 401/14

(57) ABSTRACT

The present disclosure provides compounds represented by Formula I: and the pharmaceutically acceptable salts and solvates thereof, wherein R^{1a}, R^{1b}, R^{1c}, E, G, and Q are as defined as set forth in the specification. The present disclosure also provides compounds of Formula I for use to treat cancer or any other disease, condition, or disorder that is responsive to inhibition of menin.

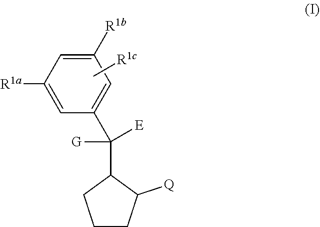

### CROSS-REFERENCE TO RELATED APPLICATIONS

This application claims the benefit of U.S. Provisional Application No. 62/740,561, filed Oct. 3, 2018, and U.S. Provisional Application No. 62/740,567, filed Oct. 3, 2018, each of which is incorporated by reference in its entirety.

### GOVERNMENT LICENSE RIGHTS

This invention was made with government support under Grant No. CA208267 awarded by the National Institutes of Health. The government has certain rights in the invention.

### BACKGROUND OF THE INVENTION

### Field of the Invention

The present disclosure provides compounds as menin inhibitors and therapeutic methods of treating conditions and diseases wherein inhibition of menin provides a benefit.

### Background Art

Mixed-lineage leukemia (MLL) is a proto-oncogene that was originally discovered at the site of chromosomal translocations in human leukemias. Due to chromosomal translocations, MLL is fused with more than 40 different partner proteins to yield a diverse collection of chimeric fusion proteins. The MLL protein is a histone methyltransferase that covalently modifies chromatin and is mutated in certain subsets of acute leukemia. Many of the fusion partners constitutively activate novel transcriptional effector properties of MLL that often correlate with its oncogenic potential in animal models of acute leukemia. MLL normally associates with a group of highly conserved cofactors to form a macromolecular complex that includes menin, a product of the MEN1 tumor suppressor gene. The MEN1 gene is mutated in heritable and sporadic endocrine tumors.

Menin is in involved in a diverse network of protein-protein interactions. Cierpicki and Grembecka, Future Med. Chem. 6:447-462 (2014). Overexpression of menin leads to inhibition of Ras-transformed cells. Menin interacts with the transcription factors JunD and NF-κB and represses their activation of gene transcription. Studies on these interacting proteins suggest that menin exerts its effects predominantly through inhibitory effects on transcription. But an alternative possibility is that menin mediates its effects through transcriptional activation of target genes. Additionally, menin interacts with RPA2, a component of a single-stranded DNA-binding protein involved in DNA repair and replication. Menin also interacts with FANCD2, a nuclear protein that plays a critical role in maintaining genome stability with breast cancer 1 gene (Brea1) product.

The mechanisms by which menin, which does not have significant homology with other proteins, functions as a tumor suppressor are not completely known. Menin plays a role in regulating cellular proliferation because Men1 knockout mice show increased proliferation in neuroendocrine tissues, down-modulation of menin in epithelial cells increases proliferation, and Men1 knockout fibroblasts proliferate more rapidly than wild-type cells as assayed by tritiated thymidine incorporation. MEN1 cells also have increased sensitivity to DNA-damaging agents. Menin interacts with promoters of HOX genes.

Certain oncogenic MLL fusion proteins stably associate with menin through a high-affinity interaction that is required for the initiation of MLL-mediated leukemogenesis. Menin is essential for maintenance of MLL-associated but no other oncogene induced myeloid transformation. Acute genetic ablation of menin reverses Hox gene expression mediated by MLL-menin promoter-associated complexes, and specifically eliminates the differentiation arrest and oncogenic properties of MLL-transformed leukemic blasts.

MLL fusion proteins, a consequence of acquired genetic aberrations, transform hematopoietic cells through two alternate mechanisms, by either constitutive transcriptional effector activity or inducing forced MLL dimerization and oligomerization. Both mechanisms result in the inappropriate expression of a subset of HOX genes, particularly HOXA9, whose consistent expression is a characteristic feature of human MLL leukemias.

Menin interacts with transcription activators, e.g., sc-Myb, MLL1, SMAD 1,3,5, Pem, Runx2, Hlbx9, ER, PPARγ, vitamin D receptor, transcription repressors, e.g., JunD, Sin3A, HDAC, EZH2, PRMT5, NFκB, Sirt1, CHES1, cell signaling proteins, e.g., AKT, SOS1/GEF, β-catenin, SMAD 1,3,5, NFκB, ER, PPARγ, vitamin D receptor, and other proteins, e.g., cell cycle: RPA2, ASK; DNA repair: FANCD2; cell structure: GFAP, vimenten, NMMHCIIA, IQGAP1; Others: HSP70, CHIP, (“menin-interacting proteins”) involved in regulating gene transcription and cell signaling. Matkar, Trends in Biochemical Sciences 38: 394-402 (2013). Targeting menin interactions, e.g., menin-MLL interaction, with small molecules represents an attractive strategy to develop new anticancer agents. See, e.g., Cierpicki and Grembecka, Future Med. Chem. 6:447-462 (2014); He et al., J. Med. Chem. 57:1543-1556 (2014); and Borkin et al., Cancer Cell 27:589-602 (2015).

Small molecules that disrupt the interaction of MLL and menin are disclosed in U.S. Pat. Nos. 9,212,180 and 9,216,993; U.S. Patent Application Publication Nos. 2011/0065690; 2014/0275070; 2016/0045504; and 2016/0046647; and International Publication Nos. WO 2017/192543; and WO 2018/183857. Peptides that disrupt the interaction of MLL and menin are disclosed in U.S. Patent Application Publication No. 2009/0298772.

There is an ongoing need for new small molecule drugs for treating cancer and other diseases responsive to menin inhibition.

### BRIEF SUMMARY OF THE INVENTION

In one aspect, the present disclosure provides cyclopentylcarbamates represented by Formulae I-VI below, and the pharmaceutically acceptable salts and solvates thereof, collectively referred to herein as “Compounds of the Disclosure.” Compounds of the Disclosure are menin inhibitors and thus are useful in treating diseases or conditions wherein inhibition of menin provides a therapeutic benefit to a patient.

In another aspect, the present disclosure provides methods of treating a condition or disease by administering a therapeutically effective amount of a Compound of the Disclosure to a subject, e.g., a human, in need thereof. The disease or condition is treatable by inhibition of menin, for example, a cancer, e.g., leukemia, a chronic autoimmune disorder, an inflammatory condition, a proliferative disorder, sepsis, or a viral infection. Also provided are methods of preventing the proliferation of unwanted proliferating cells, such as cancer, in a subject comprising administering a therapeutically effective amount of a Compound of the Disclosure to a subject at risk of developing a condition characterized by unwanted proliferating cells. In some embodiments, the Compounds of the Disclosure reduce the proliferation of unwanted cells by inducing apoptosis and/or differentiation in those cells.

In another aspect, the present disclosure provides a method of inhibiting menin in an individual, comprising administering to the individual an effective amount of at least one Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating diseases or conditions wherein inhibition of menin provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in treatment of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF DRAWINGS

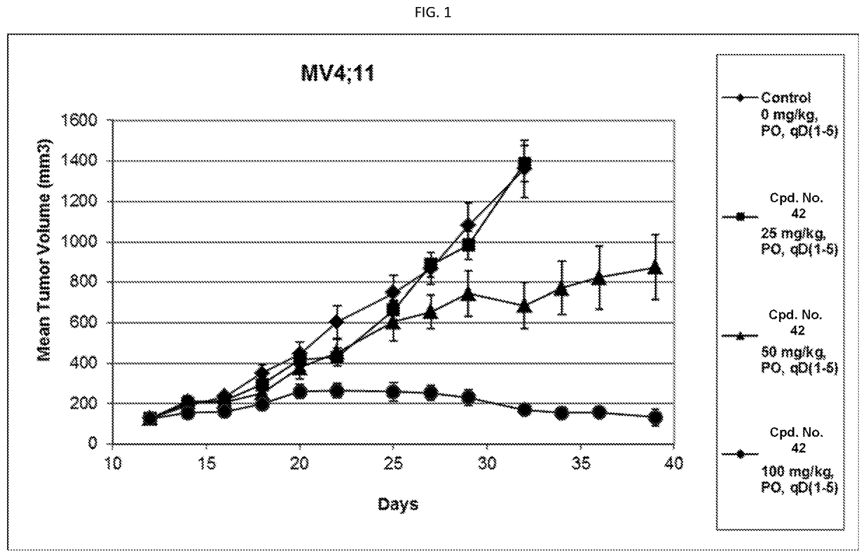

FIG. 1 is a line graph showing the activity of Cpd. No. 42 in the MV4-11 xenograft tumor model in mice.

### DETAILED DESCRIPTION OF THE INVENTION

### I. Compounds of the Disclosure

Compounds of the Disclosure are menin inhibitors.

In one embodiment, Compounds of the Disclosure are compounds of Formula I:

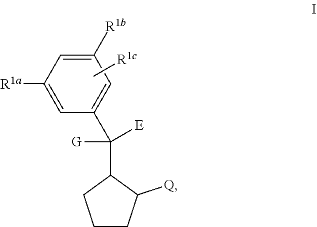

wherein:

Q is selected from the group consisting of —N(H)C(═O)OR, —N(R)C(═O)OR, —N(H)C(═O)R, —N(H)C(O)NR₂,

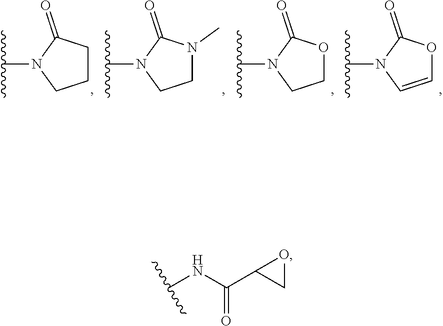

### —OR, and —OC(═O)R;

each R is independently C₁-C₄ alkyl or C₁-C₄ haloalkyl;

G is selected from the group consisting of:

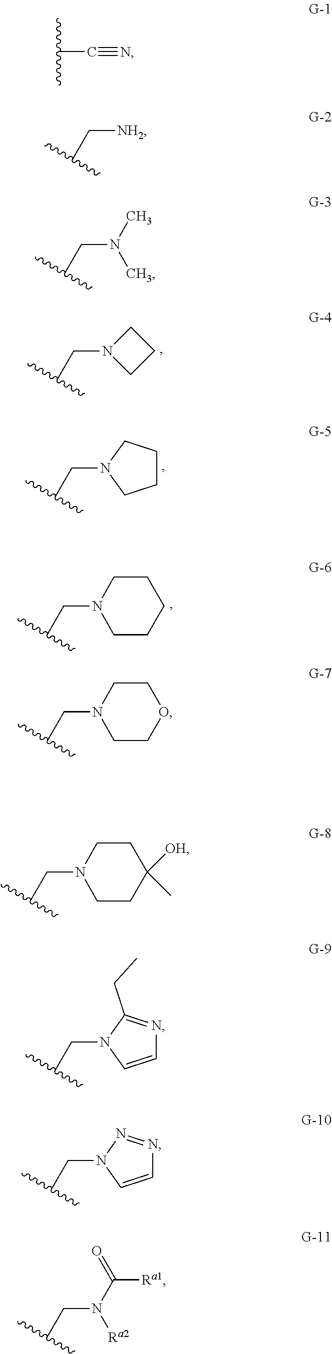

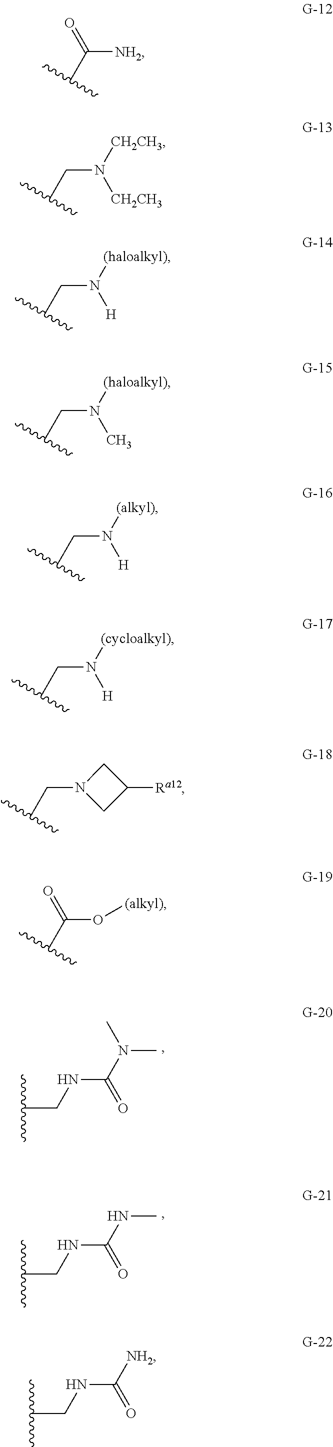

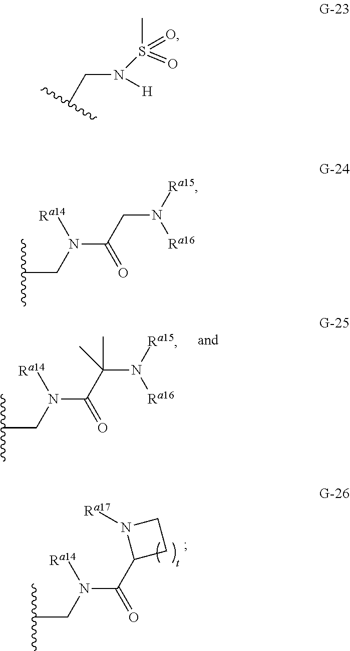

R^{a1} is selected from the group consisting of C₁-C₄ alkyl and C₁-C₄ alkoxy;

R^{a2} is selected from the group consisting of hydrogen and C₁-C₄ alkyl; or

R^{a1} and R^{a2} taken together with the atoms to which they are attached form an optionally substituted 5- or 6-membered heterocyclo;

R^{a12} is CN, C(O)OR^{a13}, C(O)N(R^{a13})₂, C₁-C₄ alkyl, OH, C₁-C₄ alkoxy, or F; each R^{a13} is independently C₁-C₄ alkyl;

R^{a14} is H or C₁-C₄ alkyl;

R^{a15} and R^{a16} are each independently H or C₁-C₄ alkyl, or R^{a14} and R^{a15} together with the nitrogen atom to which they are attached form an optionally substituted 4- to 6-membered heterocyclo;

R^{a17} is H or C₁-C₄ alkyl;

t is 1, 2, or 3;

R^{1a}, R^{1b}, and R^{1c} are each independently selected from the group consisting of hydrogen and halo;

E is selected from the group consisting of:

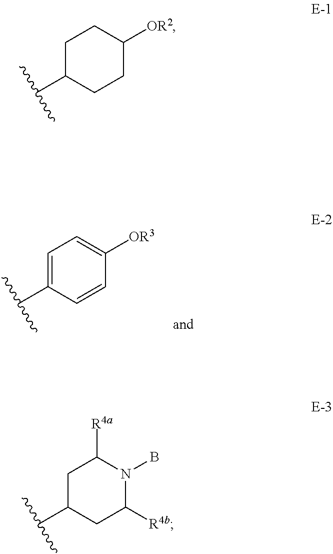

R² is selected from the group consisting of C₁-C₆ alkyl and —(CR^{5a}R^{5b})_{P}OR^{6a};

R³ is selected from the group consisting of hydrogen, —(CR^{5a}R^{5b})_{P}OR^{6b}, —CH₂C≡CR⁷, and R⁸;

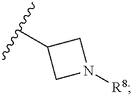

each R^{5a} and R^{5b} is independently selected from the group consisting of hydrogen and C₁-C₄ alkyl;

p is 2, 3, or 4;

R^{6a} is optionally substituted phenyl;

R^{6b} is selected from the group consisting of C₁-C₆ alkyl and optionally substituted phenyl;

R⁷ is optionally substituted phenyl;

R⁸ is optionally substituted phenyl;

R^{4a} and R^{4b} are independently selected from the group consisting of hydrogen, halo, and C₁-C₄ alkyl;

B is selected from the group consisting of C₁-C₆ alkyl, aralkyl —C(═O)R⁹, —(CR^{5c}R^{5d})ₘOR¹⁰,

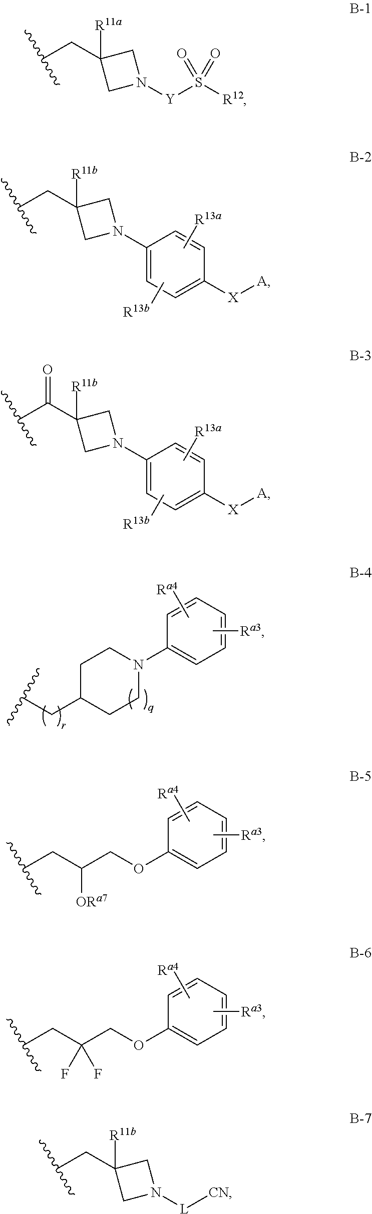

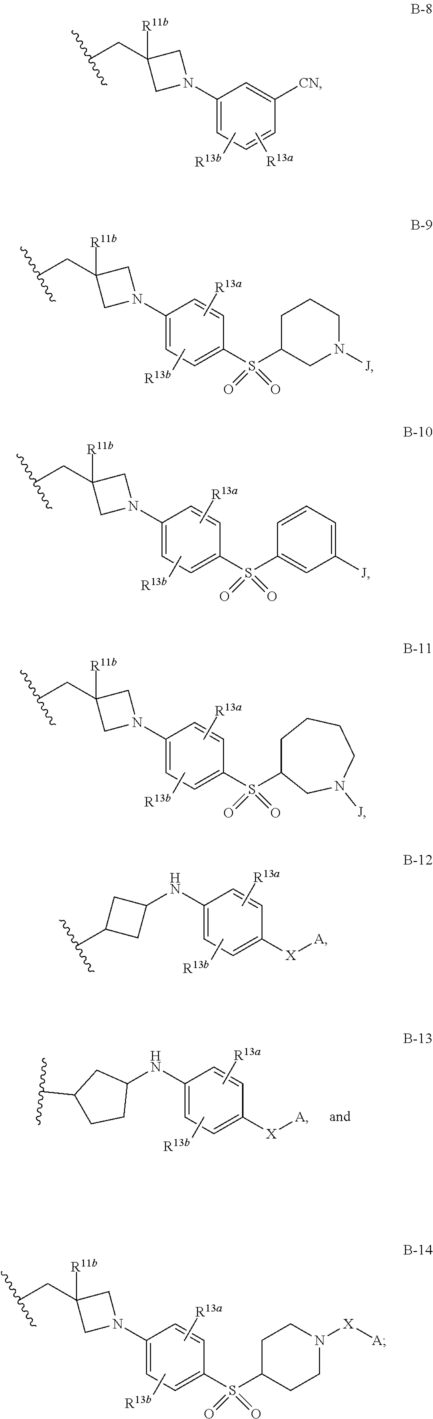

R⁹ is selected from the group consisting of C₁-C₆, alkyl, aralkyl, heteroaralkyl, and

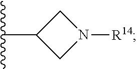

R¹⁴ is optionally substituted phenyl;

each R^{5c} and R^{5d} is independently selected from the group consisting of hydrogen and C₁-C₄ alkyl;

m is 2, 3, or 4;

R¹⁰ is optionally substituted phenyl;

R^{11a} is selected from the group consisting of hydrogen, halo, and C₁-C₄ alkyl;

Y is —(CR^{5e}R^{5f})ₒ;

each R^{5e} and R^{5f} is independently selected from the group consisting of hydrogen and C₁-C₄ alkyl;

o is 2, 3, or 4;

R¹² is optionally substituted phenyl;

R^{11b} is selected from the group consisting of hydrogen, halo, C₁-C₄ alkyl, and R^{a6};

R^{13a} and R^{13b} are independently selected from the group consisting of hydrogen, halo, C₁-C₄ alkyl, and R^{a5};

R^{a3} is selected from the group consisting of cyano, alkylsulfonyl, haloalkylsulfonyl, cycloalkylsulfonyl, arylsulfonyl, heteroarylsulfonyl, heterocyclosulfonyl, and carboxamido;

R^{a4} is selected from the group consisting of hydrogen, halo, C₁-C₄ alkyl, C₁-C₄ alkoxy, and C₁-C₄ haloalkyl;

R^{a5} is selected from the group consisting of (heterocyclo)alkyl, (heteroaryl)alkyl, (amino)alkyl, carboxamido, hydroxyalkyl, heteroaryloxy, heteroaralkyloxy, C₁-C₄ alkoxy, —OR^{a8}, and —CH₂NR^{a9}C(═O)R^{a10};

R^{a6} is selected from the group consisting of hydroxy, C₁-C₄ alkoxy, C₁-C₄ haloalkyl, C₁-C₄ hydroxyalkyl, alkoxyalkyl, carboxy, alkoxy carbonyl, and carboxamido;

R^{a7} is selected from the group consisting of hydrogen and C₁-C₄ alkyl;

R^{a8} is selected from the group consisting of heteroaryl, heteroaralkyl, alkoxyalkyl, and (heterocyclo)alkyl;

R^{a9} is selected from the group consisting of hydrogen and C₁-C₄ alkyl;

R^{a10} is C₁-C₄ alkyl;

r is 0 or 1;

q is 0, 1, 2, or 3;

L is selected from the group consisting of C₃-C₈ cycloalkylenyl, optionally substituted 5-membered heteroaryl enyl, and optionally substituted 6-membered heteroaryl enyl;

(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b},

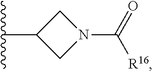

### and R^{a11}; or

(2) X is absent; and A is selected from the group consisting of cyano, C(═O)OH, C₁-C₄ alkyl, C₁-C₄ alkoxy, and C₁-C₄ haloalkyl;

J is carboxamido or C(O)CH₂CN;

R^{a11} is selected from the group consisting of hydroxyalkyl and (heterocyclo)alkyl;

R^{15a} and R^{15b} are independently selected from the group consisting of hydrogen, optionally substituted C₁-C₆ alkyl, optionally substituted C₃-C₁₂ cycloalkyl, optionally substituted 4- to 14-membered heterocyclo, optionally substituted C₆-C₁₀ aryl, and optionally substituted 5- to 14-membered heteroaryl; and

R¹⁶ is selected from the group consisting of (amino)alkyl and (heterocyclo)alkyl,

or a pharmaceutically acceptable salt or solvate thereof.

In another embodiment, Compounds of the Disclosure are compounds of Formula I, wherein:

Q is selected from the group consisting of —N(H)C(═O)OR, —OR, and —OC(═O)R;

R is a C₁-C₄ alkyl;

G is selected from the group consisting of:

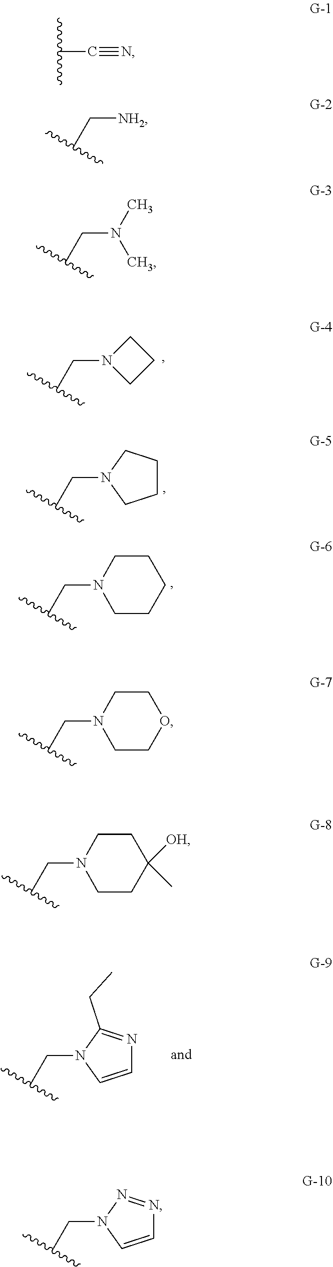

R^{1a}, R^{1b}, and R^{1c} are each independently selected from the group consisting of hydrogen and halo;

E is selected from the group consisting of:

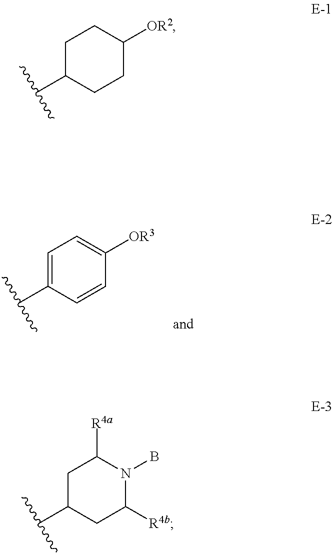

R² is selected from the group consisting of C₁-C₆ alkyl and —(CR^{5a}R^{5b})_{P}OR^{6a};

R³ is selected from the group consisting of hydrogen, —(CR^{5a}R^{5b})_{P}OR^{6b}, —CH₂C≡CR⁷, and R⁸;

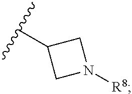

each R^{5a} and R^{5b} is independently selected from the group consisting of hydrogen and C₁-C₄ alkyl;

p is 2, 3, or 4;

R^{6a} is optionally substituted phenyl;

R^{6b} is selected from the group consisting of C₁-C₆, alkyl and optionally substituted phenyl;

R⁷ is optionally substituted phenyl;

R⁸ is optionally substituted phenyl;

R^{4a} and R^{4b} are independently selected from the group consisting of hydrogen, halo, and C₁-C₄ alkyl;

B is selected from the group consisting of C₁-C₆ alkyl, aralkyl, —C(═O)R⁹, —(CR^{5c}R^{5d})ₘOR¹⁰

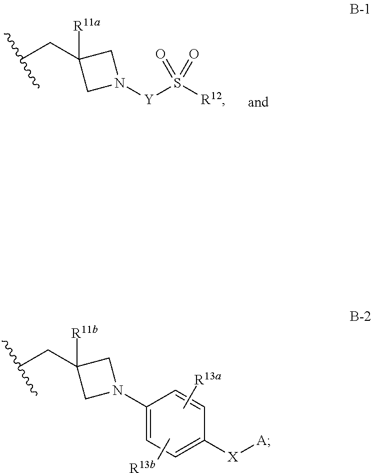

R⁹ is selected from the group consisting of C₁-C₆ alkyl, aralkyl, heteroaralkyl, and

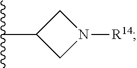

R¹⁴ is optionally substituted phenyl;

each R^{5c} and R^{5d} is independently selected from the group consisting of hydrogen and C₁-C₄ alkyl;

m is 2, 3, or 4;

R¹⁰ is optionally substituted phenyl;

R^{11a} is selected from the group consisting of hydrogen, halo, and C₁-C₄ alkyl;

Y is —(CR^{5e}R^{5f})ₒ;

each R^{5e} and R^{5f} is independently selected from the group consisting of hydrogen and C₁-C₄ alkyl;

o is 2, 3, or 4;

R¹² is optionally substituted phenyl;

R^{11b} is selected from the group consisting of hydrogen, hydroxy, halo, and C₁-C₄ alkyl;

R^{13a} and R^{13b} are independently selected from the group consisting of hydrogen, halo, and C₁-C₄ alkyl;

(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b}, and R¹⁶;

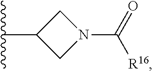

or

(2) X is absent; and A is selected from the group consisting of cyano and —C(═O)OH;

R^{15a} and R^{15b} are independently selected from the group consisting of hydrogen, optionally substituted C₁-C₆ alkyl, optionally substituted C₃-C₁₂ cycloalkyl, optionally substituted 4- to 14-membered heterocyclo, optionally substituted C₆-C₁₀ aryl, and optionally substituted 5- to 14-membered heteroaryl; and

R¹⁶ is selected from the group consisting of (amino)alkyl and (heterocyclo)alkyl, or a pharmaceutically acceptable salt or solvate thereof.

In another embodiment, Compounds of the Disclosure are compounds of Formula I, wherein Q is —N(H)C(═O)OR, or a pharmaceutically acceptable salt or solvate thereof.

In another embodiment, Compounds of the Disclosure are compounds of Formula I, wherein Q is —OR, or a pharmaceutically acceptable salt or solvate thereof.

In another embodiment, Compounds of the Disclosure are compounds of Formula I, wherein Q is —OC(═O)R, or a pharmaceutically acceptable salt or solvate thereof.

In another embodiment, Compounds of the Disclosure are compounds of Formula I, wherein R is selected from the group consisting of methyl, ethyl, and n-propyl, or a pharmaceutically acceptable salt or solvate thereof. In another embodiment, R is methyl.

In another embodiment, Compounds of the Disclosure are compounds of Formula II:

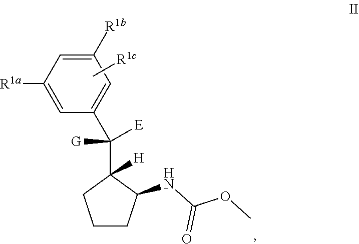

wherein R^{1a}, R^{1b}, R^{1c}, E, and G are as defined in connection with Formula I, or a pharmaceutically acceptable salt or solvate thereof.

In another embodiment, Compounds of the Disclosure are compounds of Formulae I or II, wherein E is E-1, or a pharmaceutically acceptable salt or solvate thereof. In another embodiment, R² is —(CH₂)_{P}OR^{6a}.

In another embodiment, Compounds of the Disclosure are compounds of Formulae I or II, wherein E is E-2, or a pharmaceutically acceptable salt or solvate thereof. In another embodiment, R³ is —(CH₂)ₚOR^{6b}. In another embodiment, R³ is —CH₂C≡CR⁷. In another embodiment, R³ is

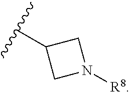

In another embodiment, Compounds of the Disclosure are compounds of Formulae I or II, wherein E is E-3, or a pharmaceutically acceptable salt or solvate thereof.

In another embodiment, Compounds of the Disclosure are compounds of Formulae I or II, wherein B is C₁-C₆, alkyl, or a pharmaceutically acceptable salt or solvate thereof.

In another embodiment, Compounds of the Disclosure are compounds of Formulae I or II, wherein B is aralkyl, or a pharmaceutically acceptable salt or solvate thereof.

In another embodiment, Compounds of the Disclosure are compounds of Formulae I or II, wherein B is —C(═O)R⁹, or a pharmaceutically acceptable salt or solvate thereof. In another embodiment, R⁹ is selected from the group consisting of aralkyl, heteroaralkyl, and

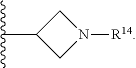

In another embodiment, R⁹ is

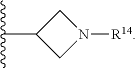

In another embodiment, Compounds of the Disclosure are compounds of Formulae I or II, wherein B is —(CH₂)ₘOR¹⁰ or a pharmaceutically acceptable salt or solvate thereof. In another embodiment, m is 2 or 3.

In another embodiment, Compounds of the Disclosure are compounds of Formulae I or II, wherein B is B-1, or a pharmaceutically acceptable salt or solvate thereof. In another embodiment, Y is —(CH₂)ₒ—.

In another embodiment, Compounds of the Disclosure are compounds of Formulae I or II, wherein B is B-2, or a pharmaceutically acceptable salt or solvate thereof. In another embodiment, X is —S(═O)₂.

In another embodiment, Compounds of the Disclosure are compounds of Formulae I or II, wherein G is —CN, or a pharmaceutically acceptable salt or solvate thereof.

In another embodiment, Compounds of the Disclosure are compounds of Formulae I or II, wherein G is —CH₂NH₂, or a pharmaceutically acceptable salt or solvate thereof.

In another embodiment, Compounds of the Disclosure are compounds of Formulae I or II, wherein G is —CF₂N(CH₃)₂, or a pharmaceutically acceptable salt or solvate thereof.

In another embodiment, Compounds of the Disclosure are compounds of Formulae I or II, wherein G is

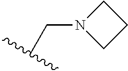 [SMILES: *CN1CCC1]

or a pharmaceutically acceptable salt or solvate thereof.

In another embodiment, Compounds of the Disclosure are compounds of Formulae I or II, wherein G is

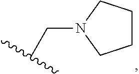 [SMILES: *CN1CCCC1]

or a pharmaceutically acceptable salt or solvate thereof.

In another embodiment, Compounds of the Disclosure are compounds of Formulae I or II, wherein G is

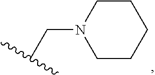 [SMILES: *CN1CCCCC1]

or a pharmaceutically acceptable salt or solvate thereof.

In another embodiment, Compounds of the Disclosure are compounds of Formulae I or II, wherein G is

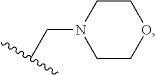 [SMILES: *CN1CCOCC1]

or a pharmaceutically acceptable salt or solvate thereof.

In another embodiment, Compounds of the Disclosure are compounds of Formulae I or II, wherein G is

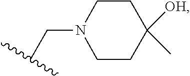 [SMILES: *CN1CCC(C)(O)CC1]

or a pharmaceutically acceptable salt or solvate thereof.

In another embodiment, Compounds of the Disclosure are compounds of Formulae I or II, wherein G is

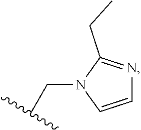 [SMILES: *Cn1ccnc1CC]

or a pharmaceutically acceptable salt or solvate thereof.

In another embodiment, Compounds of the Disclosure are compounds of Formulae I or II, wherein G is

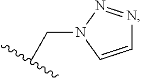 [SMILES: *Cn1ccnn1]

or a pharmaceutically acceptable salt or solvate thereof.

In another embodiment, Compounds of the Disclosure are compounds of Formula III:

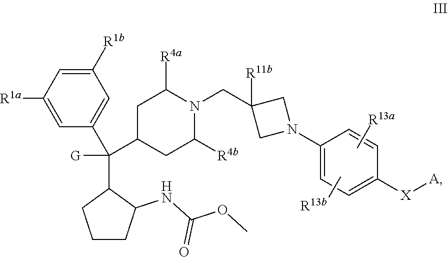

wherein:

R^{1a}, R^{1b}, R^{4a}, R^{4b}, R^{11b}, R^{13a}, R^{13b}, A, and X are as defined in connection with Formula I; and

G is selected from the group consisting of:

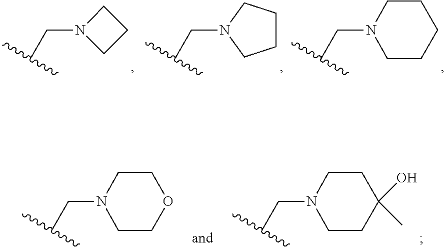

or a pharmaceutically acceptable salt or solvate thereof.

In another embodiment, Compounds of the Disclosure are compounds of Formula III wherein G is

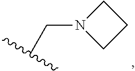 [SMILES: *CN1CCC1]

or a pharmaceutically acceptable salt or solvate thereof.

In another embodiment, Compounds of the Disclosure are compounds of Formula IV:

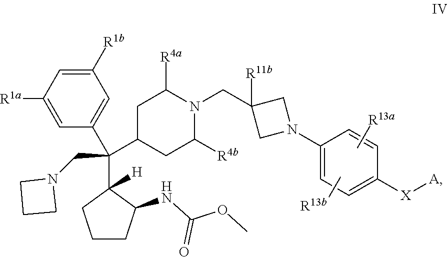

wherein R^{1a}, R^{1b}, R^{4a}, R^{4b}, R^{11b}, R^{13a}, R^{13b}, A, and X are as defined in connection with Formula I, or a pharmaceutically acceptable salt or solvate thereof.

In another embodiment, Compounds of the Disclosure are compounds of Formulae I-IV, wherein R^{4a} and R^{4b} are hydrogen, or a pharmaceutically acceptable salt or solvate thereof.

In another embodiment, Compounds of the Disclosure are compounds of Formulae I-IV, wherein R^{11b} is selected from the group consisting of hydrogen and fluoro, or a pharmaceutically acceptable salt or solvate thereof. In another embodiment, R^{11b} is hydrogen. In another embodiment, R^{11b} is fluoro.

In another embodiment, Compounds of the Disclosure are compounds of Formulae I-IV, wherein R^{13a} and R^{13b} are independently selected from the group consisting of hydrogen and fluoro, or a pharmaceutically acceptable salt or solvate thereof. In another embodiment, R^{13a} is hydrogen and R^{13b} is fluoro.

In another embodiment, Compounds of the Disclosure are compounds of Formulae I-IV, wherein X is —C(═O)—, or a pharmaceutically acceptable salt or solvate thereof.

In another embodiment, Compounds of the Disclosure are compounds of Formulae I-IV, wherein X is —S(═O)₂—, or a pharmaceutically acceptable salt or solvate thereof.

In another embodiment, Compounds of the Disclosure are compounds of Formulae I-IV, wherein A is optionally substituted C₃-C₁₂ cycloalkyl, or a pharmaceutically acceptable salt or solvate thereof. In another embodiment, A is selected from the group consisting of:

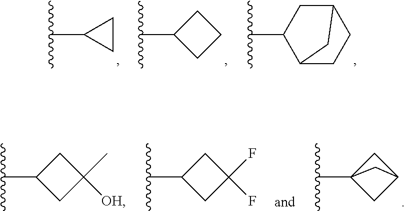

In another embodiment, Compounds of the Disclosure are compounds of Formulae I-IV, wherein A is optionally substituted 4- to 14-membered heterocyclo, or a pharmaceutically acceptable salt or solvate thereof. In another embodiment, A is selected from the group consisting of:

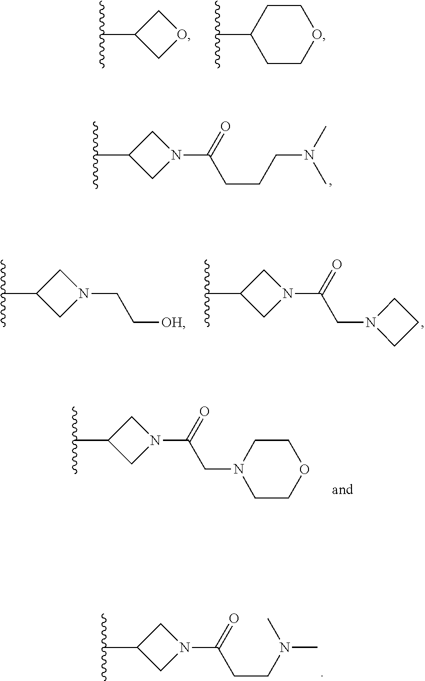

In another embodiment, Compounds of the Disclosure are compounds of Formulae I-IV, wherein A is optionally substituted phenyl, or a pharmaceutically acceptable salt or solvate thereof.

In another embodiment, Compounds of the Disclosure are compounds of Formulae I-IV, wherein A is optionally substituted 5- or 6-membered heteroaryl, or a pharmaceutically acceptable salt or solvate thereof. In another embodiment A is selected from the group consisting of:

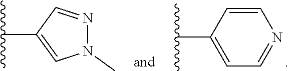

In another embodiment, Compounds of the Disclosure are compounds of Formulae I-IV, wherein A is —NR^{15a}R^{15b}, or a pharmaceutically acceptable salt or solvate thereof. In another embodiment, R^{15a} and R^{15b} are independently selected from the group consisting of hydrogen and optionally substituted C₁-C₆ alkyl.

In another embodiment, Compounds of the Disclosure are compounds of Formulae I-IV, wherein A is

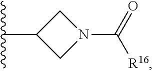

or a pharmaceutically acceptable salt or solvate thereof. In another embodiment, R¹⁶ is —CH₂CH₂CH₂N(CH₃)₂. In another embodiment, R¹⁶ is —CH₂CH₂N(CH₃)₂. In another embodiment, R¹⁶ is

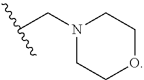 [SMILES: *CN1CCOCC1]

In another embodiment, Compounds of the Disclosure are compounds of Formulae I-IV, wherein X is absent and A is cyano, or a pharmaceutically acceptable salt or solvate thereof.

In another embodiment, Compounds of the Disclosure are compounds of Formulae I-IV, wherein R^{1a} and R^{1b} are independently selected from the group consisting of hydrogen and fluoro, or a pharmaceutically acceptable salt or solvate thereof.

In another embodiment, Compounds of the Disclosure are compounds of Formula I, wherein:

Q is selected from the group consisting of —N(H)C(═O)OR and —N(H)C(═O)R;

R is a C₁-C₄ alkyl;

G is selected from the group consisting of:

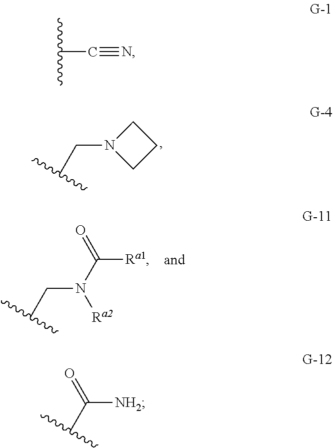

R^{a1} is selected from the group consisting of C₁-C₄ alkyl and C₁-C₄ alkoxy;

R^{a2} is selected from the group consisting of hydrogen and C₁-C₄ alkyl; or

R^{a1} and R^{a2} taken together with the atoms to which the are attached form an optionally substituted 5- or 6-membered heterocyclo;

R^{1a}, R^{1b}, and R^{1c} are each independently selected from the group consisting of hydrogen and halo;

E is:

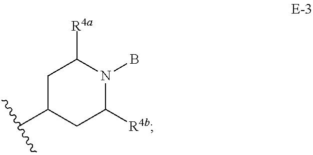

R^{4a} and R^{4b} are independently selected from the group consisting of hydrogen, halo, and C₁-C₄ alkyl;

B is selected from the group consisting of:

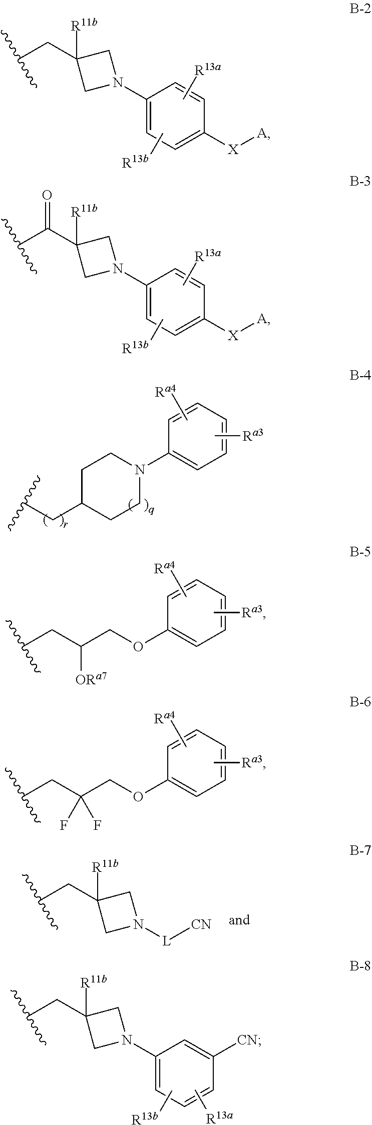

R^{11b} is selected from the group consisting of hydrogen, hydroxy, halo, C₁-C₄ alkyl, and R^{a6};

R^{13a} and R^{13b} are independently selected from the group consisting of hydrogen, halo, C₁-C₄ alkyl, and R^{a5};

R^{a3} is selected from the group consisting of cyano, alkylsulfonyl, haloalkylsulfonyl, cycloalkylsulfonyl, arylsulfonyl, heteroaryl sulfonyl, heterocyclosulfonyl, and carboxamido

R^{a4} is selected from the group consisting of hydrogen, halo, C₁-C₄ alkyl, C₁-C₄ alkoxy, and C₁-C₄ haloalkyl;

R^{a5} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10};

R^{a6} is selected from the group consisting of hydroxy, C₁-C₄ haloalkyl, alkoxyalkyl, carboxy, alkoxycarbonyl, and carboxamido;

R^{a7} is selected from the group consisting of hydrogen and C₁-C₄ alkyl;

R^{a8} is selected from the group consisting of heteroaryl, heteroaralkyl, alkoxyalkyl, and (heterocyclo)alkyl;

R^{a9} is selected from the group consisting of hydrogen and C₁-C₄ alkyl;

R^{a10} is C₁-C₄ alkyl;

r is 0 or 1;

q is 0, 1, 2, or 3;

L is selected from the group consisting of C₃-C₈ cycloalkylenyl, optionally substituted 5-membered heteroarylenyl, and optionally substituted 6-membered heteroaryl enyl;

(1) X is selected from the group consisting of —S(═O)₂— and —C(═O)—; and

A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b},

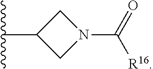

### and R^{a11}; or

(2) X is absent; and

A is selected from the group consisting of cyano and —C(═O)OH;

R^{a11} is selected from the group consisting of hydroxyalkyl and (heterocyclo)alkyl;

R^{15a} and R^{15b} are independently selected from the group consisting of hydrogen, optionally substituted C₁-C₆ alkyl, optionally substituted C₃-C₁₂ cycloalkyl, optionally substituted 4- to 14-membered heterocyclo, optionally substituted C₆-C₁₀ aryl, and optionally substituted 5- to 14-membered heteroaryl; and

R¹⁶ is selected from the group consisting of (amino)alkyl and (heterocyclo)alkyl, or a pharmaceutically acceptable salt or solvate thereof.

In another embodiment, Compounds of the Disclosure are compounds of Formula II, wherein R^{1a}, R^{1b}, R^{1c}, E, and G are as defined in connection with Formula I, or a pharmaceutically acceptable salt or solvate thereof.

In another embodiment, Compounds of the Disclosure are compounds of Formula I or Formula II, wherein B is B-2, or a pharmaceutically acceptable salt or solvate thereof.

In another embodiment, Compounds of the Disclosure are compounds of Formula I or Formula II, wherein B is B-3, or a pharmaceutically acceptable salt or solvate thereof. In another embodiment, X is —S(═O)₂. In another embodiment, X is absent and A is cyano. In another embodiment, R^{11b} is selected from the group consisting of hydrogen or fluoro, or a pharmaceutically acceptable salt or solvate thereof. In another embodiment, R^{11b} is selected from the group consisting of C₁-C₄ haloalkyl, alkoxyalkyl, carboxy, alkoxycarbonyl, and carboxamido. In another embodiment, R^{13a} is hydrogen. In another embodiment, R^{13a} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10}. In another embodiment, A is selected from the group consisting of phenyl substituted with 1 or 2 substituents independently selected from the group consisting of halo and carboxamido, and 5- or 6-membered heteroaryl substituted with 1 or 2 substituents independently selected from the group consisting of halo and carboxamido. In another embodiment, A is selected from the group consisting of unsubstituted C₃-C₆ cycloalkyl, C₃-C₆ cycloalkyl substituted with 1 or 2 substituents independently selected from the group consisting of halo, hydroxy, C₁-C₄ alkyl, and C₁-C₄ haloalkyl, unsubstituted 4- to 6-membered heterocyclo, and 4- to 6-membered heterocyclo substituted with 1 or 2 substituents independently selected from the group consisting of halo, hydroxy, C₁-C₄ alkyl, and C₁-C₄ haloalkyl, alkylcarbonyl, hydroxyalkylcarbonyl, and alkoxycarbonyl.

In another embodiment, Compounds of the Disclosure are compounds of Formula I or Formula II, wherein B is B-4, or a pharmaceutically acceptable salt or solvate thereof. In another embodiment, r is 0. In another embodiment, r is 1. In another embodiment, q is 0 or 1.

In another embodiment, Compounds of the Disclosure are compounds of Formula I or Formula II, wherein B is B-5, or a pharmaceutically acceptable salt or solvate thereof.

In another embodiment, Compounds of the Disclosure are compounds of Formula I or Formula II, wherein B is B-6, or a pharmaceutically acceptable salt or solvate thereof.

In another embodiment, Compounds of the Disclosure are compounds of Formula I or Formula II, wherein B is B-7, or a pharmaceutically acceptable salt or solvate thereof. In another embodiment, L is C₃-C₈ cycloalkylene. In another embodiment, L is 5-membered heteroaryl.

In another embodiment, Compounds of the Disclosure are compounds of Formula I or Formula II, wherein B is B-8, or a pharmaceutically acceptable salt or solvate thereof.

In another embodiment, Compounds of the Disclosure are compounds of Formula I or Formula II, wherein B is B-2 or B-3, and R^{11b} is selected from the group consisting of hydrogen or fluoro, or a pharmaceutically acceptable salt or solvate thereof.

In another embodiment, Compounds of the Disclosure are compounds of Formula I or Formula II, wherein G is G-1, or a pharmaceutically acceptable salt or solvate thereof.

In another embodiment, Compounds of the Disclosure are compounds of Formula I or Formula II, wherein G is G-4, or a pharmaceutically acceptable salt or solvate thereof.

In another embodiment, Compounds of the Disclosure are compounds of Formula I or Formula II, wherein G is G-11, or a pharmaceutically acceptable salt or solvate thereof. In another embodiment, R^{a1} selected from the group consisting of methyl and methoxy. In another embodiment, R^{a2} is hydrogen. In another embodiment, R^{a1} and R^{a2} are taken together with the atoms to which the are attached form an optionally substituted 5- or 6-membered heterocyclo, e.g., G is

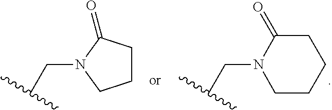 [SMILES: *CN1CCCC1=O ; *CN1CCCCC1=O]

In another embodiment, Compounds of the Disclosure are compounds of Formula I or Formula II, wherein G is G-12, or a pharmaceutically acceptable salt or solvate thereof.

In another embodiment, Compounds of the Disclosure are compounds of Formula III, wherein:

R^{1a}, R^{1b}, R^{4a}, R^{4b}, R^{11b}, R^{13a}, R^{13b}, A, and X are as defined in connection with Formula I; and

G is selected from the group consisting of G-4 and G-11, or a pharmaceutically acceptable salt or solvate thereof.

In another embodiment, Compounds of the Disclosure are compounds of Formula III, wherein R^{13b} is selected from the group consisting of hydrogen, or a pharmaceutically acceptable salt or solvate thereof.

In another embodiment, Compounds of the Disclosure are compounds of Formula V:

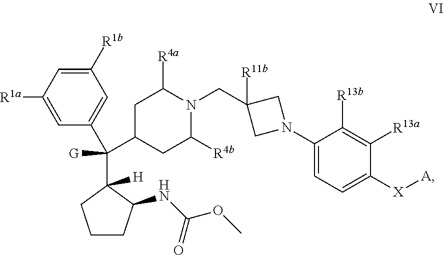

wherein R^{1a}, R^{1b}, R^{4a}, R^{4b}, R^{11b}, R^{13a}, R^{13b}, G, A, and X are as defined in connection with Formula III, or a pharmaceutically acceptable salt or solvate thereof. In another embodiment, X is —S(═O)₂—.

In another embodiment, Compounds of the Disclosure are compounds of Formula VI:

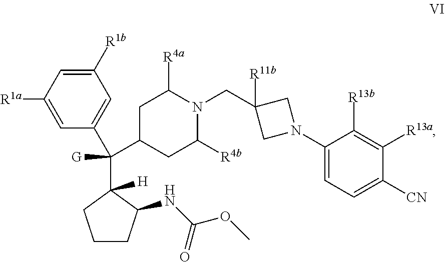

wherein R^{1a}, R^{1b}, R^{4a}, R^{4b}, R^{11b}, R^{13a}, R^{13b}, and G are as defined in connection with Formula III, or a pharmaceutically acceptable salt or solvate thereof.

In another embodiment, Compounds of the Disclosure are compounds of Formulae III, V, or VI, wherein G is G-4, or a pharmaceutically acceptable salt or solvate thereof.

In another embodiment, Compounds of the Disclosure are compounds of Formulae III, V, or VI, wherein G is G-11, or a pharmaceutically acceptable salt or solvate thereof. In another embodiment, G is —CH₂N(H)C(═O)CH₃. In another embodiment, G is —CH₂N(H)C(═O)OCH₃. In another embodiment, G is

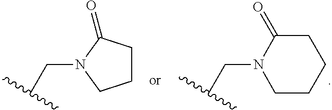 [SMILES: *CN1CCCC1=O ; *CN1CCCCC1=O]

In another embodiment, Compounds of the Disclosure are compounds of Formulae I-III, V, or VI, wherein R^{4a} and R^{4b} are hydrogen, or a pharmaceutically acceptable salt or solvate thereof.

In another embodiment, Compounds of the Disclosure are compounds of Formulae I-III, V, or VI, wherein R^{11b} is selected from the group consisting of hydrogen or fluoro, or a pharmaceutically acceptable salt or solvate thereof.

In another embodiment, Compounds of the Disclosure are compounds of Formulae I-III, V, or VI, wherein R^{11b} is selected from the group consisting of C₁-C₄ haloalkyl, alkoxyalkyl, carboxy, alkoxycarbonyl, and carboxamido, or a pharmaceutically acceptable salt or solvate thereof. In another embodiment, R^{11b} is selected from the group consisting of —C(═O)OH, —C(═O)OCH₃, —C(═O)N(H)CH₃, —CH₂F, and —CH₂OCH₃.

In another embodiment, Compounds of the Disclosure are compounds of Formulae I-III, V, or VI, wherein R^{13a} selected from the group consisting of hydrogen and fluoro, or a pharmaceutically acceptable salt or solvate thereof.

In another embodiment, Compounds of the Disclosure are compounds of Formulae I-III, V, or VI, wherein R^{13b} selected from the group consisting of hydrogen and fluoro, or a pharmaceutically acceptable salt or solvate thereof.

In another embodiment, Compounds of the Disclosure are compounds of Formulae I-III, V, or VI, wherein R^{13a} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10}, or a pharmaceutically acceptable salt or solvate thereof. In another embodiment, R^{13a} is selected from the group consisting of:

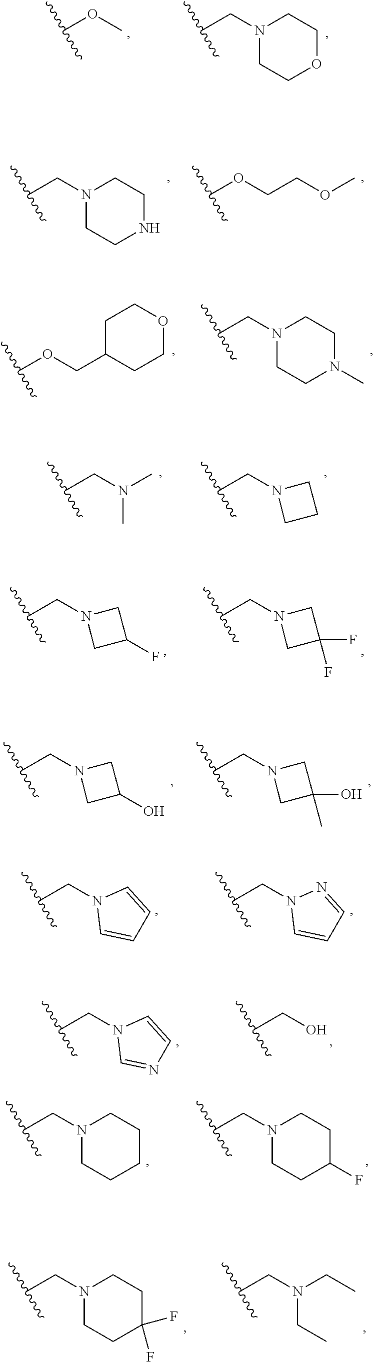

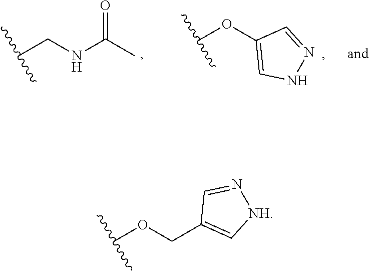

In another embodiment, Compounds of the Disclosure are compounds of Formulae I-III, or V, wherein A is selected from the group consisting of unsubstituted phenyl, phenyl substituted with 1 or 2 substituents independently selected from the group consisting of halo, carboxamido, and —N(H)C(═O)R^{19b}, and 5- or 6-membered heteroaryl substituted with 1 or 2 substituents independently selected from the group consisting of halo and carboxamido; and R^{19b} is C₁-C₆ alkyl, or a pharmaceutically acceptable salt or solvate thereof.

In another embodiment, Compounds of the Disclosure are compounds of Formulae I-III, or V, wherein A is phenyl substituted with 1 or 2 substituents independently selected from the group consisting of fluoro,

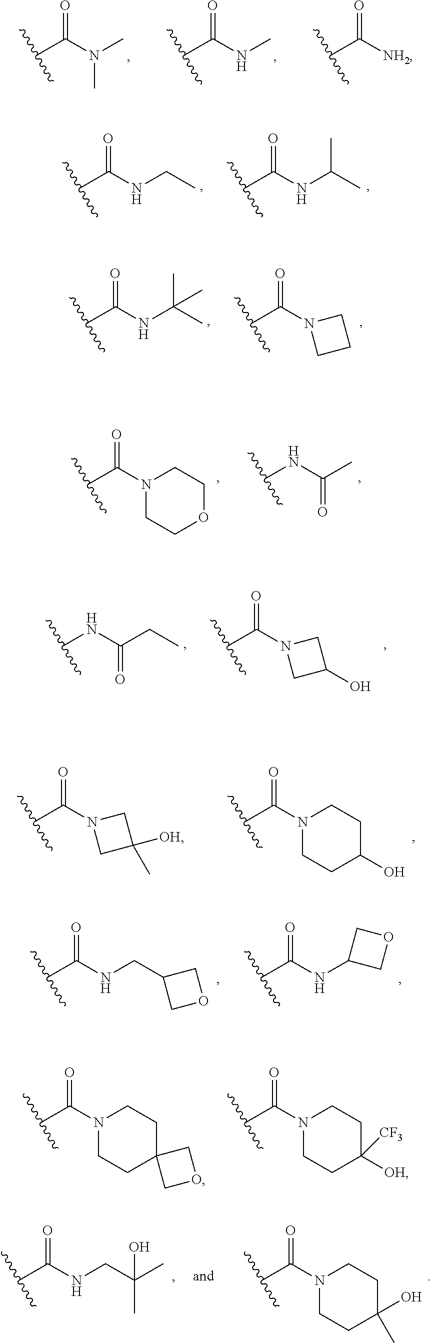

or a pharmaceutically acceptable salt or solvate thereof.

In another embodiment, Compounds of the Disclosure are compounds of Formulae I-III, or V, wherein A is 6-membered heteroaryl substituted with 1 or 2 substituents independently selected from the group consisting of fluoro,

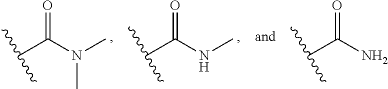

or a pharmaceutically acceptable salt or solvate thereof.

In another embodiment, Compounds of the Disclosure are compounds of Formulae I-III, or V, wherein A is selected from the group consisting of unsubstituted C₃-C₆ cycloalkyl, C₃-C₆ cycloalkyl substituted with 1 or 2 substituents independently selected from the group consisting of halo, hydroxy, C₁-C₄ alkyl, and C₁-C₄ haloalkyl, unsubstituted 4- to 6-membered heterocyclo, and 4- to 6-membered heterocyclo substituted with 1 or 2 substituents independently selected from the group consisting of halo, hydroxy, C₁-C₄ alkyl, and C₁-C₄ haloalkyl, alkylcarbonyl, hydroxyalkylcarbonyl, and alkoxycarbonyl, or a pharmaceutically acceptable salt or solvate thereof.

In another embodiment, Compounds of the Disclosure are compounds of Formulae I-III, or V, wherein A is selected from the group consisting of:

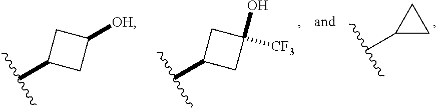

or a pharmaceutically acceptable salt or solvate thereof.

In another embodiment, Compounds of the Disclosure are compounds of Formulae I-III, or V, wherein A is selected from the group consisting of

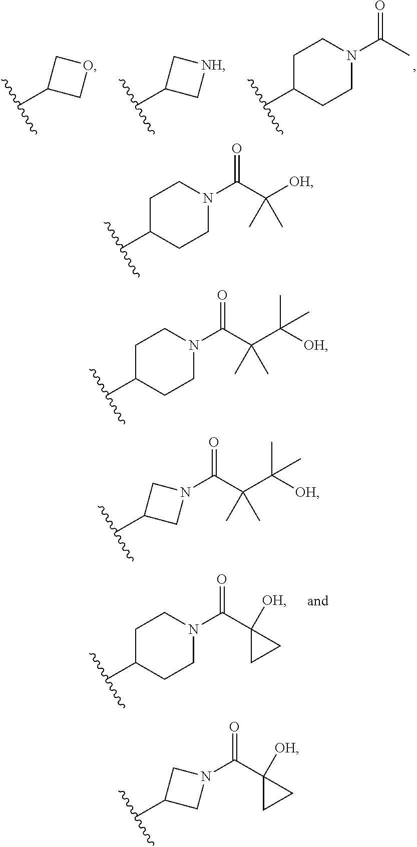

or a pharmaceutically acceptable salt or solvate thereof.

In another embodiment, Compounds of the Disclosure are compounds of Formulae I-III, V, or VI, wherein R^{1a} and R^{1b} are independently selected from the group consisting of hydrogen and fluoro, or a pharmaceutically acceptable salt or solvate thereof. In another embodiment, R^{1a} is fluoro and R^{1b} is hydrogen. In another embodiment, R^{1a} and R^{1b} are fluoro.

In another embodiment, Compounds of the Disclosure are compounds of Formula I or Formula II, wherein R^{1c} is hydrogen, or a pharmaceutically acceptable salt or solvate thereof.

In another embodiment, Compounds of the Disclosure are compounds of Formulae I, wherein Q is selected from the group consisting of —OR and —OC(═O)R, or a pharmaceutically acceptable salt or solvate thereof.

In another embodiment, Compounds of the Disclosure are compounds of Formulae I, wherein Q is —N(H)C(═O)R, or a pharmaceutically acceptable salt or solvate thereof.

In another embodiment, Compounds of the Disclosure are compounds of Formulae I, wherein Q is —N(H)C(═O)OR, or a pharmaceutically acceptable salt or solvate thereof.

In another embodiment, Compounds of the Disclosure are compounds of Formulae I, wherein Q is selected from the group consisting of —N(H)C(═O)OR, —N(H)C(═O)R, N(R)C(O)NR₂,

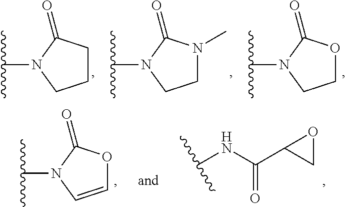

pharmaceutically acceptable salt or solvate thereof.

In another embodiment, Compounds of the Disclosure are compounds of Formulae I, wherein Q is selected from the group consisting of —N(R)C(═O)OR, —N(H)C(═O)R, —N(H)C(O)NR₂,

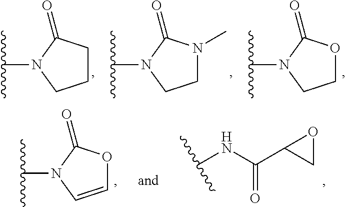

—OR, and —OC(═O)R, or a pharmaceutically acceptable salt or solvate thereof.

In another embodiment, Compounds of the Disclosure are compounds of Formulae I-III and V-VI, wherein G is selected from the group consisting of G-2, G-3, G-5, G-6, G-7, G-8, G-9, and G-10, or a pharmaceutically acceptable salt or solvate thereof.

In another embodiment, Compounds of the Disclosure are compounds of Formulae I-III and V-VI, wherein G is selected from the group consisting of G-11 and G-12, or a pharmaceutically acceptable salt or solvate thereof.

In another embodiment, Compounds of the Disclosure are compounds of Formulae I-III and V-VI, wherein G is selected from the group consisting of G-1 and G-4, or a pharmaceutically acceptable salt or solvate thereof.

In another embodiment, Compounds of the Disclosure are compounds of Formulae I-III and V-VI, wherein G is selected from the group consisting of G-13, G-14, G-15, G-16, G-17, G-18, G-19, G-20, G-21, G-22, G-23, G-24, G-25, and G-26, or a pharmaceutically acceptable salt or solvate thereof.

In another embodiment, Compounds of the Disclosure are compounds of Formulae I-III and V-VI, wherein G is selected from the group consisting of G-2, G-3, G-4, G-5, G-6, G-7, G-8, G-10, G-11, G-12, G-13, G-14, G-15, G-16, G-17, G-18, G-19, G-20, G-21, G-22, G-23, G-24, G-25, and G-26, or a pharmaceutically acceptable salt or solvate thereof.

In another embodiment, Compounds of the Disclosure are compounds of Formulae I-III and V-VI, wherein G is G-4, or a pharmaceutically acceptable salt or solvate thereof.

In another embodiment, Compounds of the Disclosure are compounds of Formulae I-III and V-VI, wherein G is selected from the group consisting of G-2, G-3, G-5, G-6, G-7, G-8, and G-10, or a pharmaceutically acceptable salt or solvate thereof.

In another embodiment, Compounds of the Disclosure are compounds of Formulae I-III and V-VI, wherein G is selected from the group consisting of selected from the group consisting of G-2, G-3, G-4, G-5, G-6, G-7, G-8, and G-10, or a pharmaceutically acceptable salt or solvate thereof.

In another embodiment, Compounds of the Disclosure are compounds of Formulae I-III and V-VI, wherein G is selected from the group consisting of G-4, G-11, and G-12, or a pharmaceutically acceptable salt or solvate thereof.

In another embodiment, Compounds of the Disclosure are compounds of Formulae I-II, wherein E is selected from the group consisting of E-1 and E-2, or a pharmaceutically acceptable salt or solvate thereof.

In another embodiment, Compounds of the Disclosure are compounds of Formulae I-II, wherein E is E-3, or a pharmaceutically acceptable salt or solvate thereof.

In another embodiment, Compounds of the Disclosure are compounds of Formulae I-II, wherein R^{4a} and R^{4b} are independently selected from the group consisting of halo and C₁-C₄ alkyl, or a pharmaceutically acceptable salt or solvate thereof.

In another embodiment, Compounds of the Disclosure are compounds of Formulae I-II, wherein R^{4a} and R^{4b} are each hydrogen, or a pharmaceutically acceptable salt or solvate thereof.

In another embodiment, Compounds of the Disclosure are compounds of Formulae I-II, wherein B is selected from the group consisting of C₁-C₆ alkyl, aralkyl, —C(═O)R⁹, —(CR^{5c}R^{5d})ₘOR¹⁰ and B-1, or a pharmaceutically acceptable salt or solvate thereof.

In another embodiment, Compounds of the Disclosure are compounds of Formulae I-II, wherein B is selected from the group consisting of B-3, B-4, B-5, B-6, B-7, and B-8, or a pharmaceutically acceptable salt or solvate thereof.

In another embodiment, Compounds of the Disclosure are compounds of Formulae I-II, wherein B is B-2, or a pharmaceutically acceptable salt or solvate thereof.

In another embodiment, Compounds of the Disclosure are compounds of Formulae I-II, wherein R^{13a} and R^{13b} are independently selected from the group consisting of halo, C₁-C₄ alkyl, and R^{a5}, or a pharmaceutically acceptable salt or solvate thereof.

In another embodiment, Compounds of the Disclosure are compounds of Formulae I-II, wherein B is selected from the group consisting of B-9, B-10, B-11, B-12, B-13, and B-14, or a pharmaceutically acceptable salt or solvate thereof.

In another embodiment, Compounds of the Disclosure are compounds of Formulae I-II, wherein B is selected from the group consisting of C₁-C₆ alkyl, —C(═O)R⁹, —(CR^{5c}R^{5d})ₘOR¹⁰, B-1, B-3, B-4, B-5, B-6, B-7, B-8, B-9, B-10, B-11, B-12, B-13, and B-14, or a pharmaceutically acceptable salt or solvate thereof.

In another embodiment, Compounds of the Disclosure are compounds of Formulae I-II, wherein B is selected from the group consisting of C₁-C₆ alkyl, —C(═O)R⁹, —(CR^{5c}R^{5d})ₘOR¹⁰, B-1, B-2, B-3, B-4, B-5, B-6, B-7, B-8, B-9, B-10, B-11, B-12, B-13, and B-14, or a pharmaceutically acceptable salt or solvate thereof.

In another embodiment, Compounds of the Disclosure are compounds of Formulae I-VI, wherein R^{a5} is carboxamido, or a pharmaceutically acceptable salt or solvate thereof.

In another embodiment, Compounds of the Disclosure are compounds of Formulae I-VI, wherein R^{a6} is selected from the group consisting of C₁-C₄ alkoxy and C₁-C₄ hydroxyalkyl, or a pharmaceutically acceptable salt or solvate thereof.

In another embodiment, Compounds of the Disclosure are compounds of Formulae I-V, wherein X is selected from the group consisting of —S(═O)₂— and —C(═O)—; and A is R^{a11}, or a pharmaceutically acceptable salt or solvate thereof.

In another embodiment, Compounds of the Disclosure are compounds of Formulae I-V, wherein:

(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b}, and R¹⁶;

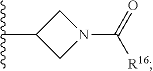

or

(2) X is absent; and A is selected from the group consisting of cyano and —C(═O)OH;

or a pharmaceutically acceptable salt or solvate thereof.

In another embodiment, Compounds of the Disclosure are compounds of Formulae I-V, wherein:

(1) X is —C(═O)—; and A is selected from the group consisting of optionally substituted C₁-C₆ alkyl, optionally substituted C₃-C₁₂ cycloalkyl, optionally substituted 4-to 14-membered heterocyclo, optionally substituted C₆-C₁₀ aryl, optionally substituted 5-to 14-membered heteroaryl, —NR^{15a}R^{15b}, R¹⁶,

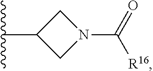

### and R^{a11}; or

(2) X is absent; and A is selected from the group consisting of C(═O)OH, C₁-C₄ alkyl, C₁-C₄ alkoxy, and C₁-C₄ haloalkyl;

or a pharmaceutically acceptable salt or solvate thereof.

In another embodiment, Compounds of the Disclosure are compounds of Formulae I-V, wherein:

(1) X is selected from the group consisting of —S(═O)₂— and —C(═O)—; and A is selected from the group consisting of optionally substituted C₁-C₆ alkyl, 4-membered heterocyclo, optionally substituted 5- to 14-membered heterocyclo, optionally substituted C₆-C₁₀ aryl, optionally substituted 5- to 14-membered heteroaryl, —NR^{15a}R^{15b},

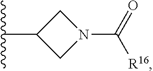

### and R^{a11}; or

(2) X is absent; and A is selected from the group consisting of cyano, C(═O)OH, C₁-C₄ alkyl, C₁-C₄ alkoxy, and C₁-C₄ haloalkyl;

or a pharmaceutically acceptable salt or solvate thereof.

In another embodiment, Compounds of the Disclosure are compounds of Formulae I-V, wherein X is absent; and A is selected from the group consisting of C₁-C₄ alkyl, C₁-C₄ alkoxy, and C₁-C₄ haloalkyl, or a pharmaceutically acceptable salt or solvate thereof.

In another embodiment, Compounds of the Disclosure are compounds of Formulae I-V, wherein X is absent; and A is C₁-C₄ haloalkyl, or a pharmaceutically acceptable salt or solvate thereof.

In another embodiment, Compounds of the Disclosure are any one or more of the compounds of Table 1.1, and the pharmaceutically acceptable salts and solvates thereof. Table 1.1A provides the chemical names of the compounds of Table 1.1 generated by Chemdraw® Professional version 17.0.0.206. In the event of any ambiguity between their chemical structure and chemical name, Compounds of the Disclosure are defined by them chemical structure.

TABLE 1.1

| | | MS |
|---|---|---|
| | | (ESI) |
| Cpd. | | m/z |
| No. | Structure | [M + H]⁺ |
| 1 | 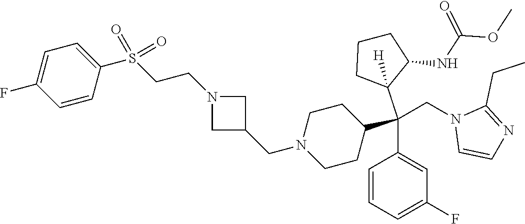 | |
| 2 | 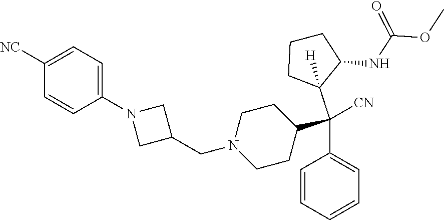 | |
| 3 | 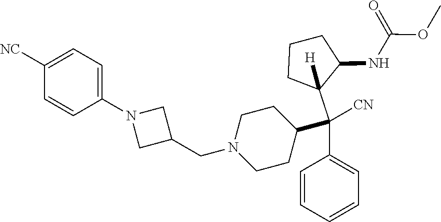 | |
| 4 | 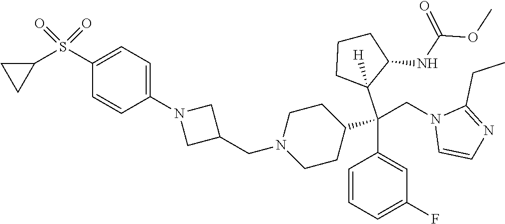 | |
| 5 | 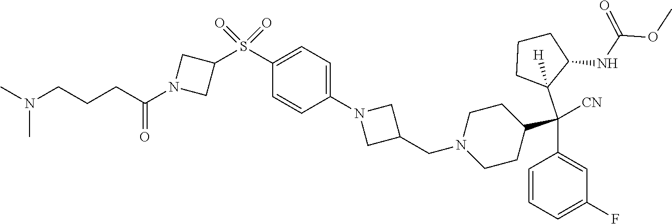 | |
| 6 | 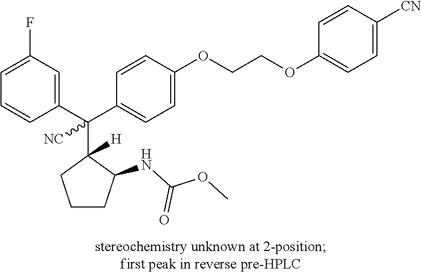 [SMILES: COC(=O)N[C@H]1CCC[C@@H]1C(C#N)(c1ccc(OCCOc2ccc(C#N)cc2)cc1)c1cccc(F)c1] | 514.26 |
| 7 | 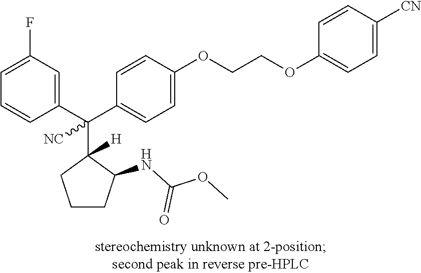 [SMILES: COC(=O)N[C@H]1CCC[C@@H]1C(C#N)(c1ccc(OCCOc2ccc(C#N)cc2)cc1)c1cccc(F)c1] | 514.30 |
| 10 | 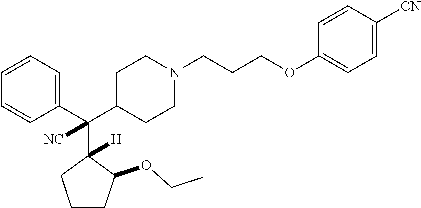 [SMILES: CCO[C@H]1CCC[C@@H]1[C@](C#N)(c1ccccc1)C1CCN(CCCOc2ccc(C#N)cc2)CC1] | |
| 11 | 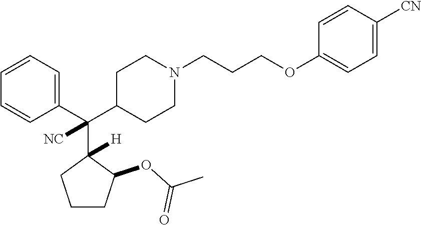 [SMILES: CC(=O)O[C@H]1CCC[C@@H]1[C@](C#N)(c1ccccc1)C1CCN(CCCOc2ccc(C#N)cc2)CC1] | |
| 12 | 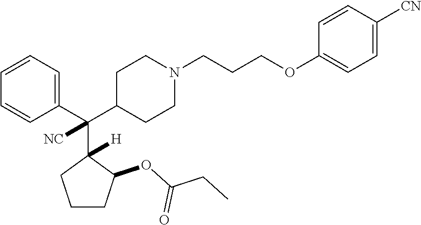 [SMILES: CCC(=O)O[C@H]1CCC[C@@H]1[C@](C#N)(c1ccccc1)C1CCN(CCCOc2ccc(C#N)cc2)CC1] | |
| 13 | 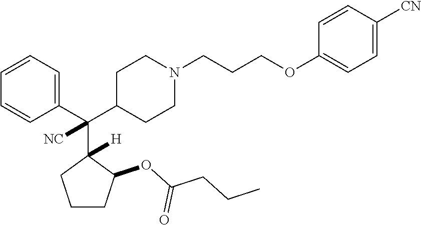 [SMILES: CCCC(=O)O[C@H]1CCC[C@@H]1[C@](C#N)(c1ccccc1)C1CCN(CCCOc2ccc(C#N)cc2)CC1] | |
| 14 | 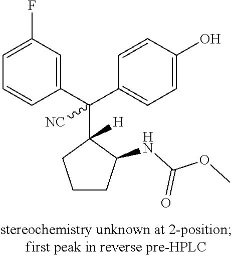 [SMILES: COC(=O)N[C@H]1CCC[C@@H]1C(C#N)(c1ccc(O)cc1)c1cccc(F)c1] | 369.20 |
| 15 | 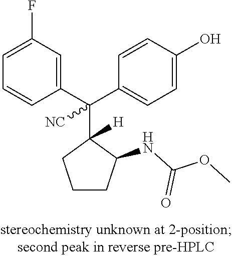 [SMILES: COC(=O)N[C@H]1CCC[C@@H]1C(C#N)(c1ccc(O)cc1)c1cccc(F)c1] | 369.20 |
| 16 | 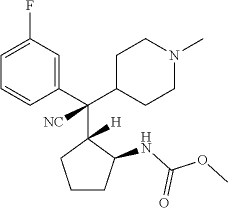 [SMILES: COC(=O)N[C@H]1CCC[C@@H]1[C@](C#N)(c1cccc(F)c1)C1CCN(C)CC1] | 374.44 |
| 17 | 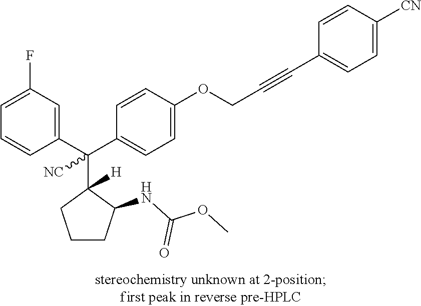 [SMILES: COC(=O)N[C@H]1CCC[C@@H]1C(C#N)(c1ccc(OCC#Cc2ccc(C#N)cc2)cc1)c1cccc(F)c1] | 508.27 |
| 18 | 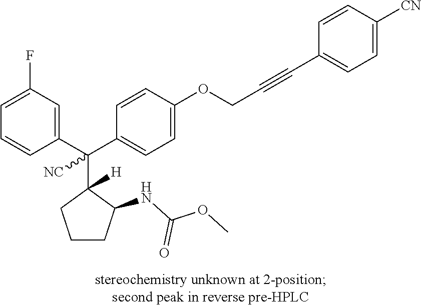 [SMILES: COC(=O)N[C@H]1CCC[C@@H]1C(C#N)(c1ccc(OCC#Cc2ccc(C#N)cc2)cc1)c1cccc(F)c1] | 508.30 |
| 19 | 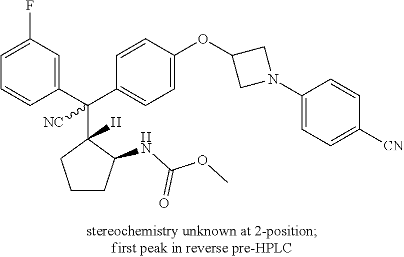 [SMILES: COC(=O)N[C@H]1CCC[C@@H]1C(C#N)(c1ccc(OC2CN(c3ccc(C#N)cc3)C2)cc1)c1cccc(F)c1] | 525.30 |
| 20 | 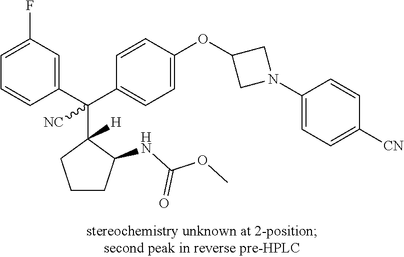 [SMILES: COC(=O)N[C@H]1CCC[C@@H]1C(C#N)(c1ccc(OC2CN(c3ccc(C#N)cc3)C2)cc1)c1cccc(F)c1] | 525.30 |
| 21 | 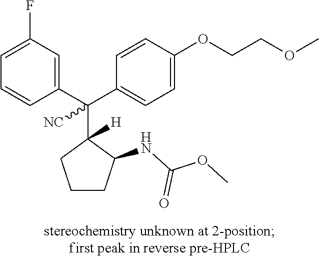 [SMILES: COCCOc1ccc(C(C#N)(c2cccc(F)c2)[C@H]2CCC[C@@H]2NC(=O)OC)cc1] | 427.28 |
| 22 | 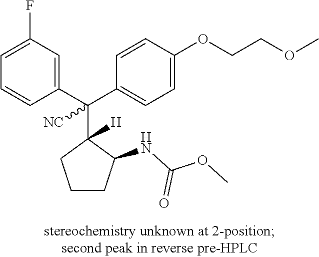 [SMILES: COCCOc1ccc(C(C#N)(c2cccc(F)c2)[C@H]2CCC[C@@H]2NC(=O)OC)cc1] | 427.40 |
| 23 | 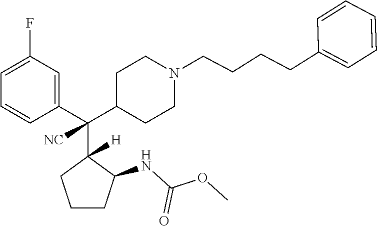 [SMILES: COC(=O)N[C@H]1CCC[C@@H]1[C@](C#N)(c1cccc(F)c1)C1CCN(CCCCc2ccccc2)CC1] | 492.43 |
| 24 | 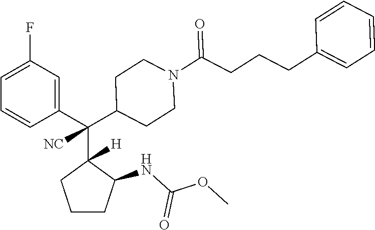 | 506.39 |
| 25 | 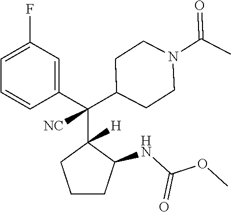 | 402.28 |
| 26 | 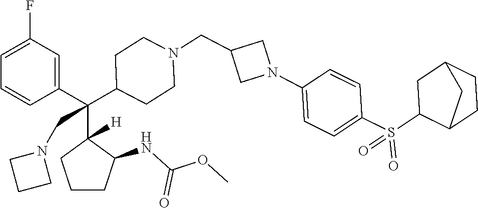 [SMILES: COC(=O)N[C@H]1CCC[C@@H]1[C@](CN1CCC1)(c1cccc(F)c1)C1CCN(CC2CN(c3ccc(S(=O)(=O)C4CC5CCC4C5)cc3)C2)CC1] | 707.59 |
| 27 | 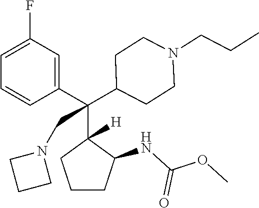 [SMILES: CCCN1CCC([C@@](CN2CCC2)(c2cccc(F)c2)[C@H]2CCC[C@@H]2NC(=O)OC)CC1] | 446.48 |
| 28 | 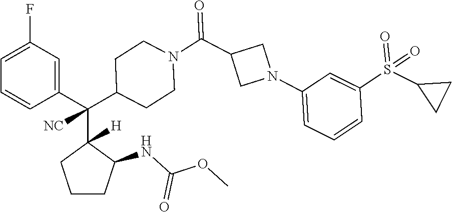 [SMILES: COC(=O)N[C@H]1CCC[C@@H]1[C@](C#N)(c1cccc(F)c1)C1CCN(C(=O)C2CN(c3cccc(S(=O)(=O)C4CC4)c3)C2)CC1] | 623.40 |
| 29 | 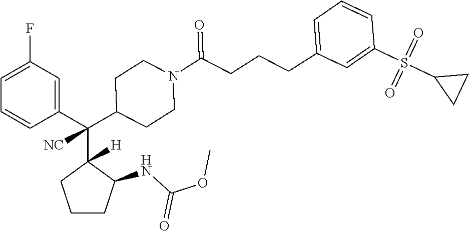 [SMILES: COC(=O)N[C@H]1CCC[C@@H]1[C@](C#N)(c1cccc(F)c1)C1CCN(C(=O)CCCc2cccc(S(=O)(=O)C3CC3)c2)CC1] | 610.37 |
| 30 | 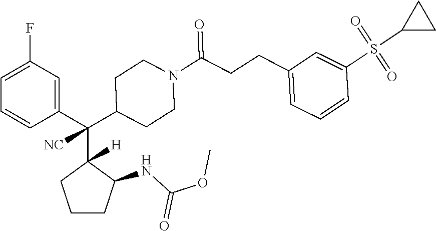 [SMILES: COC(=O)N[C@H]1CCC[C@@H]1[C@](C#N)(c1cccc(F)c1)C1CCN(C(=O)CCc2cccc(S(=O)(=O)C3CC3)c2)CC1] | 596.26 |
| 31 | 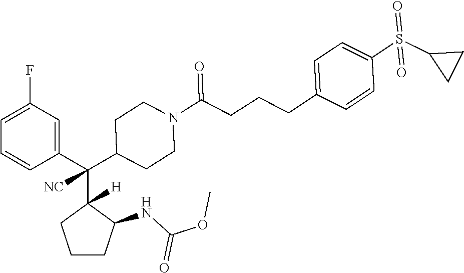 [SMILES: COC(=O)N[C@H]1CCC[C@@H]1[C@](C#N)(c1cccc(F)c1)C1CCN(C(=O)CCCc2ccc(S(=O)(=O)C3CC3)cc2)CC1] | 610.41 |
| 32 | 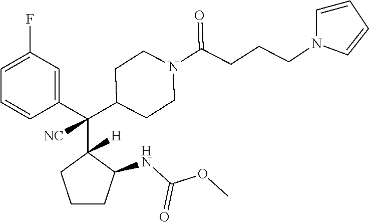 [SMILES: COC(=O)N[C@H]1CCC[C@@H]1[C@](C#N)(c1cccc(F)c1)C1CCN(C(=O)CCCn2cccc2)CC1] | 495.30 |
| 33 | 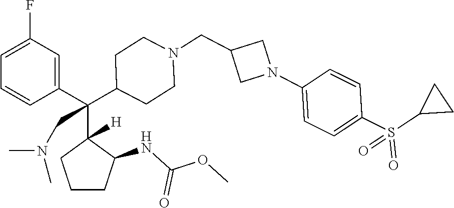 [SMILES: COC(=O)N[C@H]1CCC[C@@H]1[C@](CN(C)C)(c1cccc(F)c1)C1CCN(CC2CN(c3ccc(S(=O)(=O)C4CC4)cc3)C2)CC1] | 641.32 |
| 34 | 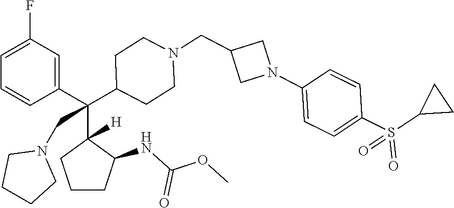 [SMILES: COC(=O)N[C@H]1CCC[C@@H]1[C@](CN1CCCC1)(c1cccc(F)c1)C1CCN(CC2CN(c3ccc(S(=O)(=O)C4CC4)cc3)C2)CC1] | 667.48 |
| 35 | 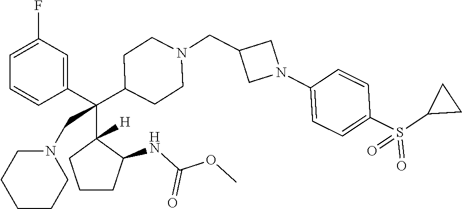 [SMILES: COC(=O)N[C@H]1CCC[C@@H]1[C@](CN1CCCCC1)(c1cccc(F)c1)C1CCN(CC2CN(c3ccc(S(=O)(=O)C4CC4)cc3)C2)CC1] | 681.54 |
| 36 | 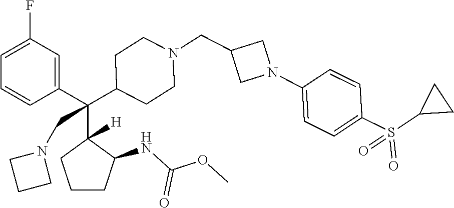 [SMILES: COC(=O)N[C@H]1CCC[C@@H]1[C@](CN1CCC1)(c1cccc(F)c1)C1CCN(CC2CN(c3ccc(S(=O)(=O)C4CC4)cc3)C2)CC1] | 653.48 |
| 37 | 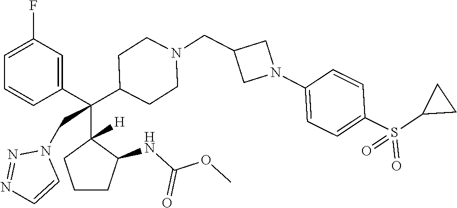 [SMILES: COC(=O)N[C@H]1CCC[C@@H]1[C@](Cn1ccnn1)(c1cccc(F)c1)C1CCN(CC2CN(c3ccc(S(=O)(=O)C4CC4)cc3)C2)CC1] | 665.47 |
| 38 | 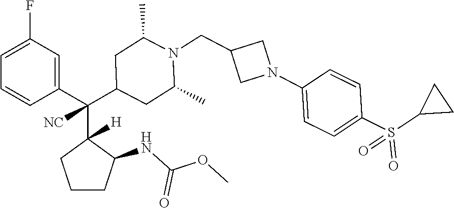 [SMILES: COC(=O)N[C@H]1CCC[C@@H]1[C@](C#N)(c1cccc(F)c1)C1C[C@@H](C)N(CC2CN(c3ccc(S(=O)(=O)C4CC4)cc3)C2)[C@@H](C)C1] | 637.49 |
| 39 | 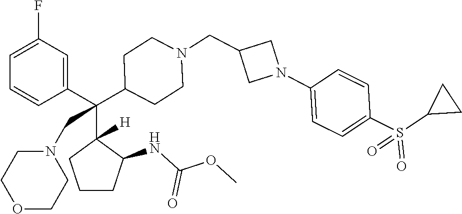 [SMILES: COC(=O)N[C@H]1CCC[C@@H]1[C@](CN1CCOCC1)(c1cccc(F)c1)C1CCN(CC2CN(c3ccc(S(=O)(=O)C4CC4)cc3)C2)CC1] | 683.48 |
| 40 | 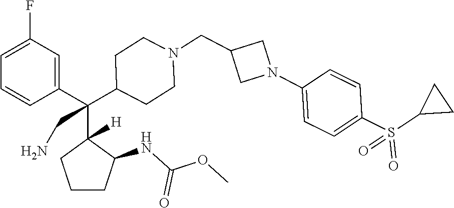 [SMILES: COC(=O)N[C@H]1CCC[C@@H]1[C@](CN)(c1cccc(F)c1)C1CCN(CC2CN(c3ccc(S(=O)(=O)C4CC4)cc3)C2)CC1] | 613.42 |
| 41 | 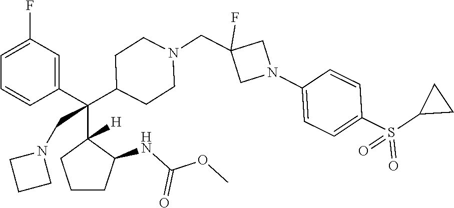 [SMILES: COC(=O)N[C@H]1CCC[C@@H]1[C@](CN1CCC1)(c1cccc(F)c1)C1CCN(CC2(F)CN(c3ccc(S(=O)(=O)C4CC4)cc3)C2)CC1] | 671.57 |
| 42 | 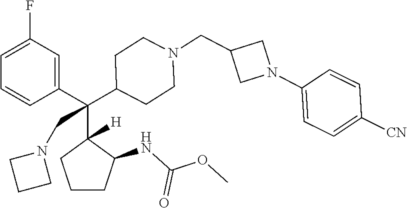 [SMILES: COC(=O)N[C@H]1CCC[C@@H]1[C@](CN1CCC1)(c1cccc(F)c1)C1CCN(CC2CN(c3ccc(C#N)cc3)C2)CC1] | 574.47 |
| 43 | 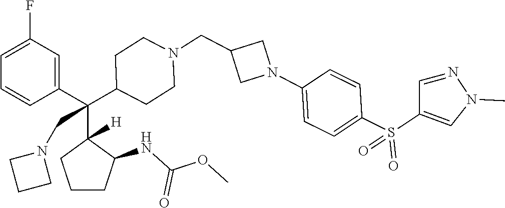 [SMILES: COC(=O)N[C@H]1CCC[C@@H]1[C@](CN1CCC1)(c1cccc(F)c1)C1CCN(CC2CN(c3ccc(S(=O)(=O)c4cnn(C)c4)cc3)C2)CC1] | 693.54 |
| 44 | 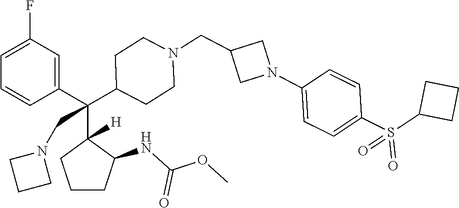 [SMILES: COC(=O)N[C@H]1CCC[C@@H]1[C@](CN1CCC1)(c1cccc(F)c1)C1CCN(CC2CN(c3ccc(S(=O)(=O)C4CCC4)cc3)C2)CC1] | 667.55 |
| 45 | 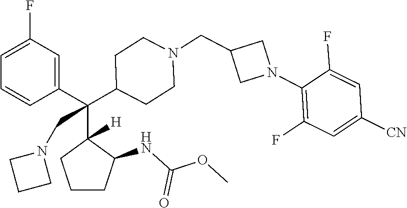 [SMILES: COC(=O)N[C@H]1CCC[C@@H]1[C@](CN1CCC1)(c1cccc(F)c1)C1CCN(CC2CN(c3c(F)cc(C#N)cc3F)C2)CC1] | 610.51 |
| 46 | 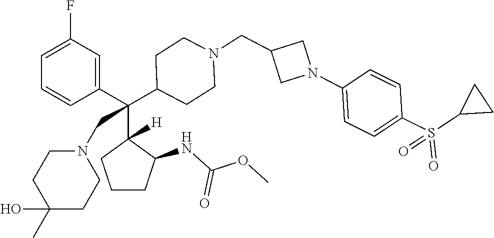 [SMILES: COC(=O)N[C@H]1CCC[C@@H]1[C@](CN1CCC(C)(O)CC1)(c1cccc(F)c1)C1CCN(CC2CN(c3ccc(S(=O)(=O)C4CC4)cc3)C2)CC1] | 711.55 |
| 47 | 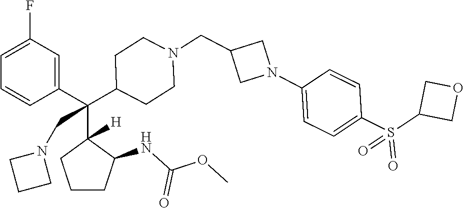 [SMILES: COC(=O)N[C@H]1CCC[C@@H]1[C@](CN1CCC1)(c1cccc(F)c1)C1CCN(CC2CN(c3ccc(S(=O)(=O)C4COC4)cc3)C2)CC1] | 669.51 |
| 48 | 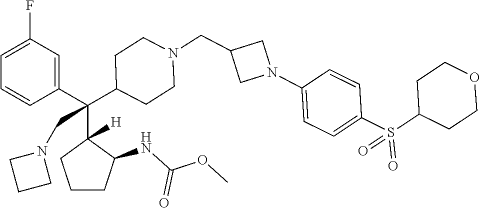 [SMILES: COC(=O)N[C@H]1CCC[C@@H]1[C@](CN1CCC1)(c1cccc(F)c1)C1CCN(CC2CN(c3ccc(S(=O)(=O)C4CCOCC4)cc3)C2)CC1] | 697.58 |
| 49 | 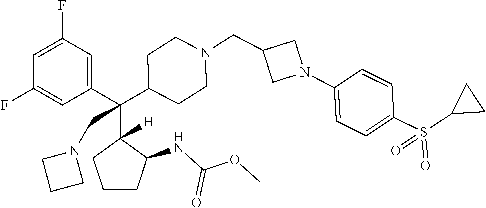 [SMILES: COC(=O)N[C@H]1CCC[C@@H]1[C@](CN1CCC1)(c1cc(F)cc(F)c1)C1CCN(CC2CN(c3ccc(S(=O)(=O)C4CC4)cc3)C2)CC1] | 671.49 |
| 50 | 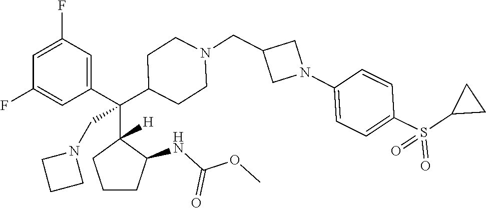 [SMILES: COC(=O)N[C@H]1CCC[C@@H]1[C@@](CN1CCC1)(c1cc(F)cc(F)c1)C1CCN(CC2CN(c3ccc(S(=O)(=O)C4CC4)cc3)C2)CC1] | 671.51 |
| 51 | 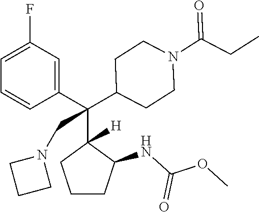 [SMILES: CCC(=O)N1CCC([C@@](CN2CCC2)(c2cccc(F)c2)[C@H]2CCC[C@@H]2NC(=O)OC)CC1] | 460.48 |
| 52 | 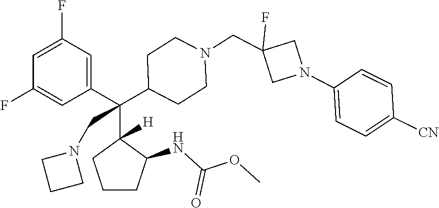 [SMILES: COC(=O)N[C@H]1CCC[C@@H]1[C@](CN1CCC1)(c1cc(F)cc(F)c1)C1CCN(CC2(F)CN(c3ccc(C#N)cc3)C2)CC1] | 610.48 |
| 53 | 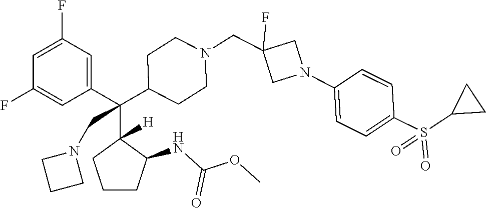 [SMILES: COC(=O)N[C@H]1CCC[C@@H]1[C@](CN1CCC1)(c1cc(F)cc(F)c1)C1CCN(CC2(F)CN(c3ccc(S(=O)(=O)C4CC4)cc3)C2)CC1] | 689.48 |
| 54 | 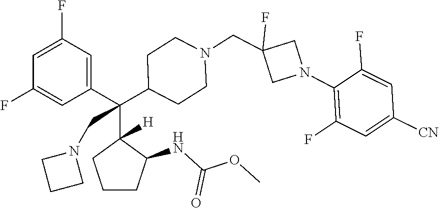 [SMILES: COC(=O)N[C@H]1CCC[C@@H]1[C@](CN1CCC1)(c1cc(F)cc(F)c1)C1CCN(CC2(F)CN(c3c(F)cc(C#N)cc3F)C2)CC1] | 646.47 |
| 55 | 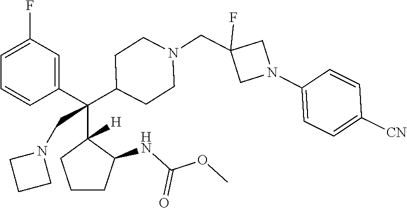 [SMILES: COC(=O)N[C@H]1CCC[C@@H]1[C@](CN1CCC1)(c1cccc(F)c1)C1CCN(CC2(F)CN(c3ccc(C#N)cc3)C2)CC1] | 592.49 |
| 56 | 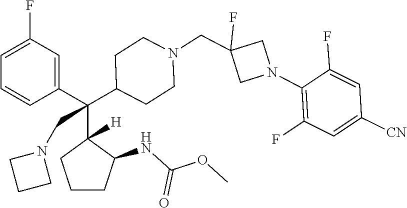 [SMILES: COC(=O)N[C@H]1CCC[C@@H]1[C@](CN1CCC1)(c1cccc(F)c1)C1CCN(CC2(F)CN(c3c(F)cc(C#N)cc3F)C2)CC1] | 628.44 |
| 57 | 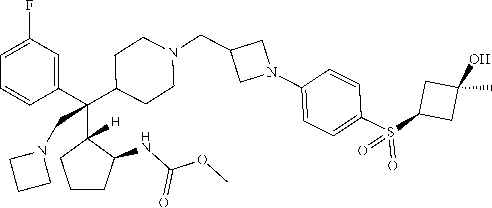 [SMILES: COC(=O)N[C@H]1CCC[C@@H]1[C@](CN1CCC1)(c1cccc(F)c1)C1CCN(CC2CN(c3ccc(S(=O)(=O)[C@H]4C[C@@](C)(O)C4)cc3)C2)CC1] | 697.54 |
| 58 | 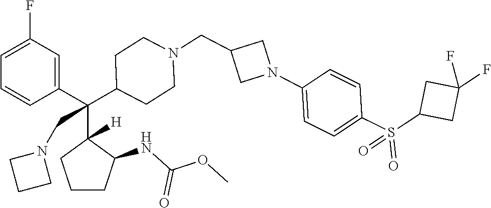 [SMILES: COC(=O)N[C@H]1CCC[C@@H]1[C@](CN1CCC1)(c1cccc(F)c1)C1CCN(CC2CN(c3ccc(S(=O)(=O)C4CC(F)(F)C4)cc3)C2)CC1] | 703.52 |
| 59 | 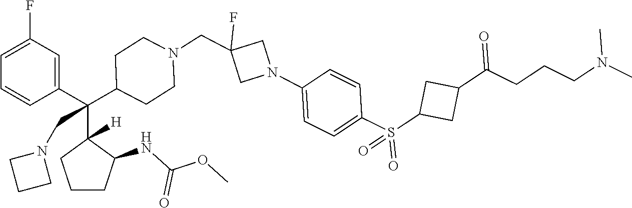 [SMILES: COC(=O)N[C@H]1CCC[C@@H]1[C@](CN1CCC1)(c1cccc(F)c1)C1CCN(CC2(F)CN(c3ccc(S(=O)(=O)C4CC(C(=O)CCCN(C)C)C4)cc3)C2)CC1] | 799.55 |
| 60 | 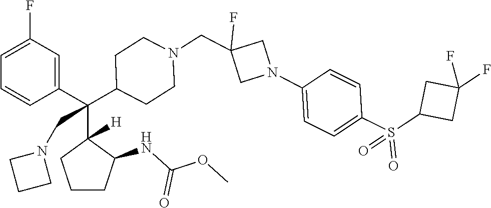 [SMILES: COC(=O)N[C@H]1CCC[C@@H]1[C@](CN1CCC1)(c1cccc(F)c1)C1CCN(CC2(F)CN(c3ccc(S(=O)(=O)C4CC(F)(F)C4)cc3)C2)CC1] | 721.54 |
| 61 | 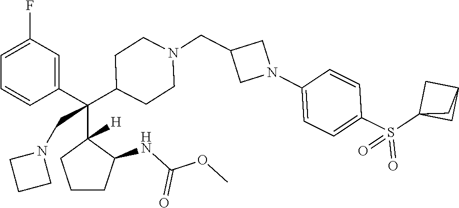 [SMILES: COC(=O)N[C@H]1CCC[C@@H]1[C@](CN1CCC1)(c1cccc(F)c1)C1CCN(CC2CN(c3ccc(S(=O)(=O)C45CC(C4)C5)cc3)C2)CC1] | 679.21 |
| 62 | 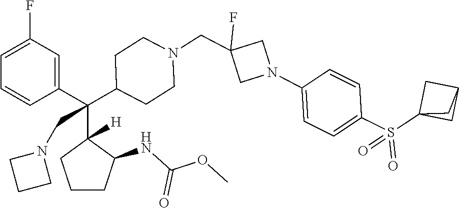 [SMILES: COC(=O)N[C@H]1CCC[C@@H]1[C@](CN1CCC1)(c1cccc(F)c1)C1CCN(CC2(F)CN(c3ccc(S(=O)(=O)C45CC(C4)C5)cc3)C2)CC1] | 697.58 |
| 63 | 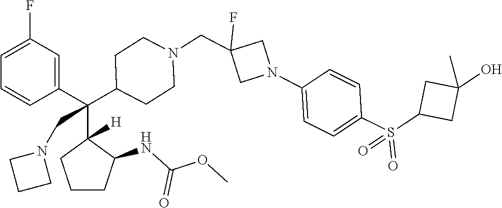 [SMILES: COC(=O)N[C@H]1CCC[C@@H]1[C@](CN1CCC1)(c1cccc(F)c1)C1CCN(CC2(F)CN(c3ccc(S(=O)(=O)C4CC(C)(O)C4)cc3)C2)CC1] | 715.61 |
| 64 | 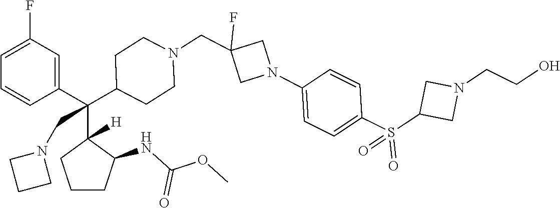 [SMILES: COC(=O)N[C@H]1CCC[C@@H]1[C@](CN1CCC1)(c1cccc(F)c1)C1CCN(CC2(F)CN(c3ccc(S(=O)(=O)C4CN(CCO)C4)cc3)C2)CC1] | 730.62 |
| 65 | 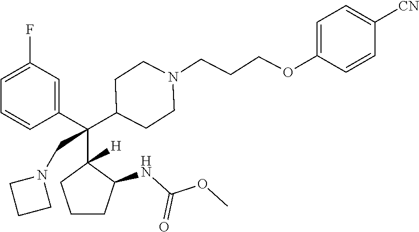 [SMILES: COC(=O)N[C@H]1CCC[C@@H]1[C@](CN1CCC1)(c1cccc(F)c1)C1CCN(CCCOc2ccc(C#N)cc2)CC1] | 563.50 |
| 66 | 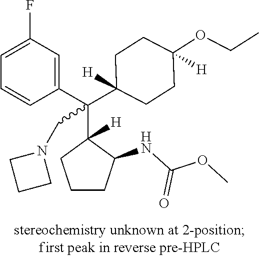 [SMILES: CCO[C@H]1CC[C@H](C(CN2CCC2)(c2cccc(F)c2)[C@H]2CCC[C@@H]2NC(=O)OC)CC1] | 447.54 |
| 67 | 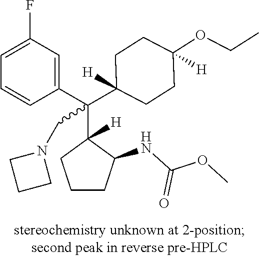 [SMILES: CCO[C@H]1CC[C@H](C(CN2CCC2)(c2cccc(F)c2)[C@H]2CCC[C@@H]2NC(=O)OC)CC1] | 447.55 |
| 68 | 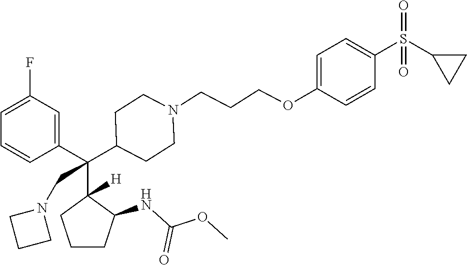 [SMILES: COC(=O)N[C@H]1CCC[C@@H]1[C@](CN1CCC1)(c1cccc(F)c1)C1CCN(CCCOc2ccc(S(=O)(=O)C3CC3)cc2)CC1] | 642.58 |
| 69 | 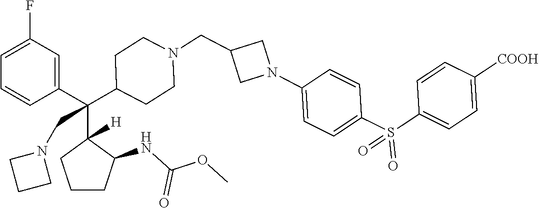 [SMILES: COC(=O)N[C@H]1CCC[C@@H]1[C@](CN1CCC1)(c1cccc(F)c1)C1CCN(CC2CN(c3ccc(S(=O)(=O)c4ccc(C(=O)O)cc4)cc3)C2)CC1] | 733.55 |
| 70 | 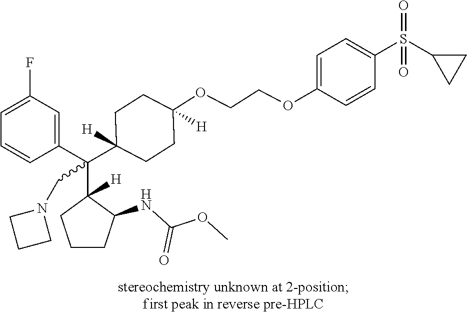 [SMILES: COC(=O)N[C@H]1CCC[C@@H]1C(CN1CCC1)(c1cccc(F)c1)[C@H]1CC[C@H](OCCOc2ccc(S(=O)(=O)C3CC3)cc2)CC1] | 643.10 |
| 71 | 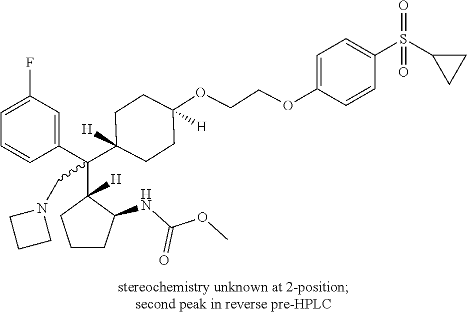 [SMILES: COC(=O)N[C@H]1CCC[C@@H]1C(CN1CCC1)(c1cccc(F)c1)[C@H]1CC[C@H](OCCOc2ccc(S(=O)(=O)C3CC3)cc2)CC1] | 643.12 |
| 72 | 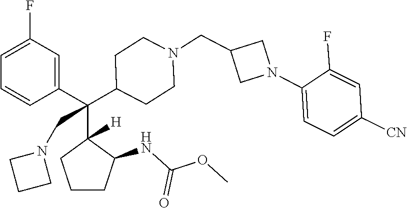 [SMILES: COC(=O)N[C@H]1CCC[C@@H]1[C@](CN1CCC1)(c1cccc(F)c1)C1CCN(CC2CN(c3ccc(C#N)cc3F)C2)CC1] | 592.13 |
| 73 | 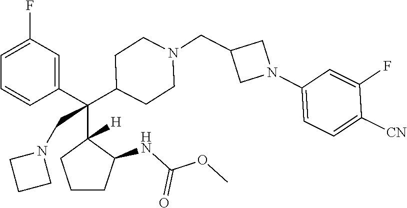 [SMILES: COC(=O)N[C@H]1CCC[C@@H]1[C@](CN1CCC1)(c1cccc(F)c1)C1CCN(CC2CN(c3ccc(C#N)c(F)c3)C2)CC1] | 592.11 |
| 74 | 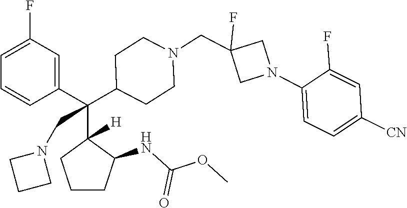 [SMILES: COC(=O)N[C@H]1CCC[C@@H]1[C@](CN1CCC1)(c1cccc(F)c1)C1CCN(CC2(F)CN(c3ccc(C#N)cc3F)C2)CC1] | 610.12 |
| 75 | 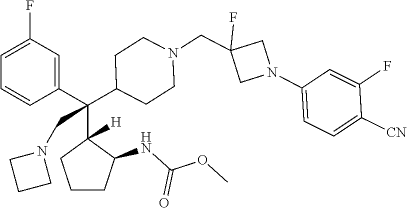 [SMILES: COC(=O)N[C@H]1CCC[C@@H]1[C@](CN1CCC1)(c1cccc(F)c1)C1CCN(CC2(F)CN(c3ccc(C#N)c(F)c3)C2)CC1] | 610.53 |
| 76 | 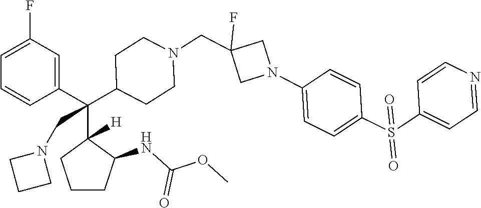 [SMILES: COC(=O)N[C@H]1CCC[C@@H]1[C@](CN1CCC1)(c1cccc(F)c1)C1CCN(CC2(F)CN(c3ccc(S(=O)(=O)c4ccncc4)cc3)C2)CC1] | 708.04 |
| 77 | 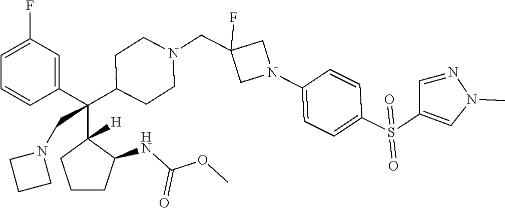 [SMILES: COC(=O)N[C@H]1CCC[C@@H]1[C@](CN1CCC1)(c1cccc(F)c1)C1CCN(CC2(F)CN(c3ccc(S(=O)(=O)c4cnn(C)c4)cc3)C2)CC1] | 711.04 |
| 78 | 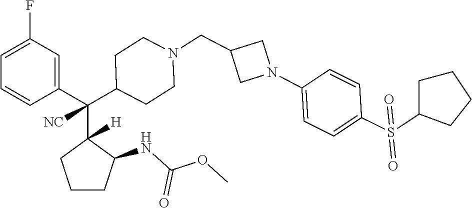 [SMILES: COC(=O)N[C@H]1CCC[C@@H]1[C@](C#N)(c1cccc(F)c1)C1CCN(CC2CN(c3ccc(S(=O)(=O)C4CCCC4)cc3)C2)CC1] | 637.53 |
| 79 | 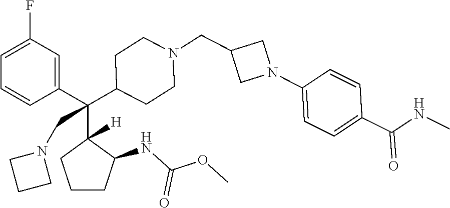 [SMILES: CNC(=O)c1ccc(N2CC(CN3CCC([C@@](CN4CCC4)(c4cccc(F)c4)[C@H]4CCC[C@@H]4NC(=O)OC)CC3)C2)cc1] | 606.14 |
| 80 | 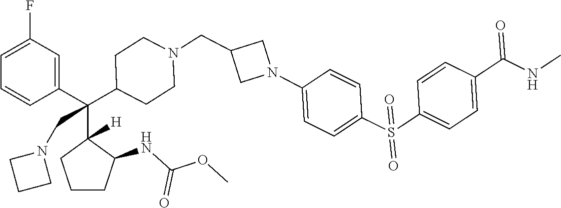 [SMILES: CNC(=O)c1ccc(S(=O)(=O)c2ccc(N3CC(CN4CCC([C@@](CN5CCC5)(c5cccc(F)c5)[C@H]5CCC[C@@H]5NC(=O)OC)CC4)C3)cc2)cc1] | 746.02 |
| 81 | 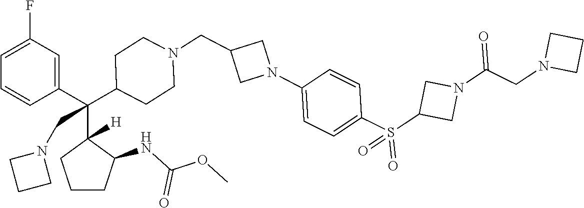 [SMILES: COC(=O)N[C@H]1CCC[C@@H]1[C@](CN1CCC1)(c1cccc(F)c1)C1CCN(CC2CN(c3ccc(S(=O)(=O)C4CN(C(=O)CN5CCC5)C4)cc3)C2)CC1] | 765.04 |
| 82 | 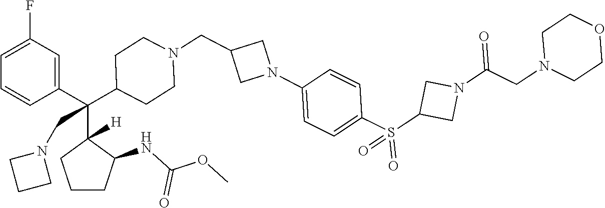 [SMILES: COC(=O)N[C@H]1CCC[C@@H]1[C@](CN1CCC1)(c1cccc(F)c1)C1CCN(CC2CN(c3ccc(S(=O)(=O)C4CN(C(=O)CN5CCOCC5)C4)cc3)C2)CC1] | 795.50 |
| 83 | 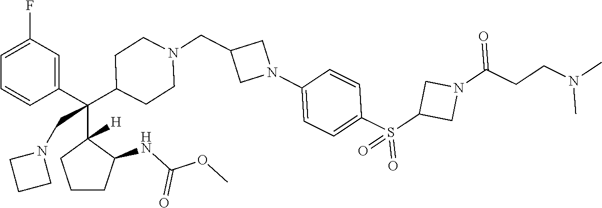 [SMILES: COC(=O)N[C@H]1CCC[C@@H]1[C@](CN1CCC1)(c1cccc(F)c1)C1CCN(CC2CN(c3ccc(S(=O)(=O)C4CN(C(=O)CCN(C)C)C4)cc3)C2)CC1] | 767.07 |

TABLE 1.1A

| Cpd. No. | Chemical Name |
|---|---|
| 1 | methyl ((1S,2R)-2-((S)-2-(2-ethyl-1H-imidazol-1-yl)-1-(3- |
| | fluorophenyl)-1-(1((1-(2-((4-fluorophenyl)sulfonyl)ethyl)azetidin- |
| | 3-yl)methyl)piperidin-4-yl)ethyl)cyclopentyl)carbamate |
| 2 | methyl ((1S,2R)-2-((S)-cyano(1-((1-(4-cyanophenyl)azetidin-3- |
| | yl)methyl)piperidin-4-yl)(phenyl)methyl)cyclopentyl)carbamate |
| 3 | methyl ((1R,2S)-2-((S)-cyano(1-((1-(4-cyanophenyl)azetidin-3- |
| | yl)methyl)piperidin-4-yl)(phenyl)methyl)cyclopentyl)carbamate |
| 4 | methyl ((1S,2R)-2-((R)-1-(1-((1-(4- |
| | (cyclopropyl sulfonyl)phenyl)azetidin-3-yl)methyl)piperidin-4-yl)- |
| | 2-(2-ethyl-1H-imidazol-1-yl)-1-(3- |
| | fluorophenyl)ethyl)cyclopentyl)carbamate |
| 5 | methyl ((1S,2R)-2-((S)-cyano(1-((1-(4-((1-(4- |
| | (dimethylamino)butanoyl)azetidin-3-yl)sulfonyl)phenyl)azetidin- |
| | 3-yl)methyl)piperidin-4-yl)(3- |
| | fluorophenyl)methyl)cyclopentyl)carbamate |
| 6 | methyl ((1S,2R)-2-(cyano(4-(2-(4-cyanophenoxy)ethoxy)phenyl)(3- |
| | fluorophenyl)methyl)cyclopentyl)carbamate |
| | (2-position stereochemistry unknown) |
| 7 | methyl ((1S,2R)-2-(cyano(4-(2-(4-cyanophenoxy)ethoxy)phenyl)(3- |
| | fluorophenyl)methyl)cyclopentyl)carbamate |
| | (2-position stereochemistry unknown) |
| 10 | 4-(3-(4-((S)-cyano((1R,2S)-2- |
| | ethoxycyclopentyl)(phenyl)methyl)piperidin-1-yl)propoxy)benzonitrile |
| 11 | (1S,2R)-2-((S)-cyano(1-(3-(4-cyanophenoxy)propyl)piperidin-4- |
| | yl)(phenyl)methyl)cyclopentyl acetate |
| 12 | (1S,2R)-2-((S)-cyano(1-(3-(4-cyanophenoxy)propyl)piperidin-4- |
| | yl)(phenyl)methyl)cyclopentyl propionate |
| 13 | (1S,2R)-2-((S)-cyano(1-(3-(4-cyanophenoxy)propyl)piperidin-4- |
| | yl)(phenyl)methyl)cyclopentyl butyrate |
| 14 | methyl ((1S,2R)-2-(cyano(3-fluorophenyl)(4- |
| | hydroxyphenyl)methyl)cyclopentyl)carbamate |
| | (2-position stereochemistry unknown) |
| 15 | methyl ((1S,2R)-2-(cyano(3-fluorophenyl)(4- |
| | hydroxyphenyl)methyl)cyclopentyl)carbamate |
| | (2-position stereochemistry unknown) |
| 16 | methyl ((1S,2R)-2((S)-cyano(3-fluorophenyl)(1-methylpiperidin-4- |
| | yl)methyl)cyclopentyl)carbamate |
| 17 | methyl ((1S,2R)-2-(cyano(4-((3-(4-cyanophenyl)prop-2-yn-1- |
| | yl)oxy)phenyl)(3-fluorophenyl)methyl)cyclopentyl)carbamate |
| | (2-position stereochemistry unknown) |
| 18 | methyl ((1S,2R)-2-(cyano(4-((3-(4-cyanophenyl)prop-2-yn-1- |
| | yl)oxy)phenyl)(3-fluorophenyl)methyl)cyclopentyl)carbamate |
| | (2-position stereochemistry unknown) |
| 19 | methyl ((1S,2R)-2-(cyano(4-((1-(4-cyanophenyl)azetidin-3- |
| | yl)oxy)phenyl)(3-fluorophenyl)methyl)cyclopentyl)carbamate |
| | (2-position stereochemistry unknown) |
| 20 | methyl ((1S,2R)-2-(cyano(4-((1-(4-cyanophenyl)azetidin-3- |
| | yl)oxy)phenyl)(3-fluorophenyl)methyl)cyclopentyl)carbamate |
| | (2-position stereochemistry unknown) |
| 21 | methyl ((1S,2R)-2-(cyano(3-fluorophenyl)(4-(2- |
| | methoxyethoxy)phenyl)methyl)cyclopentyl)carbamate |
| | (2-position stereochemistry unknown) |
| 22 | methyl ((1S,2R)-2-(cyano(3-fluorophenyl)(4-(2- |
| | methoxyethoxy)phenyl)methyl)cyclopentyl)carbamate |
| | (2-position stereochemistry unknown) |
| 23 | methyl ((1S,2R)-2-((S)-cyano(3-fluorophenyl)(1-(4- |
| | phenylbutyl)piperidin-4-yl)methyl)cyclopentyl)carbamate |
| 24 | methyl ((1S,2R)-2-((S)-cyano(3-fluorophenyl)(1-(4- |
| | phenylbutanoyl)piperidin-4-yl)methyl)cyclopentyl)carbamate |
| 25 | methyl ((1S,2R)-2-((S)-(1-acetylpiperidin-4-yl)(cyano)(3- |
| | fluorophenyl)methyl)cyclopentyl)carbamate |
| 26 | methyl ((1S,2R)-2-((1S)-2-(azetidin-1-yl)-1-(1-((1-(4- |
| | (bicyclo [ 2.2.1 ] heptan-2-ylsulfonyl)phenyl)azetidin-3- |
| | yl)methyl)piperidin-4-yl)-1-(3-fluorophenyl)ethyl)cyclopentyl)carbamate |
| 27 | methyl ((1S,2R)-2-((S)-2-(azetidin-1-yl)-1-(3-fluorophenyl)-1-(1- |
| | propylpiperidin-4-yl)ethyl)cyclopentyl)carbamate |
| 28 | methyl ((1S,2R)-2-((S)-cyano(1-(1-(3- |
| | (cyclopropylsulfonyl)phenyl)azetidine-3-carbonyl)piperidin-4-yl)(3- |
| | fluorophenyl)methyl)cyclopentyl)carbamate |
| 29 | methyl ((1S,2R)-2-((S)-cyano(1-(4-(3- |
| | (cyclopropylsulfonyl)phenyl)butanoyl)piperidin-4-yl)(3- |
| | fluorophenyl)methyl)cyclopentyl)carbamate |
| 30 | methyl ((1S,2R)-2-((S)-cyano(1-(3-(3- |
| | (cyclopropylsulfonyl)phenyl)propanoyl)piperidin-4-yl)(3- |
| | fluorophenyl)methyl)cyclopentyl)carbamate |
| 31 | methyl ((1S,2R)-2-((S)-cyano(1-(4-(4- |
| | (cyclopropylsulfonyl)phenyl)butanoyl)piperidin-4-yl)(3- |
| | fluorophenyl)methyl)cyclopentyl)carbamate |
| 32 | methyl ((1S,2R)-2-((S)-(1-(4-(1H-pyrrol-1-yl)butanoyl)piperidin-4- |
| | yl)(cyano)(3-fluorophenyl)methyl)cyclopentyl)carbamate |
| 33 | methyl ((1S,2R)-2-((S)-1-(1-((1-(4- |
| | (cyclopropylsulfonyl)phenyl)azetidin-3-yl)methyl)piperidin-4-yl)- |
| | 2-(dimethylamino)-1-(3-fluorophenyl)ethyl)cyclopentyl)carbamate |
| 34 | methyl ((1S,2R)-2-((S)-1-(1-((1-(4- |
| | (cyclopropylsulfonyl)phenyl)azetidin-3-yl)methyl)piperidin-4-yl)-1-(3- |
| | fluorophenyl)-2-(pyrrolidin-1-yl)ethyl)cyclopentyl)carbamate |
| 35 | methyl ((1S,2R)-2-((S)-1-(1-((1-(4- |
| | (cyclopropylsulfonyl)phenyl)azetidin-3-yl)methyl)piperidin-4-yl)-1-(3- |
| | fluorophenyl)-2-(piperidin-1-yl)ethyl)cyclopentyl)carbamate |
| 36 | methyl ((1S,2R)-2-((S)-2-(azetidin-1-yl)-1-(1-((1-(4- |
| | (cyclopropylsulfonyl)phenyl)azetidin-3-yl)methyl)piperidin-4-yl)-1-(3- |
| | fluorophenypethyl)cyclopentyl)carbamate |
| 37 | methyl ((1S,2R)-2-((S)-1-(1-((1-(4- |
| | (cyclopropylsulfonyl)phenyl)azetidin-3-yl)methyl)piperidin-4-yl)-1-(3- |
| | fluorophenyl)-2-(1H-1,2,3-triazol-1-yl)ethyl)cyclopentyl)carbamate |
| 38 | methyl ((1S,2R)-2-((1S)-cyano((2R,6S)-1-((1-(4- |
| | (cyclopropylsulfonyl)phenyl)azetidin-3-yl)methyl)-2,6- |
| | dimethylpiperidin-4-yl)(3-fluorophenyl)methyl)cyclopentyl)carbamate |
| 39 | methyl ((1S,2R)-2-((S)-1-(1-((1-(4- |
| | (cyclopropylsulfonyl)phenyl)azetidin-3-yl)methyl)piperidin-4-yl)-1-(3- |
| | fluorophenyl)-2-morpholinoethyl)cyclopentyl)carbamate |
| 40 | methyl ((1S,2R)-2-((S)-2-amino-1-(1-((1-(4- |
| | (cyclopropylsulfonyl)phenyl)azetidin-3-yl)methyl)piperidin-4-yl)-1-(3- |
| | fluorophenyl)ethyl)cyclopentyl)carbamate |
| 41 | methyl ((1S,2R)-2-((S)-2-(azetidin-1-yl)-1-(1-((1-(4- |
| | (cyclopropylsulfonyl)phenyl)-3-fluoroazetidin-3-yl)methyl)piperidin-4- |
| | yl)-1-(3-fluorophenyl)ethyl)cyclopentyl)carbamate |
| 42 | methyl ((1S,2R)-2-((S)-2-(azetidin-1-yl)-1-(1-((1-(4- |
| | cyanophenyl)azetidin-3-yl)methyl)piperidin-4-yl)-1-(3- |
| | fluorophenyl)ethyl)cyclopentyl)carbamate |
| 43 | methyl ((1S,2R)-2-((S)-2-(azetidin-1-yl)-1-(3-fluorophenyl)-1-(1-((1- |
| | (4-((1-methyl-1H-pyrazol-4-yl)sulfonyl)phenyl)azetidin-3- |
| | yl)methyl)piperidin-4-yl)ethyl)cyclopentyl)carbamate |
| 44 | methyl ((1S,2R)-2-((S)-2-(azetidin-1-yl)-1-(1-((1-(4- |
| | (cyclobutylsulfonyl)phenyl)azetidin-3-yl)methyl)piperidin-4-yl)-1-(3- |
| | fluorophenypethyl)cyclopentyl)carbamate |
| 45 | methyl ((1S,2R)-2-((S)-2-(azetidin-1-yl)-1-(1-((1-(4-cyano-2,6- |
| | difluorophenyl)azetidin-3-yl)methyl)piperidin-4-yl)-1-(3- |
| | fluorophenyl)ethyl)cyclopentyl)carbamate |
| 46 | methyl ((1S,2R)-2-((S)-1-(1-((1-(4- |
| | (cyclopropyl sulfonyl)phenyl)azetidin-3-yl)methyl)piperidin-4-yl)-1-(3- |
| | fluorophenyl)-2-(4-hydroxy-4-methylpiperidin-1- |
| | yl)ethyl)cyclopentyl)carbamate |
| 47 | methyl ((1S,2R)-2-((S)-2-(azetidin-1-yl)-1-(3-fluorophenyl)-1-(1-((1- |
| | (4-(oxetan-3-ylsulfonyl)phenyl)azetidin-3-yl)methyl)piperidin-4- |
| | yl)ethyl)cyclopentyl)carbamate |
| 48 | methyl ((1S,2R)-2-((S)-2-(azetidin-1-yl)-1-(3-fluorophenyl)-1-(1-((1- |
| | (4-((tetrahydro-2H-pyran-4-yl)sulfonyl)phenyl)azetidin-3- |
| | yl)methyl)piperidin-4-yl)ethyl)cyclopentyl)carbamate |
| 49 | methyl ((1S,2R)-2-((S)-2-(azetidin-1-yl)-1-(1-((1-(4- |
| | (cyclopropylsulfonyl)phenyl)azetidin-3-yl)methyl)piperidin-4-yl)-1- |
| | (3,5-difluorophenyl)ethyl)cyclopentyl)carbamate |
| 50 | methyl ((1S,2R)-2-((R)-2-(azetidin-1-yl)-1-(1-((1-(4- |
| | (cyclopropylsulfonyl)phenyl)azetidin-3-yl)methyl)piperidin-4-yl)-1- |
| | (3,5-difluorophenyl)ethyl)cyclopentyl)carbamate |
| 51 | methyl ((1S,2R)-2-((S)-2-(azetidin-1-yl)-1-(3-fluorophenyl)-1-(1- |
| | propionylpiperidin-4-yl)ethyl)cyclopentyl)carbamate |
| 52 | methyl ((1S,2R)-2-((S)-2-(azetidin-1-yl)-1-(1-((1-(4-cyanophenyl)-3- |
| | fluoroazetidin-3-yl)methyl)piperidin-4-yl)-1-(3,5- |
| | difluorophenyl)ethyl)cyclopentyl)carbamate |
| 53 | methyl ((1S,2R)-2-((S)-2-(azetidin-1-yl)-1-(1-((1-(4- |
| | (cyclopropylsulfonyl)phenyl)-3-fluoroazetidin-3-yl)methyl)piperidin-4- |
| | yl)-1-(3,5-difluorophenyl)ethyl)cyclopentyl)carbamate |
| 54 | methyl ((1S,2R)-2-((S)-2-(azetidin-1-yl)-1-(1-((1-(4-cyano-2,6- |
| | difluorophenyl)-3-fluoroazetidin-3-yl)methyl)piperidin-4-yl)-1-(3,5- |
| | difluorophenyl)ethyl)cyclopentyl)carbamate |
| 55 | methyl ((1S,2R)-2-((S)-2-(azetidin-1-yl)-1-(1-((1-(4-cyanophenyl)-3- |
| | fluoroazetidin-3-yl)methyl)piperidin-4-yl)-1-(3- |
| | fluorophenyl)ethyl)cyclopentyl)carbamate |
| 56 | methyl ((1S,2R)-2-((S)-2-(azetidin-1-yl)-1-(1-((1-(4-cyano-2,6- |
| | difluorophenyl)-3-fluoroazetidin-3-yl)methyl)piperidin-4-yl)-1-(3- |
| | fluorophenypethyl)cyclopentyl)carbamate |
| 57 | methyl ((1S,2R)-2-((S)-2-(azetidin-1-yl)-1-(3-fluorophenyl)-1-(1-((1- |
| | (4-((3-hydroxy-3-methylcyclobutyl)sulfonyl)phenyl)azetidin-3- |
| | yl)methyl)piperidin-4-yl)ethyl)cyclopentyl)carbamate |
| 58 | methyl ((1S,2R)-2-((S)-2-(azetidin-1-yl)-1-(1-((1-(4-((3,3- |
| | difluorocyclobutyl)sulfonyl)phenyl)azetidin-3-yl)methyl)piperidin-4- |
| | yl)-1-(3-fluorophenypethyl)cyclopentyl)carbamate |
| 59 | methyl ((1S,2R)-2-((S)-2-(azetidin-1-yl)-1-(1-((1-(4-((1-(4- |
| | (dimethylamino)butanoyl)azetidin-3-yl)sulfonyl)phenyl)-3- |
| | fluoroazetidin-3-yl)methyl)piperidin-4-yl)-1-(3- |
| | fluorophenyl)ethyl)cyclopentyl)carbamate |
| 60 | methyl ((1S,2R)-2-((S)-2-(azetidin-1-yl)-1-(1-((1-(4-((3,3- |
| | difluorocyclobutyl)sulfonyl)phenyl)-3-fluoroazetidin-3- |
| | yl)methyl)piperidin-4-yl)-1-(3-fluorophenyl)ethyl)cyclopentyl)carbamate |
| 61 | methyl ((1S,2R)-2-((S)-2-(azetidin-1-yl)-1-(1-((1-(4- |
| | (bicyclo [ 1.1.1 ] pentan-1-ylsulfonyl)phenyl)azetidin-3- |
| | yl)methyl)piperidin-4-yl)-1-(3-fluorophenyl)ethyl)cyclopentyl)carbamate |
| 62 | methyl ((1S,2R)-2-((S)-2-(azetidin-1-yl)-1-(1-((1-(4- |
| | (bicyclo [ 1.1.1 ] pentan-1-ylsulfonyl)phenyl)-3-fluoroazetidin-3- |
| | yl)methyl)piperidin-4-yl)-1-(3-fluorophenyl)ethyl)cyclopentyl)carbamate |
| 63 | methyl ((1S,2R)-2-((S)-2-(azetidin-1-yl)-1-(1-((3-fluoro-1-(4-((3- |
| | hydroxy-3-methylcyclobutyl)sulfonyl)phenyl)azetidin-3- |
| | yl)methyl)piperidin-4-yl)-1-(3-fluorophenyl)ethyl)cyclopentyl)carbamate |
| 64 | methyl ((1S,2R)-2-((S)-2-(azetidin-1-yl)-1-(1-((3-fluoro-1-(4-((1-(2- |
| | hydroxyethyl)azetidin-3-yl)sulfonyl)phenyl)azetidin-3- |
| | yl)methyl)piperidin-4-yl)-1-(3-fluorophenyl)ethyl)cyclopentyl)carbamate |
| 65 | methyl ((1S,2R)-2-((S)-2-(azetidin-1-yl)-1-(1-(3-(4- |
| | cyanophenoxy)propyl)piperidin-4-yl)-1-(3- |
| | fluorophenypethyl)cyclopentyl)carbamate |
| 66 | methyl ((1S,2R)-2-(2-(azetidin-1-yl)-1-((1r,4R)-4-ethoxycyclohexyl)-1- |
| | (3-fluorophenyl)ethyl)cyclopentyl)carbamate |
| | (2-position stereochemistry unknown) |
| 67 | methyl ((1S,2R)-2-(2-(azetidin-1-yl)-1-((1r,4R)-4-ethoxycyclohexyl)-1- |
| | (3-fluorophenyl)ethyl)cyclopentyl)carbamate |
| | (2-position stereochemistry unknown) |
| 68 | methyl ((1S,2R)-2-((S)-2-(azetidin-1-yl)-1-(1-(3-(4- |
| | (cyclopropylsulfonyl)phenoxy)propyl)piperidin-4-yl)-1-(3- |
| | fluorophenypethyl)cyclopentyl)carbamate |
| 69 | 4-((4-(3-((4-((S)-2-(azetidin-1-yl)-1-(3-fluorophenyl)-1-((1R,2S)-2- |
| | ((methoxycarbonyl)amino)cyclopentyl)ethyl)piperidin-1- |
| | yl)methyl)azetidin-1-yl)phenyl)sulfonyl)benzoic acid |
| 70 | methyl ((1S,2R)-2-(2-(azetidin-1-yl)-1-((1r,4R)-4-(2-(4- |
| | (cyclopropylsulfonyl)phenoxy)ethoxy)cyclohexyl)-1-(3- |
| | fluorophenypethyl)cyclopentyl)carbamate |
| | (2-position stereochemistry unknown) |
| 71 | methyl ((1S,2R)-2-(2-(azetidin-1-yl)-1-((1r,4R)-4-(2-(4- |
| | (cyclopropylsulfonyl)phenoxy)ethoxy)cyclohexyl)-1-(3- |
| | fluorophenyl)ethyl)cyclopentyl)carbamate |
| | (2-position stereochemistry unknown) |
| 72 | methyl ((1S,2R)-2-((S)-2-(azetidin-1-yl)-1-(1-((1-(4-cyano-2- |
| | fluorophenyl)azetidin-3-yl)methyl)piperidin-4-yl)-1-(3- |
| | fluorophenyl)ethyl)cyclopentyl)carbamate |
| 73 | methyl ((1S,2R)-2-((S)-2-(azetidin-1-yl)-1-(1-((1-(4-cyano-3- |
| | fluorophenyl)azetidin-3-yl)methyl)piperidin-4-yl)-1-(3- |
| | fluorophenyl)ethyl)cyclopentyl)carbamate |
| 74 | methyl ((1S,2R)-2-((S)-2-(azetidin-1-yl)-1-(1-((1-(4-cyano-2- |
| | fluorophenyl)-3-fluoroazetidin-3-yl)methyl)piperidin-4-yl)-1-(3- |
| | fluorophenyl)ethyl)cyclopentyl)carbamate |
| 75 | methyl ((1S,2R)-2-((S)-2-(azetidin-1-yl)-1-(1-((1-(4-cyano-3- |
| | fluorophenyl)-3-fluoroazetidin-3-yl)methyl)piperidin-4-yl)-1-(3- |
| | fluorophenypethyl)cyclopentyl)carbamate |
| 76 | methyl ((1S,2R)-2-((S)-2-(azetidin-1-yl)-1-(1-((3-fluoro-1-(4-(pyridin- |
| | 4-ylsulfonyl)phenyl)azetidin-3-yl)methyl)piperidin-4-yl)-1-(3- |
| | fluorophenyl)ethyl)cyclopentyl)carbamate |
| 77 | methyl ((1S,2R)-2-((S)-2-(azetidin-1-yl)-1-(1-((3-fluoro-1-(4-((1- |
| | methyl-1H-pyrazol-4-yl)sulfonyl)phenyl)azetidin-3- |
| | yl)methyl)piperidin-4-yl)-1-(3- |
| | fluorophenyl)ethyl)cyclopentyl)carbamate |
| 78 | methyl ((1S,2R)-2-((S)-cyano(1-((1-(4- |
| | (cyclopentylsulfonyl)phenyl)azetidin-3-yl)methyl)piperidin-4-yl)(3- |
| | fluorophenyl)methyl)cyclopentyl)carbamate |
| 79 | methyl ((1S,2R)-2-((S)-2-(azetidin-1-yl)-1-(3-fluorophenyl)-1-(1-((1- |
| | (4-(methylcarbamoyl)phenyl)azetidin-3-yl)methyl)piperidin-4- |
| | yl)ethyl)cyclopentyl)carbamate |
| 80 | methyl ((1S,2R)-2-((S)-2-(azetidin-1-yl)-1-(3-fluorophenyl)-1-(1-((1- |
| | (4-((4-(methylcarbamoyl)phenyl)sulfonyl)phenyl)azetidin-3- |
| | yl)methyl)piperidin-4-yl)ethyl)cyclopentyl)carbamate |
| 81 | methyl ((1S,2R)-2-((S)-2-(azetidin-1-yl)-1-(1-((1-(4-((1-(2-(azetidin-1- |
| | yl)acetyl)azetidin-3-yl)sulfonyl)phenyl)azetidin-3-yl)methyl)piperidin- |
| | 4-yl)-1-(3-fluorophenypethyl)cyclopentyl)carbamate |
| 82 | methyl ((1S,2R)-2-((S)-2-(azetidin-1-yl)-1-(3-fluorophenyl)-1-(1-((1- |
| | (4-((1-(2-morpholinoacetyl)azetidin-3-yl)sulfonyl)phenyl)azetidin-3- |
| | yl)methyl)piperidin-4-yl)ethyl)cyclopentyl)carbamate |
| 83 | methyl ((1S,2R)-2-((S)-2-(azetidin-1-yl)-1-(1-((1-(4-((1-(3- |
| | (dimethylamino)propanoyl)azetidin-3-yl)sulfonyl)phenyl)azetidin-3- |
| | yl)methyl)piperidin-4-yl)-1-(3-fluorophenyl)ethyl)cyclopentyl)carbamate |

In another embodiment, Compounds of the Disclosure are selected from the group consisting of:
methyl ((1S,2R)-2-((S)-2-(azetidin-1-yl)-1-(1-((1-(4-(cyclopropylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-(cyclopropylsulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyanophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-((1-methyl-1H-pyrazol-4-yl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-(cyclobutylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-2,6-difluorophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-(oxetan-3-ylsulfonyl)phenyl)azetidin-3-yl)methyl)piperidin-4-yl)ethyl)cyclopentyl)carbamate;
methyl ((1S,2R)-2-((S)-2-(azetidin-1-yl)-1-(3-fluorophenyl)-1-(1-((1-(4-((tetrahydro-2H-pyran-4-yl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-(cyclopropylsulfonyl)phenyl)azetidin-3-yl)methyl)piperidin-4-yl)-1-(3,5-difluorophenyl)ethyl)cyclopentyl)carbamate;
methyl ((1S,2R)-2-((R)-2-(azetidin-1-yl)-1-(1-((1-(4-(cyclopropylsulfonyl)phenyl)azetidin-3-yl)methyl)piperidin-4-yl)-1-(3,5-difluorophenyl)ethyl)cyclopentyl)carbamate;
methyl ((1S,2R)-2-((1S)-2-(azetidin-1-yl)-1-(1-((1-(4-(bicyclo[2.2.1]heptan-2-ylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyanophenyl)-3-fluoroazetidin-3-yl)methyl)piperidin-4-yl)-1-(3,5-difluorophenyl)ethyl)cyclopentyl)carbamate;
methyl ((1S,2R)-2-((S)-2-(azetidin-1-yl)-1-(1-((1-(4-(cyclopropylsulfonyl)phenyl)-3-fluoroazetidin-3-yl)methyl)piperidin-4-yl)-1-(3,5-difluorophenyl)ethyl)cyclopentyl)carbamate;
methyl ((1S,2R)-2-((S)-2-(azetidin-1-yl)-1-(1-((1-(4-cyano-2,6-difluorophenyl)-3-fluoroazetidin-3-yl)methyl)piperidin-4-yl)-1-(3,5-difluorophenyl)ethyl)cyclopentyl)carbamate;
methyl ((1S,2R)-2-((S)-2-(azetidin-1-yl)-1-(1-((1-(4-cyanophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-2,6-difluorophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-((3-hydroxy-3-methylcyclobutyl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-((3,3-difluorocyclobutyl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-((1-(4-(dimethylamino)butanoyl)azetidin-3-yl)sulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-((3,3-difluorocyclobutyl)sulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-(bicyclo[1.1.1]pentan-1-ylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-(bicyclo[1.1.1]pentan-1-yl sulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-((3-hydroxy-3-methylcyclobutyl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-((1-(2-hydroxyethyl)azetidin-3-yl)sulfonyl)phenyl)azetidin-3-yl)methyl)piperidin-4-yl)-1-(3-fluorophenyl)ethyl)cyclopentyl)carbamate;
4-((4-(3-((4-((S)-2-(azetidin-1-yl)-1-(3-fluorophenyl)-1-((1R,2S)-2-((methoxycarbonyl)amino)cyclopentyl)ethyl)piperidin-1-yl)methyl)azetidin-1-yl)phenyl)sulfonyl)benzoic acid;
methyl ((1S,2R)-2-((S)-2-(azetidin-1-yl)-1-((1r,4S)-4-(2-(4-(cyclopropylsulfonyl)phenoxy)ethoxy)cyclohexyl)-1-(3-fluorophenyl)ethyl)cyclopentyl)carbamate;
methyl ((1S,2R)-2-((R)-2-(azetidin-1-yl)-1-((1r,4R)-4-(2-(4-(cyclopropylsulfonyl)phenoxy)ethoxy)cyclohexyl)-1-(3-fluorophenyl)ethyl)cyclopentyl)carbamate;
methyl ((1S,2R)-2-((S)-2-(azetidin-1-yl)-1-(1-((1-(4-cyano-2-fluorophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-3-fluorophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-2-fluorophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-3-fluorophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-(pyridin-4-ylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-((1-methyl-1H-pyrazol-4-yl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-(methylcarbamoyl)phenyl)azetidin-3-yl)methyl)piperidin-4-yl)ethyl)cyclopentyl)carbamate;
methyl ((1S,2R)-2-((S)-2-(azetidin-1-yl)-1-(3-fluorophenyl)-1-(1-((1-(4-((4-(methylcarbamoyl)phenyl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-((1-(2-(azetidin-1-yl)acetyl)azetidin-3-yl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-((1-(2-morpholinoacetyl)azetidin-3-yl)sulfonyl)phenyl)azetidin-3-yl)methyl)piperidin-4-yl)ethyl)cyclopentyl)carbamate; and
methyl ((1S,2R)-2-((S)-2-(azetidin-1-yl)-1-(1-((1-(4-((1-(3-(dimethylamino)propanoyl)azetidin-3-yl)sulfonyl)phenyl)azetidin-3-yl)methyl)piperidin-4-yl)-1-(3-fluorophenyl)ethyl)cyclopentyl)carbamate,

and the pharmaceutically acceptable salts and solvates thereof.

In another embodiment, Compounds of the Disclosure are any one or more of the compounds of Table 1.2, and the pharmaceutically acceptable salts and solvates thereof. Table 1.2A provides the chemical names of the compounds of Table 1.2 generated by ChemCurator 19 (ChemAxon Kft.) or ChemDraw Professional 16 (PerkinElmer Informatics, Inc.). In the event of any ambiguity between their chemical structure and chemical name, Compounds of the Disclosure are defined by their chemical structure.

TABLE 1.2

| | | MS |
|---|---|---|
| | | (ESI) |
| | | m/z |
| Cpd No. | Structure | [M + H]⁺ |
| 84 | 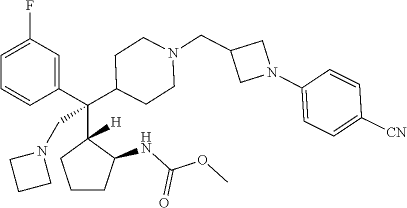 [SMILES: COC(=O)N[C@H]1CCC[C@@H]1[C@@](CN1CCC1)(c1cccc(F)c1)C1CCN(CC2CN(c3ccc(C#N)cc3)C2)CC1] | 574.07 |
| 85 | 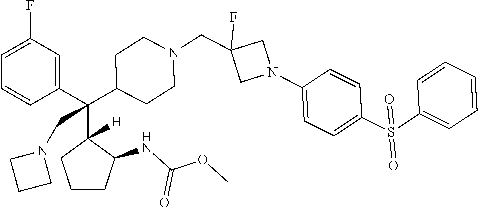 [SMILES: COC(=O)N[C@H]1CCC[C@@H]1[C@](CN1CCC1)(c1cccc(F)c1)C1CCN(CC2(F)CN(c3ccc(S(=O)(=O)c4ccccc4)cc3)C2)CC1] | 707.06 |
| 86 | 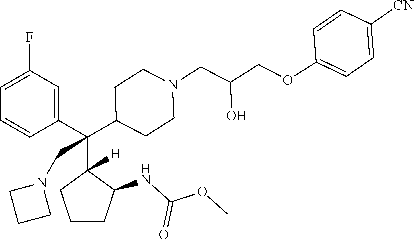 [SMILES: COC(=O)N[C@H]1CCC[C@@H]1[C@](CN1CCC1)(c1cccc(F)c1)C1CCN(CC(O)COc2ccc(C#N)cc2)CC1] | 579.09 |
| 87 | 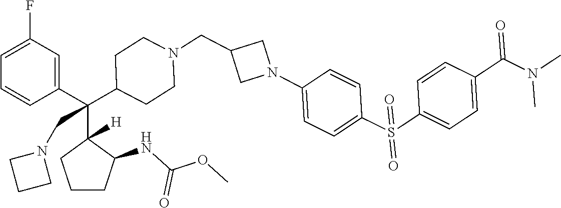 [SMILES: COC(=O)N[C@H]1CCC[C@@H]1[C@](CN1CCC1)(c1cccc(F)c1)C1CCN(CC2CN(c3ccc(S(=O)(=O)c4ccc(C(=O)N(C)C)cc4)cc3)C2)CC1] | 760.40 |
| 88 | 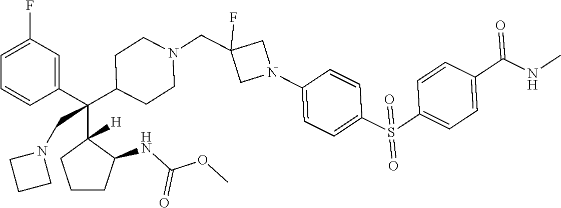 [SMILES: CNC(=O)c1ccc(S(=O)(=O)c2ccc(N3CC(F)(CN4CCC([C@@](CN5CCC5)(c5cccc(F)c5)[C@H]5CCC[C@@H]5NC(=O)OC)CC4)C3)cc2)cc1] | 764.44 |
| 89 | 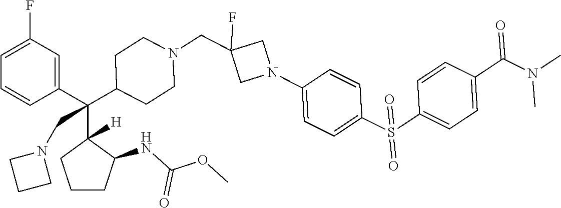 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N(C)C)cc4)cc3)C2)CC1] | 779.48 |
| 90 | 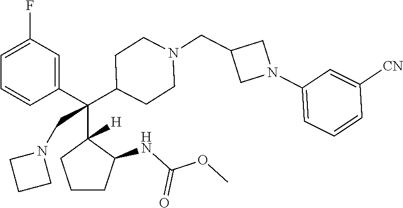 [SMILES: COC(=O)N[C@H]1CCC[C@@H]1[C@](CN1CCC1)(c1cccc(F)c1)C1CCN(CC2CN(c3cccc(C#N)c3)C2)CC1] | 574.47 |
| 91 | 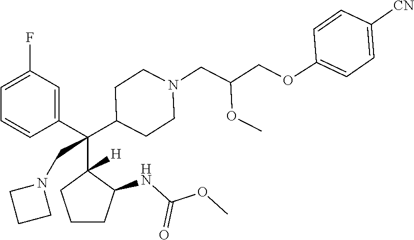 [SMILES: COC(=O)N[C@H]1CCC[C@@H]1[C@](CN1CCC1)(c1cccc(F)c1)C1CCN(CC(COc2ccc(C#N)cc2)OC)CC1] | 593.51 |
| 92 | 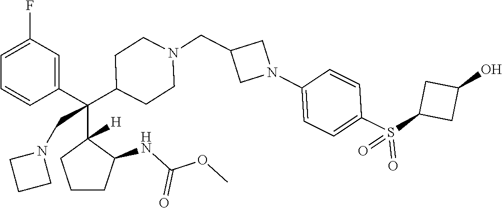 [SMILES: COC(=O)N[C@H]1CCC[C@@H]1[C@](CN1CCC1)(c1cccc(F)c1)C1CCN(CC2CN(c3ccc(S(=O)(=O)[C@H]4C[C@@H](O)C4)cc3)C2)CC1] | 683.52 |
| 93 | 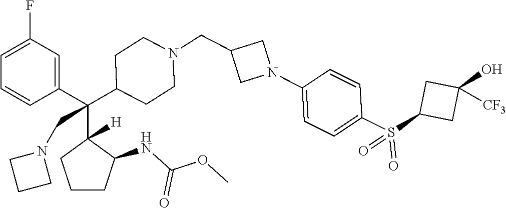 [SMILES: COC(=O)N[C@H]1CCC[C@@H]1[C@](CN1CCC1)(c1cccc(F)c1)C1CCN(CC2CN(c3ccc(S(=O)(=O)[C@H]4C[C@](O)(C(F)(F)F)C4)cc3)C2)CC1] | 751.49 |
| 94 | 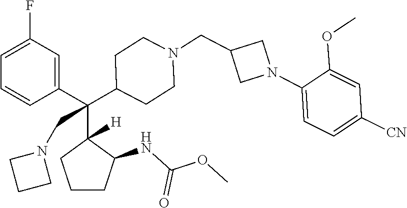 [SMILES: COC(=O)N[C@H]1CCC[C@@H]1[C@](CN1CCC1)(c1cccc(F)c1)C1CCN(CC2CN(c3ccc(C#N)cc3OC)C2)CC1] | 604.45 |
| 95 | 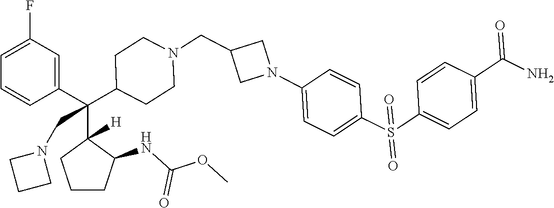 [SMILES: COC(=O)N[C@H]1CCC[C@@H]1[C@](CN1CCC1)(c1cccc(F)c1)C1CCN(CC2CN(c3ccc(S(=O)(=O)c4ccc(C(N)=O)cc4)cc3)C2)CC1] | 732.50 |
| 96 | 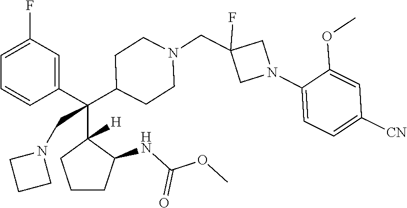 [SMILES: COC(=O)N[C@H]1CCC[C@@H]1[C@](CN1CCC1)(c1cccc(F)c1)C1CCN(CC2(F)CN(c3ccc(C#N)cc3OC)C2)CC1] | 622.44 |
| 97 | 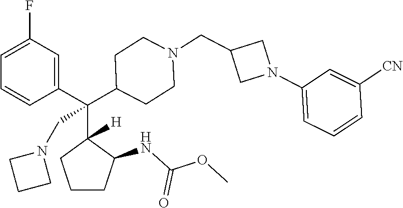 [SMILES: COC(=O)N[C@H]1CCC[C@@H]1[C@@](CN1CCC1)(c1cccc(F)c1)C1CCN(CC2CN(c3cccc(C#N)c3)C2)CC1] | 574.45 |
| 98 | 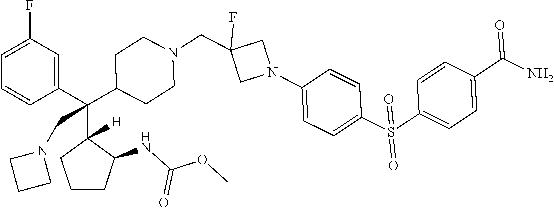 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(N)=O)cc4)cc3)C2)CC1] | 750.43 |
| 99 | 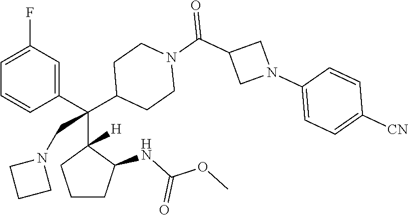 [SMILES: COC(=O)N[C@H]1CCC[C@@H]1[C@](CN1CCC1)(c1cccc(F)c1)C1CCN(C(=O)C2CN(c3ccc(C#N)cc3)C2)CC1] | 587.87 |
| 100 | 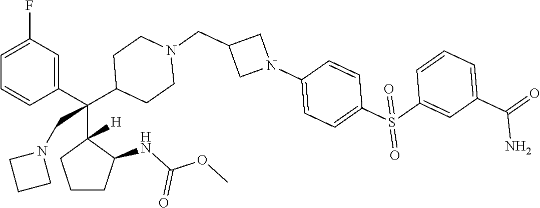 [SMILES: COC(=O)N[C@H]1CCC[C@@H]1[C@](CN1CCC1)(c1cccc(F)c1)C1CCN(CC2CN(c3ccc(S(=O)(=O)c4cccc(C(N)=O)c4)cc3)C2)CC1] | 732.56 |
| 101 | 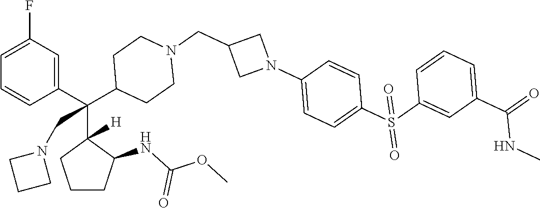 [SMILES: CNC(=O)c1cccc(S(=O)(=O)c2ccc(N3CC(CN4CCC([C@@](CN5CCC5)(c5cccc(F)c5)[C@H]5CCC[C@@H]5NC(=O)OC)CC4)C3)cc2)c1] | 746.45 |
| 102 | 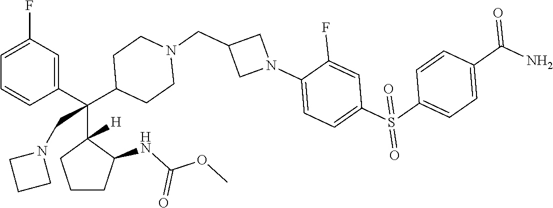 [SMILES: COC(=O)N[C@H]1CCC[C@@H]1[C@](CN1CCC1)(c1cccc(F)c1)C1CCN(CC2CN(c3ccc(S(=O)(=O)c4ccc(C(N)=O)cc4)cc3F)C2)CC1] | 750.49 |
| 103 | 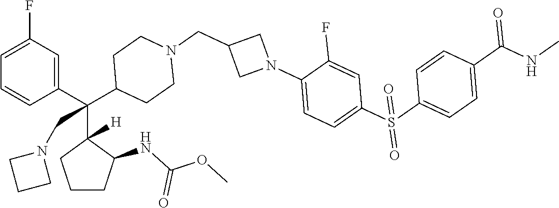 [SMILES: CNC(=O)c1ccc(S(=O)(=O)c2ccc(N3CC(CN4CCC([C@@](CN5CCC5)(c5cccc(F)c5)[C@H]5CCC[C@@H]5NC(=O)OC)CC4)C3)c(F)c2)cc1] | 764.97 |
| 104 | 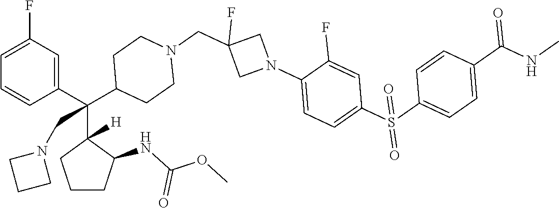 [SMILES: CNC(=O)c1ccc(S(=O)(=O)c2ccc(N3CC(F)(CN4CCC([C@@](CN5CCC5)(c5cccc(F)c5)[C@H]5CCC[C@@H]5NC(=O)OC)CC4)C3)c(F)c2)cc1] | 782.28 |
| 105 | 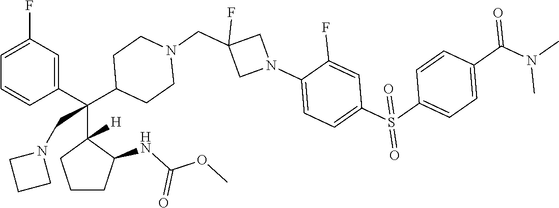 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N(C)C)cc4)cc3F)C2)CC1] | 796.88 |
| 106 | 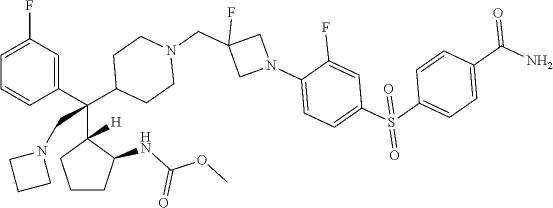 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(N)=O)cc4)cc3F)C2)CC1] | 768.29 |
| 107 | 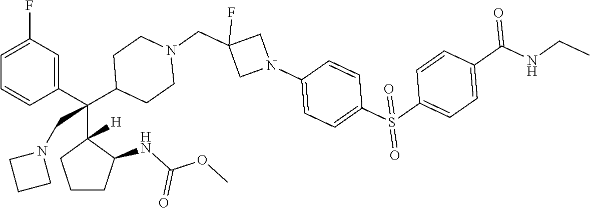 [SMILES: CCNC(=O)c1ccc(S(=O)(=O)c2ccc(N3CC(F)(CN4CCC([C@@](CN5CCC5)(c5cccc(F)c5)[C@H]5CCC[C@@H]5NC(=O)OC)CC4)C3)cc2)cc1] | 778.51 |
| 108 | 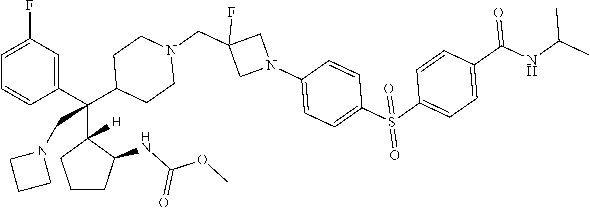 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)NC(C)C)cc4)cc3)C2)CC1] | 792.59 |
| 109 | 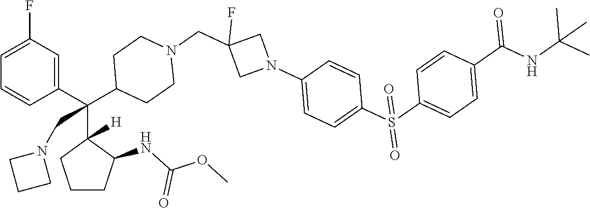 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)NC(C)(C)C)cc4)cc3)C2)CC1] | 806.55 |
| 110 | 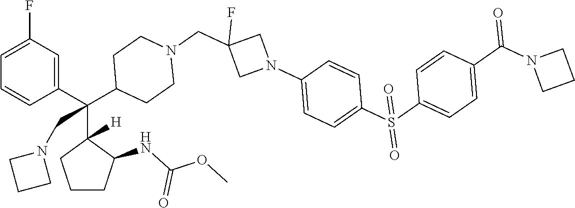 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CCC5)cc4)cc3)C2)CC1] | 790.41 |
| 111 | 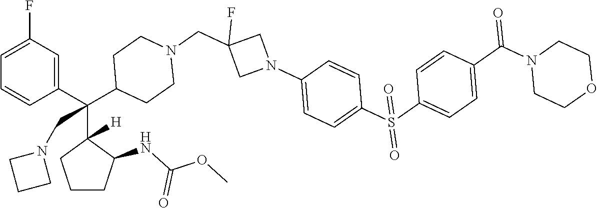 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CCOCC5)cc4)cc3)C2)CC1] | 820.65 |
| 112 | 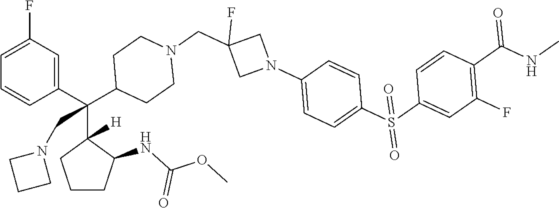 [SMILES: CNC(=O)c1ccc(S(=O)(=O)c2ccc(N3CC(F)(CN4CCC([C@@](CN5CCC5)(c5cccc(F)c5)[C@H]5CCC[C@@H]5NC(=O)OC)CC4)C3)cc2)cc1F] | 782.50 |
| 113 | 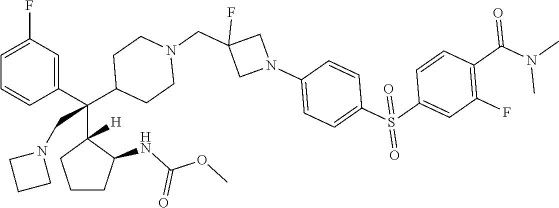 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N(C)C)c(F)c4)cc3)C2)CC1] | 796.41 |
| 114 | 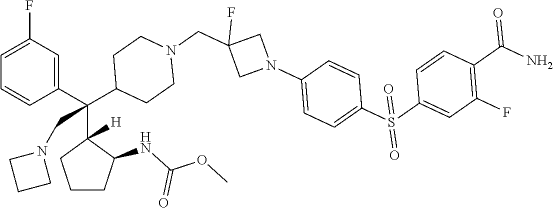 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(N)=O)c(F)c4)cc3)C2)CC1] | 768.35 |
| 115 | 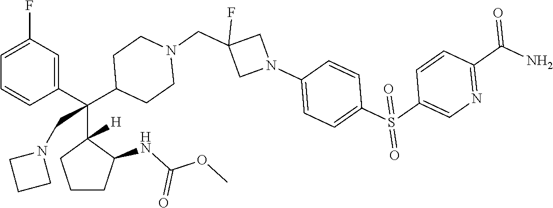 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(N)=O)nc4)cc3)C2)CC1] | 751.42 |
| 116 | 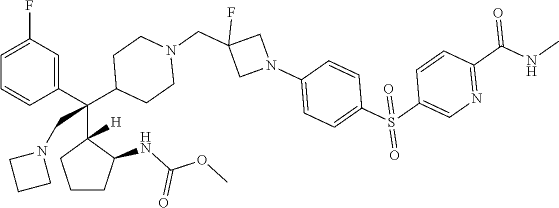 [SMILES: CNC(=O)c1ccc(S(=O)(=O)c2ccc(N3CC(F)(CN4CCC([C@@](CN5CCC5)(c5cccc(F)c5)[C@H]5CCC[C@@H]5NC(=O)OC)CC4)C3)cc2)cn1] | 765.44 |
| 117 | 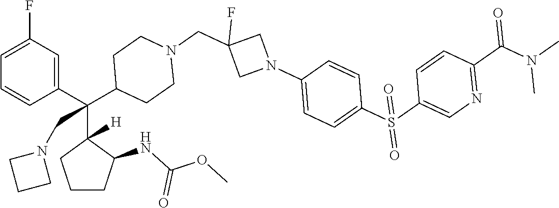 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N(C)C)nc4)cc3)C2)CC1] | 779.50 |
| 118 | 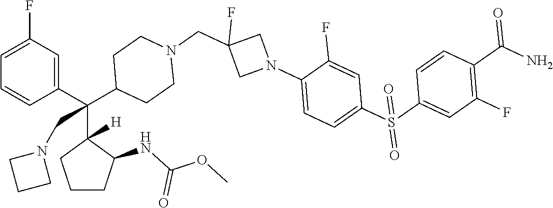 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(N)=O)c(F)c4)cc3F)C2)CC1] | 786.39 |
| 119 | 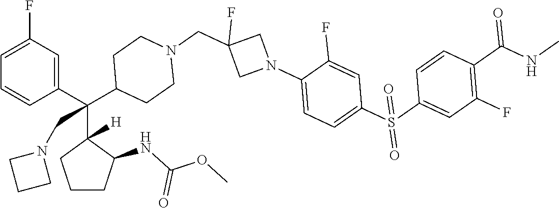 [SMILES: CNC(=O)c1ccc(S(=O)(=O)c2ccc(N3CC(F)(CN4CCC([C@@](CN5CCC5)(c5cccc(F)c5)[C@H]5CCC[C@@H]5NC(=O)OC)CC4)C3)c(F)c2)cc1F] | 800.37 |
| 120 | 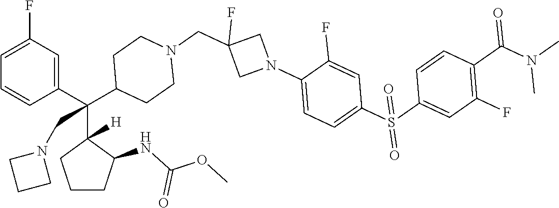 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N(C)C)c(F)c4)cc3F)C2)CC1] | 814.41 |
| 121 | 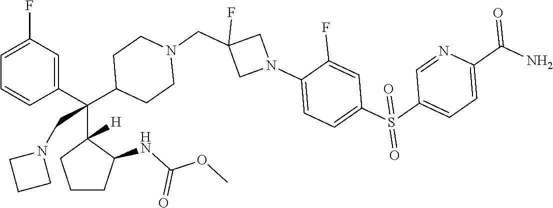 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(N)=O)nc4)cc3F)C2)CC1] | 769.29 |
| 122 | 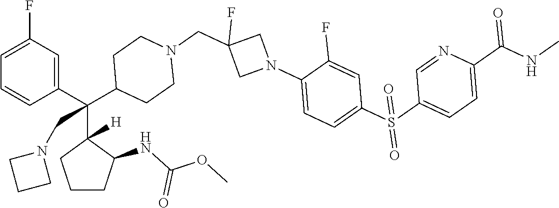 [SMILES: CNC(=O)c1ccc(S(=O)(=O)c2ccc(N3CC(F)(CN4CCC([C@@](CN5CCC5)(c5cccc(F)c5)[C@H]5CCC[C@@H]5NC(=O)OC)CC4)C3)c(F)c2)cn1] | 783.33 |
| 123 | 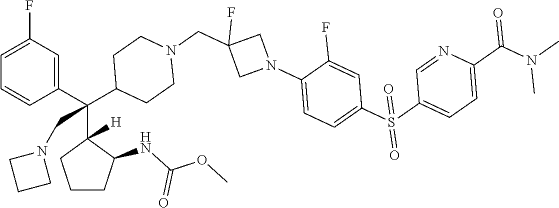 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N(C)C)nc4)cc3F)C2)CC1] | 797.38 |
| 124 | 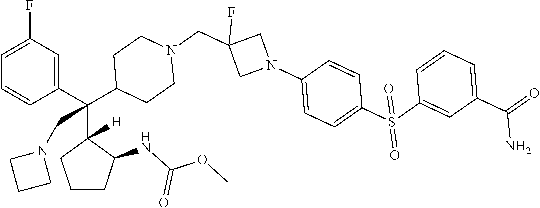 [SMILES: COC(=O)N[C@H]1CCC[C@@H]1[C@](CN1CCC1)(c1cccc(F)c1)C1CCN(CC2(F)CN(c3ccc(S(=O)(=O)c4cccc(C(N)=O)c4)cc3)C2)CC1] | 750.38 |
| 125 | 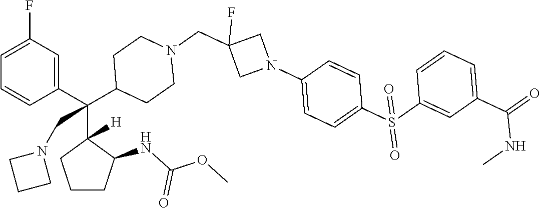 [SMILES: CNC(=O)c1cccc(S(=O)(=O)c2ccc(N3CC(F)(CN4CCC([C@@](CN5CCC5)(c5cccc(F)c5)[C@H]5CCC[C@@H]5NC(=O)OC)CC4)C3)cc2)c1] | 764.37 |
| 126 | 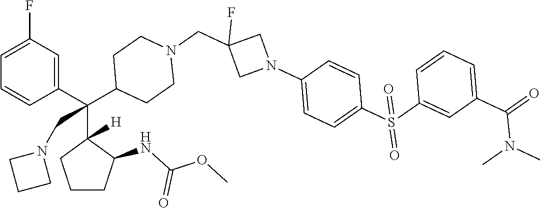 [SMILES: COC(=O)N[C@H]1CCC[C@@H]1[C@](CN1CCC1)(c1cccc(F)c1)C1CCN(CC2(F)CN(c3ccc(S(=O)(=O)c4cccc(C(=O)N(C)C)c4)cc3)C2)CC1] | 778.47 |
| 127 | 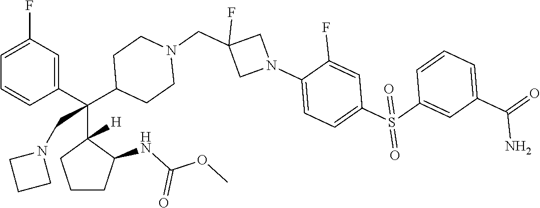 [SMILES: COC(=O)N[C@H]1CCC[C@@H]1[C@](CN1CCC1)(c1cccc(F)c1)C1CCN(CC2(F)CN(c3ccc(S(=O)(=O)c4cccc(C(N)=O)c4)cc3F)C2)CC1] | 768.30 |
| 128 | 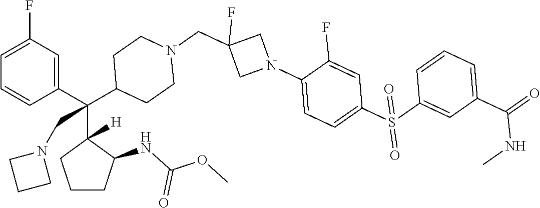 [SMILES: CNC(=O)c1cccc(S(=O)(=O)c2ccc(N3CC(F)(CN4CCC([C@@](CN5CCC5)(c5cccc(F)c5)[C@H]5CCC[C@@H]5NC(=O)OC)CC4)C3)c(F)c2)c1] | 782.27 |
| 129 | 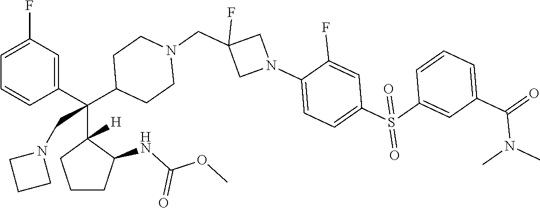 [SMILES: COC(=O)N[C@H]1CCC[C@@H]1[C@](CN1CCC1)(c1cccc(F)c1)C1CCN(CC2(F)CN(c3ccc(S(=O)(=O)c4cccc(C(=O)N(C)C)c4)cc3F)C2)CC1] | 782.27 |
| 130 | 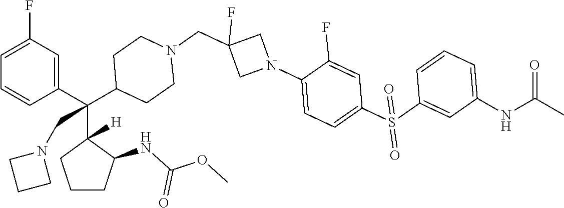 [SMILES: COC(=O)N[C@H]1CCC[C@@H]1[C@](CN1CCC1)(c1cccc(F)c1)C1CCN(CC2(F)CN(c3ccc(S(=O)(=O)c4cccc(NC(C)=O)c4)cc3F)C2)CC1] | 782.26 |
| 131 | 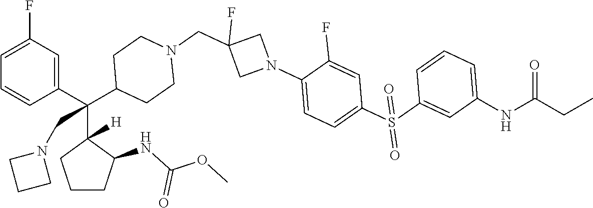 [SMILES: CCC(=O)Nc1cccc(S(=O)(=O)c2ccc(N3CC(F)(CN4CCC([C@@](CN5CCC5)(c5cccc(F)c5)[C@H]5CCC[C@@H]5NC(=O)OC)CC4)C3)c(F)c2)c1] | 796.26 |
| 132 | 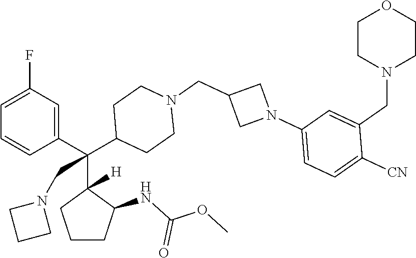 [SMILES: COC(=O)N[C@H]1CCC[C@@H]1[C@](CN1CCC1)(c1cccc(F)c1)C1CCN(CC2CN(c3ccc(C#N)c(CN4CCOCC4)c3)C2)CC1] | 673.39 |
| 133 | 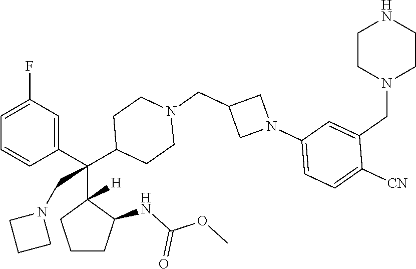 [SMILES: COC(=O)N[C@H]1CCC[C@@H]1[C@](CN1CCC1)(c1cccc(F)c1)C1CCN(CC2CN(c3ccc(C#N)c(CN4CCNCC4)c3)C2)CC1] | 672.41 |
| 134 | 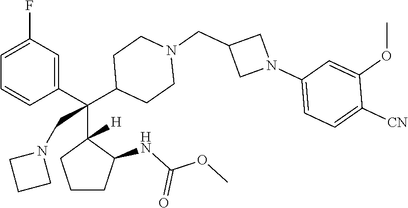 [SMILES: COC(=O)N[C@H]1CCC[C@@H]1[C@](CN1CCC1)(c1cccc(F)c1)C1CCN(CC2CN(c3ccc(C#N)c(OC)c3)C2)CC1] | 604.32 |
| 135 | 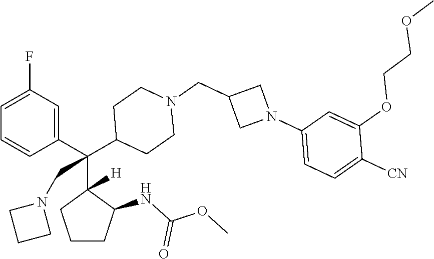 [SMILES: COCCOc1cc(N2CC(CN3CCC([C@@](CN4CCC4)(c4cccc(F)c4)[C@H]4CCC[C@@H]4NC(=O)OC)CC3)C2)ccc1C#N] | 648.36 |
| 136 | 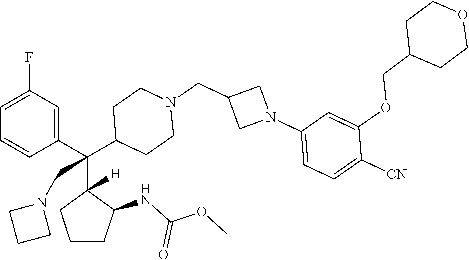 [SMILES: COC(=O)N[C@H]1CCC[C@@H]1[C@](CN1CCC1)(c1cccc(F)c1)C1CCN(CC2CN(c3ccc(C#N)c(OCC4CCOCC4)c3)C2)CC1] | 688.41 |
| 137 | 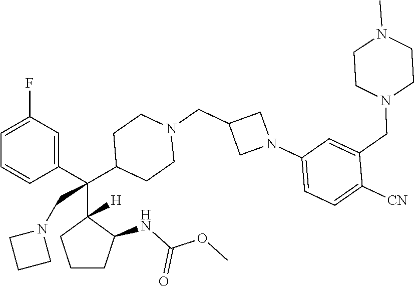 [SMILES: COC(=O)N[C@H]1CCC[C@@H]1[C@](CN1CCC1)(c1cccc(F)c1)C1CCN(CC2CN(c3ccc(C#N)c(CN4CCN(C)CC4)c3)C2)CC1] | 686.47 |
| 138 | 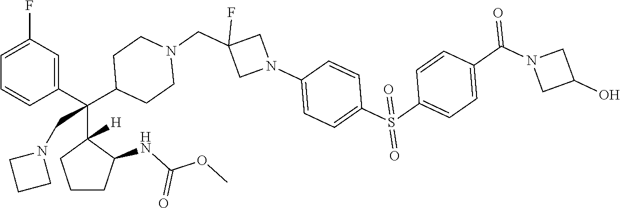 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CC(O)C5)cc4)cc3)C2)CC1] | 806.50 |
| 139 | 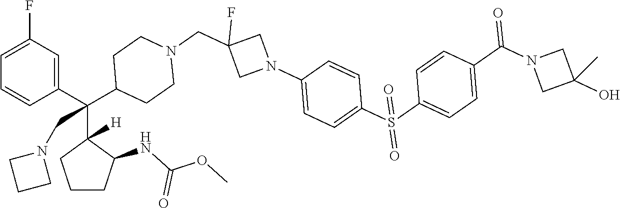 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CC(C)(O)C5)cc4)cc3)C2)CC1] | 820.35 |
| 140 | 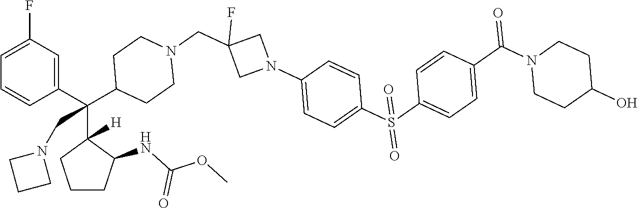 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CCC(O)CC5)cc4)cc3)C2)CC1] | 834.37 |
| 141 | 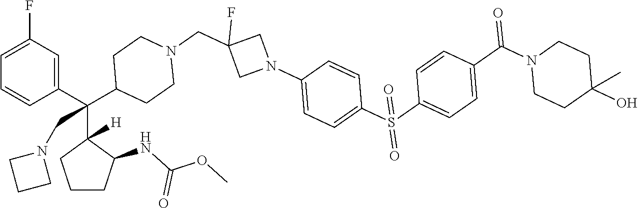 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CCC(C)(O)CC5)cc4)cc3)C2)CC1] | 848.57 |
| 142 | 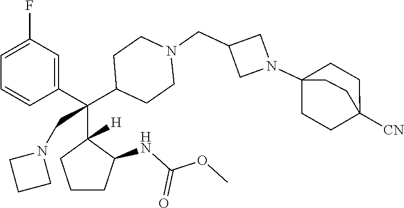 [SMILES: COC(=O)N[C@H]1CCC[C@@H]1[C@](CN1CCC1)(c1cccc(F)c1)C1CCN(CC2CN(C34CCC(C#N)(CC3)CC4)C2)CC1] | 606.51 |
| 143 | 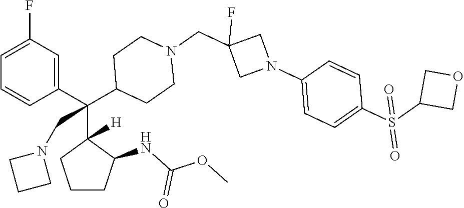 [SMILES: COC(=O)N[C@H]1CCC[C@@H]1[C@](CN1CCC1)(c1cccc(F)c1)C1CCN(CC2(F)CN(c3ccc(S(=O)(=O)C4COC4)cc3)C2)CC1] | 687.49 |
| 144 | 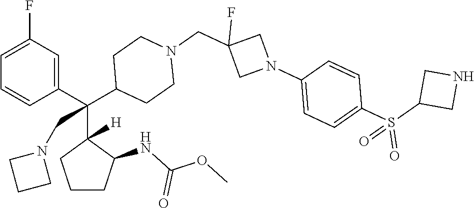 [SMILES: COC(=O)N[C@H]1CCC[C@@H]1[C@](CN1CCC1)(c1cccc(F)c1)C1CCN(CC2(F)CN(c3ccc(S(=O)(=O)C4CNC4)cc3)C2)CC1] | 708.44 |
| 145 | 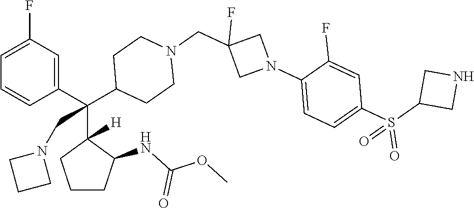 [SMILES: COC(=O)N[C@H]1CCC[C@@H]1[C@](CN1CCC1)(c1cccc(F)c1)C1CCN(CC2(F)CN(c3ccc(S(=O)(=O)C4CNC4)cc3F)C2)CC1] | 704.43 |
| 146 | 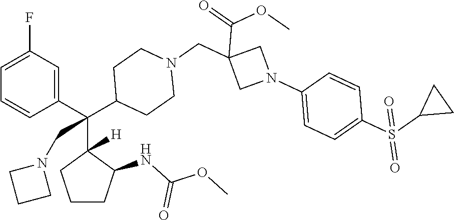 [SMILES: COC(=O)N[C@H]1CCC[C@@H]1[C@](CN1CCC1)(c1cccc(F)c1)C1CCN(CC2(C(=O)OC)CN(c3ccc(S(=O)(=O)C4CC4)cc3)C2)CC1] | 711.51 |
| 147 | 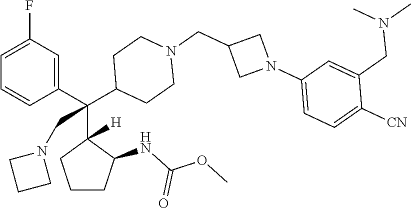 [SMILES: COC(=O)N[C@H]1CCC[C@@H]1[C@](CN1CCC1)(c1cccc(F)c1)C1CCN(CC2CN(c3ccc(C#N)c(CN(C)C)c3)C2)CC1] | 631.63 |
| 148 | 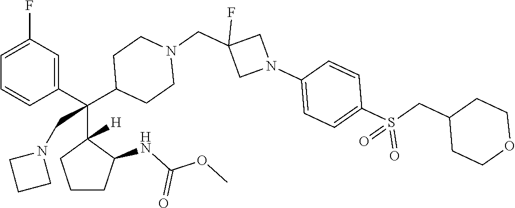 [SMILES: COC(=O)N[C@H]1CCC[C@@H]1[C@](CN1CCC1)(c1cccc(F)c1)C1CCN(CC2(F)CN(c3ccc(S(=O)(=O)CC4CCOCC4)cc3)C2)CC1] | 729.50 |
| 149 | 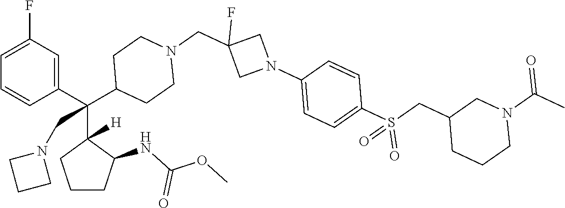 [SMILES: COC(=O)N[C@H]1CCC[C@@H]1[C@](CN1CCC1)(c1cccc(F)c1)C1CCN(CC2(F)CN(c3ccc(S(=O)(=O)CC4CCCN(C(C)=O)C4)cc3)C2)CC1] | 756.49 |
| 150 | 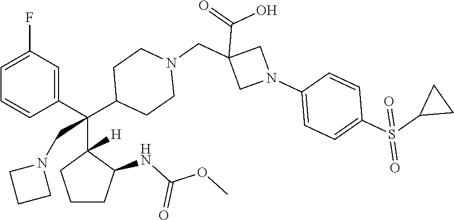 [SMILES: COC(=O)N[C@H]1CCC[C@@H]1[C@](CN1CCC1)(c1cccc(F)c1)C1CCN(CC2(C(=O)O)CN(c3ccc(S(=O)(=O)C4CC4)cc3)C2)CC1] | 697.51 |
| 151 | 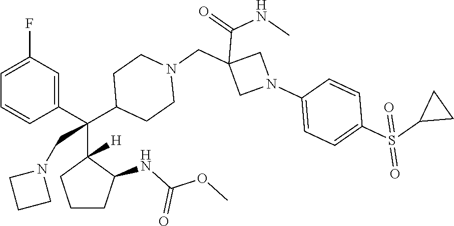 [SMILES: CNC(=O)C1(CN2CCC([C@@](CN3CCC3)(c3cccc(F)c3)[C@H]3CCC[C@@H]3NC(=O)OC)CC2)CN(c2ccc(S(=O)(=O)C3CC3)cc2)C1] | 710.30 |
| 152 | 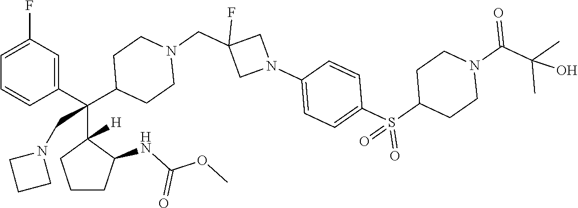 [SMILES: COC(=O)N[C@H]1CCC[C@@H]1[C@](CN1CCC1)(c1cccc(F)c1)C1CCN(CC2(F)CN(c3ccc(S(=O)(=O)C4CCN(C(=O)C(C)(C)O)CC4)cc3)C2)CC1] | 800.51 |
| 153 | 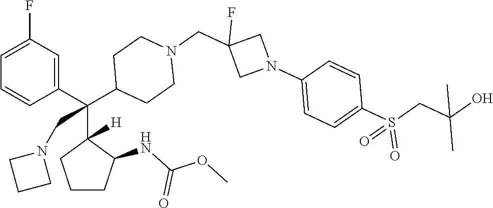 [SMILES: COC(=O)N[C@H]1CCC[C@@H]1[C@](CN1CCC1)(c1cccc(F)c1)C1CCN(CC2(F)CN(c3ccc(S(=O)(=O)CC(C)(C)O)cc3)C2)CC1] | 703.34 |
| 154 | 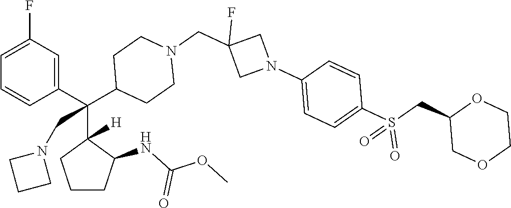 [SMILES: COC(=O)N[C@H]1CCC[C@@H]1[C@](CN1CCC1)(c1cccc(F)c1)C1CCN(CC2(F)CN(c3ccc(S(=O)(=O)C[C@@H]4COCCO4)cc3)C2)CC1] | 731.43 |
| 155 | 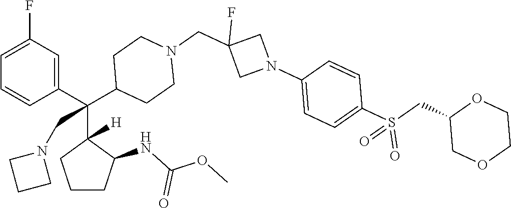 [SMILES: COC(=O)N[C@H]1CCC[C@@H]1[C@](CN1CCC1)(c1cccc(F)c1)C1CCN(CC2(F)CN(c3ccc(S(=O)(=O)C[C@H]4COCCO4)cc3)C2)CC1] | 731.44 |
| 156 | 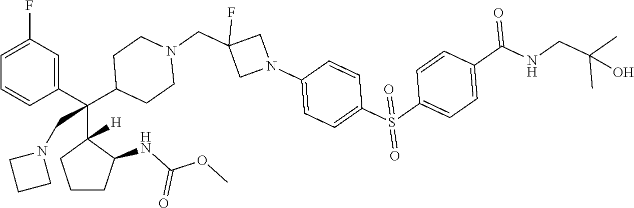 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)NCC(C)(C)O)cc4)cc3)C2)CC1] | 822.55 |
| 157 | 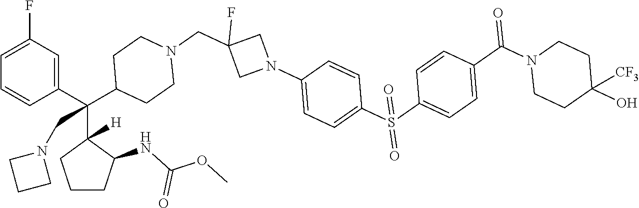 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CCC(O)(C(F)(F)F)CC5)cc4)cc3)C2)CC1] | 902.61 |
| 158 | 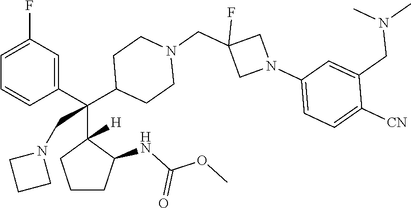 [SMILES: COC(=O)N[C@H]1CCC[C@@H]1[C@](CN1CCC1)(c1cccc(F)c1)C1CCN(CC2(F)CN(c3ccc(C#N)c(CN(C)C)c3)C2)CC1] | 649.60 |
| 159 | 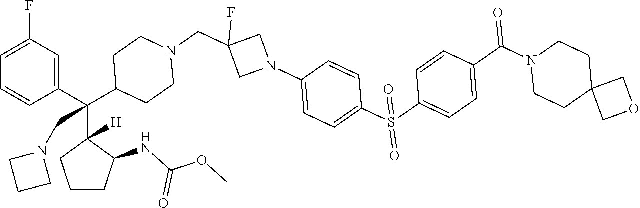 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)N5CCC6(CC5)COC6)cc4)cc3)C2)CC1] | 860.64 |
| 160 | 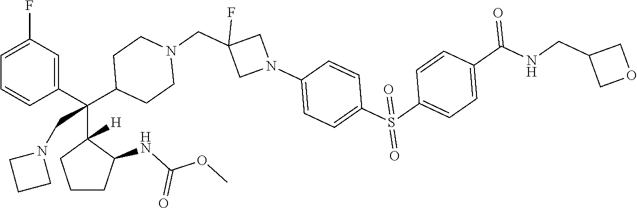 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)NCC5COC5)cc4)cc3)C2)CC1] | 820.53 |
| 161 | 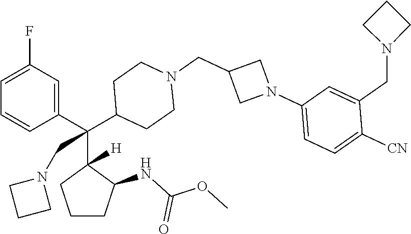 [SMILES: COC(=O)N[C@H]1CCC[C@@H]1[C@](CN1CCC1)(c1cccc(F)c1)C1CCN(CC2CN(c3ccc(C#N)c(CN4CCC4)c3)C2)CC1] | 643.60 |
| 162 | 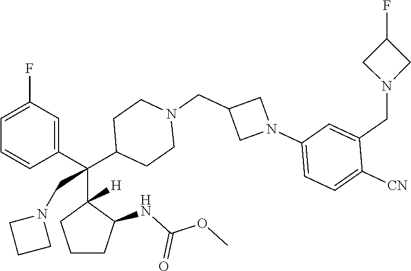 [SMILES: COC(=O)N[C@H]1CCC[C@@H]1[C@](CN1CCC1)(c1cccc(F)c1)C1CCN(CC2CN(c3ccc(C#N)c(CN4CC(F)C4)c3)C2)CC1] | 661.55 |
| 163 | 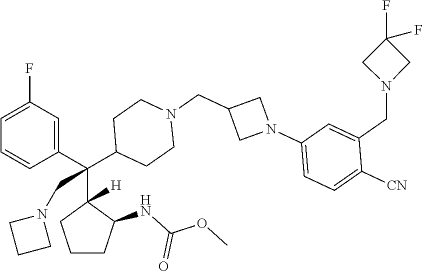 [SMILES: COC(=O)N[C@H]1CCC[C@@H]1[C@](CN1CCC1)(c1cccc(F)c1)C1CCN(CC2CN(c3ccc(C#N)c(CN4CC(F)(F)C4)c3)C2)CC1] | 679.50 |
| 164 | 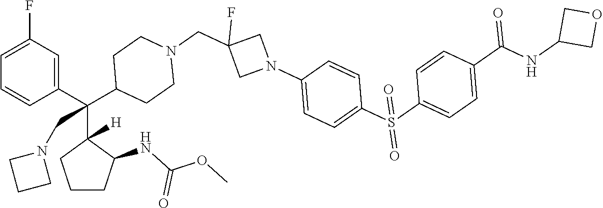 [SMILES: COC(=O)N[C@H]1CCC[C@@H]1[C@](CN1CCC1)(c1cccc(F)c1)C1CCN(CC2(F)CN(c3ccc(S(=O)(=O)c4ccc(C(=O)NC5COC5)cc4)cc3)C2)CC1] | 806.07 |
| 165 | 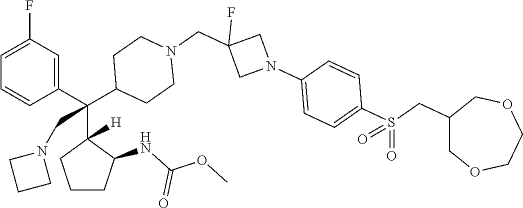 [SMILES: COC(=O)N[C@H]1CCC[C@@H]1[C@](CN1CCC1)(c1cccc(F)c1)C1CCN(CC2(F)CN(c3ccc(S(=O)(=O)CC4COCCOC4)cc3)C2)CC1] | 745.05 |
| 166 | 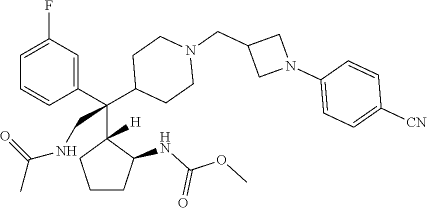 [SMILES: COC(=O)N[C@H]1CCC[C@@H]1[C@](CNC(C)=O)(c1cccc(F)c1)C1CCN(CC2CN(c3ccc(C#N)cc3)C2)CC1] | 576.48 |
| 167 | 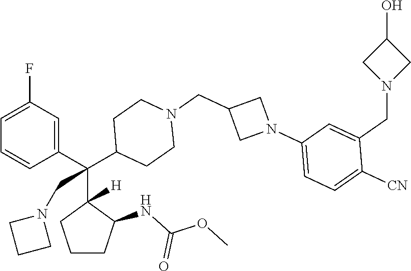 [SMILES: COC(=O)N[C@H]1CCC[C@@H]1[C@](CN1CCC1)(c1cccc(F)c1)C1CCN(CC2CN(c3ccc(C#N)c(CN4CC(O)C4)c3)C2)CC1] | 659.54 |
| 168 | 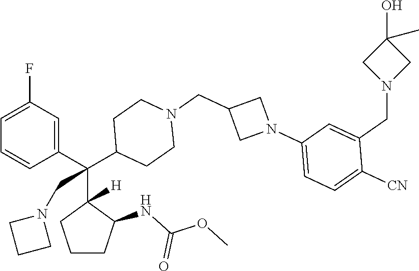 [SMILES: COC(=O)N[C@H]1CCC[C@@H]1[C@](CN1CCC1)(c1cccc(F)c1)C1CCN(CC2CN(c3ccc(C#N)c(CN4CC(C)(O)C4)c3)C2)CC1] | 673.56 |
| 169 | 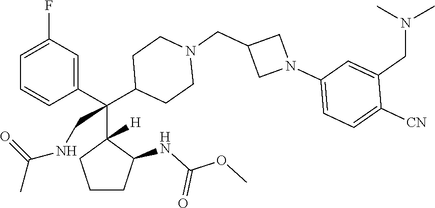 [SMILES: COC(=O)N[C@H]1CCC[C@@H]1[C@](CNC(C)=O)(c1cccc(F)c1)C1CCN(CC2CN(c3ccc(C#N)c(CN(C)C)c3)C2)CC1] | 633.55 |
| 170 | 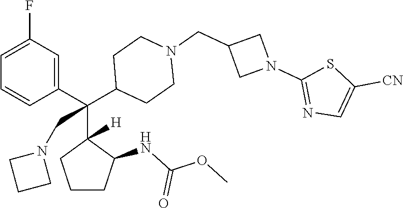 [SMILES: COC(=O)N[C@H]1CCC[C@@H]1[C@](CN1CCC1)(c1cccc(F)c1)C1CCN(CC2CN(c3ncc(C#N)s3)C2)CC1] | 581.42 |
| 171 | 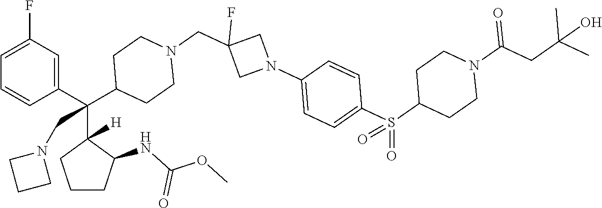 [SMILES: COC(=O)N[C@H]1CCC[C@@H]1[C@](CN1CCC1)(c1cccc(F)c1)C1CCN(CC2(F)CN(c3ccc(S(=O)(=O)C4CCN(C(=O)CC(C)(C)O)CC4)cc3)C2)CC1] | 817.31 |
| 172 | 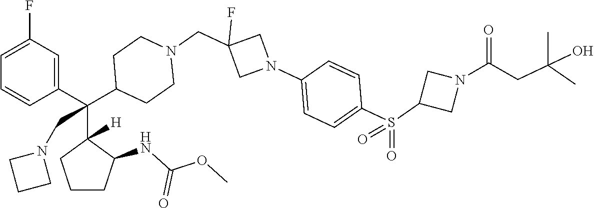 [SMILES: COC(=O)N[C@H]1CCC[C@@H]1[C@](CN1CCC1)(c1cccc(F)c1)C1CCN(CC2(F)CN(c3ccc(S(=O)(=O)C4CN(C(=O)CC(C)(C)O)C4)cc3)C2)CC1] | 786.52 |
| 173 | 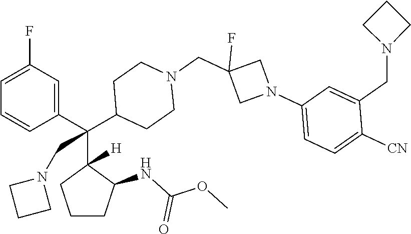 [SMILES: COC(=O)N[C@H]1CCC[C@@H]1[C@](CN1CCC1)(c1cccc(F)c1)C1CCN(CC2(F)CN(c3ccc(C#N)c(CN4CCC4)c3)C2)CC1] | 661.54 |
| 174 | 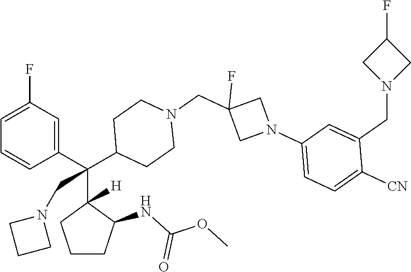 [SMILES: COC(=O)N[C@H]1CCC[C@@H]1[C@](CN1CCC1)(c1cccc(F)c1)C1CCN(CC2(F)CN(c3ccc(C#N)c(CN4CC(F)C4)c3)C2)CC1] | 679.53 |
| 175 | 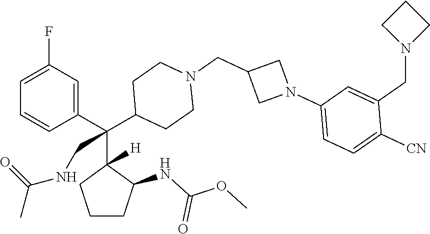 [SMILES: COC(=O)N[C@H]1CCC[C@@H]1[C@](CNC(C)=O)(c1cccc(F)c1)C1CCN(CC2CN(c3ccc(C#N)c(CN4CCC4)c3)C2)CC1] | 645.61 |
| 176 | 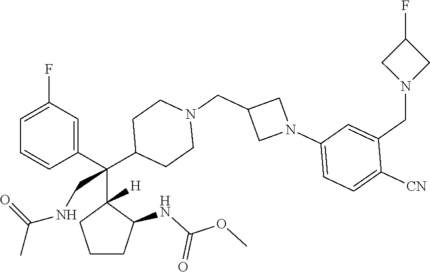 [SMILES: COC(=O)N[C@H]1CCC[C@@H]1[C@](CNC(C)=O)(c1cccc(F)c1)C1CCN(CC2CN(c3ccc(C#N)c(CN4CC(F)C4)c3)C2)CC1] | 663.53 |
| 177 | 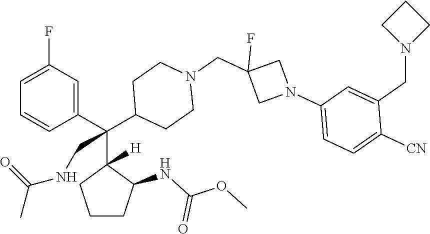 [SMILES: COC(=O)N[C@H]1CCC[C@@H]1[C@](CNC(C)=O)(c1cccc(F)c1)C1CCN(CC2(F)CN(c3ccc(C#N)c(CN4CCC4)c3)C2)CC1] | 663.50 |
| 178 | 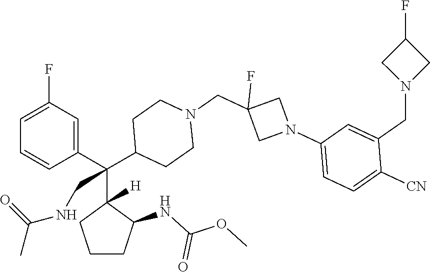 [SMILES: COC(=O)N[C@H]1CCC[C@@H]1[C@](CNC(C)=O)(c1cccc(F)c1)C1CCN(CC2(F)CN(c3ccc(C#N)c(CN4CC(F)C4)c3)C2)CC1] | 681.47 |
| 179 | 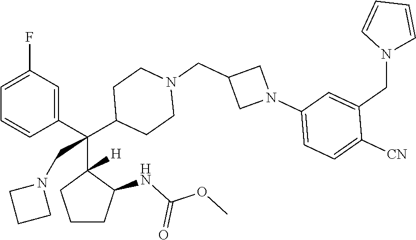 [SMILES: COC(=O)N[C@H]1CCC[C@@H]1[C@](CN1CCC1)(c1cccc(F)c1)C1CCN(CC2CN(c3ccc(C#N)c(Cn4cccc4)c3)C2)CC1] | 653.57 |
| 180 | 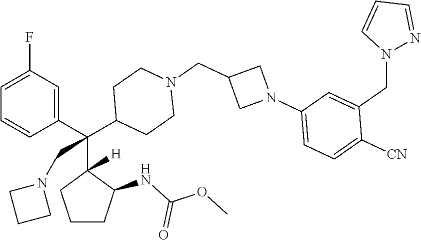 [SMILES: COC(=O)N[C@H]1CCC[C@@H]1[C@](CN1CCC1)(c1cccc(F)c1)C1CCN(CC2CN(c3ccc(C#N)c(Cn4cccn4)c3)C2)CC1] | 654.56 |
| 181 | 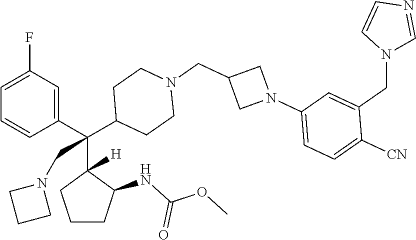 [SMILES: COC(=O)N[C@H]1CCC[C@@H]1[C@](CN1CCC1)(c1cccc(F)c1)C1CCN(CC2CN(c3ccc(C#N)c(Cn4ccnc4)c3)C2)CC1] | 654.54 |
| 182 | 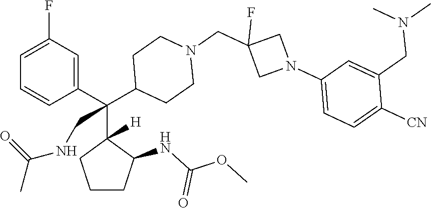 [SMILES: COC(=O)N[C@H]1CCC[C@@H]1[C@](CNC(C)=O)(c1cccc(F)c1)C1CCN(CC2(F)CN(c3ccc(C#N)c(CN(C)C)c3)C2)CC1] | 651.53 |
| 183 | 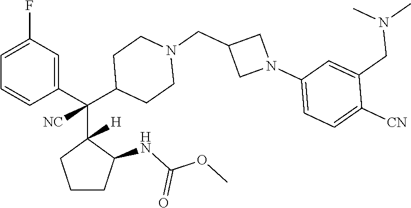 [SMILES: COC(=O)N[C@H]1CCC[C@@H]1[C@](C#N)(c1cccc(F)c1)C1CCN(CC2CN(c3ccc(C#N)c(CN(C)C)c3)C2)CC1] | 587.51 |
| 184 | 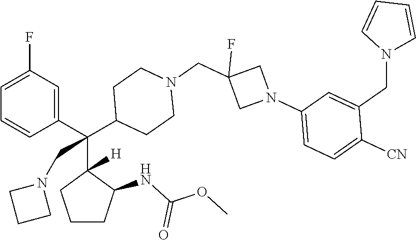 [SMILES: COC(=O)N[C@H]1CCC[C@@H]1[C@](CN1CCC1)(c1cccc(F)c1)C1CCN(CC2(F)CN(c3ccc(C#N)c(Cn4cccc4)c3)C2)CC1] | 671.54 |
| 185 | 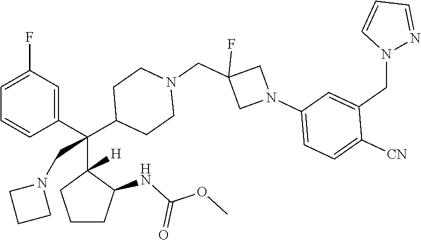 [SMILES: COC(=O)N[C@H]1CCC[C@@H]1[C@](CN1CCC1)(c1cccc(F)c1)C1CCN(CC2(F)CN(c3ccc(C#N)c(Cn4cccn4)c3)C2)CC1] | 672.56 |
| 186 | 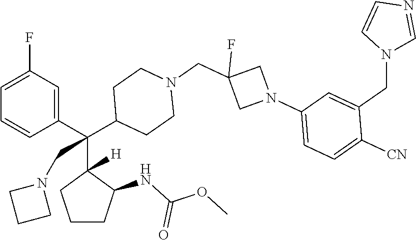 [SMILES: COC(=O)N[C@H]1CCC[C@@H]1[C@](CN1CCC1)(c1cccc(F)c1)C1CCN(CC2(F)CN(c3ccc(C#N)c(Cn4ccnc4)c3)C2)CC1] | 672.56 |
| 187 | 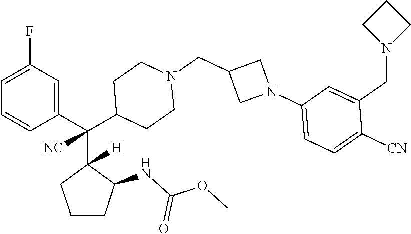 [SMILES: COC(=O)N[C@H]1CCC[C@@H]1[C@](C#N)(c1cccc(F)c1)C1CCN(CC2CN(c3ccc(C#N)c(CN4CCC4)c3)C2)CC1] | 599.50 |
| 188 | 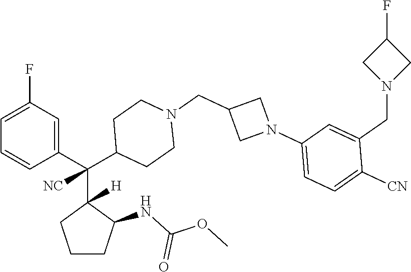 [SMILES: COC(=O)N[C@H]1CCC[C@@H]1[C@](C#N)(c1cccc(F)c1)C1CCN(CC2CN(c3ccc(C#N)c(CN4CC(F)C4)c3)C2)CC1] | 617.50 |
| 189 | 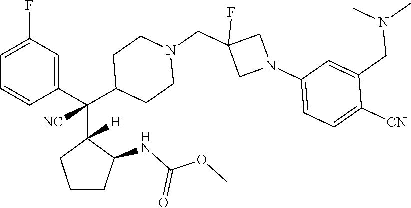 [SMILES: COC(=O)N[C@H]1CCC[C@@H]1[C@](C#N)(c1cccc(F)c1)C1CCN(CC2(F)CN(c3ccc(C#N)c(CN(C)C)c3)C2)CC1] | 605.47 |
| 190 | 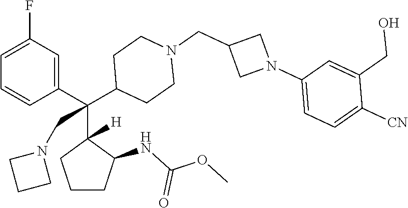 [SMILES: COC(=O)N[C@H]1CCC[C@@H]1[C@](CN1CCC1)(c1cccc(F)c1)C1CCN(CC2CN(c3ccc(C#N)c(CO)c3)C2)CC1] | 605.48 |
| 191 | 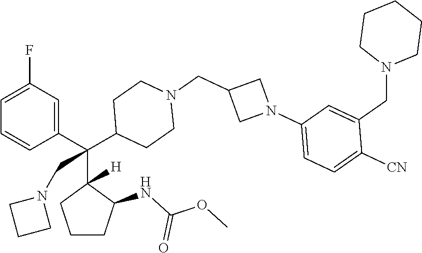 [SMILES: COC(=O)N[C@H]1CCC[C@@H]1[C@](CN1CCC1)(c1cccc(F)c1)C1CCN(CC2CN(c3ccc(C#N)c(CN4CCCCC4)c3)C2)CC1] | 671.60 |
| 192 | 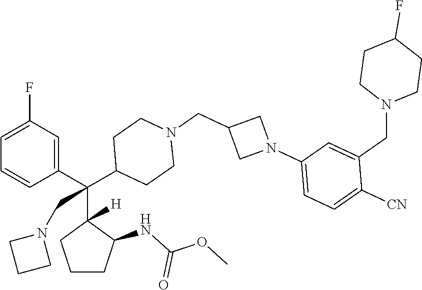 [SMILES: COC(=O)N[C@H]1CCC[C@@H]1[C@](CN1CCC1)(c1cccc(F)c1)C1CCN(CC2CN(c3ccc(C#N)c(CN4CCC(F)CC4)c3)C2)CC1] | 689.61 |
| 193 | 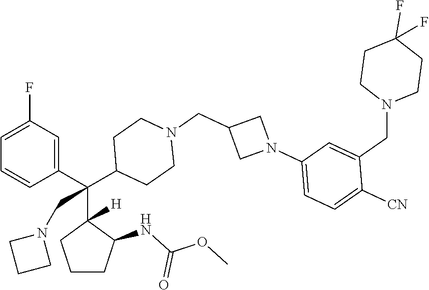 [SMILES: COC(=O)N[C@H]1CCC[C@@H]1[C@](CN1CCC1)(c1cccc(F)c1)C1CCN(CC2CN(c3ccc(C#N)c(CN4CCC(F)(F)CC4)c3)C2)CC1] | 707.56 |
| 194 | 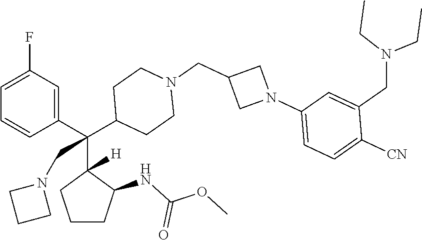 [SMILES: CCN(CC)Cc1cc(N2CC(CN3CCC([C@@](CN4CCC4)(c4cccc(F)c4)[C@H]4CCC[C@@H]4NC(=O)OC)CC3)C2)ccc1C#N] | 659.59 |
| 195 | 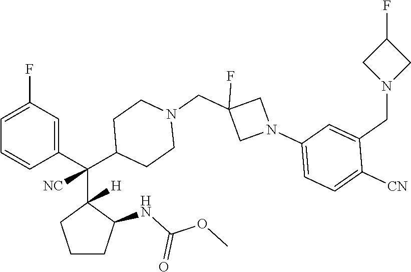 [SMILES: COC(=O)N[C@H]1CCC[C@@H]1[C@](C#N)(c1cccc(F)c1)C1CCN(CC2(F)CN(c3ccc(C#N)c(CN4CC(F)C4)c3)C2)CC1] | 635.50 |
| 196 | 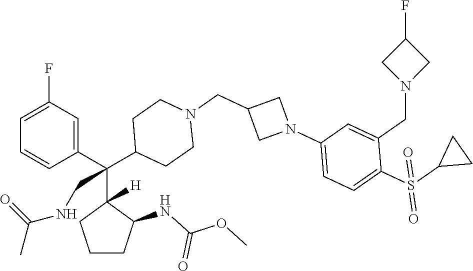 [SMILES: COC(=O)N[C@H]1CCC[C@@H]1[C@](CNC(C)=O)(c1cccc(F)c1)C1CCN(CC2CN(c3ccc(S(=O)(=O)C4CC4)c(CN4CC(F)C4)c3)C2)CC1] | 742.48 |
| 197 | 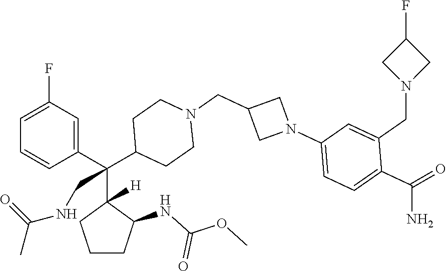 [SMILES: COC(=O)N[C@H]1CCC[C@@H]1[C@](CNC(C)=O)(c1cccc(F)c1)C1CCN(CC2CN(c3ccc(C(N)=O)c(CN4CC(F)C4)c3)C2)CC1] | 681.54 |
| 198 | 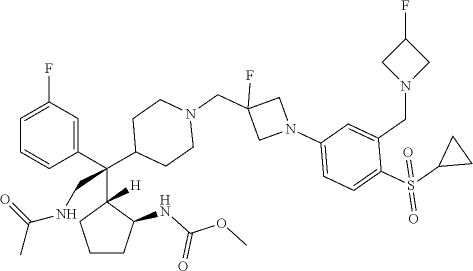 [SMILES: COC(=O)N[C@H]1CCC[C@@H]1[C@](CNC(C)=O)(c1cccc(F)c1)C1CCN(CC2(F)CN(c3ccc(S(=O)(=O)C4CC4)c(CN4CC(F)C4)c3)C2)CC1] | 760.29 |
| 199 | 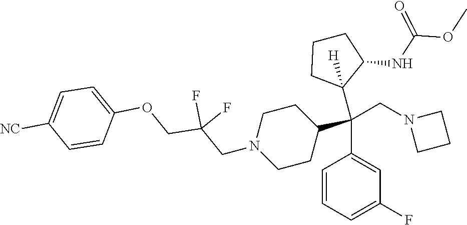 | 599.46 |
| 200 | 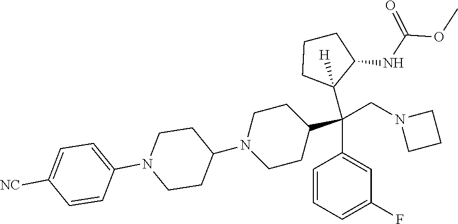 | 588.53 |
| 201 | 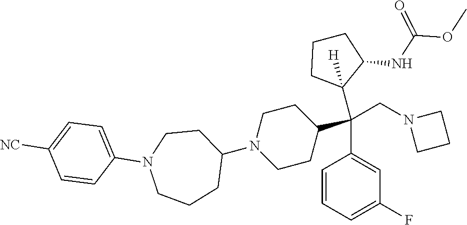 | 602.51 |
| 202 | 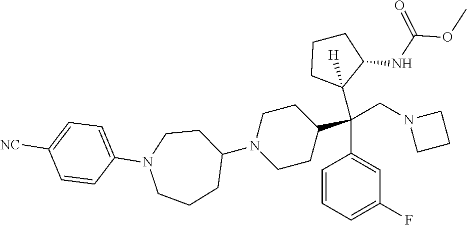 | 602.56 |
| 203 | 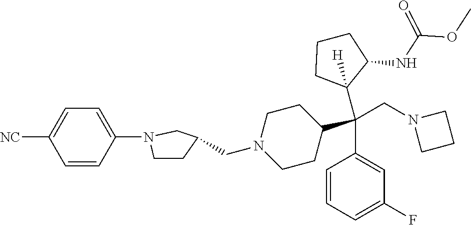 | 588.53 |
| 204 | 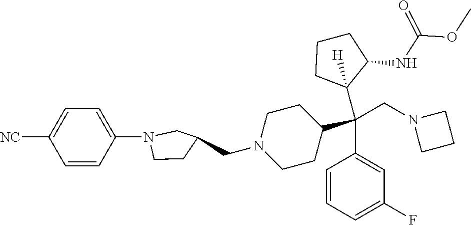 | 588.53 |
| 205 | 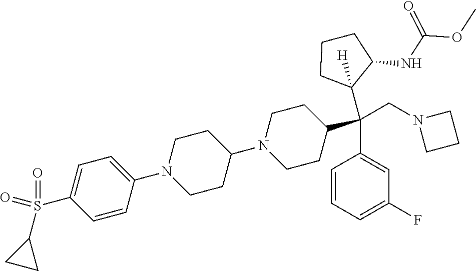 | 667.53 |
| 206 | 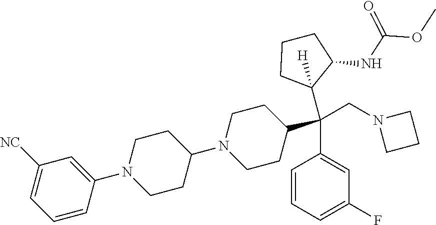 | 588.51 |
| 207 | 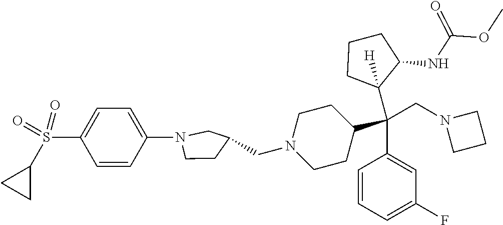 | 667.55 |
| 208 | 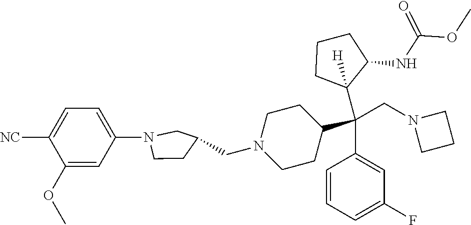 | 618.52 |
| 209 | 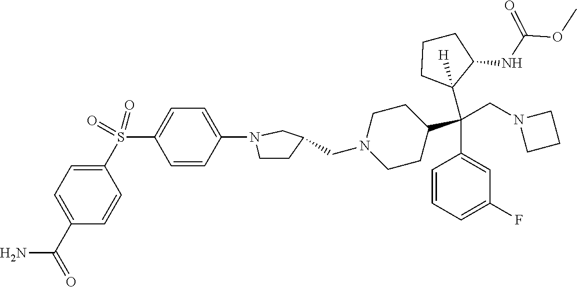 | 746.51 |
| 210 | 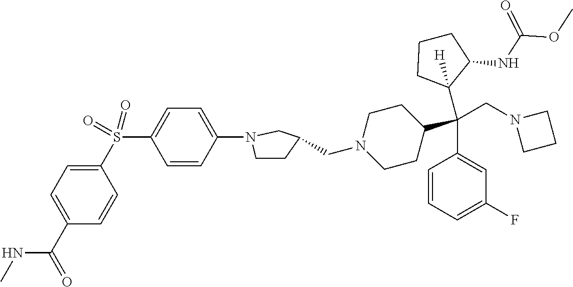 | 760.51 |
| 211 | 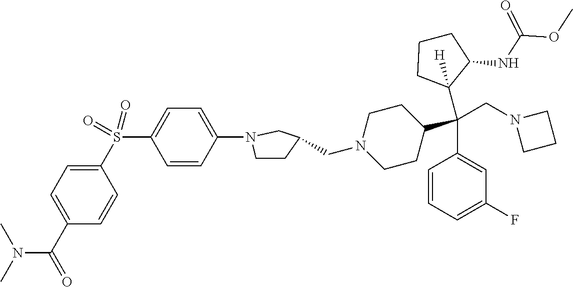 | 774.54 |
| 212 | 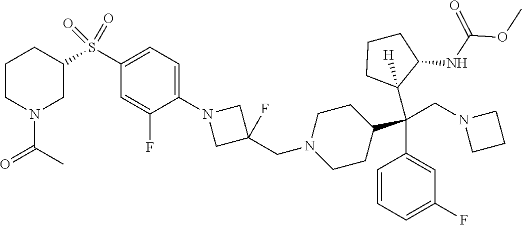 | 774.52 |
| 213 | 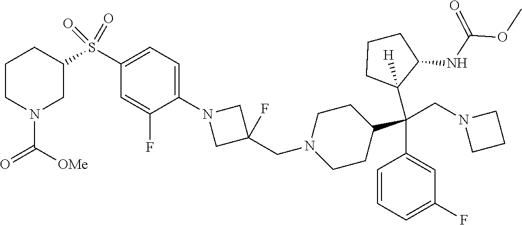 | 790.41 |
| 214 | 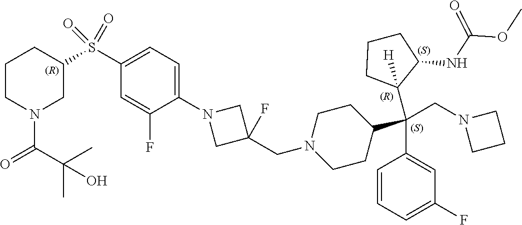 | 818.54 |
| 215 | 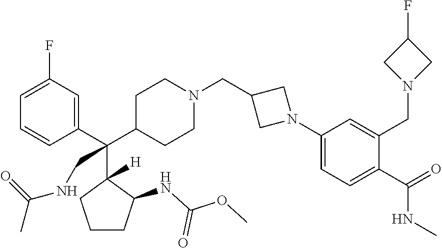 [SMILES: CNC(=O)c1ccc(N2CC(CN3CCC([C@@](CNC(C)=O)(c4cccc(F)c4)[C@H]4CCC[C@@H]4NC(=O)OC)CC3)C2)cc1CN1CC(F)C1] | |
| 216 | 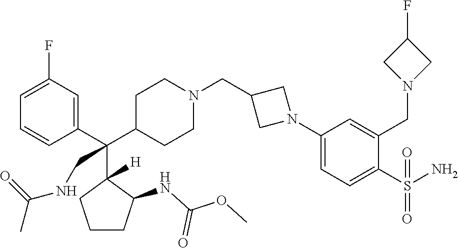 [SMILES: COC(=O)N[C@H]1CCC[C@@H]1[C@](CNC(C)=O)(c1cccc(F)c1)C1CCN(CC2CN(c3ccc(S(N)(=O)=O)c(CN4CC(F)C4)c3)C2)CC1] | |
| 217 | 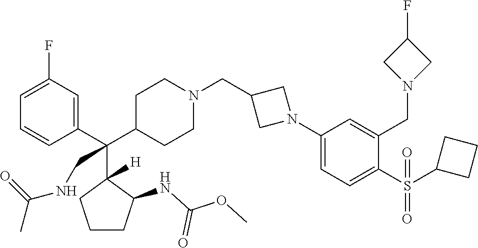 [SMILES: COC(=O)N[C@H]1CCC[C@@H]1[C@](CNC(C)=O)(c1cccc(F)c1)C1CCN(CC2CN(c3ccc(S(=O)(=O)C4CCC4)c(CN4CC(F)C4)c3)C2)CC1] | |
| 218 | 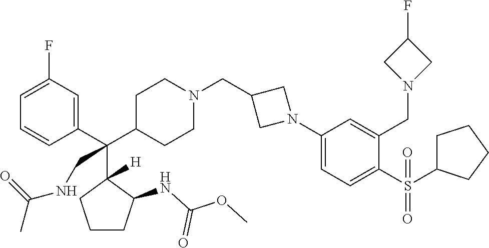 [SMILES: COC(=O)N[C@H]1CCC[C@@H]1[C@](CNC(C)=O)(c1cccc(F)c1)C1CCN(CC2CN(c3ccc(S(=O)(=O)C4CCCC4)c(CN4CC(F)C4)c3)C2)CC1] | |
| 219 | 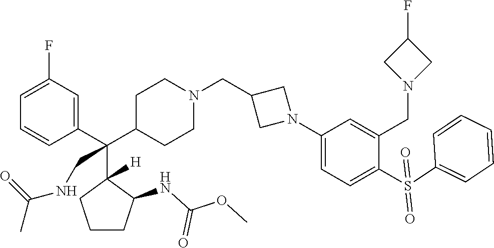 [SMILES: COC(=O)N[C@H]1CCC[C@@H]1[C@](CNC(C)=O)(c1cccc(F)c1)C1CCN(CC2CN(c3ccc(S(=O)(=O)c4ccccc4)c(CN4CC(F)C4)c3)C2)CC1] | |
| 220 | 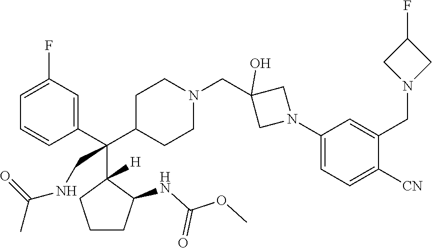 [SMILES: COC(=O)N[C@H]1CCC[C@@H]1[C@](CNC(C)=O)(c1cccc(F)c1)C1CCN(CC2(O)CN(c3ccc(C#N)c(CN4CC(F)C4)c3)C2)CC1] | |
| 221 | 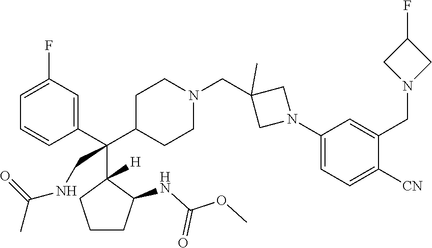 [SMILES: COC(=O)N[C@H]1CCC[C@@H]1[C@](CNC(C)=O)(c1cccc(F)c1)C1CCN(CC2(C)CN(c3ccc(C#N)c(CN4CC(F)C4)c3)C2)CC1] | |
| 222 | 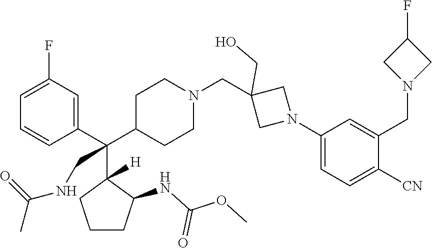 [SMILES: COC(=O)N[C@H]1CCC[C@@H]1[C@](CNC(C)=O)(c1cccc(F)c1)C1CCN(CC2(CO)CN(c3ccc(C#N)c(CN4CC(F)C4)c3)C2)CC1] | |
| 223 | 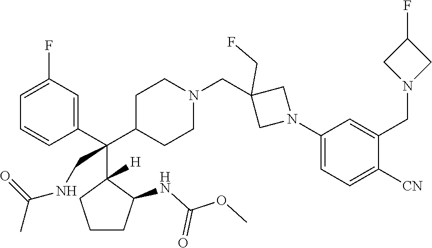 [SMILES: COC(=O)N[C@H]1CCC[C@@H]1[C@](CNC(C)=O)(c1cccc(F)c1)C1CCN(CC2(CF)CN(c3ccc(C#N)c(CN4CC(F)C4)c3)C2)CC1] | |
| 224 | 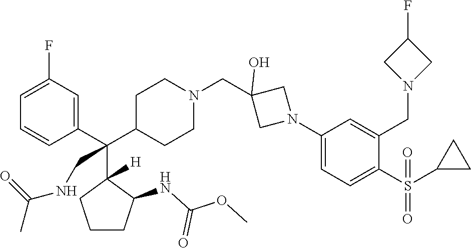 [SMILES: COC(=O)N[C@H]1CCC[C@@H]1[C@](CNC(C)=O)(c1cccc(F)c1)C1CCN(CC2(O)CN(c3ccc(S(=O)(=O)C4CC4)c(CN4CC(F)C4)c3)C2)CC1] | |
| 225 | 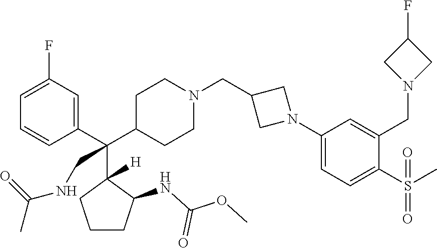 [SMILES: COC(=O)N[C@H]1CCC[C@@H]1[C@](CNC(C)=O)(c1cccc(F)c1)C1CCN(CC2CN(c3ccc(S(C)(=O)=O)c(CN4CC(F)C4)c3)C2)CC1] | |
| 226 | 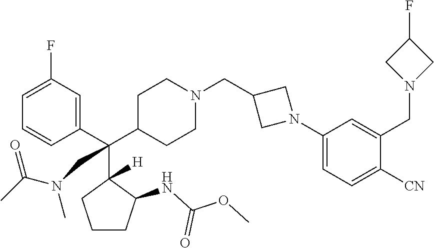 | |
| 227 | 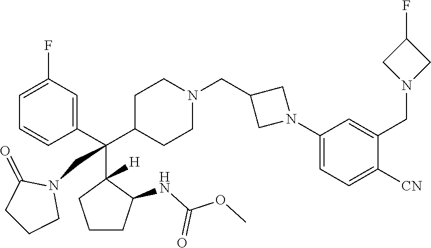 | |
| 228 | 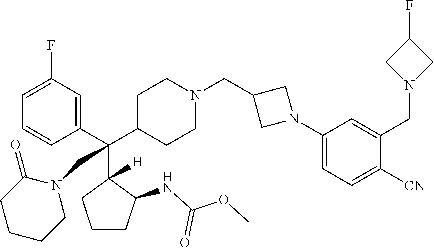 | |
| 229 | 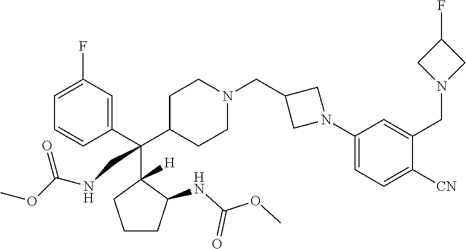 | |
| 230 | 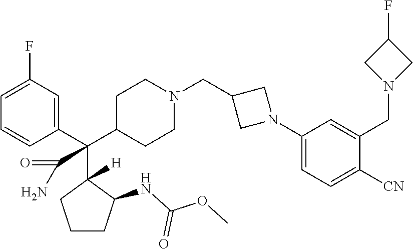 [SMILES: COC(=O)N[C@H]1CCC[C@@H]1[C@](C(N)=O)(c1cccc(F)c1)C1CCN(CC2CN(c3ccc(C#N)c(CN4CC(F)C4)c3)C2)CC1] | |
| 231 | 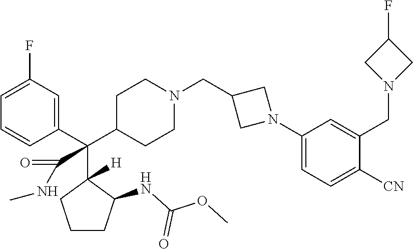 [SMILES: CNC(=O)[C@@](c1cccc(F)c1)(C1CCN(CC2CN(c3ccc(C#N)c(CN4CC(F)C4)c3)C2)CC1)[C@H]1CCC[C@@H]1NC(=O)OC] | |
| 232 | 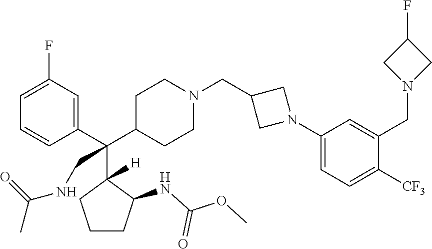 [SMILES: COC(=O)N[C@H]1CCC[C@@H]1[C@](CNC(C)=O)(c1cccc(F)c1)C1CCN(CC2CN(c3ccc(C(F)(F)F)c(CN4CC(F)C4)c3)C2)CC1] | |
| 233 | 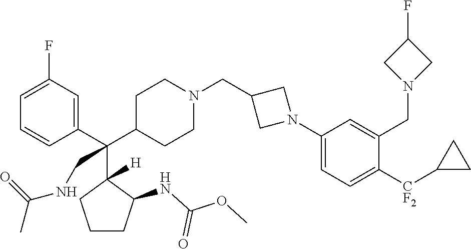 [SMILES: COC(=O)N[C@H]1CCC[C@@H]1[C@](CNC(C)=O)(c1cccc(F)c1)C1CCN(CC2CN(c3ccc(C(F)(F)C4CC4)c(CN4CC(F)C4)c3)C2)CC1] | |
| 234 | 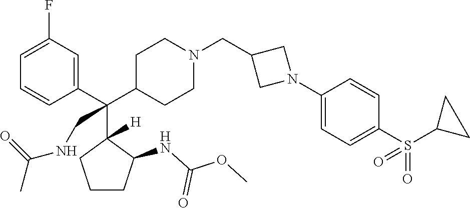 [SMILES: COC(=O)N[C@H]1CCC[C@@H]1[C@](CNC(C)=O)(c1cccc(F)c1)C1CCN(CC2CN(c3ccc(S(=O)(=O)C4CC4)cc3)C2)CC1] | |
| 235 | 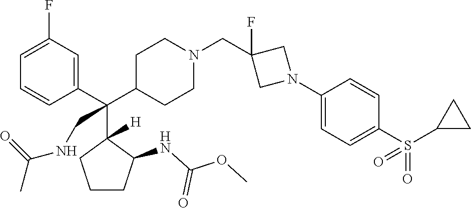 [SMILES: COC(=O)N[C@H]1CCC[C@@H]1[C@](CNC(C)=O)(c1cccc(F)c1)C1CCN(CC2(F)CN(c3ccc(S(=O)(=O)C4CC4)cc3)C2)CC1] | |
| 236 | 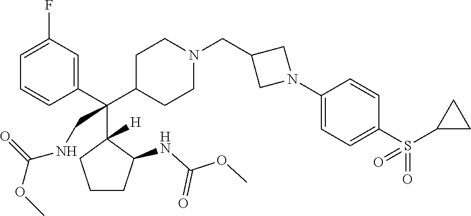 [SMILES: COC(=O)NC[C@@](c1cccc(F)c1)(C1CCN(CC2CN(c3ccc(S(=O)(=O)C4CC4)cc3)C2)CC1)[C@H]1CCC[C@@H]1NC(=O)OC] | |
| 237 | 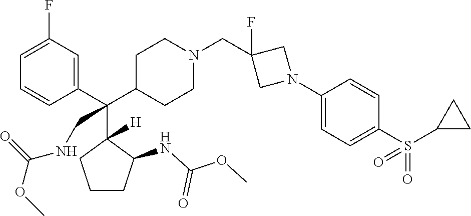 [SMILES: COC(=O)NC[C@@](c1cccc(F)c1)(C1CCN(CC2(F)CN(c3ccc(S(=O)(=O)C4CC4)cc3)C2)CC1)[C@H]1CCC[C@@H]1NC(=O)OC] | |
| 238 | 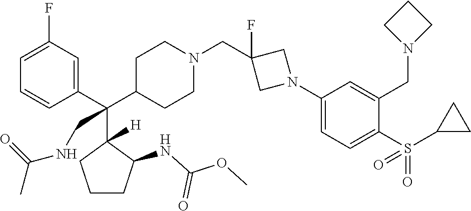 [SMILES: COC(=O)N[C@H]1CCC[C@@H]1[C@](CNC(C)=O)(c1cccc(F)c1)C1CCN(CC2(F)CN(c3ccc(S(=O)(=O)C4CC4)c(CN4CCC4)c3)C2)CC1] | |
| 239 | 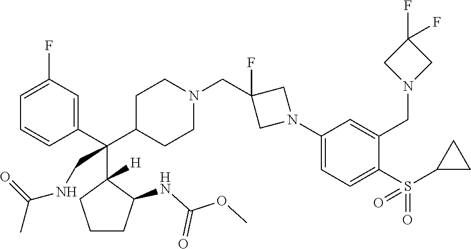 [SMILES: COC(=O)N[C@H]1CCC[C@@H]1[C@](CNC(C)=O)(c1cccc(F)c1)C1CCN(CC2(F)CN(c3ccc(S(=O)(=O)C4CC4)c(CN4CC(F)(F)C4)c3)C2)CC1] | |
| 241 | 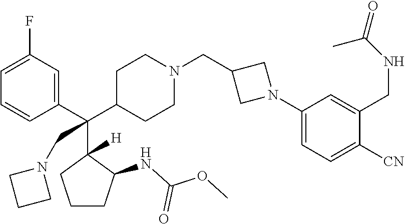 [SMILES: COC(=O)N[C@H]1CCC[C@@H]1[C@](CN1CCC1)(c1cccc(F)c1)C1CCN(CC2CN(c3ccc(C#N)c(CNC(C)=O)c3)C2)CC1] | |
| 242 | 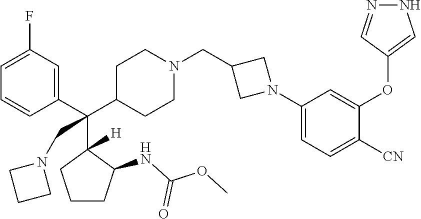 [SMILES: COC(=O)N[C@H]1CCC[C@@H]1[C@](CN1CCC1)(c1cccc(F)c1)C1CCN(CC2CN(c3ccc(C#N)c(Oc4cn[nH]c4)c3)C2)CC1] | |
| 243 | 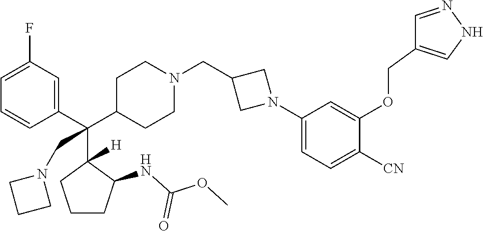 [SMILES: COC(=O)N[C@H]1CCC[C@@H]1[C@](CN1CCC1)(c1cccc(F)c1)C1CCN(CC2CN(c3ccc(C#N)c(OCc4cn[nH]c4)c3)C2)CC1] | |
| 244 | 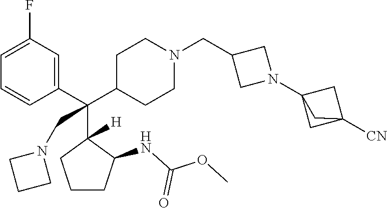 [SMILES: COC(=O)N[C@H]1CCC[C@@H]1[C@](CN1CCC1)(c1cccc(F)c1)C1CCN(CC2CN(C34CC(C#N)(C3)C4)C2)CC1] | |
| 245 | 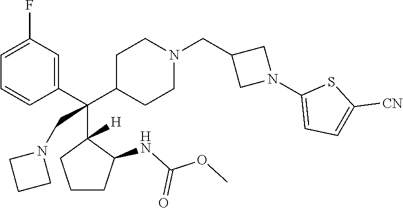 [SMILES: COC(=O)N[C@H]1CCC[C@@H]1[C@](CN1CCC1)(c1cccc(F)c1)C1CCN(CC2CN(c3ccc(C#N)s3)C2)CC1] | |
| 246 | 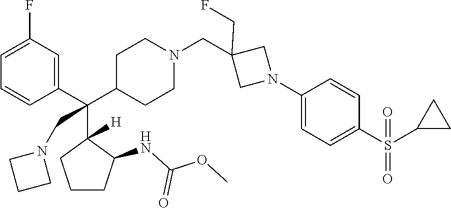 [SMILES: COC(=O)N[C@H]1CCC[C@@H]1[C@](CN1CCC1)(c1cccc(F)c1)C1CCN(CC2(CF)CN(c3ccc(S(=O)(=O)C4CC4)cc3)C2)CC1] | |
| 247 | 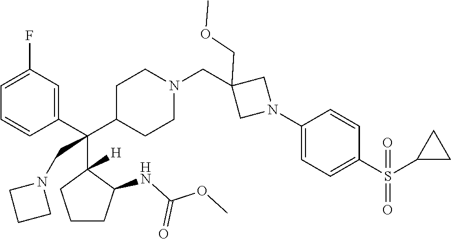 [SMILES: COCC1(CN2CCC([C@@](CN3CCC3)(c3cccc(F)c3)[C@H]3CCC[C@@H]3NC(=O)OC)CC2)CN(c2ccc(S(=O)(=O)C3CC3)cc2)C1] | |
| 248 | 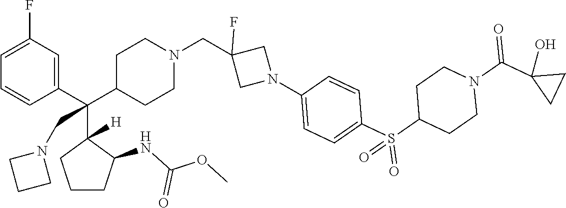 [SMILES: COC(=O)N[C@H]1CCC[C@@H]1[C@](CN1CCC1)(c1cccc(F)c1)C1CCN(CC2(F)CN(c3ccc(S(=O)(=O)C4CCN(C(=O)C5(O)CC5)CC4)cc3)C2)CC1] | |
| 249 | 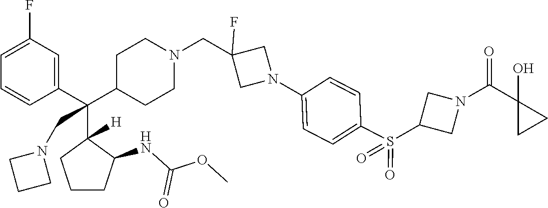 [SMILES: COC(=O)N[C@H]1CCC[C@@H]1[C@](CN1CCC1)(c1cccc(F)c1)C1CCN(CC2(F)CN(c3ccc(S(=O)(=O)C4CN(C(=O)C5(O)CC5)C4)cc3)C2)CC1] | |

TABLE 1.2A

| Cpd. No. | Chemical Name |
|---|---|
| 84 | methyl N-[(1S,2R)-2-[(1R)-2-(azetidin-1-yl)-1-(1-{[1-(4- |
| | cyanophenyl)azetidin-3-yl]methyl}piperidin-4-yl)-1-(3- |
| | fluorophenyl)ethyl]cyclopentyl]carbamate |
| 85 | methyl N-[(1S,2R)-2-[(1S)-2-(azetidin-1-yl)-1-({1-[4- |
| | (benzenesulfonyl)phenyl]-3-fluoroazetidin-3-yl}methyl)piperidin-4- |
| | yl]-1-(3-fluorophenyl)ethyl]cyclopentyl ]carbamate |
| 86 | methyl N-[(1S,2R)-2-[(1S)-2-(azetidin-1-yl)-1-{1-[3-(4- |
| | cyanophenoxy)-2-hydroxypropyl]piperidin-4-yl}-1-(3- |
| | fluorophenyl)ethyl]cyclopentyl]carbamate |
| 87 | methyl N-[(1S,2R)-2-[(1S)-2-(azetidin-1-yl)-1-{1-[(1-{4-[4- |
| | (dimethylcarbamoyl)benzenesulfonyl]phenyl}azetidin-3- |
| | yl)methyl]piperidin-4-yl}-1-(3-fluorophenyl)ethyl]cyclopentyl]carbamate |
| 88 | methyl N-[(1S,2R)-2-[(1S)-2-(azetidin-1-yl)-1-{1-[(3-fluoro-1-{4-[4- |
| | (methylcarbamoyl)benzenesulfonyl]phenyl}azetidin-3- |
| | yl)methyl]piperidin-4-yl}-1-(3-fluorophenyl)ethyl]cyclopentyl]carbamate |
| 89 | methyl N-[(1S,2R)-2-[(1S)-2-(azetidin-1-yl)-1-{1-[(1-{4 -[4- |
| | (dimethylcarbamoyl)benzenesulfonyl]phenyl}-3-fluoroazetidin-3- |
| | yl)methyl]piperidin-4-yl}-1-(3-fluorophenyl)ethyl]cyclopentyl]carbamate |
| 90 | methyl N-[(1S,2R)-2-[(1S)-2-(azetidin-1-yl)-1-(1-{[1-(3- |
| | cyanophenyl)azetidin-3-yl]methyl}piperidin-4-yl)-1-(3- |
| | fluorophenyl)ethyl]cyclopentyl]carbamate |
| 91 | methyl N-[(1S,2R)-2-[(1S)-2-(azetidin-1-yl)-1-{1-[3-(4- |
| | cyanophenoxy)-2-methoxypropyl]piperidin-4-yl}-1-(3- |
| | fluorophenyl)ethyl]cyclopentyl]carbamate |
| 92 | methyl N-[(1S,2R)-2-[(1S)-2-(azetidin-1-yl)-1-(3-fluorophenyl)-1-(1- |
| | {[1-(4-{[(1s,3s)-3-hydroxycyclobutyl]sulfonyl}phenyl)azetidin-3- |
| | yl]methyl}piperidin-4-yl)ethyl]cyclopentyl]carbamate |
| 93 | methyl N-[(1S,2R)-2-[(1S)-2-(azetidin-l-yl)-1-(3-fluorophenyl)-1-(1- |
| | {[1-(4-{[(3s)-3-hydroxy-3- |
| | (trifluoromethyl)cyclobutyl]sulfonyl}phenyl)azetidin-3- |
| | yl]methyl}piperidin-4-yl)ethyl]cyclopentyl]carbamate |
| 94 | methyl N-[(1S,2R)-2-[(1S)-2-(azetidin-1-yl)-1-(1-{[1-(4-cyano-2- |
| | methoxyphenyl)azetidin-3-yl]methyl}piperidin-4-yl)-1-(3- |
| | fluorophenyl)ethyl]cyclopentyl]carbamate |
| 95 | methyl N-[(1S,2R)-2-[(1S)-2-(azetidin-1-yl)-1-[1-({1-[4-(4- |
| | carbamoylbenzenesulfonyl)phenyl]azetidin-3-yl}methyl)piperidin-4- |
| | yl]-1-(3-fluorophenyl)ethyl]cyclopentyl]carbamate |
| 96 | methyl N-[(1S,2R)-2-[(1S)-2-(azetidin-1-yl)-1-(1-{[1-(4-cyano-2- |
| | methoxyphenyl)-3-fluoroazetidin-3-yl]methyl}piperidin-4-yl)-1-(3- |
| | fluorophenyl)ethyl]cyclopentyl]carbamate |
| 97 | methyl N-[(1S,2R)-2-[(1R)-2-(azetidin-1-yl)-1-(1-{[1-(3- |
| | cyanophenyl)azetidin-3-yl]methyl}piperidin-4-yl)-1-(3- |
| | fluorophenyl)ethyl]cyclopentyl]carbamate |
| 98 | methyl N-[(1S,2R)-2-[(1S)-2-(azetidin-1-yl)-1-[1-({1-[4-(4- |
| | carbamoylbenzenesulfonyl)phenyl]-3-fluoroazetidin-3- |
| | yl}methyl)piperidin-4-yl]-1-(3- |
| | fluorophenyl)ethyl]cyclopentyl]carbamate |
| 99 | methyl N-[(1S,2R)-2-[(1S)-2-(azetidin-1-yl)-1-{1-[1-(4- |
| | cyanophenyl)azetidine-3-carbonyl]piperidin-4-yl}-1-(3- |
| | fluorophenyl)ethyl]cyclopentyl]carbamate |
| 100 | methyl N-[(1S,2R)-2-[(1S)-2-(azetidin-1-yl)-1-[1-({1-[4-(3- |
| | carbamoylbenzenesulfonyl)phenyl]azetidin-3-yl}methyl)piperidin-4- |
| | yl]-1-(3-fluorophenyl)ethyl]cyclopentyl ]carbamate |
| 101 | methyl N-[(1S,2R)-2-[(1S)-2-(azetidin-1-yl)-1-(3-fluorophenyl)-1-{1- |
| | [(1-{4-[3-(methylcarbamoyl)benzenesulfonyl]phenyl}azetidin-3- |
| | yl)methyl]piperidin-4-yl}ethyl]cyclopentyl]carbamate |
| 102 | methyl N-[(1S,2R)-2-[(1S)-2-(azetidin-1-yl)-1-[1-(1{1-[4-(4- |
| | carbamoylbenzenesulfonyl)-2-fluorophenyl]azetidin-3- |
| | yl}methyl)piperidin-4-yl]-1-(3- |
| | fluorophenyl)ethyl]cyclopentyl]carbamate |
| 103 | methyl N-[(1S,2R)-2-[(1S)-2-(azetidin-1-yl)-1-{1-[(1-{2-fluoro-4-[4- |
| | (methylcarbamoyl)benzenesulfonyl]phenyl}azetidin-3- |
| | yl)methyl]piperidin-4-yl}-1-(3- |
| | fluorophenyl)ethyl]cyclopentyl]carbamate |
| 104 | methyl N-[(1S,2R)-2-[(1S)-2-(azetidin-1-yl)-1-{1-[(3-fluoro-1-{2- |
| | fluoro-4-[4-(methylcarbamoyl)benzenesulfonyl]phenyl}azetidin-3- |
| | yl)methyl]piperidin-4-yl}-1-(3- |
| | fluorophenyl)ethyl]cyclopentyl]carbamate |
| 105 | methyl N-[(1S,2R)-2-[(1S)-2-(azetidin-1-yl)-1-{1-[(1-{4-[4- |
| | (dimethylcarbamoyl)benzenesulfonyl]-2-fluorophenyl}-3- |
| | fluoroazetidin-3-yl)methyl]piperidin-4-yl}-1-(3- |
| | fluorophenyl)ethyl]cyclopentyl]carbamate |
| 106 | methyl N-[(1S,2R)-2-[(1S)-2-(azetidin-1-yl)-1-[1-({1-[4-(4- |
| | carbamoylbenzene sulfonyl)-2-fluorophenyl]-3-fluoroazetidin-3- |
| | yl}methyl)piperidin-4-yl]-1-(3- |
| | fluorophenyl)ethyl]cyclopentyl]carbamate |
| 107 | methyl N-[(1S,2R)-2-[(1S)-2-(azetidin-1-yl)-1-{1-[(1-{4-[4- |
| | (ethylcarbamoyl)benzenesulfonyl]phenyl}-3-fluoroazetidin-3- |
| | yl)methyl]piperidin-4-yl}-1-(3- |
| | fluorophenyl)ethyl]cyclopentyl]carbamate |
| 108 | methyl N-[(1S,2R)-2-[(1S)-2-(azetidin-1-yl)-1-(1-{[3-fluoro-1-(4-{4- |
| | [(propan-2-yl)carbamoy]benzenesulfonyl}phenyl)azetidin-3- |
| | yl]methyl}piperidin-4-yl)-1-(3- |
| | fluorophenyl)ethyl]cyclopentyl]carbamate |
| 109 | methyl N-[(1S,2R)-2-[(1S)-2-(azetidin-1-yl)-1-{1-[(1-{4-[4-(tert- |
| | butylcarbamoyl)benzenesulfonyl]phenyl}-3-fluoroazetidin-3- |
| | yl)methyl]piperidin-4-yl}-1-(3- |
| | fluorophenyl)ethyl]cyclopentyl]carbamate |
| 110 | methyl N-[(1S,2R)-2-[(1S)-2-(azetidin-1-yl)- 1-{1-[(1-{4-[4-(azetidine- |
| | 1-carbonyl)benzenesulfonyl]phenyl}-3-fluoroazetidin-3- |
| | yl)methyl]piperidin-4-yl}-1-(3- |
| | fluorophenyl)ethyl]cyclopentyl]carbamate |
| 111 | methyl N-[(1S,2R)-2-[(1S)-2-(azetidin-1-yl)-1-{1-[(3-fluoro-1-{4-[4- |
| | (morpholine-4-carbonyl)benzenesulfonyl]phenyl}azetidin-3- |
| | yl)methyl ]piperidin-4-yl}-1-(3- |
| | fluorophenyl)ethyl]cyclopentyl]carbamate |
| 112 | methyl N-[(1S,2R)-2-[(1S)-2-(azetidin-1-yl)-1-{1-[(3-fluoro-1-{4-[3- |
| | fluoro-4-(methylcarbamoyl)benzenesulfonyl]phenyl}azetidin-3- |
| | yl)methyl]piperidin-4-yl}-1-(3- |
| | fluorophenyl)ethyl]cyclopentyl]carbamate |
| 113 | methyl N-[(1S,2R)-2-[(1S)-2-(azetidin-1-yl)-1-{1-[(1-{4-[4- |
| | (dimethylcarbamoyl)-3-fluorobenzenesulfonyl]phenyl}-3- |
| | fluoroazetidin-3-yl)methyl]piperidin-4-yl}-1-(3 - |
| | fluorophenyl)ethyl]cyclopentyl]carbamate |
| 114 | methyl N-[(1S,2R)-2-[(1S)-2-(azetidin-1-yl)-1-[1-({1-[4-(4-carbamoyl- |
| | 3-fluorobenzenesulfonyl)phenyl]-3-fluoroazetidin-3- |
| | yl}methyl)piperidin-4-yl]-1-(3- |
| | fluorophenyl)ethyl]cyclopentyl]carbamate |
| 115 | methyl N-[(1S,2R)-2-[(1S)-2-(azetidin-1-yl)-1-{1-[(1-{4-[(6- |
| | carbamoylpyridin-3-yl)sulfonyl]phenyl}-3-fluoroazetidin-3 - |
| | yl)methyl]piperidin-4-yl}-1-(3- |
| | fluorophenyl)ethyl]cyclopentyl]carbamate |
| 116 | methyl N-[(1S,2R)-2-[(1S)-2-(azetidin-1-yl)-1-(1-{[3-fluoro-1-(4-{[6- |
| | (methylcarbamoyl)pyridin-3-yl]sulfonyl}phenyl)azetidin-3- |
| | yl]methyl}piperidin-4-yl)-1-(3- |
| | fluorophenyl)ethyl]cyclopentyl]carbamate |
| 117 | methyl N-[(1S,2R)-2-[(1S)-2-(azetidin-1-yl)-1-(1-{[1-(4-{[6- |
| | (dimethylcarbamoyl)pyridin-3-yl]sulfonyl}phenyl)-3-fluoroazetidin-3- |
| | yl]methyl}piperidin-4-yl)-1-(3- |
| | fluorophenyl)ethyl]cyclopentyl]carbamate |
| 118 | methyl N-[(1S,2R)-2-[(1S)-2-(azetidin-1-yl)-1-[1-({1-[4-(4-carbamoyl- |
| | 3-fluorobenzenesulfonyl)-2-fluorophenyl]-3-fluoroazetidin-3- |
| | yl}methyl)piperidin-4-yl]-1-(3- |
| | fluorophenyl)ethyl]cyclopentyl]carbamate |
| 119 | methyl N-[(1S,2R)-2-[(1S)-2-(azetidin-1-yl)-1-{1-[(3-fluoro-1-{2- |
| | fluoro-4-[3-fluoro-4-(methylcarbamoyl)benzenesulfonyl]phenyl}azetidin- |
| | 3-yl)methyl]piperidin-4-yl}-1-(3- |
| | fluorophenyl)ethyl]cyclopentyl]carbamate |
| 120 | methyl N-[(1S,2R)-2-[(1S)-2-(azetidin-1-yl)-1-{1-[(1-{4-[4- |
| | (dimethylcarbamoyl)-3-fluorobenzenesulfonyl]-2-fluorophenyl}-3- |
| | fluoroazetidin-3-yl)methyl]piperidin-4-yl}-1-(3- |
| | fluorophenyl)ethyl]cyclopentyl]carbamate |
| 121 | methyl N-[(1S,2R)-2-[(1S)-2-(azetidin-1-yl)-1-{1-[(1-{4-[(6- |
| | carbamoylpyridin-3-yl)sulfonyl]-2-fluorophenyl}-3-fluoroazetidin-3- |
| | yl)methyl]piperidin-4-yl}-1-(3- |
| | fluorophenyl)ethyl]cyclopentyl]carbamate |
| 122 | methyl N-[(1S,2R)-2-[(1S)-2-(azetidin-1-yl)-1-(1-{[3-fluoro-1-(2- |
| | fluoro-4-{[6-(methylcarbamoyl)pyridin-3-yl]sulfonyl}phenyl)azetidin- |
| | 3-yl]methyl}piperidin-4-yl)-1-(3- |
| | fluorophenyl)ethyl]cyclopenty]carbamate |
| 123 | methyl N-[(1S,2R)-2-[(1S)-2-(azetidin-1-yl)-1-(1-{[1-(4-{[6- |
| | (dimethylcarbamoyl)pyridin-3-yl]sulfonyl}-2-fluorophenyl)-3- |
| | fluoroazetidin-3-yl]methyl}piperidin-4-yl)-1-(3- |
| | fluorophenyl)ethyl]cyclopentyl]carbamate |
| 124 | methyl N-[(1S,2R)-2-[(1S)-2-(azetidin-1-yl)-1-[1-({1-[4-(3- |
| | carbamoylbenzenesulfonyl)phenyl]-3 -fluoroazetidin-3- |
| | yl}methyl)piperidin-4-yl]-1-(3- |
| | fluorophenyl)ethyl]cyclopentyl]carbamate |
| 125 | methyl N-[(1S,2R)-2-[(1S)-2-(azetidin-1-yl)-1-{1-[(3-fluoro-1-{4-[3- |
| | (methylcarbamoyl)benzenesulfonyl]phenyl}azetidin-3- |
| | yl)methyl]piperidin-4-yl}-1-(3- |
| | fluorophenyl)ethyl]cyclopentyl]carbamate |
| 126 | methyl N-[(1S,2R)-2-[(1S)-2-(azetidin-1-yl)-1-{1-[(1-{4-[3- |
| | (dimethylcarbamoyl)benzenesulfonyl]phenyl}-3-fluoroazetidin-3- |
| | yl)methyl]piperidin-4-yl}-1-(3- |
| | fluorophenyl)ethyl]cyclopentyl]carbamate |
| 127 | methyl N-[(1S,2R)-2-[(1S)-2-(azetidin-1-yl)-1-[1-({1-[4-(3- |
| | carbamoylbenzene sulfonyl)-2-fluorophenyl]-3-fluoroazetidin-3- |
| | yl}methyl)piperidin-4-yl]-1-(3- |
| | fluorophenyl)ethyl]cyclopentyl]carbamate |
| 128 | methyl N-[(1S,2R)-2-[(1S)-2-(azetidin-1-yl)-1-{1-[(3-fluoro-1-{2- |
| | fluoro-4-[3-(methylcarbamoyl)benzenesulfonyl]phenyl}azetidin-3- |
| | yl)methyl]piperidin-4-yl}-1-(3- |
| | fluorophenyl)ethyl]cyclopentyl]carbamate |
| 129 | methyl N-[(1S,2R)-2-[(1S)-2-(azetidin-1-yl)-1-{1-[(1-{4-[3- |
| | (dimethylcarbamoyl)benzenesulfonyl]-2-fluorophenyl}-3- |
| | fluoroazetidin-3-yl)methyl]piperidin-4-yl}-1-(3- |
| | fluorophenyl)ethyl]cyclopentyl]carbamate |
| 130 | methyl N-[(1S,2R)-2-[(1S)-2-(azetidin-1-yl)-1-[1-({1-[4-(3- |
| | acetamidobenzenesulfonyl)-2-fluorophenyl]-3-fluoroazetidin-3- |
| | yl}methyl)piperidin-4-yl]-1-(3- |
| | fluorophenyl)ethyl]cyclopentyl]carbamate |
| 131 | methyl N-[(1S,2R)-2-[(1S)-2-(azetidin-1-yl)-1-[1-({3-fluoro-1-[2- |
| | fluoro-4-(3-propanamidobenzenesulfonyl)phenyl]azetidin-3- |
| | yl}methyl)piperidin-4-yl]-1-(3- |
| | fluorophenyl)ethyl]cyclopentyl]carbamate |
| 132 | methyl N-[(1S,2R)-2-[(1S)-2-(azetidin-1-yl)-1-{1-[(1-{4-cyano-3- |
| | [(morpholin-4-yl)methyl]phenyl}azetidin-3-yl)methyl]piperidin-4-yl}- |
| | 1-(3-fluorophenyl)ethyl]cyclopentyl]carbamate |
| 133 | methyl N-[(1S,2R)-2-[(1S)-2-(azetidin-1-yl)-1-{1-[(1-{4-cyano-3- |
| | [(piperazin-1-yl)methyl]phenyl}azetidin-3-yl)methyl]piperidin-4-yl}-1- |
| | (3-fluorophenyl)ethyl]cyclopentyl]carbamate |
| 134 | methyl N-[(1S,2R)-2-[(1S)-2-(azetidin-1-yl)-1-(1-{[1-(4-cyano-3- |
| | methoxyphenyl)azetidin-3-yl]methyl}piperidin-4-yl)-1-(3- |
| | fluorophenyl)ethyl]cyclopentyl]carbamate |
| 135 | methyl N-[(1S,2R)-2-[(1S)-2-(azetidin-1-yl)-1-[1-({1-[4-cyano-3-(2- |
| | methoxyethoxy)phenyl]azetidin-3-yl}methyl)piperidin-4-yl]-1-(3- |
| | fluorophenyl)ethyl]cyclopentyl]carbamate |
| 136 | methyl N-[(1S,2R)-2-[(1S)-2-(azetidin-1-yl)-1-{1-[(1-{4-cyano-3- |
| | [(oxan-4-yl)methoxy]phenyl}azetidin-3-yl)methyl]piperidin-4-yl}-1- |
| | (3-fluorophenyl)ethyl]cyclopentyl]carbamate |
| 137 | methyl N-[(1S,2R)-2-[(1S)-2-(azetidin-1-yl)-1-{1-[(1-{4-cyano-3-[(4- |
| | methylpiperazin-1-yl)methyl]phenyl}azetidin-3-yl)methyl]piperidin-4- |
| | yl}-1-(3-fluorophenyl)ethyl]cyclopentyl]carbamate |
| 138 | methyl N-[(1S,2R)-2-[(1S)-2-(azetidin-1-yl)-1-{1-[(3-fluoro-1-{4-[4- |
| | (3-hydroxyazetidine-1-carbonyl)benzenesulfonyl]phenyl}azetidin-3- |
| | yl)methyl]piperidin-4-yl}-1-(3- |
| | fluorophenyl)ethyl]cyclopentyl]carbamate |
| 139 | methyl N-[(1S,2R)-2-[(1S)-2-(azetidin-1-yl)-1-{1-[(3-fluoro-1-{4-[4- |
| | (3-hydroxy-3-methylazetidine-1- |
| | carbonyl)benzenesulfonyl]phenyl}azetidin-3-yl)methyl]piperidin-4- |
| | yl}-1-(3-fluorophenyl)ethyl]cyclopentyl]carbamate |
| 140 | methyl N-[(1S,2R)-2-[(1S)-2-(azetidin-1-yl)-1-{1-[(3-fluoro-1-{4-[4- |
| | (4-hydroxypiperidine-1-carbonyl)benzenesulfonyl]phenyl}azetidin-3- |
| | yl)methyl]piperidin-4-yl}-1-(3- |
| | fluorophenyl)ethyl]cyclopentyl]carbamate |
| 141 | methyl N-[(1S,2R)-2-[(1S)-2-(azetidin-1-yl)-1-{1-[(3-fluoro-1-{4-[4- |
| | (4-hydroxy-4-methylpiperidine-1- |
| | carbonyl)benzenesulfonyl]phenyl}azetidin-3-yl)methyl]piperidin-4- |
| | yl}-1-(3-fluorophenyl)ethyl]cyclopentyl]carbamate |
| 142 | methyl N-[(1S,2R)-2-[(1S)-2-(azetidin-1-yl)-1-{1-[(1-{4- |
| | cyanobicyclo[2.2.2]octan-1-yl}azetidin-3-yl)methyl]piperidin-4-yl}-1- |
| | (3-fluorophenyl)ethyl]cyclopentyl]carbamate |
| 143 | methyl N-[(1S,2R)-2-[(1S)-2-(azetidin-1-yl)-1-[1-({3-fluoro-1-[4- |
| | (oxetane-3-sulfonyl)phenyl]azetidin-3-yl}methyl)piperidin-4-yl]-1-(3- |
| | fluorophenyl)ethyl]cyclopentyl]carbamate |
| 144 | methyl N-[(1S,2R)-2-[(1S)-2-(azetidin-1-yl)-1-[1-({1-[4-(azetidine-3- |
| | sulfonyl)phenyl]-3-fluoroazetidin-3-yl}methyl)piperidin-4-yl]-1-(3- |
| | fluorophenyl)ethyl]cyclopentyl]carbamate |
| 145 | methyl N-[(1S,2R)-2-[(1S)-2-(azetidin-1-yl)-1-[1-({1-[4-(azetidine-3- |
| | sulfonyl)-2-fluorophenyl]-3-fluoroazetidin-3-yl}methyl)piperidin-4- |
| | yl]-1-(3-fluorophenyl)ethyl]cyclopentyl]carbamate |
| 146 | methyl 3-({4-[(1S)-2-(azetidin-1-yl)-1-(3-fluorophenyl)-1-[(1R,2S)-2- |
| | [(methoxycarbonyl)amino]cyclopentyl]ethyl]piperidin-1-yl}methyl)-1- |
| | [4-(cyclopropanesulfonyl)phenyl]azetidine-3-carboxylate |
| 147 | methyl N-[(1S,2R)-2-[(1S)-2-(azetidin-1-yl)-1-{1-[(1-{4-cyano-3- |
| | [(dimethylamino)methyl]phenyl}azetidin-3-yl)methyl]piperidin-4-yl}- |
| | 1-(3-fluorophenyl)ethyl]cyclopentyl]carbamate |
| 148 | methyl N-[(1S,2R)-2-[(1S)-2-(azetidin-1-yl)-1-{1-[(3-fluoro-1-{4- |
| | [(oxan-4-yl)methanesulfonyl]phenyl}azetidin-3-yl)methyl]piperidin-4- |
| | yl}-1-(3-fluorophenyl)ethyl]cyclopentyl]carbamate |
| 149 | methyl N-[(1S,2R)-2-[(1S)-1-{1-[(1-{4-[(1-acetylpiperidin-4- |
| | yl)sulfonyl]phenyl}-3-fluoroazetidin-3-yl)methyl]piperidin-4-yl}-2- |
| | (azetidin-1-yl)-1-(3-fluorophenyl)ethyl]cyclopentyl]carbamate |
| 150 | 3-({4-[(1S)-2-(azetidin-1-yl)-1-(3-fluorophenyl)-1-[(1R,2S)-2- |
| | [(methoxycarbonyl)amino]cyclopentyl]ethyl]piperidin-1-yl}methyl)-1- |
| | [4-(cyclopropanesulfonyl)phenyl]azetidine-3-carboxylic acid |
| 151 | methyl N-[(1S,2R)-2-[(1S)-2-(azetidin-1-yl)-1-[1-({1-[4- |
| | (cyclopropanesulfonyl)phenyl]-3-(methylcarbamoyl)azetidin-3- |
| | yl}methyl)piperidin-4-yl]-1-(3- |
| | fluorophenyl)ethyl]cyclopentyl]carbamate |
| 152 | methyl N-[(1S,2R)-2-[(1S)-2-(azetidin-1-yl)-1-(1-{[3-fluoro-1-(4-{[1- |
| | (2-hydroxy-2-methylpropanoyl)piperidin-4- |
| | yl]sulfonyl}phenyl)azetidin-3-yl]methyl}piperidin-4-yl)-1-(3- |
| | fluorophenyl)ethyl]cyclopentyl]carbamate |
| 153 | methyl N-[(1S,2R)-2-[(1S)-2-(azetidin-1-yl)-1-[1-({3-fluoro-1-[4-(2- |
| | hydroxy-2-methylpropanesulfonyl)phenyl]azetidin-3- |
| | yl}methyl)piperidin-4-yl]-1-(3- |
| | fluorophenyl)ethyl]cyclopentyl]carbamate |
| 154 | methyl N-[(1S,2R)-2-[(1S)-2-(azetidin-1-yl)-1-(1-{[1-(4-{[(2S)-1,4- |
| | dioxan-2-yl]methanesulfonyl}phenyl)-3-fluoroazetidin-3- |
| | yl]methyl}piperidin-4-yl)-1-(3- |
| | fluorophenyl)ethyl]cyclopentyl]carbamate |
| 155 | methyl N-[(1S,2R)-2-[(1S)-2-(azetidin-1-yl)-1-(1-{[1-(4-{[(2R)-1,4- |
| | dioxan-2-yl]methanesulfonyl}phenyl)-3-fluoroazetidin-3- |
| | yl]methyl}piperidin-4-yl)-1-(3- |
| | fluorophenyl)ethyl]cyclopentyl]carbamate |
| 156 | methyl N-[(1S,2R)-2-[(1S)-2-(azetidin-1-yl)-1-(1-{[3-fluoro-1-(4-{4- |
| | [(2-hydroxy-2- |
| | methylpropyl)carbamoyl]benzenesulfonyl}phenyl)azetidin-3- |
| | yl]methyl}piperidin-4-yl)-1-(3- |
| | fluorophenyl)ethyl]cyclopentyl]carbamate |
| 157 | methyl N-[(1S,2R)-2-[(1S)-2-(azetidin-1-yl)-1-(1-{[3-fluoro-1-(4-{4- |
| | [4-hydroxy-4-(trifluoromethyl)piperidine-1- |
| | carbonyl]benzenesulfonyl}phenyl)azetidin-3-yl]methyl}piperidin-4- |
| | yl)-1-(3-fluorophenyl)ethyl]cyclopentyl]carbamate |
| 158 | methyl N-[(1S,2R)-2-[(1S)-2-(azetidin-1-yl)-1-{1-[(1-{4-cyano-3- |
| | [(dimethylamino)methyl]phenyl}-3-fluoroazetidin-3- |
| | yl)methyl]piperidin-4-yl}-1-(3- |
| | fluorophenyl)ethyl]cyclopentyl]carbamate |
| 159 | methyl N-[(1S,2R)-2-[(1S)-2-(azetidin-1-yl)-1-[1-({3-fluoro-1-[4-(4- |
| | {2-oxa-7-azaspiro[3.5]nonane-7- |
| | carbonyl}benzenesulfonyl)phenyl]azetidin-3-yl}methyl)piperidin-4- |
| | yl]-1-(3-fluorophenyl)ethyl]cyclopentyl]carbamate |
| 160 | methyl N-[(1S,2R)-2-[(1S)-2-(azetidin-1-yl)-1-[1-({3-fluoro-1-[4-(4- |
| | {[(oxetan-3-yl)methyl]carbamoyl}benzenesulfonyl)phenyl]azetidin-3- |
| | yl}methyl)piperidin-4-yl]-1-(3- |
| | fluorophenyl)ethyl]cyclopentyl]carbamate |
| 161 | methyl N-[(1S,2R)-2-[(1S)-2-(azetidin-1-yl)-1-{1-[(1-{3-[(azetidin-1- |
| | yl)methyl]-4-cyanophenyl}azetidin-3-yl)methyl]piperidin-4-yl}-1-(3- |
| | fluorophenyl)ethyl]cyclopentyl]carbamate |
| 162 | methyl N-[(1S,2R)-2-[(1S)-2-(azetidin-1-yl)-1-{1-[(1-{4-cyano-3-[(3- |
| | fluoroazetidin-1-yl)methyl]phenyl}azetidin-3-yl)methyl]piperidin-4- |
| | yl}-1-(3-fluorophenyl)ethyl]cyclopentyl]carbamate |
| 163 | methyl N-[(1S,2R)-2-[(1S)-2-(azetidin-1-yl)-1-{1-[(1-{4-cyano-3- |
| | [(3,3-difluoroazetidin-1-yl)methyl]phenyl}azetidin-3- |
| | yl)methyl]piperidin-4-yl}-1-(3- |
| | fluorophenyl)ethyl]cyclopentyl]carbamate |
| 164 | methyl N-[(1S,2R)-2-[(1S)-2-(azetidin-1-yl)-1-(1-{[3-fluoro-1-(4-{4- |
| | [(oxetan-3-yl)carbamoyl]benzenesulfonyl}phenyl)azetidin-3 - |
| | yl]methyl}piperidin-4-yl)-1-(3- |
| | fluorophenyl)ethyl]cyclopentyl]carbamate |
| 165 | methyl N-[(1S,2R)-2-[(1S)-2-(azetidin-1-yl)-1-{1-[(1-{4-[(1,4- |
| | dioxepan-6-yl)methanesulfonyl]phenyl}-3-fluoroazetidin-3- |
| | yl)methyl ]piperidin-4-yl}-1-(3- |
| | fluorophenyl)ethyl]cyclopentyl]carbamate |
| 166 | methyl N-[(1S,2R)-2-[(1S)-1-(1-{[1-(4-cyanophenyl)azetidin-3- |
| | yl]methyl}piperidin-4-yl)-2-acetamido-1-(3- |
| | fluorophenyl)ethyl]cyclopentyl]carbamate |
| 167 | methyl N-[(1S,2R)-2-[(1S)-2-(azetidin-1-yl)-1-{1-[(1-{4-cyano-3-[(3- |
| | hydroxyazetidin-1-yl)methyl]phenyl}azetidin-3-yl)methyl]piperidin-4- |
| | yl}-1-(3-fluorophenyl)ethyl]cyclopentyl]carbamate |
| 168 | methyl N-[(1S,2R)-2-[(1S)-2-(azetidin-1-yl)-1-{1-[(1-{4-cyano-3-[(3- |
| | hydroxy-3-methylazetidin-1-yl)methyl]phenyl}azetidin-3- |
| | yl)methyl]piperidin-4-yl}-1-(3- |
| | fluorophenyl)ethyl]cyclopentyl]carbamate |
| 169 | methyl N-[(1S,2R)-2-[(1S)-1-{1-[(1-{4-cyano-3- |
| | [(dimethylamino)methyl]phenyl}azetidin-3-yl)methyl]piperidin-4-yl}- |
| | 2-acetamido-1-(3-fluorophenyl)ethyl]cyclopentyl]carbamate |
| 170 | methyl N-[(1S,2R)-2-[(1S)-2-(azetidin-1-yl)-1-(1-{[1-(5-cyano-1,3- |
| | thiazol-2-yl)azetidin-3-yl ]methyl}piperidin-4-yl)-1-(3- |
| | fluorophenyl)ethyl]cyclopentyl]carbamate |
| 171 | methyl N-[(1S,2R)-2-[(1S)-2-(azetidin-1-yl)-1-(1-{[3-fluoro-1-(4-{[1- |
| | (3-hydroxy-3 -methylbutanoyl)piperidin-4-yl]sulfonyl}phenyl)azetidin- |
| | 3-yl]methyl}piperidin-4-yl)-1-(3- |
| | fluorophenyl)ethyl]cyclopentyl]carbamate |
| 172 | methyl N-[(1S,2R)-2-[(1S)-2-(azetidin-1-yl)-1-(1-{[3-fluoro-1-(4-{[1- |
| | (3-hydroxy-3-methylbutanoyl)azetidin-3-yl]sulfonyl}phenyl)azetidin- |
| | 3-yl]methyl}piperidin-4-yl)-1-(3- |
| | fluorophenyl)ethyl]cyclopentyl]carbamate |
| 173 | methyl N-[(1S,2R)-2-[(1S)-2-(azetidin-1-yl)-1-{1-[(1-{3-[(azetidin-1- |
| | yl)methyl]-4-cyanophenyl}-3-fluoroazetidin-3-yl)methyl]piperidin-4- |
| | yl}-1-(3-fluorophenyl)ethyl]cyclopentyl]carbamate |
| 174 | methyl N-[(1S,2R)-2-[(1S)-2-(azetidin-1-yl)-1-{1-[(1-{4-cyano-3-[(3- |
| | fluoroazetidin-1-yl)methyl]phenyl}-3-fluoroazetidin-3- |
| | yl)methyl]piperidin-4-yl}-1-(3- |
| | fluorophenyl)ethyl]cyclopentyl]carbamate |
| 175 | methyl N-[(1S,2R)-2-[(1S)-1-{1-[(1-{3-[(azetidin-1-yl)methyl]-4- |
| | cyanophenyl}azetidin-3-yl)methyl]piperidin-4-yl}-2-acetamido-1-(3- |
| | fluorophenyl)ethyl]cyclopentyl]carbamate |
| 176 | methyl N-[(1S,2R)-2-[(1S)-1-{1-[(1-{4-cyano-3-[(3-fluoroazetidin-1- |
| | yl)methyl]phenyl}azetidin-3-yl)methyl]piperidin-4-yl}-2-acetamido-1- |
| | (3-fluorophenyl)ethyl]cyclopentyl]carbamate |
| 177 | methyl N-[(1S,2R)-2-[(1S)-1-{1-[(1-{3-[(azetidin-1-yl)methyl]-4- |
| | cyanophenyl}-3-fluoroazetidin-3-yl)methyl]piperidin-4-yl}-2- |
| | acetamido-1-(3-fluorophenyl)ethyl]cyclopentyl]carbamate |
| 178 | methyl N-[(1S,2R)-2-[(1S)-1-{1-[(1-{4-cyano-3-[(3-fluoroazetidin-1- |
| | yl)methyl]phenyl}-3-fluoroazetidin-3-yl)methyl]piperidin-4-yl}-2- |
| | acetamido-1-(3-fluorophenyl)ethyl]cyclopentyl]carbamate |
| 179 | methyl N-[(1S,2R)-2-[(1S)-2-(azetidin-1-yl)-1-{14(1-{4-cyano-3- |
| | [(1H-pyrrol-1-yl)methyl]phenyl}azetidin-3-yl)methyl]piperidin-4-yl}- |
| | 1-(3-fluorophenyl)ethyl]cyclopentyl]carbamate |
| 180 | methyl N-[(1S,2R)-2-[(1S)-2-(azetidin-1-yl)-1-{1-[(1-{4-cyano-3- |
| | [(1H-pyrazol-1-yl)methyl]phenyl}azetidin-3-yl)methyl]piperidin-4-yl}- |
| | 1-(3-fluorophenyl)ethyl]cyclopentyl]carbamate |
| 181 | methyl N-[(1S,2R)-2-[(1S)-2-(azetidin-1-yl)-1-{1-[(1-{4-cyano-3- |
| | [(1H-imidazol-1-yl)methyl]phenyl}azetidin-3-yl)methyl]piperidin-4- |
| | yl}-1-(3-fluorophenyl)ethyl]cyclopentyl]carbamate |
| 182 | methyl N-[(1S,2R)-2-[(1S)-1-{1-[(1-{4-cyano-3- |
| | [(dimethylamino)methyl]phenyl}-3-fluoroazetidin-3- |
| | yl)methyl]piperidin-4-yl}-2-acetamido-1-(3- |
| | fluorophenyl)ethyl]cyclopentyl]carbamate |
| 183 | methyl N-[(1S,2R)-2-[(S)-cyano({1-[(1-{4-cyano-3- |
| | [(dimethylamino)methyl]phenyl}azetidin-3-yl)methyl]piperidin-4- |
| | yl})(3-fluorophenyl)methyl]cyclopentyl]carbamate |
| 184 | methyl N-[(1S,2R)-2-[(1S)-2-(azetidin-1-yl)-1-{1-[(1-{4-cyano-3- |
| | [(1H-pyrrol-1-yl)methyl]phenyl}-3-fluoroazetidin-3- |
| | yl)methyl]piperidin-4-yl}-1-(3- |
| | fluorophenyl)ethyl]cyclopentyl]carbamate |
| 185 | methyl N-[(1S,2R)-2-[(1S)-2-(azetidin-1-yl)-1-{1-[(1-{4-cyano-3- |
| | [(1H-pyrazol-1-yl)methyl]phenyl}-3-fluoroazetidin-3- |
| | yl)methyl]piperidin-4-yl}-1-(3-fluorophenyl)ethyl]cyclopentyl]carbamate |
| 186 | methyl N-[(1S,2R)-2-[(1S)-2-(azetidin-1-yl)-1-{1-[(1-{4-cyano-3- |
| | [(1H-imidazol-1-yl)methyl]phenyl}-3-fluoroazetidin-3- |
| | yl)methyl]piperidin-4-yl}-1-(3-fluorophenyl)ethyl]cyclopentyl]carbamate |
| 187 | methyl N-[(1S,2R)-2-[(S)-{1-[(1-{3-[(azetidin-1-yl)methyl]-4- |
| | cyanophenyl}azetidin-3-yl)methyl]piperidin-4-yl}(cyano)(3- |
| | fluorophenyl)methyl]cyclopentyl]carbamate |
| 188 | methyl N-[(1S,2R)-2-[(S)-cyano({1-[(1-{4-cyano-3 -[(3-fluoroazetidin- |
| | 1-yl)methyl]phenyl}azetidin-3-yl)methyl]piperidin-4-yl})(3- |
| | fluorophenyl)methyl]cyclopentyl]carbamate |
| 189 | methyl N-[(1S,2R)-2-[(S)-cyano({1-[(1-{4-cyano-3- |
| | [(dimethylamino)methyl]phenyl}-3-fluoroazetidin-3- |
| | yl)methyl]piperidin-4-yl})(3- |
| | fluorophenyl)methyl]cyclopentyl]carbamate |
| 190 | methyl N-[(1S,2R)-2-[(1S)-2-(azetidin-1-yl)-1-[1-({1-[4-cyano-3- |
| | (hydroxymethyl)phenyl]azetidin-3-yl}methyl)piperidin-4-yl]-1-(3- |
| | fluorophenyl)ethyl]cyclopentyl]carbamate |
| 191 | methyl N-[(1S,2R)-2-[(1S)-2-(azetidin-1-yl)-1-{1-[(1-{4-cyano-3- |
| | [(piperidin-1-yl)methyl]phenyl}azetidin-3-yl)methyl]piperidin-4-yl}-1- |
| | (3-fluorophenyl)ethyl]cyclopentyl]carbamate |
| 192 | methyl N-[(1S,2R)-2-[(1S)-2-(azetidin-1-yl)-1-{1-[(1-{4-cyano-3-[(4- |
| | fluoropiperidin-1-yl)methyl]phenyl}azetidin-3-yl)methyl]piperidin-4- |
| | yl}-1-(3-fluorophenyl)ethyl]cyclopentyl]carbamate |
| 193 | methyl N-[(1S,2R)-2-[(1S)-2-(azetidin-1-yl)-1-{1-[(1-{4-cyano-3- |
| | [(4,4-difluoropiperidin-1-yl)methyl]phenyl}azetidin-3- |
| | yl)methyl]piperidin-4-yl}-1-(3 - |
| | fluorophenyl)ethyl]cyclopentyl]carbamate |
| 194 | methyl N-[(1S,2R)-2-[(1S)-2-(azetidin-1-yl)-1-{1-[(1-{4-cyano-3- |
| | [(diethylamino)methyl]phenyl}azetidin-3-yl)methyl]piperidin-4-yl}-1- |
| | (3-fluorophenyl)ethyl]cyclopentyl]carbamate |
| 195 | methyl N-[(1S,2R)-2-[(S)-cyano({1-[(1-{4-cyano-3-[(3-fluoroazetidin- |
| | 1-yl)methyl]phenyl}-3-fluoroazetidin-3-yl)methyl]piperidin-4-yl})(3- |
| | fluorophenyl)methyl]cyclopentyl]carbamate |
| 196 | methyl N-[(1S,2R)-2-[(1S)-1-[1-({1-[4-(cyclopropanesulfonyl)-3-[(3- |
| | fluoroazetidin-1-yl)methyl]phenyl]azetidin-3-yl}methyl)piperidin-4- |
| | yl]-2-acetamido-1-(3-fluorophenyl)ethyl]cyclopentyl]carbamate |
| 197 | methyl N-[(1S,2R)-2-[(1S)-1-{l-[(1-{4-carbamoyl-3-[(3- |
| | fluoroazetidin-1-yl)methyl]phenyl}azetidin-3-yl)methyl]piperidin-4- |
| | yl}-2-acetamido-1-(3-fluorophenyl)ethyl]cyclopentyl]carbamate |
| 198 | methyl N-[(1S,2R)-2-[(1S)-1-[1-({1-[4-(cyclopropanesulfonyl)-3-[(3- |
| | fluoroazetidin-1-yl)methyl]phenyl]-3-fluoroazetidin-3- |
| | yl}methyl)piperidin-4-yl]-2-acetamido-1-(3- |
| | fluorophenyl)ethyl]cyclopentyl]carbamate |
| 199 | methyl N-[(1S,2R)-2-[(1S)-2-(azetidin-1-yl)-1-{1-[3-(4- |
| | cyanophenoxy)-2,2-difluoropropyl]piperidin-4-yl}-1-(3- |
| | fluorophenyl)ethyl]cyclopentyl]carbamate |
| 200 | methyl N-[(1S,2R)-2-[(1S)-2-(azetidin-1-yl)-1[1′-(4-cyanophenyl)- |
| | [1,4′-bipiperidin]-4-yl]-1-(3-fluorophenyl)ethyl]cyclopentyl]carbamate |
| 201 | methyl N-[(1S,2R)-2-[(1S)-2-(azetidin-1-yl)-1-{1-[1-(4- |
| | cyanophenyl)azepan-4-yl]piperidin-4-yl}-1-(3- |
| | fluorophenyl)ethyl]cyclopentyl]carbamate |
| 202 | methyl ((1S,2R)-2-((1S)-2-(azetidin-1-yl)-1-(1-(1-(4- |
| | cyanophenyl)azepan-4-yl)piperidin-4-yl)-1-(3- |
| | fluorophenyl)ethyl)cyclopentyl)carbamate |
| 203 | methyl N-[(1S,2R)-2-[(1S)-2-(azetidin-1-yl)-1-(1-{[(3S)-1-(4- |
| | cyanophenyl)pyrrolidin-3-yl]methyl}piperidin-4-yl)-1-(3- |
| | fluorophenyl)ethyl]cyclopentyl]carbamate |
| 204 | methyl N-[(1S,2R)-2-[(1S)-2-(azetidin-1-yl)-1-(1-{[(3R)-1-(4- |
| | cyanophenyl)pyrrolidin-3-yl]methyl}piperidin-4-yl)-1-(3- |
| | fluorophenyl)ethyl]cyclopentyl]carbamate |
| 205 | methyl N-[(1S,2R)-2-[(1S)-2-(azetidin-1-yl)-1-{1′-[4- |
| | (cyclopropanesulfonyl)phenyl]-[1,4′-bipiperidin]-4-yl}-1-(3- |
| | fluorophenyl)ethyl]cyclopentyl]carbamate |
| 206 | methyl N-[(1S,2R)-2-[(1S)-2-(azetidin-1-yl)-1-[1′-3-cyanophenyl)- |
| | [1,4′-bipiperidin]-4-yl]-1-(3-fluorophenyl)ethyl]cyclopentyl]carbamate |
| 207 | methyl N-[(1S,2R)-2-[(1S)-2-(azetidin-1-yl)-1-(1-{[(3S)-1-[4- |
| | (cyclopropanesulfonyl)phenyl]pyrrolidin-3-yl]methyl}piperidin-4-yl)- |
| | 1-(3-fluorophenyl)ethyl]cyclopentyl]carbamate |
| 208 | methyl N-[(1S,2R)-2-[(1S)-2-(azetidin-1-yl)-1-(1-{[(3S)-1-(4-cyano-3- |
| | methoxyphenyl)pyrrolidin-3-yl]methyl}piperidin-4-yl)-1-(3- |
| | fluorophenyl)ethyl]cyclopentyl]carbamate |
| 209 | methyl N-[(1S,2R)-2-[(1S)-2-(azetidin-1-yl)-1-(1-{[(3S)-1-[4-(4- |
| | carbamoylbenzenesulfonyl)phenyl]pyrrolidin-3-yl]methyl}piperidin-4- |
| | yl)-1-(3-fluorophenyl)ethyl]cyclopentyl]carbamate |
| 210 | methyl N-[(1S,2R)-2-[(1S)-2-(azetidin-1-yl)-1-(3-fluorophenyl)-1-(1- |
| | {[(3S)-1-{4-[4-(methylcarbamoyl)benzenesulfonyl]phenyl}pyrrolidin- |
| | 3-yl]methyl}piperidin-4-yl)ethyl]cyclopentyl]carbamate |
| 211 | methyl N-[(1S,2R)-2-[(1S)-2-(azetidin-1-yl)-1-(1-{[(3S)-1-{4-[4- |
| | (dimethylcarbamoyl)benzenesulfonyl]phenyl}pyrrolidin-3- |
| | yl]methyl}piperidin-4-yl)-1-(3- |
| | fluorophenyl)ethyl]cyclopentyl]carbamate |
| 212 | methyl N-[(1S,2R)-2-[(1S)-1-{1-[(1-{4-[(1-acetylpiperidin-3- |
| | yl)sulfonyl]-2-fluorophenyl}-3-fluoroazetidin-3-yl)methyl]piperidin-4- |
| | yl}-2-(azetidin-1-yl)-1-(3-fluorophenyl)ethyl]cyclopentyl]carbamate |
| 213 | methyl 3-{4-[3-({4-[(1S)-2-(azetidin-1-yl)-1-(3-fluorophenyl)-1- |
| | [(1R,2S)-2-[(methoxycarbonyl)amino]cyclopentyl]ethyl]piperidin-1- |
| | yl}methyl)-3-fluoroazetidin-1-yl]-3-fluorobenzenesulfonyl}piperidine- |
| | 1-carboxylate |
| 214 | methyl N-[(1S,2R)-2-[(1S)-2-(azetidin-1-yl)-1-(1-{[3-fluoro-1-(2- |
| | fluoro-4-{[1-(2-hydroxy-2-methylpropanoyl)piperidin-3- |
| | yl]sulfonyl}phenyl)azetidin-3-yl]methyl}piperidin-4-yl)-1-(3- |
| | fluorophenyl)ethyl]cyclopentyl]carbamate |
| 215 | methyl N-[(1S,2R)-2-[(1S)-2-acetamido-1-{1-[(1-{3-[(3- |
| | fluoroazetidin-1-yl)methyl]-4-(methylcarbamoyl)phenyl}azetidin-3 - |
| | yl)methyl]piperidin-4-yl}-1-(3- |
| | fluorophenyl)ethyl]cyclopentyl]carbamate |
| 216 | methyl N-[(1S,2R)-2-[(1S)-2-acetamido-1-{1-[(1-{3-[(3- |
| | fluoroazetidin-1-yl)methyl]-4-sulfamoylphenyl}azetidin-3- |
| | yl)methyl]piperidin-4-yl}-1-(3- |
| | fluorophenyl)ethyl]cyclopentyl]carbamate |
| 217 | methyl N-[(1S,2R)-2-[(1S)-1-[1-({1-[4-(cyclobutanesulfonyl)-3-[(3- |
| | fluoroazetidin-1-yl)methyl]phenyl]azetidin-3-yl}methyl)piperidin-4- |
| | yl]-2-acetamido-1-(3-fluorophenyl)ethyl]cyclopentyl]carbamate |
| 218 | methyl N-[(1S,2R)-2-[(1S)-1-[1-({1-[4-(cyclopentanesulfonyl)-3-[(3- |
| | fluoroazetidin-1-yl)methyl]phenyl]azetidin-3-yl}methyl)piperidin-4- |
| | yl]-2-acetamido-1-(3-fluorophenyl)ethyl]cyclopentyl]carbamate |
| 219 | methyl N-[(1 S,2R)-2-[(1S)-1-[1-({1-[4-(benzenesulfonyl)-3-[(3- |
| | fluoroazetidin-1-yl)methyl]phenyl]azetidin-3-yl}methyl)piperidin-4- |
| | yl]-2-acetamido-1-(3-fluorophenyl)ethyl]cyclopentyl]carbamate |
| 220 | methyl N-[(1S,2R)-2-[(1S)-1-{1-[1-{4-cyano-3-[(3-fluoroazetidin-1- |
| | yl)methyl]phenyl}-3-hydroxyazetidin-3-yl)methyl]piperidin-4-yl}-2- |
| | acetamido-1-(3-fluorophenyl)ethyl]cyclopentyl]carbamate |
| 221 | methyl N-[(1S,2R)-2-[(1S)-1-{1-[(1-{4-cyano-3-[(3-fluoroazetidin-1- |
| | yl)methyl]phenyl}-3-methylazetidin-3-yl)methyl]piperidin-4-yl}-2- |
| | acetamido-1-(3-fluorophenyl)ethyl]cyclopentyl]carbamate |
| 222 | methyl N-[(1S,2R)-2-[(1S)-1-{1-[1-{4-cyano-3-[(3-fluoroazetidin-1- |
| | yl)methyl]phenyl}-3-(hydroxymethyl)azetidin-3-yl)methyl]piperidin-4- |
| | yl}-2-acetamido-1-(3-fluorophenyl)ethyl]cyclopentyl]carbamate |
| 223 | methyl N-[(1S,2R)-2-[(1S)-1-{1-[(1-{4-cyano-3-[(3-fluoroazetidin-1- |
| | yl)methyl]phenyl}-3-(fluoromethyl)azetidin-3-yl)methyl]piperidin-4- |
| | yl}-2-acetamido-1-(3-fluorophenyl)ethyl]cyclopentyl]carbamate |
| 224 | methyl N-[(1S,2R)-2-[(1S)-1-[1-({1-[4-(cyclopropanesulfonyl)-3-[(3- |
| | fluoroazetidin-1-yl)methyl]phenyl]-3-hydroxyazetidin-3- |
| | yl}methyl)piperidin-4-yl]-2-acetamido-1-(3- |
| | fluorophenyl)ethyl]cyclopentyl]carbamate |
| 225 | methyl N-[(1S,2R)-2-[(1S)-2-acetamido-1-{1-[(1-{3-[(3- |
| | fluoroazetidin-1-yl)methyl]-4-methanesulfonylphenyl}azetidin-3- |
| | yl)methyl]piperidin-4-yl}-1-(3- |
| | fluorophenyl)ethyl]cyclopentyl]carbamate |
| 226 | methyl N-[(1S,2R)-2-[(1S)-1-{1-[(1-{4-cyano-3-[(3-fluoroazetidin-1- |
| | yl)methyl]phenyl}azetidin-3-yl)methyl]piperidin-4-yl}-1-(3- |
| | fluorophenyl)-2-(N-methylacetamido)ethyl]cyclopentyl]carbamate |
| 227 | methyl ((1S,2R)-2-((S)-1-(1-((1-(4-cyano-3-((3-fluoroazetidin-1- |
| | yl)methyl)phenyl)azetidin-3-yl)methyl)piperidin-4-yl)-1-(3- |
| | fluorophenyl)-2-(2-oxopyrrolidin-1-yl)ethyl)cyclopentyl)carbamate |
| 228 | methyl ((1S,2R)-2-((S)-1-(1-((1-(4-cyano-3-((3-fluoroazetidin-1- |
| | yl)methyl)phenyl)azetidin-3-yl)methyl)piperidin-4-yl)-1-(3- |
| | fluorophenyl)-2-(2-oxopiperidin-l-yl)ethyl)cyclopentyl)carbamate |
| 229 | methyl ((1S,2R)-2-((S)-1-(1-((1-(4-cyano-3-((3-fluoroazetidin-1- |
| | yl)methyl)phenyl)azetidin-3-yl)methyl)piperidin-4-yl)-1-(3- |
| | fluorophenyl)-2- |
| | ((methoxycarbonyl)amino)ethyl)cyclopentyl)carbamate |
| 230 | methyl N-[(1S,2R)-2-[(S)-carbamoyl({1-[(1-{4-cyano-3-[(3- |
| | fluoroazetidin-1-yl)methyl]phenyl}azetidin-3-yl)methyl]piperidin-4- |
| | yl})(3-fluorophenyl)methyl]cyclopentyl]carbamate |
| 231 | methyl N-[(1S,2R)-2-[(S)-{1-[(1-{4-cyano-3-[(3-fluoroazetidin-1- |
| | yl)methyl]phenyl}azetidin-3-yl)methyl]piperidin-4-yl}(3- |
| | fluorophenyl)(methylcarbamoyl)methyl]cyclopentyl]carbamate |
| 232 | methyl N-[(1S,2R)-2-[(1S)-2-acetamido-1-{1-[(1-{3-[(3- |
| | fluoroazetidin-1-yl)methyl]-4-(trifluoromethyl)phenyl}azetidin-3- |
| | yl)methyl]piperidin-4-yl}-1-(3- |
| | fluorophenyl)ethyl]cyclopentyl]carbamate |
| 233 | methyl N-[(1S,2R)-2-[(1S)-1-[1-({1-[4-(cyclopropyldifluoromethyl)-3- |
| | [(3-fluoroazetidin-1-yl)methyl]phenyl]azetidin-3-yl}methyl)piperidin- |
| | 4-yl]-2-acetamido-1-(3-fluorophenyl)ethyl]cyclopentyl]carbamate |
| 234 | methyl N-[(1S,2R)-2-[(1S)-1-[1-({1-[4- |
| | (cyclopropanesulfonyl)phenyl]azetidin-3-yl}methyl)piperidin-4-yl]-2- |
| | acetamido-1-(3-fluorophenyl)ethyl]cyclopentyl]carbamate |
| 235 | methyl N-[(1S,2R)-2-[(1S)-1-[1-({1-[4-(cyclopropanesulfonyl)phenyl]-3- |
| | fluoroazetidin-3-yl}methyl)piperidin-4-yl]-2-acetamido-1-(3- |
| | fluorophenyl)ethyl]cyclopentyl]carbamate |
| 236 | methyl N-[(2S)-2-[1-({1-[4-(cyclopropanesulfonyl)phenyl]azetidin-3- |
| | yl}methyl)piperidin-4-yl]-2-(3-fluorophenyl)-2-[(1R,2S)-2- |
| | [(methoxycarbonyl)amino]cyclopentyl]ethyl]carbamate |
| 237 | methyl N-[(2S)-2-[1-({1-[4-(cyclopropanesulfonyl)phenyl]-3- |
| | fluoroazetidin-3-yl}methyl)piperidin-4-yl]-2-(3-fluorophenyl)-2- |
| | [(1R,2S)-2-[(methoxycarbonyl)amino]cyclopentyl]ethyl]carbamate |
| 238 | methyl N-[(1S,2R)-2-[(1S)-1-{1-[(1-{3-[(azetidin-1-yl)methyl]-4- |
| | (cyclopropanesulfonyl)phenyl}-3-fluoroazetidin-3-yl)methyl]piperidin- |
| | 4-yl}-2-acetamido-1-(3-fluorophenyl)ethyl]cyclopentyl]carbamate |
| 239 | methyl N-[(1S,2R)-2-[(1S)-1-[1-({1-[4-(cyclopropanesulfonyl)-3-[(3,3- |
| | difluoroazetidin-1-yl)methyl]phenyl]-3-fluoroazetidin-3- |
| | yl}methyl)piperidin-4-yl]-2-acetamido-1-(3- |
| | fluorophenyl)ethyl]cyclopentyl]carbamate |
| 241 | methyl N-[(1S,2R)-2-[(1S)-2-(azetidin-1-yl)-1-[1-({1-[4-cyano-3- |
| | (acetamidomethyl)phenyl]azetidin-3-yl}methyl)piperidin-4-yl]-1-(3- |
| | fluorophenyl)ethyl]cyclopentyl]carbamate |
| 242 | methyl N-[(1S,2R)-2-[(1S)-2-(azetidin-1-yl)-1-[1-({1-[4-cyano-3-(1H- |
| | pyrazol-4-yloxy)phenyl]azetidin-3-yl}methyl)piperidin-4-yl]-1-(3- |
| | fluorophenyl)ethyl]cyclopentyl]carbamate |
| 243 | methyl N-[(1S,2R)-2-[(1S)-2-(azetidin-1-yl)-1-{1-[(1-{4-cyano-3- |
| | [(1H-pyrazol-4-yl)methoxy]phenyl}azetidin-3-yl)methyl]piperidin-4- |
| | yl}-1-(3-fluorophenyl)ethyl]cyclopentyl]carbamate |
| 244 | methyl N-[(1S,2R)-2-[(1S)-2-(azetidin-1-yl)-1-{1-[(1-{3- |
| | cyanobicyclo[1.1.1]pentan-1-yl}azetidin-3-yl)methyl]piperidin-4-yl}- |
| | 1-(3-fluorophenyl)ethyl]cyclopentyl]carbamate |
| 245 | methyl N-[(1S,2R)-2-[(1S)-2-(azetidin-1-yl)-1-(1-{[1-(5- |
| | cyanothiophen-2-yl)azetidin-3-yl]methyl}piperidin-4-yl)-1-(3- |
| | fluorophenyl)ethyl]cyclopentyl]carbamate |
| 246 | methyl N-[(1S,2R)-2-[(1S)-2-(azetidin-1-yl)-1-[1-({1-[4- |
| | (cyclopropanesulfonyl)phenyl]-3-(fluoromethyl)azetidin-3- |
| | yl}methyl)piperidin-4-yl]-1-(3- |
| | fluorophenyl)ethyl]cyclopentyl]carbamate |
| 247 | methyl N-[(1S,2R)-2-[(1S)-2-(azetidin-1-yl)-1-[1-({1-[4- |
| | (cyclopropanesulfonyl)phenyl]-3-(methoxymethyl)azetidin-3- |
| | yl}methyl)piperidin-4-yl]-1-(3- |
| | fluorophenyl)ethyl]cyclopentyl]carbamate |
| 248 | methyl N-[(1S,2R)-2-[(1S)-2-(azetidin-1-yl)-1-(1-{[3-fluoro-1-(4-{[1- |
| | (1-hydroxycyclopropanecarbonyl)piperidin-4- |
| | yl]sulfonyl}phenyl)azetidin-3-yl]methyl}piperidin-4-yl)-1-(3- |
| | fluorophenyl)ethyl]cyclopentyl]carbamate |
| 249 | methyl N-[(1S,2R)-2-[(1S)-2-(azetidin-1-yl)-1-(1-{[3-fluoro-1-(4-{[1- |
| | (1-hydroxycyclopropanecarbonyl)azetidin-3- |
| | yl]sulfonyl}phenyl)azetidin-3-yl]methyl}piperidin-4-yl)-1-(3- |
| | fluorophenyl)ethyl]cyclopentyl]carbamate |

In another embodiment, Compounds of the Disclosure are any one or more of the compounds of Table 1.3, and the pharmaceutically acceptable salts and solvates thereof. Table 1.3A provides the chemical names of the compounds of Table 1.3 generated by ChemCurator 19 (ChemAxon Kft.) or ChemDraw Professional 16 (PerkinElmer Informatics, Inc.). In the event of any ambiguity between their chemical structure and chemical name, Compounds of the Disclosure are defined by their chemical structure.

TABLE 1.3

| | | MS |
|---|---|---|
| | | (ESI) |
| | | m/z |
| Cpd No. | Structure | [M + H]⁺ |
| 250 | 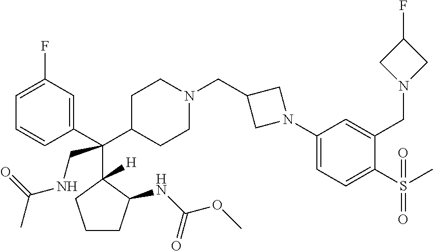 [SMILES: COC(=O)N[C@H]1CCC[C@@H]1[C@](CNC(C)=O)(c1cccc(F)c1)C1CCN(CC2CN(c3ccc(S(C)(=O)=O)c(CN4CC(F)C4)c3)C2)CC1] | 716.47 |
| 251 | 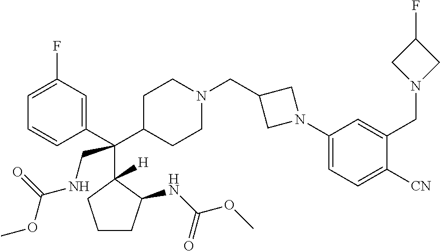 [SMILES: COC(=O)NC[C@@](c1cccc(F)c1)(C1CCN(CC2CN(c3ccc(C#N)c(CN4CC(F)C4)c3)C2)CC1)[C@H]1CCC[C@@H]1NC(=O)OC] | 679.53 |
| 252 | 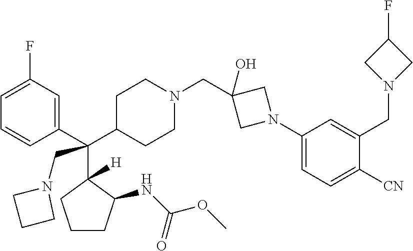 [SMILES: COC(=O)N[C@H]1CCC[C@@H]1[C@](CN1CCC1)(c1cccc(F)c1)C1CCN(CC2(O)CN(c3ccc(C#N)c(CN4CC(F)C4)c3)C2)CC1] | 677.54 |
| 253 | 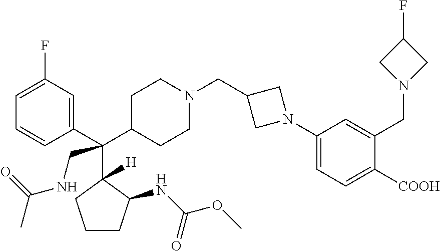 [SMILES: COC(=O)N[C@H]1CCC[C@@H]1[C@](CNC(C)=O)(c1cccc(F)c1)C1CCN(CC2CN(c3ccc(C(=O)O)c(CN4CC(F)C4)c3)C2)CC1] | 682.56 |
| 254 | 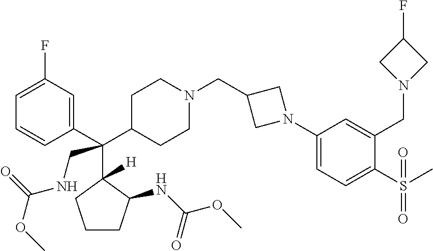 [SMILES: COC(=O)NC[C@@](c1cccc(F)c1)(C1CCN(CC2CN(c3ccc(S(C)(=O)=O)c(CN4CC(F)C4)c3)C2)CC1)[C@H]1CCC[C@@H]1NC(=O)OC] | 732.48 |
| 255 | 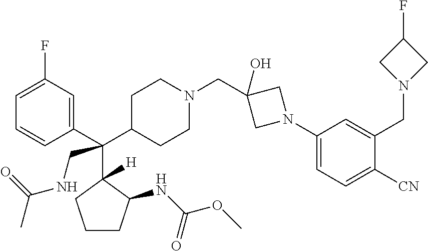 [SMILES: COC(=O)N[C@H]1CCC[C@@H]1[C@](CNC(C)=O)(c1cccc(F)c1)C1CCN(CC2(O)CN(c3ccc(C#N)c(CN4CC(F)C4)c3)C2)CC1] | 679.50 |
| 256 | 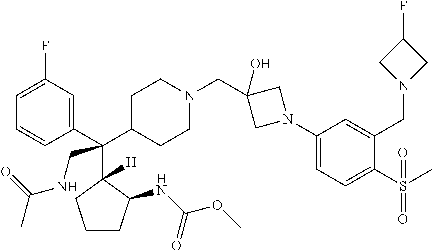 [SMILES: COC(=O)N[C@H]1CCC[C@@H]1[C@](CNC(C)=O)(c1cccc(F)c1)C1CCN(CC2(O)CN(c3ccc(S(C)(=O)=O)c(CN4CC(F)C4)c3)C2)CC1] | 732.50 |
| 257 | 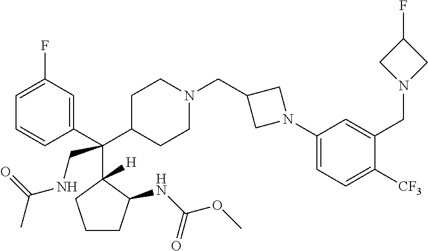 [SMILES: COC(=O)N[C@H]1CCC[C@@H]1[C@](CNC(C)=O)(c1cccc(F)c1)C1CCN(CC2CN(c3ccc(C(F)(F)F)c(CN4CC(F)C4)c3)C2)CC1] | 706.47 |
| 258 | 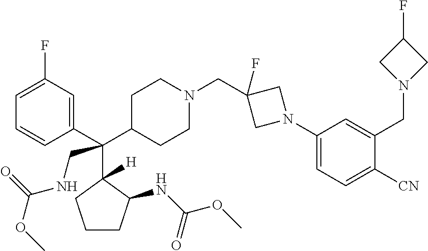 [SMILES: COC(=O)NC[C@@](c1cccc(F)c1)(C1CCN(CC2(F)CN(c3ccc(C#N)c(CN4CC(F)C4)c3)C2)CC1)[C@H]1CCC[C@@H]1NC(=O)OC] | 697.49 |
| 259 | 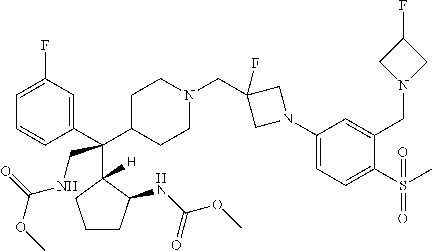 [SMILES: COC(=O)NC[C@@](c1cccc(F)c1)(C1CCN(CC2(F)CN(c3ccc(S(C)(=O)=O)c(CN4CC(F)C4)c3)C2)CC1)[C@H]1CCC[C@@H]1NC(=O)OC] | 750.49 |
| 260 | 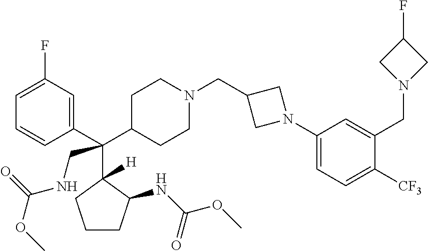 [SMILES: COC(=O)NC[C@@](c1cccc(F)c1)(C1CCN(CC2CN(c3ccc(C(F)(F)F)c(CN4CC(F)C4)c3)C2)CC1)[C@H]1CCC[C@@H]1NC(=O)OC] | 722.41 |
| 261 | 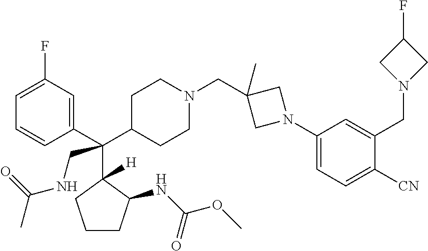 [SMILES: COC(=O)N[C@H]1CCC[C@@H]1[C@](CNC(C)=O)(c1cccc(F)c1)C1CCN(CC2(C)CN(c3ccc(C#N)c(CN4CC(F)C4)c3)C2)CC1] | 677.51 |
| 262 | 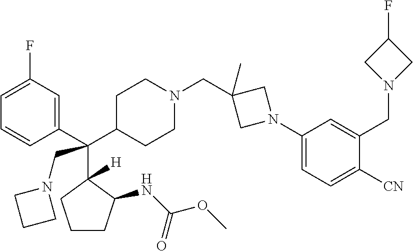 [SMILES: COC(=O)N[C@H]1CCC[C@@H]1[C@](CN1CCC1)(c1cccc(F)c1)C1CCN(CC2(C)CN(c3ccc(C#N)c(CN4CC(F)C4)c3)C2)CC1] | 675.54 |
| 263 | 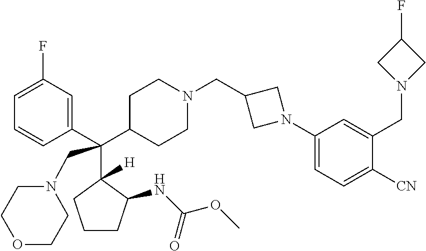 [SMILES: COC(=O)N[C@H]1CCC[C@@H]1[C@](CN1CCOCC1)(c1cccc(F)c1)C1CCN(CC2CN(c3ccc(C#N)c(CN4CC(F)C4)c3)C2)CC1] | 691.53 |
| 264 | 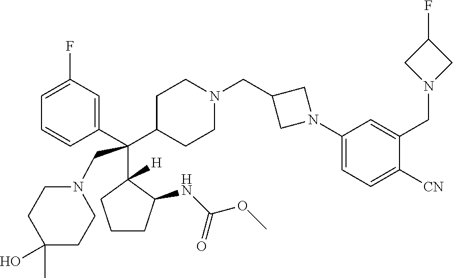 [SMILES: COC(=O)N[C@H]1CCC[C@@H]1[C@](CN1CCC(C)(O)CC1)(c1cccc(F)c1)C1CCN(CC2CN(c3ccc(C#N)c(CN4CC(F)C4)c3)C2)CC1] | 719.54 |
| 265 | 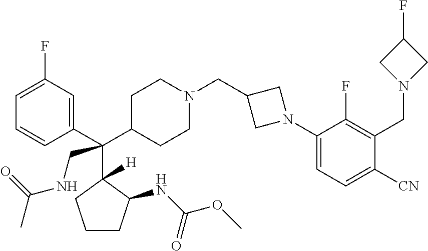 [SMILES: COC(=O)N[C@H]1CCC[C@@H]1[C@](CNC(C)=O)(c1cccc(F)c1)C1CCN(CC2CN(c3ccc(C#N)c(CN4CC(F)C4)c3F)C2)CC1] | 681.48 |
| 266 | 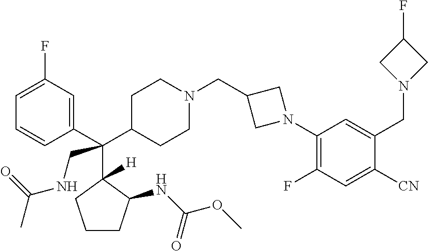 [SMILES: COC(=O)N[C@H]1CCC[C@@H]1[C@](CNC(C)=O)(c1cccc(F)c1)C1CCN(CC2CN(c3cc(CN4CC(F)C4)c(C#N)cc3F)C2)CC1] | 681.48 |
| 267 | 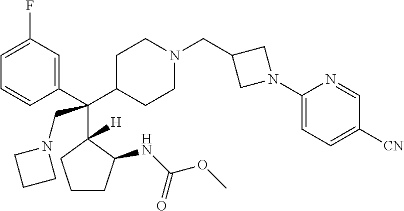 [SMILES: COC(=O)N[C@H]1CCC[C@@H]1[C@](CN1CCC1)(c1cccc(F)c1)C1CCN(CC2CN(c3ccc(C#N)cn3)C2)CC1] | 575.46 |
| 268 | 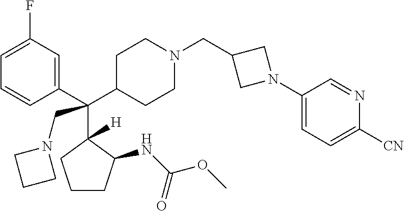 [SMILES: COC(=O)N[C@H]1CCC[C@@H]1[C@](CN1CCC1)(c1cccc(F)c1)C1CCN(CC2CN(c3ccc(C#N)nc3)C2)CC1] | 575.48 |
| 269 | 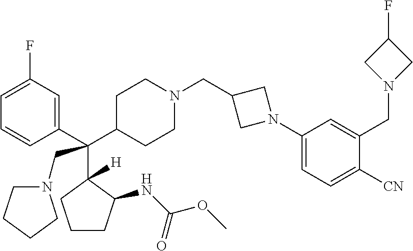 [SMILES: COC(=O)N[C@H]1CCC[C@@H]1[C@](CN1CCCC1)(c1cccc(F)c1)C1CCN(CC2CN(c3ccc(C#N)c(CN4CC(F)C4)c3)C2)CC1] | 675.52 |
| 270 | 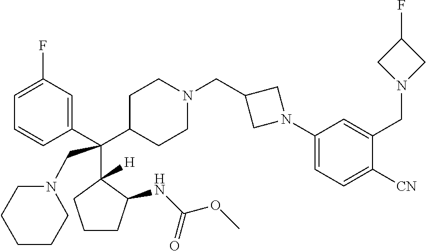 [SMILES: COC(=O)N[C@H]1CCC[C@@H]1[C@](CN1CCCCC1)(c1cccc(F)c1)C1CCN(CC2CN(c3ccc(C#N)c(CN4CC(F)C4)c3)C2)CC1] | 689.56 |
| 271 | 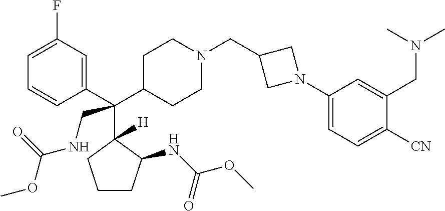 | 649.55 |
| 272 | 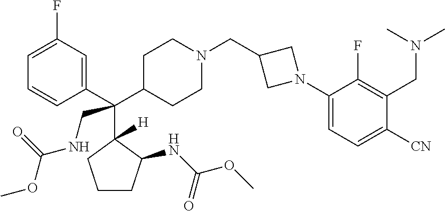 | 667.39 |
| 273 | 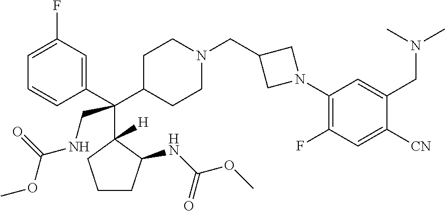 | 667.35 |
| 274 | 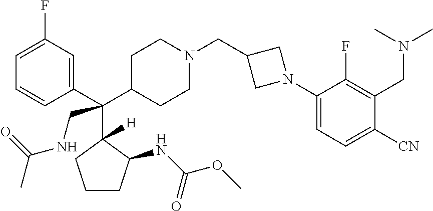 [SMILES: COC(=O)N[C@H]1CCC[C@@H]1[C@](CNC(C)=O)(c1cccc(F)c1)C1CCN(CC2CN(c3ccc(C#N)c(CN(C)C)c3F)C2)CC1] | 651.48 |
| 275 | 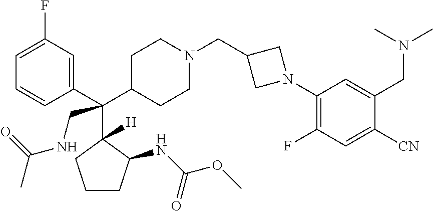 [SMILES: COC(=O)N[C@H]1CCC[C@@H]1[C@](CNC(C)=O)(c1cccc(F)c1)C1CCN(CC2CN(c3cc(CN(C)C)c(C#N)cc3F)C2)CC1] | 651.48 |
| 276 | 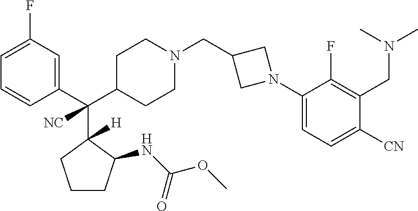 [SMILES: COC(=O)N[C@H]1CCC[C@@H]1[C@](C#N)(c1cccc(F)c1)C1CCN(CC2CN(c3ccc(C#N)c(CN(C)C)c3F)C2)CC1] | 605.49 |
| 277 | 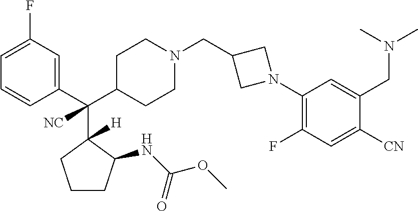 [SMILES: COC(=O)N[C@H]1CCC[C@@H]1[C@](C#N)(c1cccc(F)c1)C1CCN(CC2CN(c3cc(CN(C)C)c(C#N)cc3F)C2)CC1] | 605.51 |
| 278 | 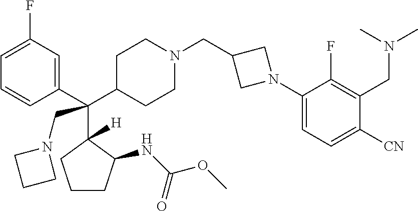 [SMILES: COC(=O)N[C@H]1CCC[C@@H]1[C@](CN1CCC1)(c1cccc(F)c1)C1CCN(CC2CN(c3ccc(C#N)c(CN(C)C)c3F)C2)CC1] | 649.52 |
| 279 | 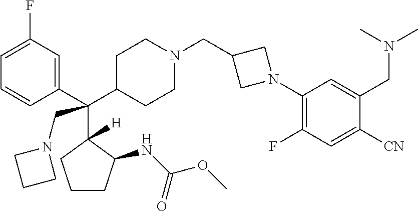 [SMILES: COC(=O)N[C@H]1CCC[C@@H]1[C@](CN1CCC1)(c1cccc(F)c1)C1CCN(CC2CN(c3cc(CN(C)C)c(C#N)cc3F)C2)CC1] | 649.52 |
| 280 | 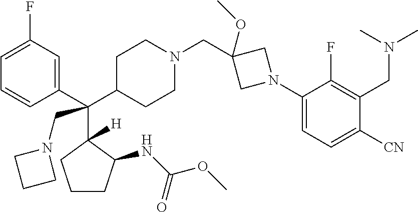 [SMILES: COC(=O)N[C@H]1CCC[C@@H]1[C@](CN1CCC1)(c1cccc(F)c1)C1CCN(CC2(OC)CN(c3ccc(C#N)c(CN(C)C)c3F)C2)CC1] | 679.49 |
| 281 | 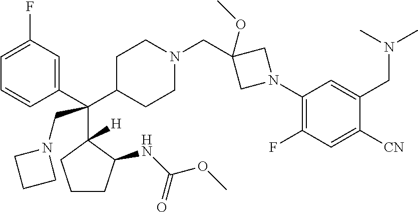 [SMILES: COC(=O)N[C@H]1CCC[C@@H]1[C@](CN1CCC1)(c1cccc(F)c1)C1CCN(CC2(OC)CN(c3cc(CN(C)C)c(C#N)cc3F)C2)CC1] | 679.56 |
| 282 | 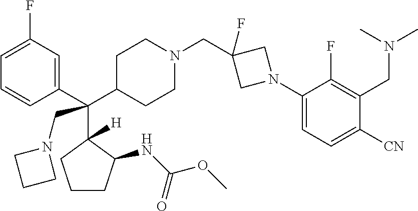 [SMILES: COC(=O)N[C@H]1CCC[C@@H]1[C@](CN1CCC1)(c1cccc(F)c1)C1CCN(CC2(F)CN(c3ccc(C#N)c(CN(C)C)c3F)C2)CC1] | 667.47 |
| 283 | 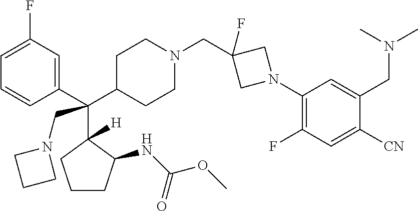 [SMILES: COC(=O)N[C@H]1CCC[C@@H]1[C@](CN1CCC1)(c1cccc(F)c1)C1CCN(CC2(F)CN(c3cc(CN(C)C)c(C#N)cc3F)C2)CC1] | 667.48 |
| 284 | 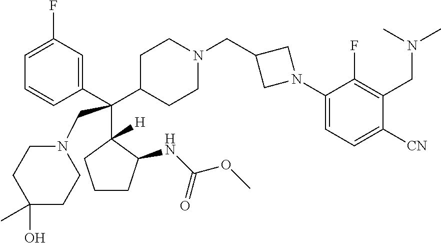 | 707.55 |
| 285 | 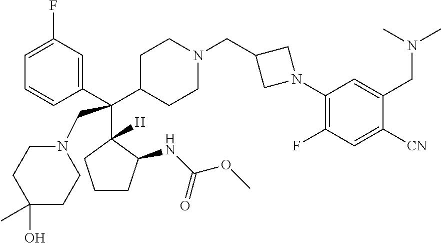 | 707.56 |
| 286 | 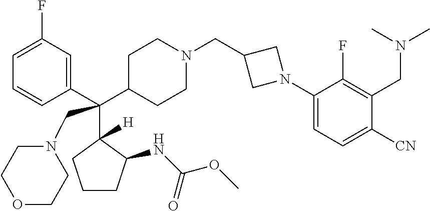 [SMILES: COC(=O)N[C@H]1CCC[C@@H]1[C@](CN1CCOCC1)(c1cccc(F)c1)C1CCN(CC2CN(c3ccc(C#N)c(CN(C)C)c3F)C2)CC1] | 679.54 |
| 287 | 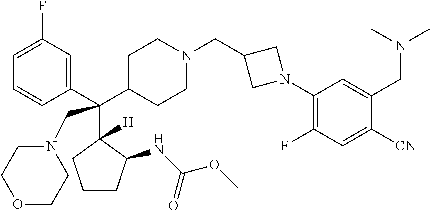 [SMILES: COC(=O)N[C@H]1CCC[C@@H]1[C@](CN1CCOCC1)(c1cccc(F)c1)C1CCN(CC2CN(c3cc(CN(C)C)c(C#N)cc3F)C2)CC1] | 679.54 |
| 288 | 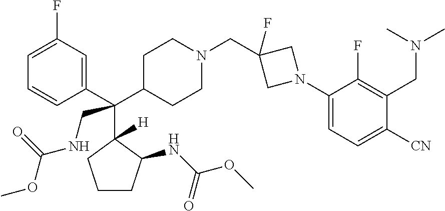 | 685.48 |
| 289 | 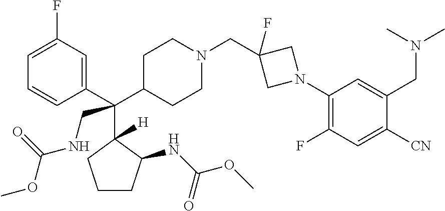 | 685.50 |
| 290 | 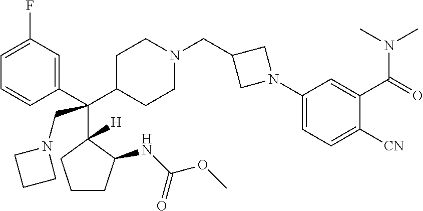 [SMILES: COC(=O)N[C@H]1CCC[C@@H]1[C@](CN1CCC1)(c1cccc(F)c1)C1CCN(CC2CN(c3ccc(C#N)c(C(=O)N(C)C)c3)C2)CC1] | 645.57 |
| 291 | 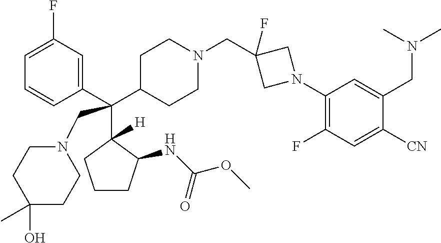 | 725.52 |
| 292 | 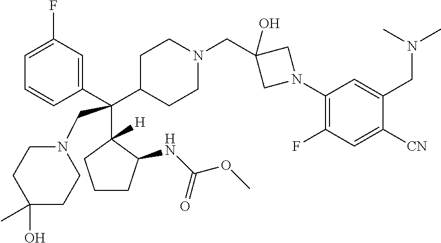 | 723.51 |
| 293 | 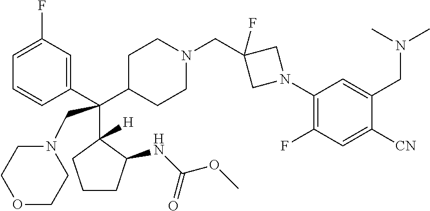 [SMILES: COC(=O)N[C@H]1CCC[C@@H]1[C@](CN1CCOCC1)(c1cccc(F)c1)C1CCN(CC2(F)CN(c3cc(CN(C)C)c(C#N)cc3F)C2)CC1] | 697.51 |
| 294 | 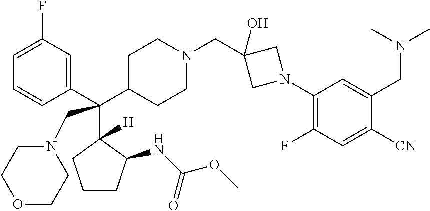 [SMILES: COC(=O)N[C@H]1CCC[C@@H]1[C@](CN1CCOCC1)(c1cccc(F)c1)C1CCN(CC2(O)CN(c3cc(CN(C)C)c(C#N)cc3F)C2)CC1] | 695.48 |
| 295 | 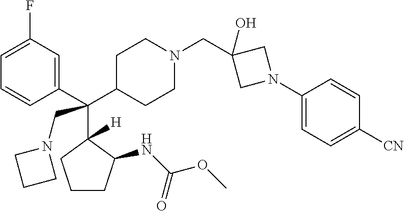 [SMILES: COC(=O)N[C@H]1CCC[C@@H]1[C@](CN1CCC1)(c1cccc(F)c1)C1CCN(CC2(O)CN(c3ccc(C#N)cc3)C2)CC1] | 590.39 |
| 296 | 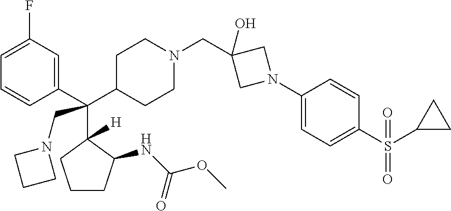 [SMILES: COC(=O)N[C@H]1CCC[C@@H]1[C@](CN1CCC1)(c1cccc(F)c1)C1CCN(CC2(O)CN(c3ccc(S(=O)(=O)C4CC4)cc3)C2)CC1] | 669.42 |
| 297 | 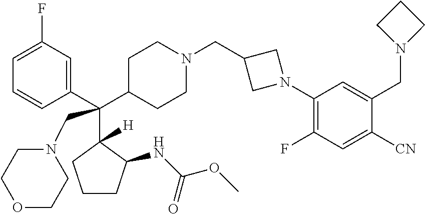 [SMILES: COC(=O)N[C@H]1CCC[C@@H]1[C@](CN1CCOCC1)(c1cccc(F)c1)C1CCN(CC2CN(c3cc(CN4CCC4)c(C#N)cc3F)C2)CC1] | 691.45 |
| 298 | 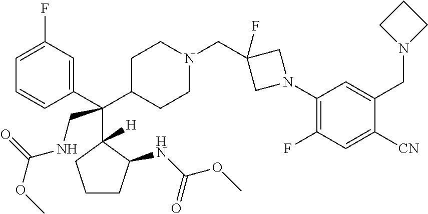 [SMILES: COC(=O)NC[C@@](c1cccc(F)c1)(C1CCN(CC2(F)CN(c3cc(CN4CCC4)c(C#N)cc3F)C2)CC1)[C@H]1CCC[C@@H]1NC(=O)OC] | 697.39 |
| 299 | 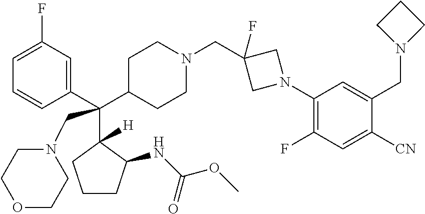 [SMILES: COC(=O)N[C@H]1CCC[C@@H]1[C@](CN1CCOCC1)(c1cccc(F)c1)C1CCN(CC2(F)CN(c3cc(CN4CCC4)c(C#N)cc3F)C2)CC1] | 709.43 |
| 300 | 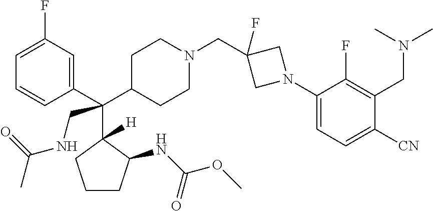 [SMILES: COC(=O)N[C@H]1CCC[C@@H]1[C@](CNC(C)=O)(c1cccc(F)c1)C1CCN(CC2(F)CN(c3ccc(C#N)c(CN(C)C)c3F)C2)CC1] | 669.57 |
| 301 | 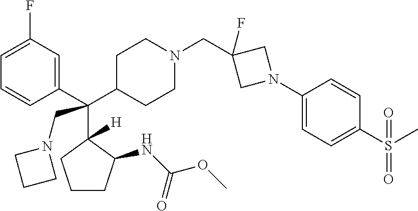 [SMILES: COC(=O)N[C@H]1CCC[C@@H]1[C@](CN1CCC1)(c1cccc(F)c1)C1CCN(CC2(F)CN(c3ccc(S(C)(=O)=O)cc3)C2)CC1] | 645.58 |
| 302 | 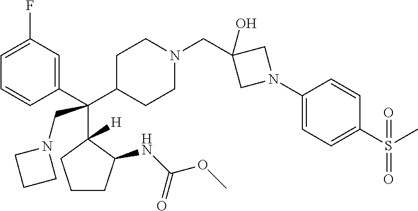 [SMILES: COC(=O)N[C@H]1CCC[C@@H]1[C@](CN1CCC1)(c1cccc(F)c1)C1CCN(CC2(O)CN(c3ccc(S(C)(=O)=O)cc3)C2)CC1] | 643.47 |
| 303 | 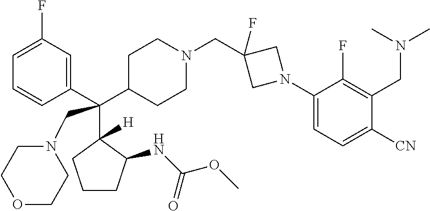 [SMILES: COC(=O)N[C@H]1CCC[C@@H]1[C@](CN1CCOCC1)(c1cccc(F)c1)C1CCN(CC2(F)CN(c3ccc(C#N)c(CN(C)C)c3F)C2)CC1] | 697.57 |
| 304 | 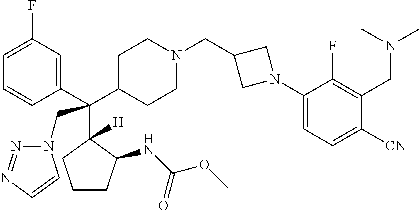 [SMILES: COC(=O)N[C@H]1CCC[C@@H]1[C@](Cn1ccnn1)(c1cccc(F)c1)C1CCN(CC2CN(c3ccc(C#N)c(CN(C)C)c3F)C2)CC1] | 661.50 |
| 305 | 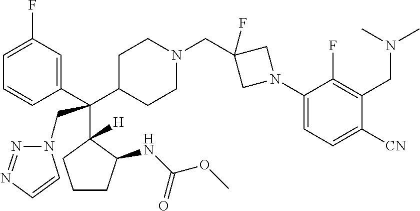 [SMILES: COC(=O)N[C@H]1CCC[C@@H]1[C@](Cn1ccnn1)(c1cccc(F)c1)C1CCN(CC2(F)CN(c3ccc(C#N)c(CN(C)C)c3F)C2)CC1] | 679.48 |
| 306 | 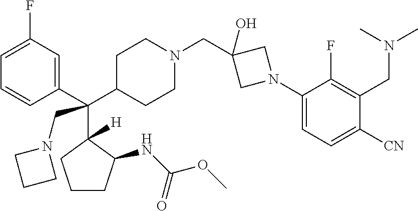 [SMILES: COC(=O)N[C@H]1CCC[C@@H]1[C@](CN1CCC1)(c1cccc(F)c1)C1CCN(CC2(O)CN(c3ccc(C#N)c(CN(C)C)c3F)C2)CC1] | 665.39 |
| 307 | 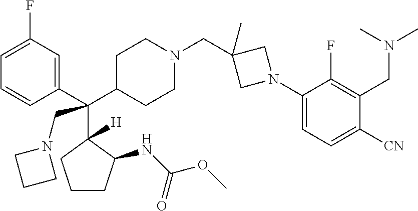 [SMILES: COC(=O)N[C@H]1CCC[C@@H]1[C@](CN1CCC1)(c1cccc(F)c1)C1CCN(CC2(C)CN(c3ccc(C#N)c(CN(C)C)c3F)C2)CC1] | 663.54 |
| 308 | 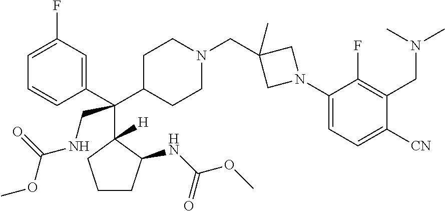 | 681.54 |
| 309 | 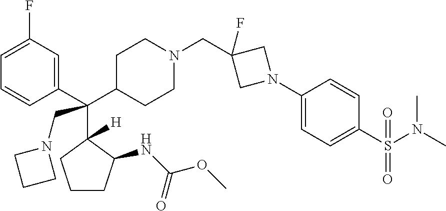 [SMILES: COC(=O)N[C@H]1CCC[C@@H]1[C@](CN1CCC1)(c1cccc(F)c1)C1CCN(CC2(F)CN(c3ccc(S(=O)(=O)N(C)C)cc3)C2)CC1] | 674.53 |
| 310 | 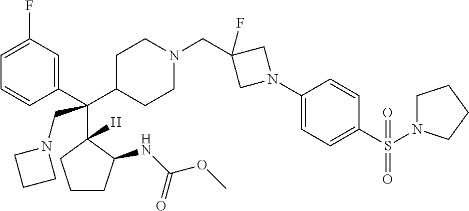 [SMILES: COC(=O)N[C@H]1CCC[C@@H]1[C@](CN1CCC1)(c1cccc(F)c1)C1CCN(CC2(F)CN(c3ccc(S(=O)(=O)N4CCCC4)cc3)C2)CC1] | 700.56 |
| 311 | 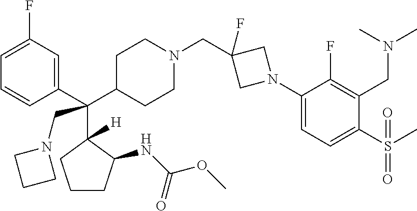 [SMILES: COC(=O)N[C@H]1CCC[C@@H]1[C@](CN1CCC1)(c1cccc(F)c1)C1CCN(CC2(F)CN(c3ccc(S(C)(=O)=O)c(CN(C)C)c3F)C2)CC1] | 720.46 |
| 312 | 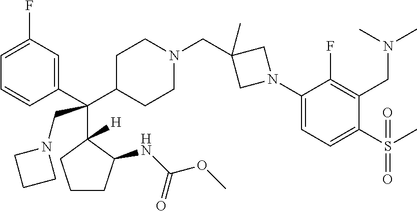 [SMILES: COC(=O)N[C@H]1CCC[C@@H]1[C@](CN1CCC1)(c1cccc(F)c1)C1CCN(CC2(C)CN(c3ccc(S(C)(=O)=O)c(CN(C)C)c3F)C2)CC1] | 716.46 |
| 313 | 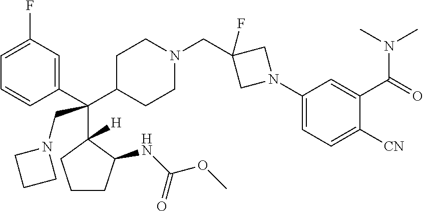 [SMILES: COC(=O)N[C@H]1CCC[C@@H]1[C@](CN1CCC1)(c1cccc(F)c1)C1CCN(CC2(F)CN(c3ccc(C#N)c(C(=O)N(C)C)c3)C2)CC1] | 663.57 |
| 314 | 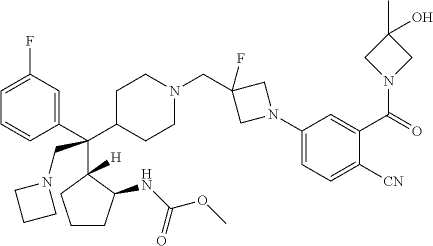 [SMILES: COC(=O)N[C@H]1CCC[C@@H]1[C@](CN1CCC1)(c1cccc(F)c1)C1CCN(CC2(F)CN(c3ccc(C#N)c(C(=O)N4CC(C)(O)C4)c3)C2)CC1] | 705.41 |
| 315 | 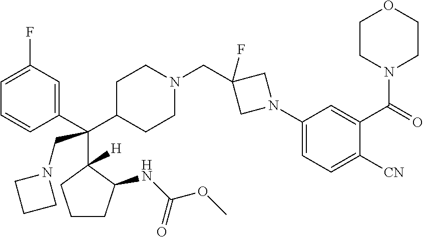 [SMILES: COC(=O)N[C@H]1CCC[C@@H]1[C@](CN1CCC1)(c1cccc(F)c1)C1CCN(CC2(F)CN(c3ccc(C#N)c(C(=O)N4CCOCC4)c3)C2)CC1] | 705.46 |
| 316 | 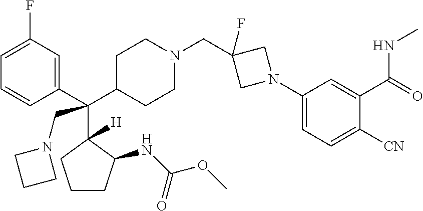 [SMILES: CNC(=O)c1cc(N2CC(F)(CN3CCC([C@@](CN4CCC4)(c4cccc(F)c4)[C@H]4CCC[C@@H]4NC(=O)OC)CC3)C2)ccc1C#N] | 649.47 |
| 317 | 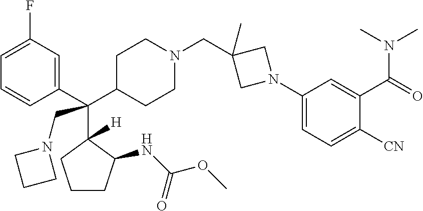 [SMILES: COC(=O)N[C@H]1CCC[C@@H]1[C@](CN1CCC1)(c1cccc(F)c1)C1CCN(CC2(C)CN(c3ccc(C#N)c(C(=O)N(C)C)c3)C2)CC1] | 659.38 |
| 318 | 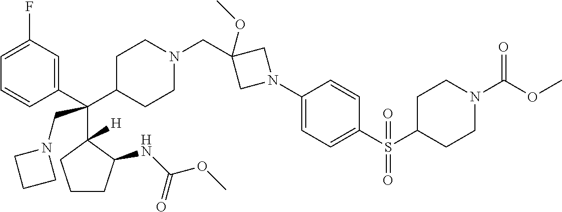 [SMILES: COC(=O)N[C@H]1CCC[C@@H]1[C@](CN1CCC1)(c1cccc(F)c1)C1CCN(CC2(OC)CN(c3ccc(S(=O)(=O)C4CCN(C(=O)OC)CC4)cc3)C2)CC1] | 784.51 |
| 319 | 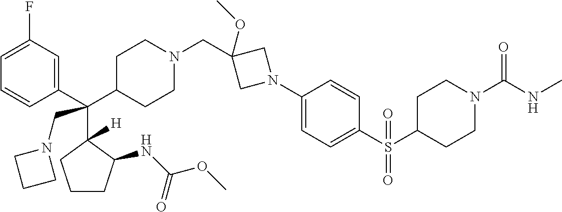 [SMILES: CNC(=O)N1CCC(S(=O)(=O)c2ccc(N3CC(CN4CCC([C@@](CN5CCC5)(c5cccc(F)c5)[C@H]5CCC[C@@H]5NC(=O)OC)CC4)(OC)C3)cc2)CC1] | 783.52 |
| 320 | 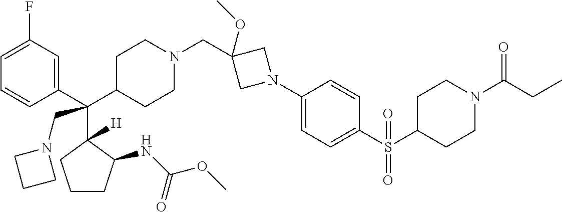 [SMILES: CCC(=O)N1CCC(S(=O)(=O)c2ccc(N3CC(CN4CCC([C@@](CN5CCC5)(c5cccc(F)c5)[C@H]5CCC[C@@H]5NC(=O)OC)CC4)(OC)C3)cc2)CC1] | 782.55 |
| 321 | 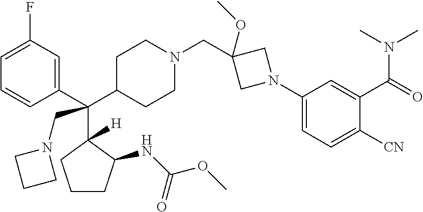 [SMILES: COC(=O)N[C@H]1CCC[C@@H]1[C@](CN1CCC1)(c1cccc(F)c1)C1CCN(CC2(OC)CN(c3ccc(C#N)c(C(=O)N(C)C)c3)C2)CC1] | 675.55 |
| 322 | 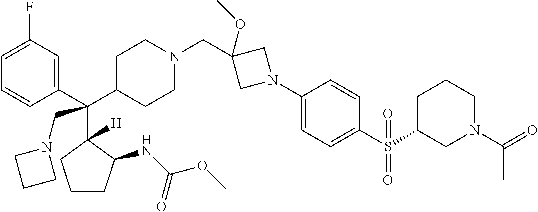 [SMILES: COC(=O)N[C@H]1CCC[C@@H]1[C@](CN1CCC1)(c1cccc(F)c1)C1CCN(CC2(OC)CN(c3ccc(S(=O)(=O)[C@@H]4CCCN(C(C)=O)C4)cc3)C2)CC1] | 768.45 |
| 323 | 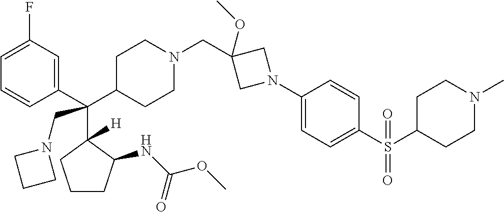 [SMILES: COC(=O)N[C@H]1CCC[C@@H]1[C@](CN1CCC1)(c1cccc(F)c1)C1CCN(CC2(OC)CN(c3ccc(S(=O)(=O)C4CCN(C)CC4)cc3)C2)CC1] | 740.53 |
| 324 | 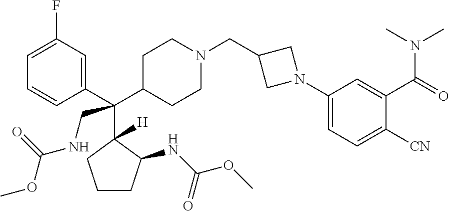 [SMILES: COC(=O)NC[C@@](c1cccc(F)c1)(C1CCN(CC2CN(c3ccc(C#N)c(C(=O)N(C)C)c3)C2)CC1)[C@H]1CCC[C@@H]1NC(=O)OC] | 663.55 |
| 325 | 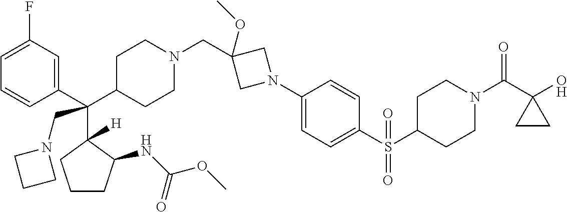 [SMILES: COC(=O)N[C@H]1CCC[C@@H]1[C@](CN1CCC1)(c1cccc(F)c1)C1CCN(CC2(OC)CN(c3ccc(S(=O)(=O)C4CCN(C(=O)C5(O)CC5)CC4)cc3)C2)CC1] | 810.50 |
| 326 | 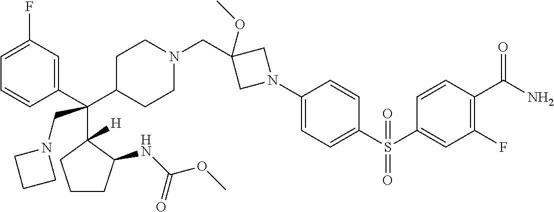 [SMILES: COC(=O)N[C@H]1CCC[C@@H]1[C@](CN1CCC1)(c1cccc(F)c1)C1CCN(CC2(OC)CN(c3ccc(S(=O)(=O)c4ccc(C(N)=O)c(F)c4)cc3)C2)CC1] | 806.46 |
| 327 | 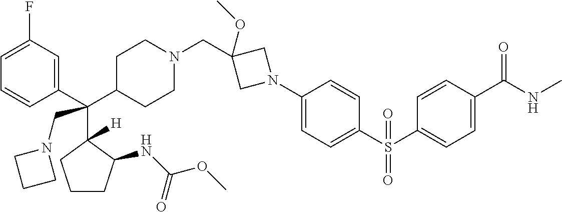 [SMILES: CNC(=O)c1ccc(S(=O)(=O)c2ccc(N3CC(CN4CCC([C@@](CN5CCC5)(c5cccc(F)c5)[C@H]5CCC[C@@H]5NC(=O)OC)CC4)(OC)C3)cc2)cc1] | 776.49 |
| 328 | 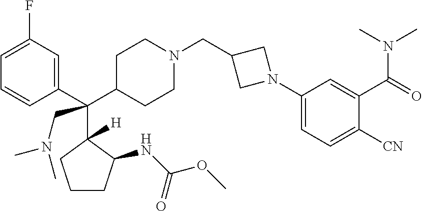 [SMILES: COC(=O)N[C@H]1CCC[C@@H]1[C@](CN(C)C)(c1cccc(F)c1)C1CCN(CC2CN(c3ccc(C#N)c(C(=O)N(C)C)c3)C2)CC1] | 633.56 |
| 329 | 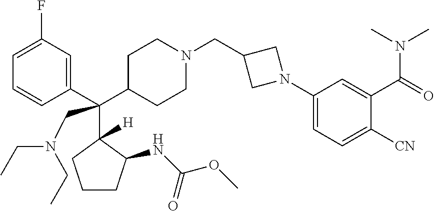 [SMILES: CCN(CC)C[C@@](c1cccc(F)c1)(C1CCN(CC2CN(c3ccc(C#N)c(C(=O)N(C)C)c3)C2)CC1)[C@H]1CCC[C@@H]1NC(=O)OC] | 661.57 |
| 330 | 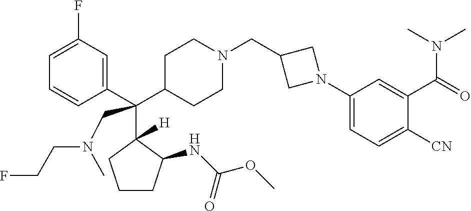 [SMILES: COC(=O)N[C@H]1CCC[C@@H]1[C@](CN(C)CCF)(c1cccc(F)c1)C1CCN(CC2CN(c3ccc(C#N)c(C(=O)N(C)C)c3)C2)CC1] | 665.56 |
| 331 | 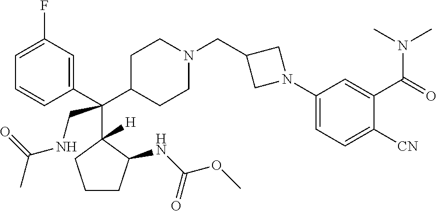 [SMILES: COC(=O)N[C@H]1CCC[C@@H]1[C@](CNC(C)=O)(c1cccc(F)c1)C1CCN(CC2CN(c3ccc(C#N)c(C(=O)N(C)C)c3)C2)CC1] | 647.54 |
| 332 | 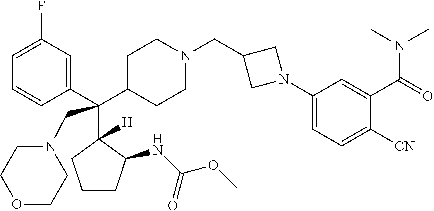 [SMILES: COC(=O)N[C@H]1CCC[C@@H]1[C@](CN1CCOCC1)(c1cccc(F)c1)C1CCN(CC2CN(c3ccc(C#N)c(C(=O)N(C)C)c3)C2)CC1] | 675.55 |
| 333 | 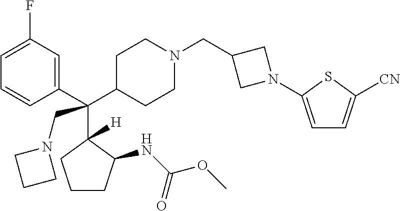 [SMILES: COC(=O)N[C@H]1CCC[C@@H]1[C@](CN1CCC1)(c1cccc(F)c1)C1CCN(CC2CN(c3ccc(C#N)s3)C2)CC1] | 580.34 |
| 334 | 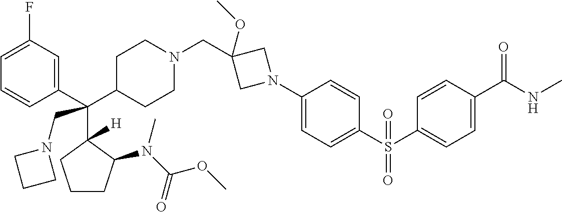 [SMILES: CNC(=O)c1ccc(S(=O)(=O)c2ccc(N3CC(CN4CCC([C@@](CN5CCC5)(c5cccc(F)c5)[C@H]5CCC[C@@H]5N(C)C(=O)OC)CC4)(OC)C3)cc2)cc1] | 790.07 |
| 335 | 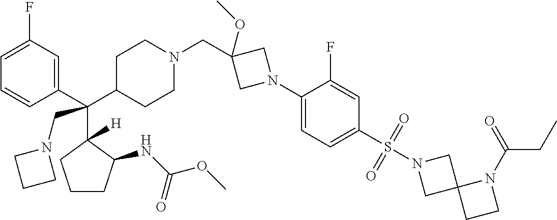 [SMILES: CCC(=O)N1CCC12CN(S(=O)(=O)c1ccc(N3CC(CN4CCC([C@@](CN5CCC5)(c5cccc(F)c5)[C@H]5CCC[C@@H]5NC(=O)OC)CC4)(OC)C3)c(F)c1)C2] | 813.07 |
| 336 | 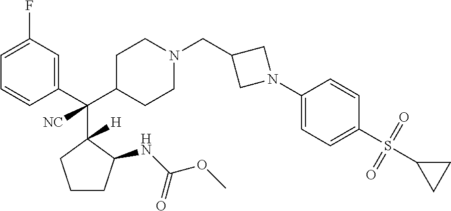 [SMILES: COC(=O)N[C@H]1CCC[C@@H]1[C@](C#N)(c1cccc(F)c1)C1CCN(CC2CN(c3ccc(S(=O)(=O)C4CC4)cc3)C2)CC1] | 609.37 |
| 337 | 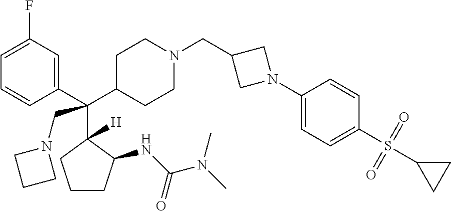 [SMILES: CN(C)C(=O)N[C@H]1CCC[C@@H]1[C@](CN1CCC1)(c1cccc(F)c1)C1CCN(CC2CN(c3ccc(S(=O)(=O)C4CC4)cc3)C2)CC1] | 666.63 |
| 338 | 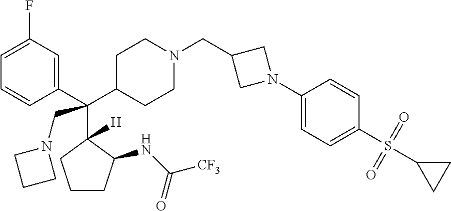 [SMILES: O=C(N[C@H]1CCC[C@@H]1[C@](CN1CCC1)(c1cccc(F)c1)C1CCN(CC2CN(c3ccc(S(=O)(=O)C4CC4)cc3)C2)CC1)C(F)(F)F] | 691.55 |
| 339 | 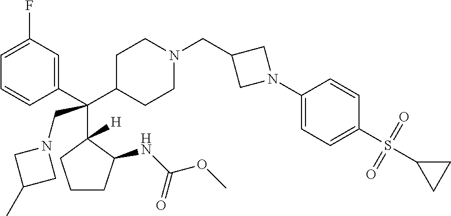 [SMILES: COC(=O)N[C@H]1CCC[C@@H]1[C@](CN1CC(C)C1)(c1cccc(F)c1)C1CCN(CC2CN(c3ccc(S(=O)(=O)C4CC4)cc3)C2)CC1] | 667.68 |
| 340 | 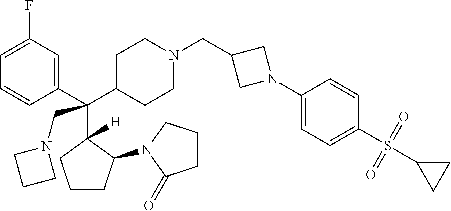 [SMILES: O=C1CCCN1[C@H]1CCC[C@@H]1[C@](CN1CCC1)(c1cccc(F)c1)C1CCN(CC2CN(c3ccc(S(=O)(=O)C4CC4)cc3)C2)CC1] | 663.51 |
| 341 | 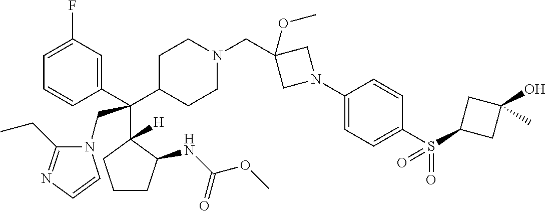 [SMILES: CCc1nccn1C[C@@](c1cccc(F)c1)(C1CCN(CC2(OC)CN(c3ccc(S(=O)(=O)[C@H]4C[C@@](C)(O)C4)cc3)C2)CC1)[C@H]1CCC[C@@H]1NC(=O)OC] | 766.49 |
| 342 | 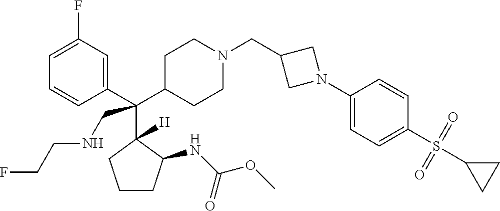 [SMILES: COC(=O)N[C@H]1CCC[C@@H]1[C@](CNCCF)(c1cccc(F)c1)C1CCN(CC2CN(c3ccc(S(=O)(=O)C4CC4)cc3)C2)CC1] | 659.41 |
| 343 | 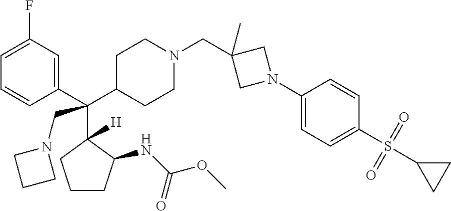 [SMILES: COC(=O)N[C@H]1CCC[C@@H]1[C@](CN1CCC1)(c1cccc(F)c1)C1CCN(CC2(C)CN(c3ccc(S(=O)(=O)C4CC4)cc3)C2)CC1] | 667.53 |
| 344 | 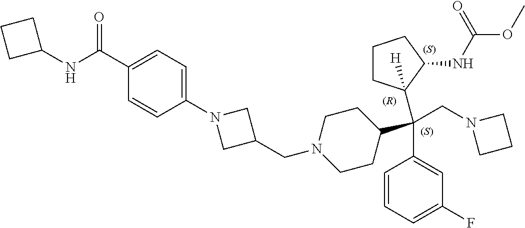 | 646.75 |
| 345 | 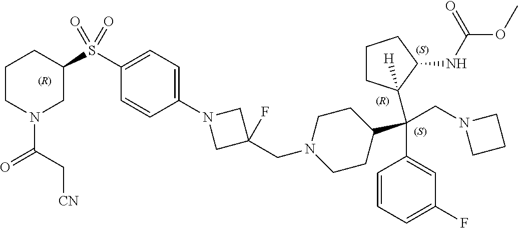 | 781.83 |
| 346 | 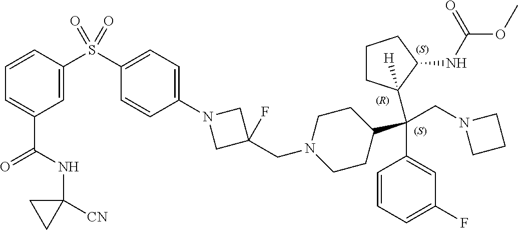 | 815.79 |
| 347 | 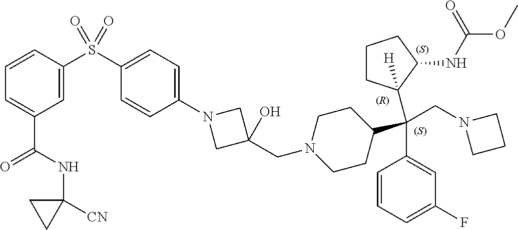 | 813.83 |
| 348 | 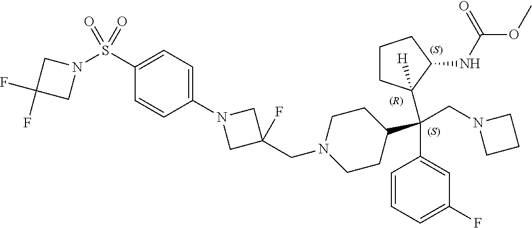 | 722.53 |
| 349 | 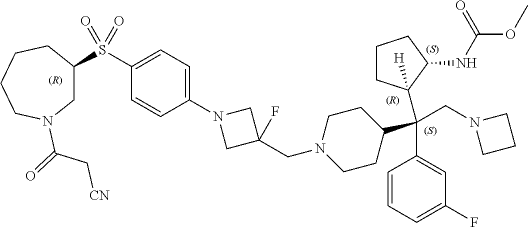 | 795.53 |
| 350 | 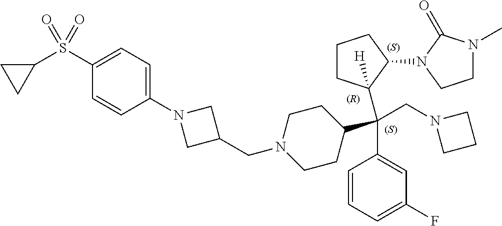 | 678.51 |
| 351 | 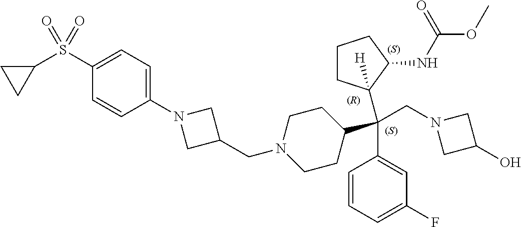 | 669.49 |
| 352 | 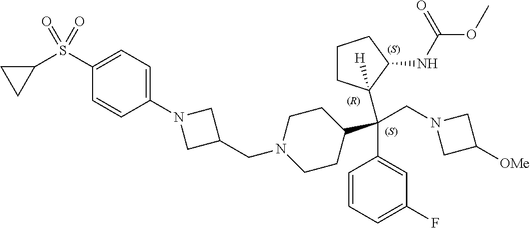 | 683.45 |
| 353 | 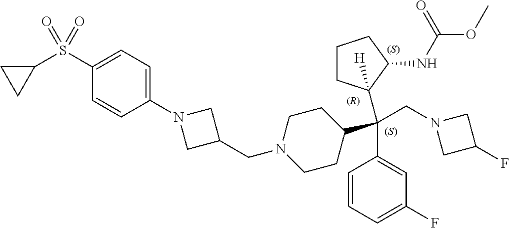 | 671.48 |
| 354 | 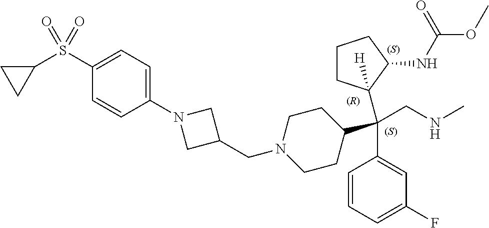 | 627.39 |
| 355 | 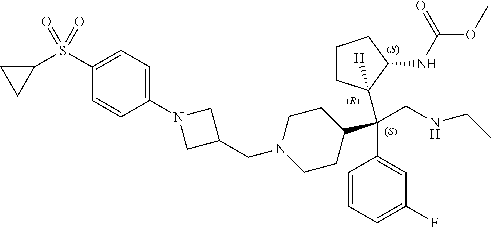 | 641.45 |
| 356 | 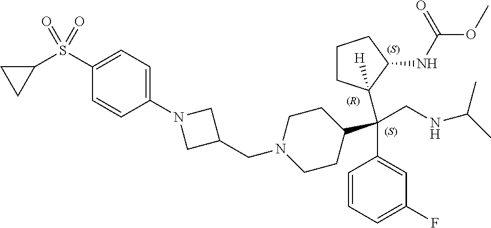 | 655.68 |
| 357 | 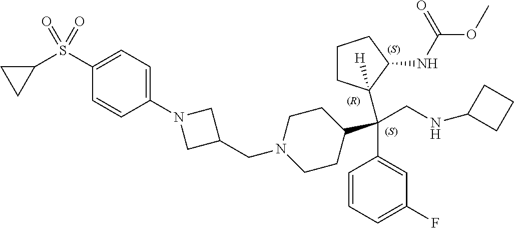 | 667.65 |
| 358 | 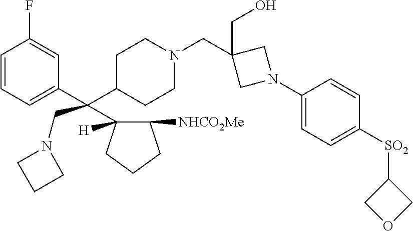 | 699.45 |
| 359 | 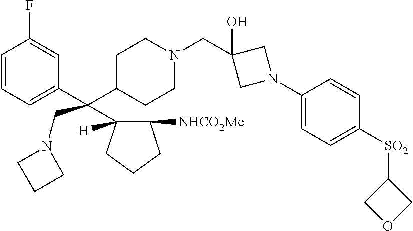 | 685.47 |
| 360 | 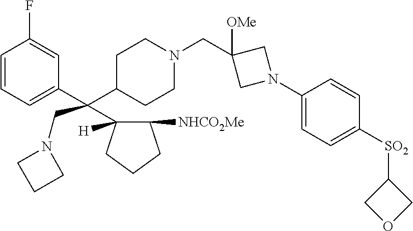 | 699.43 |
| 361 | 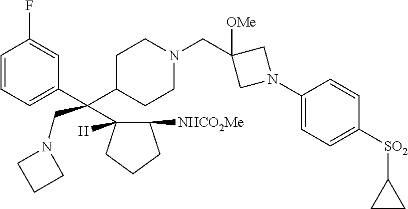 | 683.51 |
| 362 | 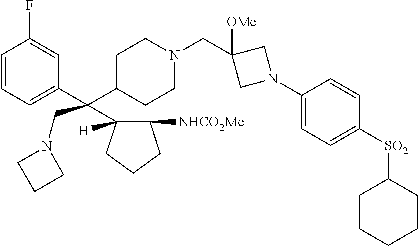 | 725.56 |
| 363 | 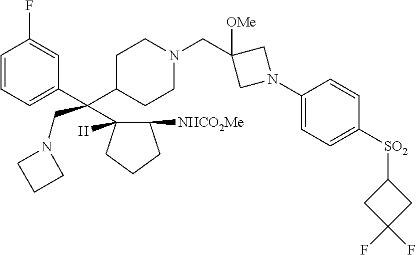 | 733.51 |
| 364 | 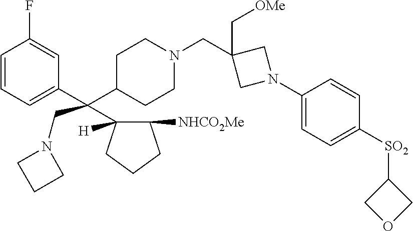 | 713.49 |
| 365 | 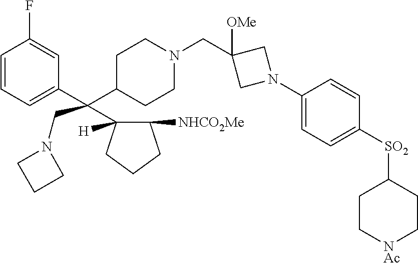 | 768.49 |
| 366 | 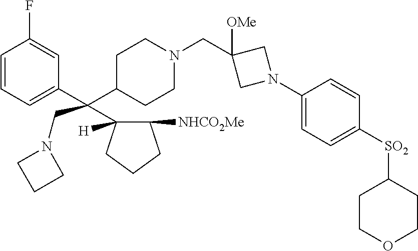 | 727.46 |
| 367 | 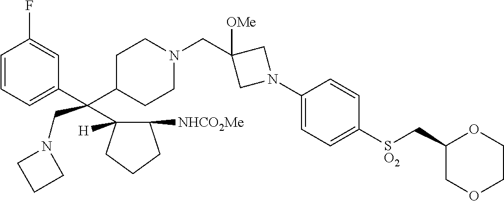 | 743.51 |
| 368 | 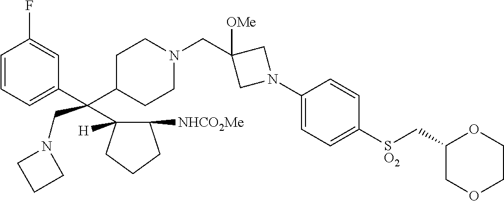 | 743.56 |
| 369 | 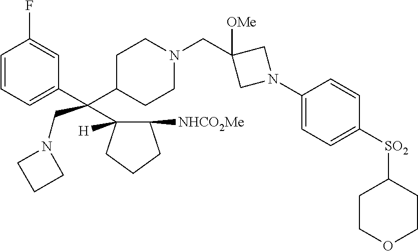 | 727.49 |
| 370 | 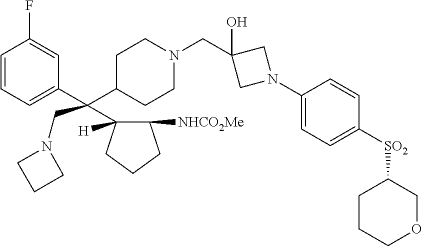 | 713.49 |
| 371 | 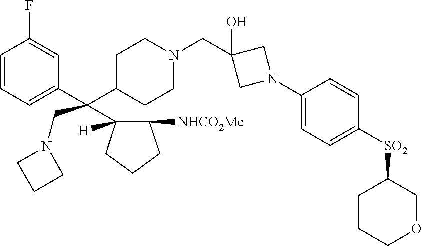 | 713.54 |
| 372 | 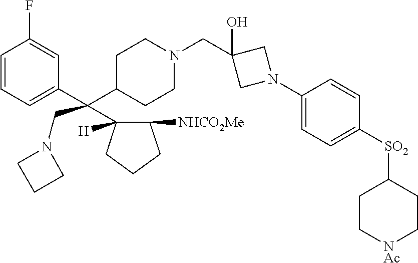 | 754.47 |
| 373 | 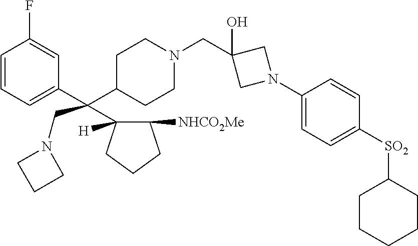 | 711.51 |
| 374 | 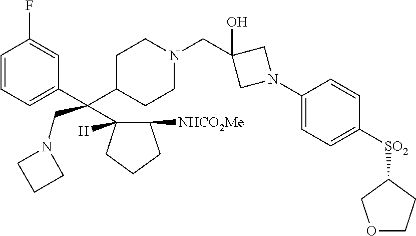 | 699.51 |
| 375 | 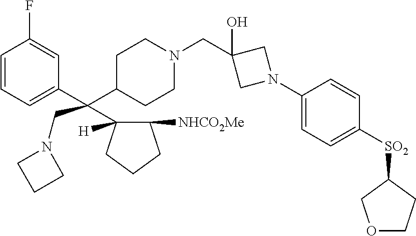 | 699.51 |
| 376 | 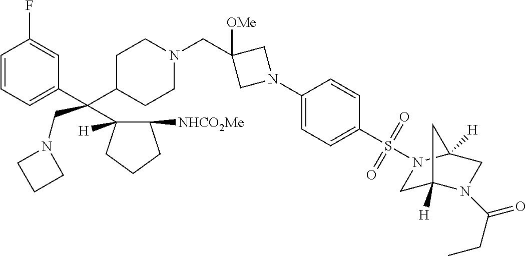 | 795.62 |
| 377 | 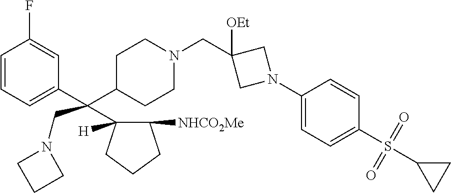 | 697.44 |
| 378 | 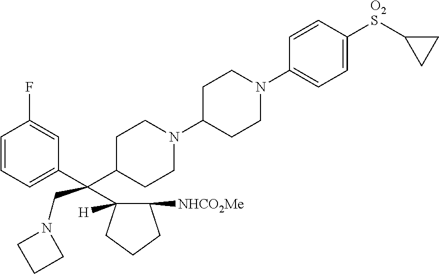 | 667.45 |
| 379 | 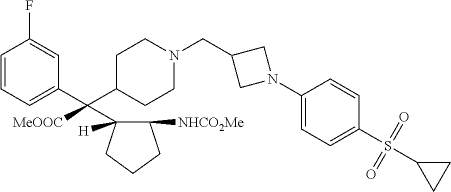 | 642.39 |
| 380 | 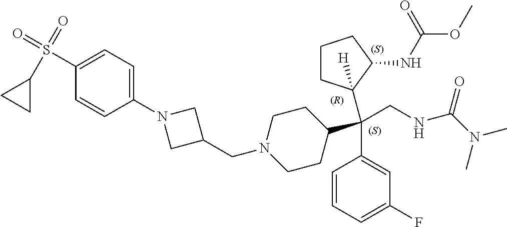 | 684.62 |
| 381 | 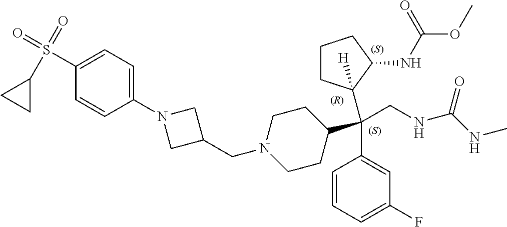 | 670.57 |
| 382 | 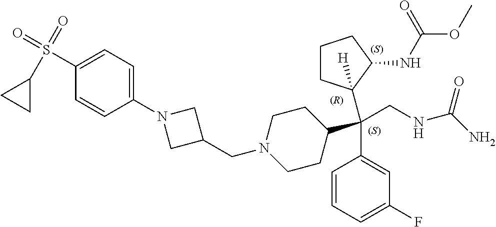 | 656.54 |
| 383 | 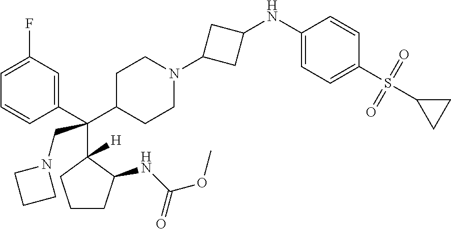 [SMILES: COC(=O)N[C@H]1CCC[C@@H]1[C@](CN1CCC1)(c1cccc(F)c1)C1CCN(C2CC(Nc3ccc(S(=O)(=O)C4CC4)cc3)C2)CC1] | 653.32 |
| 384 | 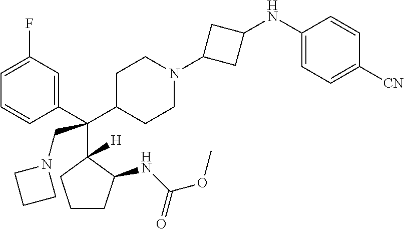 [SMILES: COC(=O)N[C@H]1CCC[C@@H]1[C@](CN1CCC1)(c1cccc(F)c1)C1CCN(C2CC(Nc3ccc(C#N)cc3)C2)CC1] | 574.35 |
| 385 | 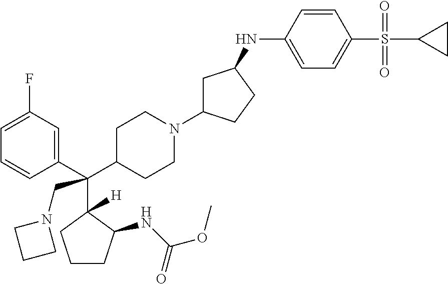 [SMILES: COC(=O)N[C@H]1CCC[C@@H]1[C@](CN1CCC1)(c1cccc(F)c1)C1CCN(C2CC[C@H](Nc3ccc(S(=O)(=O)C4CC4)cc3)C2)CC1] | 667.82 |
| 386 | 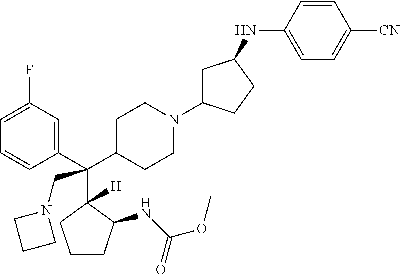 [SMILES: COC(=O)N[C@H]1CCC[C@@H]1[C@](CN1CCC1)(c1cccc(F)c1)C1CCN(C2CC[C@H](Nc3ccc(C#N)cc3)C2)CC1] | 589.03 |
| 387 | 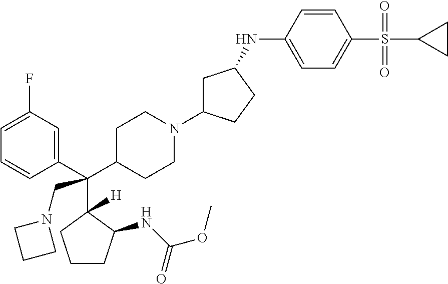 [SMILES: COC(=O)N[C@H]1CCC[C@@H]1[C@](CN1CCC1)(c1cccc(F)c1)C1CCN(C2CC[C@@H](Nc3ccc(S(=O)(=O)C4CC4)cc3)C2)CC1] | 667.81 |
| 388 | 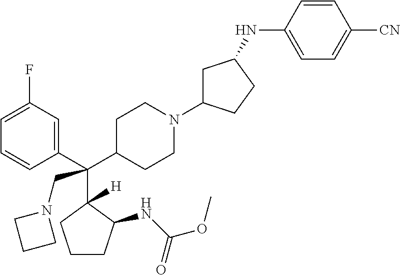 [SMILES: COC(=O)N[C@H]1CCC[C@@H]1[C@](CN1CCC1)(c1cccc(F)c1)C1CCN(C2CC[C@@H](Nc3ccc(C#N)cc3)C2)CC1] | 588.86 |
| 389 | 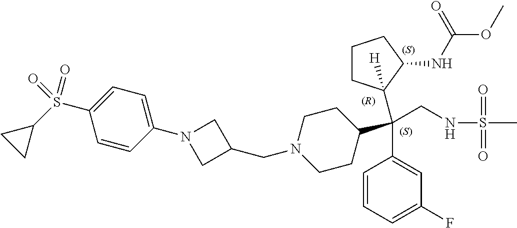 | 691.4 |
| 390 | 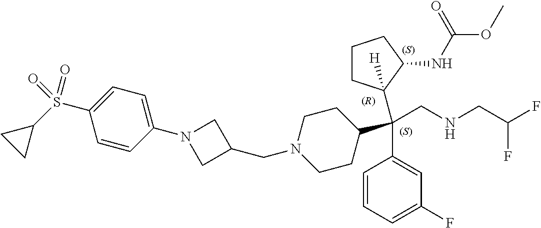 | 677.8 |
| 391 | 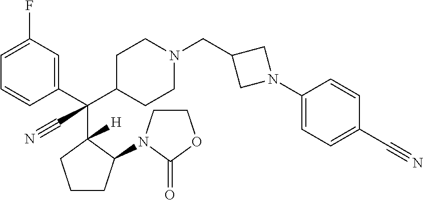 [SMILES: N#Cc1ccc(N2CC(CN3CCC([C@@](C#N)(c4cccc(F)c4)[C@H]4CCC[C@@H]4N4CCOC4=O)CC3)C2)cc1] | 542.0 |
| 392/393 | 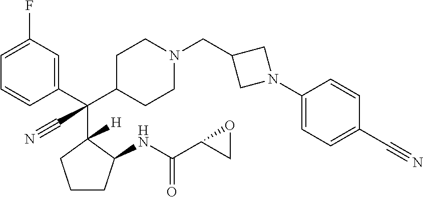 [SMILES: N#Cc1ccc(N2CC(CN3CCC([C@@](C#N)(c4cccc(F)c4)[C@H]4CCC[C@@H]4NC(=O)[C@H]4CO4)CC3)C2)cc1] | 542.6 |
| 392/393 | 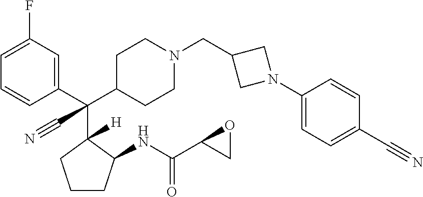 [SMILES: N#Cc1ccc(N2CC(CN3CCC([C@@](C#N)(c4cccc(F)c4)[C@H]4CCC[C@@H]4NC(=O)[C@@H]4CO4)CC3)C2)cc1] | 542.3 |
| 394 | 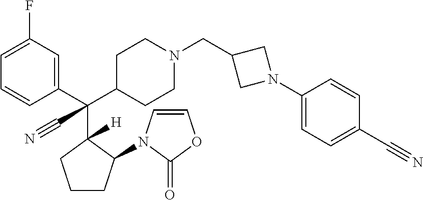 [SMILES: N#Cc1ccc(N2CC(CN3CCC([C@@](C#N)(c4cccc(F)c4)[C@H]4CCC[C@@H]4n4ccoc4=O)CC3)C2)cc1] | 540.2 |
| 395 | 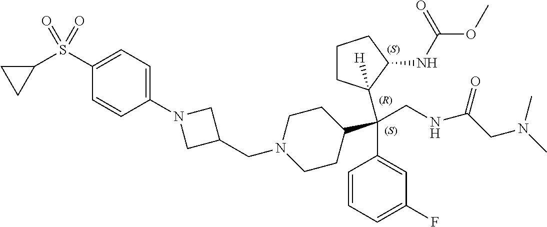 | 698.62 |
| 396 | 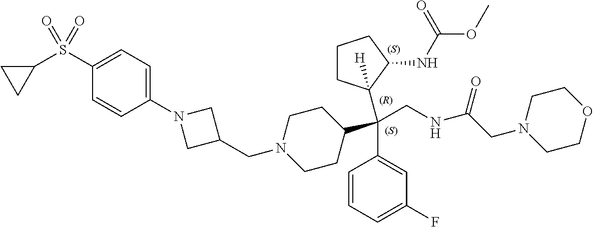 | 740.57 |
| 397 | 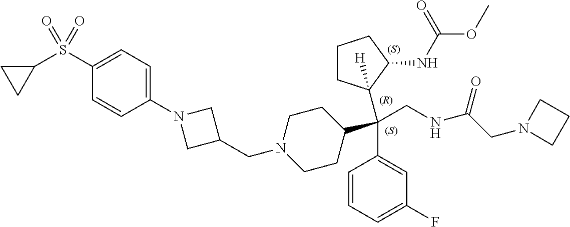 | 710.66 |
| 398 | 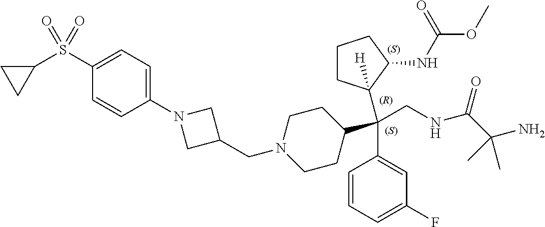 | 698.68 |
| 399 | 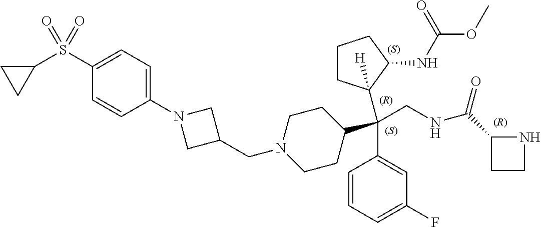 | 696.62 |
| 400 | 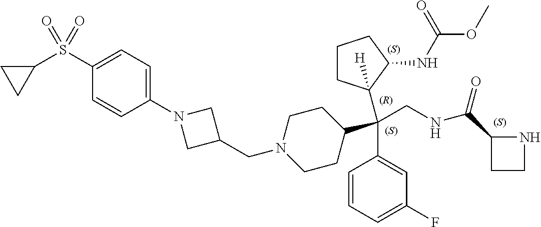 | 696.63 |

TABLE 1.3A

| Cpd. No. | Chemical Name |
|---|---|
| 250 | methyl ((1S,2R)-2-((S)-2-acetamido-1-(1-((1-(3-((3-fluoroazetidin-1- |
| | yl)methyl)-4-(methylsulfonyl)phenyl)azetidin-3-yl)methyl)piperidin-4- |
| | yl)-1-(3-fluorophenyl)ethyl)cyclopentyl)carbamate |
| 251 | methyl N-[2S)-2-{1-[(1-{4-cyano-3-[(3-fluoroazetidin-1-yl)methyl] |
| | phenyl}azetidin-3-yl)methyl]piperidin-4-yl}-2-(3-fluorophenyl)-2- |
| | [(1R,2S)-2-[(methoxycarbonyl)amino]cyclopentyl]ethyl]carbamate |
| 252 | methyl N-[(1S,2R)-2-[(1S)-2-(azetidin-1-yl)-1-{1-[(1-{4-cyano-3-[(3- |
| | fluoroazetidin-1-yl)methyl]phenyl}-3-hydroxyazetidin-3- |
| | yl)methyl]piperidin-4-yl}-1-(3-fluorophenyl)ethyl]cyclopentyl]carbamate |
| 253 | 4-[3-({4-[(1S)-2-acetamido-1-(3-fluorophenyl)-1-[(1R,2S)-2- |
| | [(methoxycarbonyl)amino]cyclopentyl]ethyl]piperidin-1- |
| | yl}methyl)azetidin-1-yl]-2-[(3-fluoroazetidin-1-yl)methyl]benzoic acid |
| 254 | methyl N-[(2S)-2-{1-[(1-{3-[(3-fluoroazetidin-1-yl)methyl]-4- |
| | methanesulfonylphenyl]azetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 255 | methyl N-[(1S,2R)-2-[(1S)-1-{1-[(1-{4-cyano-3-[(3-fluoroazetidin-1- |
| | yl)methyl]phenyl}-3-hydroxyazetidin-3-yl)methyl]piperidin-4-yl}-2- |
| | acetamido-1-(3-fluorophenyl)ethyl]cyclopentyl]carbamate |
| 256 | methyl N-[(1S,2R)-2-[(1S)-2-acetamido-1-{1-[(1-{3-[(3-fluoroazetidin-1- |
| | yl)methyl]-4-methanesulfonylphenyl}-3-hydroxyazetidin-3- |
| | yl)methyl]piperidin-4-yl}-1-(3-fluorophenyl)ethyl]cyclopentyl]carbamate |
| 257 | methyl ((1S,2R)-2-((S)-2-acetamido-1-(1-((1-(3-((3-fluoroazetidin-1- |
| | yl)methyl)-4-(trifluoromethyl)phenyl)azetidin-3-yl)methyl)piperidin-4- |
| | yl)-1-(3-fluorophenyl)ethyl)cyclopentyl)carbamate |
| 258 | methyl N-[(2S)-2-{1-[(1-{4-cyano-3-[(3-fluoroazetidin-1- |
| | yl)methyl]phenyl}-3-fluoroazetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 259 | methyl N-[(2S)-2-{1-[(3-fluoro-1-{3-[(3-fluoroazetidin-1-yl)methyl]-4- |
| | methanesulfonylphenyl}azetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 260 | methyl N-[(2S)-2-{1-[(1-{3-[(3-fluoroazetidin-1-yl)methyl]-4- |
| | (trifluoromethyl)phenyl}azetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 261 | methyl N-[(1S,2R)-2-[(1S)-1-{1-[(1-{4-cyano-3-[(3-fluoroazetidin-1- |
| | yl)methyl]phenyl}-3-methylazetidin-3-yl)methyl]piperidin-4-yl}-2- |
| | acetamido-1-(3-fluorophenyl)ethyl]cyclopentyl]carbamate |
| 262 | methyl N-[(1S,2R)-2-[(1S)-2-(azetidin-1-yl)-1-{1-[(1-{4-cyano-3-[(3- |
| | fluoroazetidin-1-yl)methyl]phenyl}-3-methylazetidin-3- |
| | yl)methyl]piperidin-4-yl}-1-(3- |
| | fluorophenyl)ethyl]cyclopentyl]carbamate |
| 263 | methyl N-[(1S,2R)-2-[(1S)-1-{1-[(1-{4-cyano-3-[(3-fluoroazetidin-1- |
| | yl)methyl]phenyl}azetidin-3-yl)methyl]piperidin-4-yl}-1-(3- |
| | fluorophenyl)-2-(morpholin-4-yl)ethyl]cyclopentyl]carbamate |
| 264 | methyl N-[(1S,2R)-2-[(1S)-1-{1-[(1-{4-cyano-3-[(3-fluoroazetidin-1- |
| | yl)methyl]phenyl}azetidin-3-yl)methyl]piperidin-4-yl}-1-(3- |
| | fluorophenyl)-2-(4-hydroxy-4-methylpiperidin-1- |
| | yl)ethyl]cyclopentyl]carbamate |
| 265 | methyl N-[(1S,2R)-2-[(1S)-1-{1-[(1-{4-cyano-2-fluoro-3-[(3- |
| | fluoroazetidin-1-yl)methyl]phenyl}azetidin-3-yl)methyl]piperidin-4- |
| | yl}-2-acetamido-1-(3-fluorophenyl)ethyl]cyclopentyl]carbamate |
| 266 | methyl N-[(1S,2R)-2-[(1S)-1-{1-[(1-{4-cyano-2-fluoro-5-[(3- |
| | fluoroazetidin-1-yl)methyl]phenyl}azetidin-3-yl)methyl]piperidin-4- |
| | yl}-2-acetamido-1-(3-fluorophenyl)ethyl]cyclopentyl]carbamate |
| 267 | methyl N-[(1S,2R)-2-[(1S)-2-(azetidin-1-yl)-1-(1-{[1-(5-cyanopyridin- |
| | 2-yl)azetidin-3-yl]methyl}piperidin-4-yl)-1-(3- |
| | fluorophenyl)ethyl]cyclopentyl]carbamate |
| 268 | methyl N-[(1S,2R)-2-[(1S)-2-(azetidin-1-yl)-1-(1-{[1-(6-cyanopyridin- |
| | 3-yl)azetidin-3-yl]methyl}piperidin-4-yl)-1-(3- |
| | fluorophenyl)ethyl]cyclopentyl]carbamate |
| 269 | methyl N-[(1S,2R)-2-[(1S)-1-{1-[(1-{4-cyano-3-[(3-fluoroazetidin-1- |
| | yl)methyl]phenyl}azetidin-3-yl)methyl]piperidin-4-yl}-1-(3- |
| | fluorophenyl)-2-(pyrrolidin-1-yl)ethyl]cyclopentyl]carbamate |
| 270 | methyl N-[(1S,2R)-2-[(1S)-1-{1-[(1-{4-cyano-3-[(3-fluoroazetidin-1- |
| | yl)methyl]phenyl}azetidin-3-yl)methyl]piperidin-4-yl}-1-(3- |
| | fluorophenyl)-2-(piperidin-1-yl)ethyl]cyclopentyl]carbamate |
| 271 | methyl N-[(2S)-2-{1-[(1-{4-cyano-3- |
| | [(dimethylamino)methyl]phenyl}azetidin-3-yl)methyl]piperidin-4-yl}-2- |
| | (3-fluorophenyl)-2-[(1R,2S)-2- |
| | [(methoxycarbonyl)amino]cyclopentyl]ethyl]carbamate |
| 272 | methyl N-[(2S)-2-{1-[(1-{4-cyano-3-[(dimethylamino)methyl]-2- |
| | fluorophenyl}azetidin-3-yl)methyl]piperidin-4-yl}-2-(3-fluorophenyl)- |
| | 2-[(1R,2S)-2-[(methoxycarbonyl)amino]cyclopentyl]ethyl]carbamate |
| 273 | methyl N-[(2S)-2-{1-[(1-{4-cyano-5-[(dimethylamino)methyl]-2- |
| | fluorophenyl}azetidin-3-yl)methyl]piperidin-4-yl}-2-(3-fluorophenyl)- |
| | 2-[(1R,2S)-2-[(methoxycarbonyl)amino]cyclopentyl]ethyl]carbamate |
| 274 | methyl N-[(1S,2R)-2-[(1S)-1-{1-[(1-{4-cyano-3- |
| | [(dimethylamino)methyl]-2-fluorophenyl}azetidin-3- |
| | yl)methyl]piperidin-4-yl}-2-acetamido-1-(3- |
| | fluorophenyl)ethyl]cyclopentyl]carbamate |
| 275 | methyl N-[(1S,2R)-2-[(1S)-1-{1-[(1-{4-cyano-5- |
| | [(dimethylamino)methyl]-2-fluorophenyl}azetidin-3- |
| | yl)methyl]piperidin-4-yl}-2-acetamido-1-(3- |
| | fluorophenyl)ethyl]cyclopentyl]carbamate |
| 276 | methyl N-[(1S,2R)-2-[(S)-cyano({1-[(1-{4-cyano-3- |
| | [(dimethylamino)methyl]-2-fluorophenyl}azetidin-3- |
| | yl)methyl]piperidin-4-yl})(3- |
| | fluorophenyl)methyl]cyclopentyl]carbamate |
| 277 | methyl N-[(1S,2R)-2-[(S)-cyano({1-[(1-{4-cyano-5- |
| | [(dimethylamino)methyl]-2-fluorophenyl}azetidin-3- |
| | yl)methyl]piperidin-4-yl})(3- |
| | fluorophenyl)methyl]cyclopentyl]carbamate |
| 278 | methyl N-[(1S,2R)-2-[(1S)-2-(azetidin-1-yl)-1-{1-[(1-{4-cyano-3- |
| | [(dimethylamino)methyl]-2-fluorophenyl}azetidin-3- |
| | yl)methyl]piperidin-4-yl}-1-(3- |
| | fluorophenyl)ethyl]cyclopentyl]carbamate |
| 279 | methyl N-[(1S,2R)-2-[(1S)-2-(azetidin-1-yl)-1-{1-[(1-{4-cyano-5- |
| | [(dimethylamino)methyl]-2-fluorophenyl}azetidin-3- |
| | yl)methyl]piperidin-4-yl}-1-(3- |
| | fluorophenyl)ethyl]cyclopentyl]carbamate |
| 280 | methyl N-[(1S,2R)-2-[(1S)-2-(azetidin-1-yl)-1-{1-[(1-{4-cyano-3- |
| | [(dimethylamino)methyl]-2-fluorophenyl}-3-methoxyazetidin-3- |
| | yl)methyl]piperidin-4-yl}-1-(3- |
| | fluorophenyl)ethyl]cyclopentyl]carbamate |
| 281 | methyl N-[(1S,2R)-2-[(1S)-2-(azetidin-1-yl)-1-{1-[(1-{4-cyano-5- |
| | [(dimethylamino)methyl]-2-fluorophenyl}-3-methoxyazetidin-3- |
| | yl)methyl]piperidin-4-yl}-1-(3- |
| | fluorophenyl)ethyl]cyclopentyl]carbamate |
| 282 | methyl N-[(1S,2R)-2-[(1S)-2-(azetidin-1-yl)-1-{1-[(1-{4-cyano-3- |
| | [(dimethylamino)methyl]-2-fluorophenyl}-3-fluoroazetidin-3- |
| | yl)methyl]piperidin-4-yl}-1-(3- |
| | fluorophenyl)ethyl]cyclopentyl]carbamate |
| 283 | methyl N-[(1S,2R)-2-[(1S)-2-(azetidin-1-yl)-1-{1-[(1-{4-cyano-5- |
| | [(dimethylamino)methyl]-2-fluorophenyl}-3-fluoroazetidin-3- |
| | yl)methyl]piperidin-4-yl}-1-(3- |
| | fluorophenyl)ethyl]cyclopentyl]carbamate |
| 284 | methyl N-[(1S,2R)-2-[(1S)-1-{1-[(1-{4-cyano-3- |
| | [(dimethylamino)methyl]-2-fluorophenyl}azetidin-3- |
| | yl)methyl]piperidin-4-yl}-1-(3-fluorophenyl)-2-(4-hydroxy-4- |
| | methylpiperidin-1-yl)ethyl]cyclopentyl]carbamate |
| 285 | methyl N-[(1S,2R)-2-[(1S)-1-{1-[(1-{4-cyano-5- |
| | [(dimethylamino)methyl]-2-fluorophenyl}azetidin-3- |
| | yl)methyl]piperidin-4-yl}-1-(3-fluorophenyl)-2-(4-hydroxy-4- |
| | methylpiperidin-1-yl)ethyl]cyclopentyl]carbamate |
| 286 | methyl N-[(1S,2R)-2-[(1S)-1-{1-[(1-{4-cyano-3- |
| | [(dimethylamino)methyl]-2-fluorophenyl}azetidin-3- |
| | yl)methyl]piperidin-4-yl}-1-(3-fluorophenyl)-2-(morpholin-4- |
| | yl)ethyl]cyclopentyl]carbamate |
| 287 | methyl N-[(1S,2R)-2-[(1S)-1-{1-[(1-{4-cyano-5- |
| | [(dimethylamino)methyl]-2-fluorophenyl}azetidin-3- |
| | yl)methyl]piperidin-4-yl}-1-(3-fluorophenyl)-2-(morpholin-4- |
| | yl)ethyl]cyclopentyl]carbamate |
| 288 | methyl N-[(2S)-2-{1-[(1-{4-cyano-3-[(dimethylamino)methyl]-2- |
| | fluorophenyl}-3-fluoroazetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 289 | methyl N-[(2S)-2-{1-[(1-{4-cyano-5-[(dimethylamino)methyl]-2- |
| | fluorophenyl}-3-fluoroazetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 290 | methyl N-[(1S,2R)-2-[(1S)-2-(azetidin-1-yl)-1-[1-({1-[4-cyano-3- |
| | (dimethylcarbamoyl)phenyl]azetidin-3-yl}methyl)piperidin-4-yl]-1-(3- |
| | fluorophenypethyl]cyclopentyl]carbamate |
| 291 | methyl N-[(1S,2R)-2-[(1S)-1-{1-[(1-{4-cyano-5- |
| | [(dimethylamino)methyl]-2-fluorophenyl}-3-fluoroazetidin-3- |
| | yl)methyl]piperidin-4-yl}-1-(3-fluorophenyl)-2-(4-hydroxy-4- |
| | methylpiperidin-1-yl)ethyl]cyclopentyl]carbamate |
| 292 | methyl N-[(1S,2R)-2-[(1S)-1-{1-[(1-{4-cyano-5- |
| | [(dimethylamino)methyl]-2-fluorophenyl}-3-hydroxyazetidin-3- |
| | yl)methyl]piperidin-4-yl}-1-(3-fluorophenyl)-2-(4-hydroxy-4- |
| | methylpiperidin-1-yl)ethyl]cyclopentyl]carbamate |
| 293 | methyl N-[(1S,2R)-2-[(1S)-1-{1-[(1-{4-cyano-5- |
| | [(dimethylamino)methyl]-2-fluorophenyl}-3-fluoroazetidin-3- |
| | yl)methyl]piperidin-4-yl}-1-(3-fluorophenyl)-2-(morpholin-4- |
| | yl)ethyl]cyclopentyl]carbamate |
| 294 | methyl N-[(1S,2R)-2-[(1S)-1-{1-[(1-{4-cyano-5- |
| | [(dimethylamino)methyl]-2-fluorophenyl}-3-hydroxyazetidin-3- |
| | yl)methyl]piperidin-4-yl}-1-(3-fluorophenyl)-2-(morpholin-4- |
| | yl)ethyl]cyclopentyl]carbamate |
| 295 | methyl N-[(1S,2R)-2-[(1S)-2-(azetidin-1-yl)-1-(1-{[1-(4-cyanophenyl)- |
| | 3-hydroxyazetidin-3-yl]methyl}piperidin-4-yl)-1-(3- |
| | fluorophenyl)ethyl]cyclopentyl]carbamate |
| 296 | methyl N-[(1S,2R)-2-[(1S)-2-(azetidin-1-yl)-1-[-({1-[4- |
| | (cyclopropanesulfonyl)phenyl]-3-hydroxyazetidin-3-yl}methyl)piperidin- |
| | 4-yl]-1-(3-fluorophenyl)ethyl]cyclopentyl]carbamate |
| 297 | methyl N-[(1S,2R)-2-[(1S)-1-{1-[(1-{5-[(azetidin-1-yl)methyl]- |
| | 4-cyano-2-fluorophenyl}azetidin-3-yl)methyl]piperidin-4-yl}-1-(3- |
| | fluorophenyl)-2-(morpholin-4-yl)ethyl]cyclopentyl]carbamate |
| 298 | methyl N-[(2S)-2-{1-[(1-{5-[(azetidin-1-yl)methyl]-4-cyano-2- |
| | fluorophenyl}-3-fluoroazetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 299 | methyl N-[(1S,2R)-2-[(1S)-1-{1-[(1-{5-[(azetidin-1-yl)methyl]-4- |
| | cyano-2-fluorophenyl}-3-fluoroazetidin-3-yl)methyl]piperidin-4-yl}-1-(3- |
| | fluorophenyl)-2-(morpholin-4-yl)ethyl]cyclopentyl]carbamate |
| 300 | methyl N-[(1S,2R)-2-[(1S)-1-{1-[(1-{4-cyano-3-[(dimethylamino)methyl]- |
| | 2-fluorophenyl}-3-fluoroazetidin-3-yl)methyl]piperidin-4-yl}-2-acetamido-1- |
| | (3-fluorophenyl)ethyl]cyclopentyl]carbamate |
| 301 | methyl N-[(1S,2R)-2-[(1S)-2-(azetidin-1-yl)-1-(1-{[3-fluoro-1-(4- |
| | methanesulfonylphenyl)azetidin-3-yl]methyl}piperidin-4-yl)-1-(3- |
| | fluorophenyl)ethyl]cyclopentyl]carbamate |
| 302 | methyl N-[(1S,2R)-2-[(1S)-2-(azetidin-1-yl)-1-(3-fluorophenyl)-1-(1- |
| | {[3-hydroxy-1-(4-methanesulfonylphenyl)azetidin-3- |
| | yl]methyl}piperidin-4-yl)ethyl]cyclopentyl]carbamate |
| 303 | methyl N-[(1S,2R)-2-[(1S)-1-{1-[(1-{4-cyano-3- |
| | [(dimethylamino)methyl]-2-fluorophenyl}-3-fluoroazetidin-3- |
| | yl)methyl]piperidin-4-yl}-1-(3-fluorophenyl)-2-(morpholin-4- |
| | yl)ethyl]cyclopentyl]carbamate |
| 304 | methyl N-[(1S,2R)-2-[(1S)-1-{1-[(1-{4-cyano-3- |
| | [(dimethylamino)methyl]-2-fluorophenyl}azetidin-3- |
| | yl)methyl]piperidin-4-yl}-1-(3-fluorophenyl)-2-(1H-1,2,3-triazol-1- |
| | yl)ethyl]cyclopentyl]carbamate |
| 305 | methyl N-[(1S,2R)-2-[(1S)-1-{1-[(1-{4-cyano-3- |
| | [(dimethylamino)methyl]-2-fluorophenyl}-3-fluoroazetidin-3- |
| | yl)methyl]piperidin-4-yl}-1-(3-fluorophenyl)-2-(1H-1,2,3-triazol-1- |
| | yl)ethyl]cyclopentyl]carbamate |
| 306 | methyl N-[(1S,2R)-2-[(1S)-2-(azetidin-1-yl)-1-{1-[(1-{4-cyano-3- |
| | [(dimethylamino)methyl]-2-fluorophenyl}-3-hydroxyazetidin-3- |
| | yl)methyl]piperidin-4-yl}-1-(3- |
| | fluorophenyl)ethyl]cyclopentyl]carbamate |
| 307 | methyl N-[(1S,2R)-2-[(1S)-2-(azetidin-1-yl)-1-{1-[(1-{4-cyano-3- |
| | [(dimethylamino)methyl]-2-fluorophenyl}-3-methylazetidin-3- |
| | yl)methyl]piperidin-4-yl}-1-(3- |
| | fluorophenyl)ethyl]cyclopentyl]carbamate |
| 308 | methyl N-[(2S)-2-{1-[(1-{4-cyano-3-[(dimethylamino)methyl]-2- |
| | fluorophenyl}-3-methylazetidin-3-yl)methyl]piperidin-4-yl}-2-(3- |
| | fluorophenyl)-2-[(1R,2S)-2- |
| | [(methoxycarbonyl)amino]cyclopentyl]ethyl]carbamate |
| 309 | methyl N-[(1S,2R)-2-[(1S)-2-(azetidin-1-yl)-1-[1-(1-{1-[4- |
| | (dimethylsulfamoyl)phenyl]-3-fluoroazetidin-3-yl}methyl)piperidin-4- |
| | yl]-1-(3-fluorophenyl)ethyl]cyclopentyl]carbamate |
| 310 | methyl N-[(1S,2R)-2-[(1S)-2-(azetidin-1-yl)-1-[1-({3-fluoro-1-[4- |
| | (pyrrolidine-1-sulfonyl)phenyl]azetidin-3-yl}methyl)piperidin-4-yl]-1- |
| | (3-fluorophenyl)ethyl]cyclopentyl]carbamate |
| 311 | methyl N-[(1S,2R)-2-[(1S)-2-(azetidin-1-yl)-1-{1-[(1-{3- |
| | [(dimethylamino)methyl]-2-fluoro-4-methanesulfonylphenyl}-3- |
| | fluoroazetidin-3-yl)methyl]piperidin-4-yl}-1-(3- |
| | fluorophenyl)ethyl]cyclopentyl]carbamate |
| 312 | methyl N-[(1S,2R)-2-[(1S)-2-(azetidin-1-yl)-1-{1-[(1-{3- |
| | [(dimethylamino)methyl]-2-fluoro-4-methanesulfonylphenyl}-3- |
| | methylazetidin-3-yl)methyl]piperidin-4-yl}-1-(3- |
| | fluorophenyl)ethyl]cyclopentyl]carbamate |
| 313 | methyl N-[(1S,2R)-2-[(1S)-2-(azetidin-1-yl)-1-[1-({1-[4-cyano-3- |
| | (dimethylcarbamoyl)phenyl]-3-fluoroazetidin-3-yl}methyl)piperidin-4- |
| | yl]-1-(3-fluorophenyl)ethyl]cyclopentyl]carbamate |
| 314 | methyl N-[(1S,2R)-2-[(1S)-2-(azetidin-1-yl)-1-[1-({1-[4-cyano-3-(3- |
| | hydroxy-3-methylazetidine-1-carbonyl)phenyl]-3-fluoroazetidin-3- |
| | yl}methyl)piperidin-4-yl]-1-(3- |
| | fluorophenyl)ethyl]cyclopentyl]carbamate |
| 315 | methyl N-[(1S,2R)-2-[(1S)-2-(azetidin-1-yl)-1-[1-({1-[4-cyano-3- |
| | (morpholine-4-carbonyl)phenyl]-3-fluoroazetidin-3- |
| | yl}methyl)piperidin-4-yl]-1-(3- |
| | fluorophenyl)ethyl]cyclopentyl]carbamate |
| 316 | methyl N-[(1S,2R)-2-[(1S)-2-(azetidin-1-yl)-1-[1-({1-[4-cyano-3- |
| | (methylcarbamoyl)phenyl]-3-fluoroazetidin-3-yl}methyl)piperidin-4- |
| | yl]-1-(3-fluorophenyl)ethyl]cyclopentyl]carbamate |
| 317 | methyl N-[(1S,2R)-2-[(1S)-2-(azetidin-1-yl)-1-[1-({1-[4-cyano-3- |
| | (dimethylcarbamoyl)phenyl]-3-methylazetidin-3-yl}methyl)piperidin- |
| | 4-yl]-1-(3-fluorophenyl)ethyl]cyclopentyl]carbamate |
| 318 | methyl 4-{4-[3-({4-[(1S)-2-(azetidin-1-yl)-1-(3-fluorophenyl)-1- |
| | [(1R,2S)-2-[(methoxycarbonyl)amino]cyclopentyl]ethyl]piperidin-1- |
| | yl}methyl)-3-methoxyazetidin-1-yl]benzenesulfonyl}piperidine-1- |
| | carboxylate |
| 319 | methyl N-[(1S,2R)-2-[(1S)-2-(azetidin-1-yl)-1-(3-fluorophenyl)-1-(1- |
| | {[3-methoxy-1-(4-{[1-(methylcarbamoyl)piperidin-4- |
| | yl]sulfonyl}phenyl)azetidin-3-yl]methyl}piperidin-4- |
| | yl)ethyl]cyclopentyl]carbamate |
| 320 | methyl ((1S,2R)-2-((S)-2-(azetidin-1-yl)-1-(3-fluorophenyl)-1-(1-((3- |
| | methoxy-1-(4-((1-propionylpiperidin-4-yl)sulfonyl)phenyl)azetidin-3- |
| | yl)methyl)piperidin-4-yl)ethyl)cyclopentyl)carbamate |
| 321 | methyl ((1S,2R)-2-((S)-2-(azetidin-1-yl)-1-(1-((1-(4-cyano-3- |
| | (dimethylcarbamoyl)phenyl)-3-methoxyazetidin-3-yl)methyl)piperidin- |
| | 4-yl)-1-(3-fluorophenyl)ethyl)cyclopentyl)carbamate |
| 322 | methyl N-[(1S,2R)-2-[(1S)-1-(1-{[1-(4-{[(3R)-1-acetylpiperidin-3- |
| | yl]sulfonyl}phenyl)-3-methoxyazetidin-3-yl]methyl}piperidin-4-yl)-2- |
| | (azetidin-1-yl)-1-(3-fluorophenyl)ethyl]cyclopentyl]carbamate |
| 323 | methyl N-[(1S,2R)-2-[(1S)-2-(azetidin-1-yl)-1-(3-fluorophenyl)-1-{1- |
| | [(3-methoxy-1-{4-[(1-methylpiperidin-4-yl)sulfonyl]phenyl}azetidin- |
| | 3-yl)methyl]piperidin-4-yl}ethyl]cyclopentyl]carbamate |
| 324 | methyl N-[(2S)-2-[1-({1-[4-cyano-3- |
| | (dimethylcarbamoyl)phenyl]azetidin-3-yl}methyl)piperidin-4-yl]-2-(3- |
| | fluorophenyl)-2-[(1R,2S)-2- |
| | [(methoxycarbonyl)amino]cyclopentyl]ethyl]carbamate |
| 325 | methyl N-[(1S,2R)-2-[(1S)-2-(azetidin-1-yl)-1-(3-fluorophenyl)-1-(1- |
| | {[1-(4-{[1-(1-hydroxycyclopropanecarbonyl)piperidin-4- |
| | yl]sulfonyl}phenyl)-3-methoxyazetidin-3-yl]methyl}piperidin-4- |
| | yl)ethyl]cyclopentyl]carbamate |
| 326 | methyl N-[(1S,2R)-2-[(1S)-2-(azetidin-1-yl)-1-[1-({1-[4-(4-carbamoyl- |
| | 3-fluorobenzenesulfonyl)phenyl]-3-methoxyazetidin-3-yl}methyl)piperidin- |
| | 4-yl]-1-(3-fluorophenyl)ethyl]cyclopentyl]carbamate |
| 327 | methyl N-[(1S,2R)-2-[(1S)-2-(azetidin-1-yl)-1-(3-fluorophenyl)-1-{1-[(3- |
| | methoxy-1-{4-[4-(methylcarbamoyl)benzenesulfonyl]phenyl}azetidin-3- |
| | yl)methyl]piperidin-4-yl}ethyl]cyclopentyl]carbamate |
| 328 | methyl N-[(1S,2R)-2-[(1S)-1-[1-({1-[4-cyano-3- |
| | (dimethylcarbamoyl)phenyl]azetidin-3-yl}methyl)piperidin-4-yl]-2- |
| | (dimethylamino)-1-(3-fluorophenyl)ethyl]cyclopent-3-en-1-yl]carbamate |
| 329 | methyl N-[(1S,2R)-2-[(1S)-1-[1-({1-[4-cyano-3- |
| | (dimethylcarbamoyl)phenyl]azetidin-3-yl}methyl)piperidin-4-yl]-2- |
| | (diethylamino)-1-(3-fluorophenyl)ethyl]cyclopentyl]carbamate |
| 330 | methyl N-[(1S,2R)-2-[(1S)-1-[1-({1-[4-cyano-3- |
| | (dimethylcarbamoyl)phenyl]azetidin-3-yl}methyl)piperidin-4-yl]-2-[(2- |
| | fluoroethyl)(methyl)amino]-1-(3-fluorophenyl)ethyl]cyclopent-3-en-1- |
| | yl]carbamate |
| 331 | methyl N-[(1S,2R)-2-[(1S)-1-[1-({1-[4-cyano-3- |
| | (dimethylcarbamoyl)phenyl]azetidin-3-yl}methyl)piperidin-4-yl]-2- |
| | acetamido-1-(3-fluorophenyl)ethyl]cyclopentyl]carbamate |
| 332 | methyl N-[(1S,2R)-2-[(1S)-1-[1-({1-[4-cyano-3- |
| | (dimethylcarbamoyl)phenyl]azetidin-3-yl}methyl)piperidin-4-yl]-1-(3- |
| | fluorophenyl)-2-(morpholin-4-yl)ethyl]cyclopentyl]carbamate |
| 333 | methyl ((1S,2R)-2-((S)-2-(azetidin-1-yl)-1-(1-((1-(5-cyanothiophen-2- |
| | yl)azetidin-3-yl)methyl)piperidin-4-yl)-1-(3- |
| | fluorophenypethyl)cyclopentyl)carbamate |
| 334 | methyl N-[(1S,2R)-2-[(1S)-2-(azetidin-1-yl)-1-(3-fluorophenyl)-1-{1-[(3- |
| | methoxy-1-{4-[4-(methylcarbamoyl)benzenesulfonyl]phenyl}azetidin-3- |
| | yl)methyl]piperidin-4-yl}ethyl]cyclopentyl]-N-methylcarbamate |
| 335 | methyl N-[(1S,2R)-2-[(1S)-2-(azetidin-1-yl)-1-[1-({1-[2-fluoro-4-({1- |
| | propanoyl-1,6-diazaspiro[3.3]heptan-6-yl}sulfonyl)phenyl]-3-methoxyazetidin- |
| | 3-yl}methyl)piperidin-4-yl]-1-(3-fluorophenyl)ethyl]cyclopentyl]carbamate |
| 336 | methyl N-[(1S,2R)-2-[(S)-cyano[1-({1-[4- |
| | (cyclopropanesulfonyl)phenyl]azetidin-3-yl}methyl)piperidin-4-yl](3- |
| | fluorophenyl)methyl]cyclopentyl]carbamate |
| 337 | 1-[(1S,2R)-2-[(1S)-2-(azetidin-1-yl)-1[1-({1-[4- |
| | (cyclopropanesulfonyl)phenyl]azetidin-3-yl}methyl)piperidin-4-yl]-1- |
| | (3-fluorophenyl)ethyl]cyclopentyl]-3,3-dimethylurea |
| 338 | N-[(1S,2R)-2-[(1S)-2-(azetidin-1-yl)-1-[1-({1-[4- |
| | (cyclopropanesulfonyl)phenyl]azetidin-3-yl}methyl)piperidin-4-yl]-1- |
| | (3-fluorophenyl)ethyl]cyclopentyl]-2,2,2-trifluoroacetamide |
| 339 | methyl N-[(1S,2R)-2-[(1S)-1-[1-({1-[4- |
| | (cyclopropanesulfonyl)phenyl]azetidin-3-yl}methyl)piperidin-4-yl]-1- |
| | (3-fluorophenyl)-2-(3-methylazetidin-1-yl)ethyl]cyclopemtyl]carbamate |
| 340 | 1-[(1S,2R)-2-[(1S)-2-(azetidin-1-yl)-1-[1-({1-[4- |
| | (cyclopropanesulfonyl)phenyl]azetidin-3-yl}methyl)piperidin-4-yl]-1- |
| | (3-fluorophenyl)ethyl]cyclopentyl]pyrrolidin-2-one |
| 341 | methyl N-[(1S,2R)-2-[(1S)-2-(2-ethyl-1H-imidazol-1-yl)-1-(3- |
| | fluorophenyl)-1-(1-{[3-methoxy-1-(4-{[(1r,3s)-3-hydroxy-3- |
| | methylcyclobutyl]sulfonyl}phenyl)azetidin-3-yl]methyl}piperidin-4- |
| | yl)ethyl]cyclopentyl]carbamate |
| 342 | methyl N-[(1S,2R)-2-[(1S)-1-[1-({1-[4- |
| | (cyclopropanesulfonyl)phenyl]azetidin-3-yl}methyl)piperidin-4-yl]-2- |
| | [(2-fluoroethyl)amino]-1-(3-fluorophenyl)ethyl]cyclopentyl]carbamate |
| 343 | methyl N-[(1S,2R)-2-[(1S)-2-(azetidin-1-yl)-1-[1-({1-[4- |
| | (cyclopropanesulfonyl)phenyl]-3-methylazetidin-3- |
| | yl}methyl)piperidin-4-yl]-1-(3- |
| | fluorophenyl)ethyl]cyclopentyl]carbamate |
| 344 | methyl N-[(1S,2R)-2-[(1S)-2-(azetidin-1-yl)-1-[1-({1-[4- |
| | (cyclobutylcarbamoyl)phenyl]azetidin-3-yl}methyl)piperidin-4-yl]-1- |
| | (3-fluorophenyl)ethyl]cyclopentyl]carbamate |
| 345 | methyl N-[(1S,2R)-2-[(1S)-2-(azetidin-1-yl)-1-(1-{[1-(4-{[(3R)-1-(2- |
| | cyanoacetyl)piperidin-3-yl]sulfonyl}phenyl)-3-fluoroazetidin-3- |
| | yl]methyl}piperidin-4-yl)-1-(3-fluorophenyl)ethyl]cyclopentyl]carbamate |
| 346 | methyl N-[(1S,2R)-2-[(1S)-2-(azetidin-1-yl)-1-(1-{[1-(4-{3-[(1- |
| | cyanocyclopropyl)carbamoyl]benzenesulfonyl}phenyl)-3- |
| | fluoroazetidin-3-yl]methyl}piperidin-4-yl)-1-(3- |
| | fluorophenyl)ethyl]cyclopentyl]carbamate |
| 347 | methyl N-[(1S,2R)-2-[(1S)-2-(azetidin-1-yl)-1-(1-{[1-(4-{3-[(1- |
| | cyanocyclopropyl)carbamoyl]benzenesulfonyl}phenyl)-3- |
| | hydroxyazetidin-3-yl]methyl}piperidin-4-yl)-1-(3- |
| | fluorophenyl)ethyl]cyclopentyl]carbamate |
| 348 | methyl N-[(1S,2R)-2-[(1S)-2-(azetidin-1-yl)-1-{1-[(1-{4-[(3,3- |
| | difluoroazetidin-1-yl)sulfonyl]phenyl}-3-fluoroazetidin-3- |
| | yl)methyl]piperidin-4-yl}-1-(3- |
| | fluorophenyl)ethyl]cyclopentyl]carbamate |
| 349 | methyl N-[(1S,2R)-2-[(1S)-2-(azetidin-1-yl)-1-(1-{[1-(4-{[(3R)-1-(2- |
| | cyanoacetyl)azepan-3-yl]sulfonyl}phenyl)-3-fluoroazetidin-3- |
| | yl]methyl}piperidin-4-yl)-1-(3-fluorophenyl)ethyl]cyclopentyl]carbamate |
| 350 | 1-[(1S,2R)-2-[(1S)-2-(azetidin-1-yl)-1-[1-({1-[(4- |
| | (cyclopropanesulfonyl)phenyl]azetidin-3-yl}methyl)piperidin-4-yl]-1- |
| | (3-fluorophenyl)ethyl]cyclopentyl]-3-methylimidazolidin-2-one |
| 351 | methyl N-[(1S,2R)-2-[(1S)-1-[1-({1-[4- |
| | (cyclopropanesulfonyl)phenyl]azetidin-3-yl}methyl)piperidin-4-yl]-1- |
| | (3-fluorophenyl)-2-(3-hydroxyazetidin-1- |
| | yl)ethyl]cyclopentyl]carbamate |
| 352 | methyl N-[(1S,2R)-2-[(1S)-1-[1-({1-[4- |
| | (cyclopropanesulfonyl)phenyl]azetidin-3-yl}methyl)piperidin-4-yl]-1- |
| | (3-fluorophenyl)-2-(3-methoxyazetidin-1- |
| | yl)ethyl]cyclopentyl]carbamate |
| 353 | methyl N-[(1S,2R)-2-[(1S)-1-[1-({1-[4- |
| | (cyclopropanesulfonyl)phenyl]azetidin-3-yl}methyl)piperidin-4-yl]-2- |
| | (3-fluoroazetidin-1-yl)-1-(3-fluorophenyl)ethyl]cyclopentyl]carbamate |
| 354 | methyl N-[(1S,2R)-2-[(1S)-1-[1-({1-[4- |
| | (cyclopropanesulfonyl)phenyl]azetidin-3-yl}methyl)piperidin-4-yl]-1- |
| | (3-fluorophenyl)-2-(methylamino)ethyl]cyclopentyl]carbamate |
| 355 | methyl N-[(1S,2R)-2-[(1S)-1-[1-({1-[4- |
| | (cyclopropanesulfonyl)phenyl]azetidin-3-yl}methyl)piperidin-4-yl]-2- |
| | (ethylamino)-1-(3-fluorophenyl)ethyl]cyclopentyl]carbamate |
| 356 | methyl N-[(1S,2R)-2-[(1S)-1-[1-({1-[4- |
| | (cyclopropanesulfonyl)phenyl]azetidin-3-yl}methyl)piperidin-4-yl]-1- |
| | (3-fluorophenyl)-2-[(propan-2-yl)amino]ethyl]cyclopentyl]carbamate |
| 357 | methyl N-[(1S,2R)-2-[(1S)-2-(cyclobutylamino)-1-[1-({1-[4- |
| | (cyclopropanesulfonyl)phenyl]azetidin-3-yl}methyl)piperidin-4-yl]-1- |
| | (3-fluorophenypethyl]cyclopentyl]carbamate |
| 358 | methyl ((1S,2R)-2-(2-(azetidin-1-yl)-1-(3-fluorophenyl)-1-(1-((3- |
| | (hydroxymethyl)-1-(4-(oxetan-3-ylsulfonyl)phenyl)azetidin-3- |
| | yl)methyl)piperidin-4-yl)ethyl)cyclopentyl)carbamate |
| 359 | methyl ((1S,2R)-2-(2-(azetidin-1-yl)-1-(3-fluorophenyl)-1-(1-((3-hydroxy- |
| | 1-(4-(oxetan-3-ylsulfonyl)phenyl)azetidin-3-yl)methyl)piperidin-4- |
| | yl)ethyl)cyclopentyl)carbamate |
| 360 | methyl ((1S,2R)-2-(2-(azetidin-1-yl)-1-(3-fluorophenyl)-1-(1-((3-methoxy- |
| | 1-(4-(oxetan-3-ylsulfonyl)phenyl)azetidin-3-yl)methyl)piperidin-4- |
| | yl)ethyl)cyclopentyl)carbamate |
| 361 | methyl ((1S,2R)-2-(2-(azetidin-1-yl)-1-(1-((1-(4- |
| | (cyclopropylsulfonyl)phenyl)-3-methoxyazetidin-3- |
| | yl)methyl)piperidin-4-yl)-1-(3- |
| | fluorophenyl)ethyl)cyclopentyl)carbamate |
| 362 | methyl ((1S,2R)-2-(2-(azetidin-1-yl)-1-(1-((1-(4- |
| | (cyclohexylsulfonyl)phenyl)-3-methoxyazetidin-3-yl)methyl)piperidin-4- |
| | yl)-1-(3-fluorophenyl)ethyl)cyclopentyl)carbamate |
| 363 | methyl ((1S,2R)-2-(2-(azetidin-1-yl)-1-(1-((1-(4-((3,3- |
| | difluorocyclobutyl)sulfonyl)phenyl)-3-methoxyazetidin-3- |
| | yl)methyl)piperidin-4-yl)-1-(3- |
| | fluorophenyl)ethyl)cyclopentyl)carbamate |
| 364 | methyl ((1S,2R)-2-(2-(azetidin-1-yl)-1-(3-fluorophenyl)-1-(1-((3- |
| | (methoxymethyl)-1-(4-(oxetan-3-ylsulfonyl)phenyl)azetidin-3- |
| | yl)methyl)piperidin-4-yl)ethyl)cyclopentyl)carbamate |
| 365 | methyl ((1S,2R)-2-(1-(1-((1-(4-((1-acetylpiperidin-4- |
| | yl)sulfonyl)phenyl)-3-methoxyazetidin-3-yl)methyl)piperidin-4-yl)-2- |
| | (azetidin-1-yl)-1-(3-fluorophenyl)ethyl)cyclopentyl)carbamate |
| 366 | methyl ((1S,2R)-2-(2-(azetidin-1-yl)-1-(3-fluorophenyl)-1-(1-((3- |
| | methoxy-1-(4-((tetrahydro-2H-pyran-4-yl)sulfonyl)phenyl)azetidin-3- |
| | yl)methyl)piperidin-4-yl)ethyl)cyclopentyl)carbamate |
| 367 | methyl ((1S,2R)-2-(1-(1-((1-(4-((((S)-1,4-dioxan-2- |
| | yl)methyl)sulfonyl)phenyl)-3-methoxyazetidin-3-yl)methyl)piperidin- |
| | 4-yl)-2-(azetidin-1-yl)-1-(3-fluorophenyl)ethyl)cyclopentyl)carbamate |
| 368 | methyl ((1S,2R)-2-(1-(1-((1-(4-((((R)-1,4-dioxan-2- |
| | yl)methyl)sulfonyl)phenyl)-3-methoxyazetidin-3-yl)methyl)piperidin- |
| | 4-yl)-2-(azetidin-1-yl)-1-(3-fluorophenyl)ethyl)cyclopentyl)carbamate |
| 369 | methyl ((1S,2R)-2-(2-(azetidin-1-yl)-1-(3-fluorophenyl)-1-(1-((3- |
| | methoxy-1-(4-((tetrahydro-2H-pyran-4-yl)sulfonyl)phenyl)azetidin-3- |
| | yl)methyl)piperidin-4-yl)ethyl)cyclopentyl)carbamate |
| 370 | methyl ((1S,2R)-2-(2-(azetidin-1-yl)-1-(3-fluorophenyl)-1-(1-((3- |
| | hydroxy-1-(4-(((S)-tetrahydro-2H-pyran-3-yl)sulfonyl)phenyl)azetidin- |
| | 3-yl)methyl)piperidin-4-yl)ethyl)cyclopentyl)carbamate |
| 371 | methyl ((1S,2R)-2-(2-(azetidin-1-yl)-1-(3-fluorophenyl)-1-(1-((3- |
| | hydroxy-1-(4-(((R)-tetrahydro-2H-pyran-3-yl)sulfonyl)phenyl)azetidin- |
| | 3-yl)methyl)piperidin-4-yl)ethyl)cyclopentyl)carbamate |
| 372 | methyl ((1S,2R)-2-(1-(1-((1-(4-((1-acetylpiperidin-4- |
| | yl)sulfonyl)phenyl)-3-hydroxyazetidin-3-yl)methyl)piperidin-4-yl)-2- |
| | (azetidin-1-yl)-1-(3-fluorophenyl)ethyl)cyclopentyl)carbamate |
| 373 | methyl ((1S,2R)-2-(2-(azetidin-1-yl)-1-(1-((1-(4- |
| | (cyclohexylsulfonyl)phenyl)-3-hydroxyazetidin-3-yl)methyl)piperidin- |
| | 4-yl)-1-(3-fluorophenyl)ethyl)cyclopentyl)carbamate |
| 374 | methyl ((1S,2R)-2-(2-(azetidin-1-yl)-1-(3-fluorophenyl)-1-(1-((3- |
| | hydroxy-1-(4-(((R)-tetrahydrofuran-3-yl)sulfonyl)phenyl)azetidin-3- |
| | yl)methyl)piperidin-4-yl)ethyl)cyclopentyl)carbamate |
| 375 | methyl ((1S,2R)-2-(2-(azetidin-1-yl)-1-(3-fluorophenyl)-1-(1-((3- |
| | hydroxy-1-(4-(((S)-tetrahydrofuran-3-yl)sulfonyl)phenyl)azetidin-3- |
| | yl)methyl)piperidin-4-yl)ethyl)cyclopentyl)carbamate |
| 376 | methyl ((1S,2R)-2-(2-(azetidin-1-yl)-1-(3-fluorophenyl)-1-(1-((3- |
| | methoxy-1-(4-(((1S,4S)-5-propionyl-2,5-diazabicyclo[2.2.1]heptan-2- |
| | yl)sulfonyl)phenyl)azetidin-3-yl)methyl)piperidin-4- |
| | yl)ethyl)cyclopentyl)carbamate |
| 377 | methyl ((1S,2R)-2-(2-(azetidin-1-yl)-1-(1-((1-(4- |
| | (cyclopropylsulfonyl)phenyl)-3-ethoxyazetidin-3-yl)methyl)piperidin- |
| | 4-yl)-1-(3-fluorophenyl)ethyl)cyclopentyl)carbamate |
| 378 | methyl ((1S,2R)-2-(2-(azetidin-1-yl)-1-(1′-(4- |
| | (cyclopropylsulfonyl)phenyl)-[1,4′-bipiperidin]-4-yl)-1-(3- |
| | fluorophenypethyl)cyclopentyl)carbamate |
| 379 | methyl 2-(1-((1-(4-(cyclopropylsulfonyl)phenyl)azetidin-3- |
| | yl)methyl)piperidin-4-yl)-2-(3-fluorophenyl)-2-((1R,2S)-2- |
| | ((methoxycarbonyl)amino)cyclopentyl)acetate |
| 380 | methyl N-[(1S,2R)-2-[(1S)-1-[1-({1-[4- |
| | (cyclopropanesulfonyl)phenyl]azetidin-3-yl}methyl)piperidin-4-yl]-2- |
| | [(dimethylcarbamoyl)amino]-1-(3- |
| | fluorophenyl)ethyl]cyclopentyl]carbamate |
| 381 | methyl N-[(1S,2R)-2-[(1S)-1-[1-({1-[4- |
| | (cyclopropanesulfonyl)phenyl]azetidin-3-yl}methyl)piperidin-4-yl]-1- |
| | (3-fluorophenyl)-2- |
| | [(methylcarbamoyl)amino]ethyl]cyclopentyl]carbamate |
| 382 | methyl N-[(1S,2R)-2-[(1S)-2-(carbamoylamino)-1-[1-({1-[4- |
| | (cyclopropanesulfonyl)phenyl]azetidin-3-yl}methyl)piperidin-4-yl]-1- |
| | (3-fluorophenyl)ethyl]cyclopentyl]carbamate |
| 383 | methyl N-[(1S,2R)-2-[(1S)-2-(azetidin-1-yl)-1-[1-(3-{[4- |
| | (cyclopropanesulfonyl)phenyl]amino}cyclobutyl)piperidin-4-yl]-1-(3- |
| | fluorophenyl)ethyl]cyclopentyl]carbamate |
| 384 | methyl N-[(1S,2R)-2-[(1S)-2-(azetidin-1-yl)-1-(1-{3-[(4- |
| | cyanophenyl)amino]cyclobutyl}piperidin-4-yl)-1-(3- |
| | fluorophenyl)ethyl]cyclopentyl]carbamate |
| 385 | methyl ((1S,2R)-2-((1S)-2-(azetidin-1-yl)-1-(1-((3S)-3-((4- |
| | (cyclopropylsulfonyl)phenyl)amino)cyclopentyl)piperidin-4-yl)-1-(3- |
| | fluorophenypethyl)cyclopentyl)carbamate |
| 386 | methyl ((1S,2R)-2-((1S)-2-(azetidin-1-yl)-1-(1-((3S)-3-((4- |
| | cyanophenyl)amino)cyclopentyl)piperidin-4-yl)-1-(3- |
| | fluorophenyl)ethyl)cyclopentyl)carbamate |
| 387 | methyl ((1S,2R)-2-((1S)-2-(azetidin-1-yl)-1-(1-((3R)-3-((4- |
| | (cyclopropylsulfonyl)phenyl)amino)cyclopentyl)piperidin-4-yl)-1-(3- |
| | fluorophenyl)ethyl)cyclopentyl)carbamate |
| 388 | methyl ((1S,2R)-2-((1S)-2-(azetidin-1-yl)-1-(1-((3R)-3-((4- |
| | cyanophenyl)amino)cyclopentyl)piperidin-4-yl)-1-(3- |
| | fluorophenyl)ethyl)cyclopentyl)carbamate |
| 389 | methyl N-[(1S,2R)-2-[(1S)-1-[1-({1-[4- |
| | (cyclopropanesulfonyl)phenyl]azetidin-3-yl}methyl)piperidin-4-yl]-1- |
| | (3-fluorophenyl)-2-methanesulfonamidoethyl]cyclopentyl]carbamate |
| 390 | methyl N-[(1S,2R)-2-[(1S)-1-[1-({1-[4- |
| | (cyclopropanesulfonyl)phenyl]azetidin-3-yl}methyl)piperidin-4-yl]-2- |
| | [(2,2-difluoroethyl)amino]-1-(3- |
| | fluorophenyl)ethyl]cyclopentyl]carbamate |
| 391 | 4-(3-((4-((S)-cyano(3-fluorophenyl)((1R,2S)-2-(2-oxooxazolidin-3- |
| | yl)cyclopentyl)methyl)piperidin-1-yl)methyl)azetidin-1-yl)benzonitrile |
| 392 | (R)-N-((1S,2R)-2-((S)-cyano(1-((1-(4-cyanophenyl)azetidin-3- |
| | yl)methyl)piperidin-4-yl)(3-fluorophenyl)methyl)cyclopentyl)oxirane- |
| | 2-carboxamide |
| 393 | (S)-N-((1S,2R)-2-((S)-cyano(1-((1-(4-cyanophenyl)azetidin-3- |
| | yl)methyl)piperidin-4-yl)(3-fluorophenyl)methyl)cyclopentyl)oxirane- |
| | 2-carboxamide |
| 394 | 4-(3-((4-((S)-cyano(3-fluorophenyl)((1R,2S)-2-(2-oxooxazol-3(2H)- |
| | yl)cyclopentyl)methyl)piperidin-1-yl)methyl)azetidin-1-yl)benzonitrile |
| 395 | methyl ((1S,2R)-2-((S)-1-(1-((1-(4- |
| | (cyclopropylsulfonyl)phenyl)azetidin-3-yl)methyl)piperidin-4-yl)-2-(2- |
| | (dimethylamino)acetamido)-1-(3- |
| | fluorophenyl)ethyl)cyclopentyl)carbamate |
| 396 | methyl ((1S,2R)-2-((S)-1-(1-((1-(4- |
| | (cyclopropylsulfonyl)phenyl)azetidin-3-yl)methyl)piperidin-4-yl)-1-(3- |
| | fluorophenyl)-2-(2-morpholinoacetamido)ethyl)cyclopentyl)carbamate |
| 397 | methyl ((1S,2R)-2-((S)-2-(2-(azetidin-1-yl)acetamido)-1-(1-((1-(4- |
| | (cyclopropylsulfonyl)phenyl)azetidin-3-yl)methyl)piperidin-4-yl)-1-(3- |
| | fluorophenyl)ethyl)cyclopentyl)carbamate |
| 398 | methyl ((1S,2R)-2-((S)-2-(2-amino-2-methylpropanamido)-1-(1-((1-(4- |
| | (cyclopropylsulfonyl)phenyl)azetidin-3-yl)methyl)piperidin-4-yl)-1-(3- |
| | fluorophenyl)ethyl)cyclopentyl)carbamate |
| 399 | methyl ((1S,2R)-2-((S)-2-((R)-azetidine-2-carboxamido)-1-(1-((1-(4- |
| | (cyclopropylsulfonyl)phenyl)azetidin-3-yl)methyl)piperidin-4-yl)-1-(3- |
| | fluorophenyl)ethyl)cyclopentyl)carbamate |
| 400 | methyl ((1S,2R)-2-((S)-2-((S)-azetidine-2-carboxamido)-1-(1-((1-(4- |
| | (cyclopropylsulfonyl)phenyl)azetidin-3-yl)methyl)piperidin-4-yl)-1-(3- |
| | fluorophenyl)ethyl)cyclopentyl)carbamate |

In another embodiment, the disclosure provides a pharmaceutical composition comprising a Compound of the Disclosure and a pharmaceutically acceptable carrier.

In another embodiment, Compounds of the Disclosure are enantiomerically enriched, e.g., the enantiomeric excess or “ee” of the compound is about 5% or more as measured by chiral HPLC. In another embodiment, the ee is about 10%. In another embodiment, the ee is about 20%. In another embodiment, the ee is about 30%. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

The present disclosure encompasses the preparation and use of salts of Compounds of the Disclosure. As used herein, the pharmaceutical “pharmaceutically acceptable salt” refers to salts or zwitterionic forms of Compounds of the Disclosure. Salts of Compounds of the Disclosure can be prepared during the final isolation and purification of the compounds or separately by reacting the compound with a suitable acid. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemi sulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al, AAPS Pharm. Sci. Tech, 569: Article 12 (2004), and A. L. Bingham et al, Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Therapeutic Methods of the Disclosure

Compounds of the Disclosure inhibit menin and are useful in the treatment of a variety of diseases and conditions. In particular, Compounds of the Disclosure are useful in methods of treating a disease or condition wherein inhibition of menin provides a benefit, for example, cancers and proliferative diseases. Methods of the disclosure comprise administering a therapeutically effective amount of a Compound of the Disclosure to a subject in need thereof. The present methods also encompass administering a second therapeutic agent to the subject in addition to the Compound of the Disclosure. The second therapeutic agent is selected from drugs known as useful in treating the disease or condition afflicting the subject in need thereof, e.g., a chemotherapeutic agent and/or radiation known as useful in treating a particular cancer.

The present disclosure provides Compounds of the Disclosure as menin inhibitors for the treatment of diseases and conditions wherein inhibition of menin has a beneficial effect. Compounds of the Disclosure typically have a binding affinity (IC₅₀) to menin of less than 100 μM, e.g., less than 50 μM, less than 25 μM, and less than 5 μM, less than about 1 μM, less than about 0.5 μM, less than about 0.1 μM, less than about 0.05 μM, or less than about 0.01 μM. In one embodiment, the present disclosure relates to a method of treating an individual suffering from a disease or condition wherein inhibition of menin provides a benefit comprising administering a therapeutically effective amount of a Compound of the Disclosure to an individual in need thereof.

Since Compounds of the Disclosure are inhibitors of menin protein, a number of diseases and conditions mediated by menin can be treated by employing these compounds. The present disclosure is thus directed generally to a method for treating a condition or disorder responsive to menin inhibition in an animal, e.g., a human, suffering from, or at risk of suffering from, the condition or disorder, the method comprising administering to the animal an effective amount of one or more Compounds of the Disclosure.

The present disclosure is further directed to a method of inhibiting menin in a subject in need thereof, said method comprising administering to the animal an effective amount of at least one Compound of the Disclosure.

The methods of the present disclosure can be accomplished by administering a Compound of the Disclosure as the neat compound or as a pharmaceutical composition. Administration of a pharmaceutical composition, or neat compound of a Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a Compound of the Disclosure and, optionally, a second therapeutic agent, packaged separately or together, and an insert having instructions for using these active agents.

In one embodiment, a Compound of the Disclosure is administered in conjunction with a second therapeutic agent useful in the treatment of a disease or condition wherein inhibition of menin provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered from a single composition or two separate compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more dose of the second therapeutic agent can be administered. The Compound of the Disclosure therefore can be used in conjunction with one or more second therapeutic agents, for example, but not limited to, anticancer agents.

Diseases and conditions treatable by the methods of the present disclosure include, but are not limited to, cancer and other proliferative disorders, inflammatory diseases, sepsis, autoimmune disease, and viral infection. In one embodiment, a human patient is treated with a Compound of the Disclosure, or a pharmaceutical composition comprising a Compound of the Disclosure, wherein the compound is administered in an amount sufficient to inhibit menin activity in the patient.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by inhibiting menin. Examples of treatable cancers include, but are not limited to, any one or more of the cancers of Table 2,

TABLE 2

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ | medullary |
| lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In another embodiment, the cancer is a solid tumor. In another embodiment, the cancer a hematological cancer. Exemplary hematological cancers include, but are not limited to, the cancers listed in Table 3. In another embodiment, the hematological cancer is acute lymphocytic leukemia, chronic lymphocytic leukemia (including B-cell chronic lymphocytic leukemia), or acute myeloid leukemia.

TABLE 3

acute lymphocytic leukemia (ALL)
acute myeloid leukemia (AML)
chronic lymphocytic leukemia (CLL)
small lymphocytic lymphoma (SLL)
multiple myeloma (MM)
Hodgkins lymphoma (HL)
non-Hodgkin's lymphoma (NHL)
mantle cell lymphoma (MCL)
marginal zone B-cell lymphoma
splenic marginal zone lymphoma
follicular lymphoma (FL)
Waldenstrom's macroglobulinemia (WM)
diffuse large B-cell lymphoma (DLBCL)
marginal zone lymphoma (MZL)
hairy cell leukemia (HCL)
Burkitt's lymphoma (BL)
Richter's transformation
acute eosinophilic leukemia
acute erythroid leukemia
acute lymphoblastic leukemia
acute megakaryoblastic leukemia
acute monocytic leukemia
acute promyelocytic leukemia
acute myelogeous leukemia
B-cell prolymphocytic leukemia
B-cell lymphoma
MALT lymphoma
precursor T-lymphoblastic lymphoma
T-cell lymphoma
mast cell leukemia
adult T cell leukemia/lymphoma
aggressive NK-cell leukemia
angioimmunoblastic T-cell lymphoma

In another embodiment, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the present disclosure provides a method of treating a benign proliferative disorder, such as, but are not limited to, benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

Compounds of the Disclosure can also treat infectious and noninfectious inflammatory events and autoimmune and other inflammatory diseases by administration of an effective amount of a present compound to a mammal, in particular a human in need of such treatment. Examples of autoimmune and inflammatory diseases, disorders, and syndromes treated using the compounds and methods described herein include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, Type I diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituatarism, Guillain-Barre syndrome, Behcet's disease, scleracierma, mycosis fungoides, acute inflammatory responses (such as acute respiratory distress syndrome and ischemia/reperfusion injury), and Graves' disease.

In another embodiment, the present disclosure provides a method of treating systemic inflammatory response syndromes, such as LPS-induced endotoxic shock and/or bacteria-induced sepsis by administration of an effective amount of a Compound of the Disclosure to a mammal, in particular a human in need of such treatment.

In another embodiment, the present disclosure provides a method for treating viral infections and diseases. Examples of viral infections and diseases treated using the compounds and methods described herein include episome-based DNA viruses including, but not limited to, human papillomavirus, Herpesvirus, Epstein-Barr virus, human immunodeficiency virus, hepatitis B virus, and hepatitis C virus.

In another embodiment, the present disclosure provides therapeutic method of modulating protein methylation, gene expression, cell proliferation, cell differentiation and/or apoptosis in vivo in diseases mentioned above, in particular cancer, inflammatory disease, and/or viral disease is provided by administering a therapeutically effective amount of a Compound of the Disclosure to a subject in need of such therapy.

In another embodiment, the present disclosure provides a method of regulating endogenous or heterologous promoter activity by contacting a cell with a Compound of the Disclosure.

In methods of the present disclosure, a therapeutically effective amount of a Compound of the Disclosure, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the menin inhibitor that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

As stated above, a Compound of the Disclosure can be administered in combination with a second therapeutically active agent. In some embodiments, the second therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, chemotherapeutic agents or other anti-proliferative agents can be combined with Compound of the Disclosure to treat proliferative diseases and cancer. Examples of therapies and anticancer agents that can be used in combination with Compounds of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DMl, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; ALK inhibitors, which are compounds that target, decrease, or inhibit anaplastic lymphoma kinase; and BH3 mimetics, which are compounds that target, decrease, or inhibit antiapoptotic proteins from the BCL-2 family.

Exemplary nonlimiting Flt-3 inhibitors include gilteritinib, PKC412, midostaurin, a staurosporine derivative, SU11248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino, 17-demethoxygeldanamycin (17AAG), a geldanamycin derivative; other geldanamycin related compounds; radicicol and HDAC inhibitors.

Exemplary nonlimiting BH3 mimetics include venetoclax.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SU101, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAR, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, C₁₋₁₀33, EKB-569, GW-2016, antibodies ELI, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Compound of the Disclosure, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, l-(4-chloroanilino)-4-(4-pyridylmethyl)phthalazine or a pharmaceutically acceptable salt thereof, l-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

Other examples of second therapeutic agents, one or more of which a Compound of the Disclosure also can be combined, include, but are not limited to: a treatment for Alzheimer's Disease, such as donepezil and rivastigmine; a treatment for Parkinson's Disease, such as L-DOPA/carbidopa, entacapone, ropinrole, pramipexole, bromocriptine, pergolide, trihexephendyl, and amantadine; an agent for treating multiple sclerosis (MS) such as beta interferon (e.g., AVONEX® and REBIF®), glatiramer acetate, and mitoxantrone; a treatment for asthma, such as albuterol and montelukast; an agent for treating schizophrenia, such as zyprexa, risperdal, seroquel, and haloperidol; an anti-inflammatory agent, such as a corticosteroid, a TNF blocker, IL-1 RA, azathioprine, cyclophosphamide, and sulfasalazine; an immunomodulatory agent, including immunosuppressive agents, such as cyclosporin, tacrolimus, rapamycin, mycophenolate mofetil, an interferon, a corticosteroid, cyclophosphamide, azathioprine, and sulfasalazine; a neurotrophic factor, such as an acetylcholinesterase inhibitor, an MAO inhibitor, an interferon, an anti-convulsant, an ion channel blocker, riluzole, or an anti-Parkinson's agent; an agent for treating cardiovascular disease, such as a beta-blocker, an ACE inhibitor, a diuretic, a nitrate, a calcium channel blocker, or a statin; an agent for treating liver disease, such as a corticosteroid, cholestyramine, an interferon, and an anti-viral agent; an agent for treating blood disorders, such as a corticosteroid, an anti-leukemic agent, or a growth factor; or an agent for treating immunodeficiency disorders, such as gamma globulin.

The above-mentioned second therapeutically active agents, one or more of which can be used in combination with a Compound of the Disclosure, are prepared and administered as described in the art.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

The disclosure provides the following particular embodiments in connection with treating a disease in a subject.

Embodiment 1. A method of treating a subject in need thereof, the method comprising administering to the subject a therapeutically effective amount a Compound of the Disclosure, wherein the subject has cancer.

Embodiment 2. The method of Embodiment 1, wherein the cancer is any one or more of the cancers of Table 2.

Embodiment 3. The method of Embodiment 2, wherein the cancer is a hematological cancer.

Embodiment 4. The method of Embodiment 3, wherein the hematological cancer is any one or more of the cancers of Table 3.

Embodiment 5. The method of any one of Embodiments 1-4 further comprising administering a therapeutically effective amount of a second therapeutic agent useful in the treatment of cancer.

Embodiment 6. A pharmaceutical composition comprising a Compound of the Disclosure and a pharmaceutically acceptable carrier for use in treating cancer.

Embodiment 7. The pharmaceutical composition of Embodiment 6, wherein the cancer is any one or more of the cancers of Table 2.

Embodiment 8. The pharmaceutical composition of Embodiment 7, wherein the cancer is a hematological cancer.

Embodiment 9. The pharmaceutical composition of Embodiment 8, wherein the hematological cancer is any one or more of the cancers of Table 3.

Embodiment 10. A Compound of the Disclosure for use in treatment of cancer.

Embodiment 11. The compound for use of Embodiment 10, wherein the cancer is any one or more of the cancers of Table 2.

Embodiment 12. The compound for use of Embodiment 11, wherein the cancer is a hematological cancer.

Embodiment 13. The compound for use of Embodiment 12, wherein the hematological cancer is any one or more of the cancers of Table 3.

Embodiment 14. Use of a Compound of the Disclosure for the manufacture of a medicament for treatment of cancer.

Embodiment 15. The use of Embodiment 14, wherein the cancer is any one or more of the cancers of Table 2.

Embodiment 16. The use of Embodiment 15, wherein the cancer is a hematological cancer.

Embodiment 17. The use of Embodiment 16, wherein the hematological cancer is any one or more of the cancers of Table 3.

### III. Kits of the Disclosure

In another embodiment, the present disclosure provides kits which comprise a Compound of the Disclosure (or a composition comprising a Compound of the Disclosure) packaged in a manner that facilitates their use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

### IV. Definitions

In the present disclosure, the term “halo” as used by itself or as part of another group refers to —Cl, —F, —Br, or —I.

In the present disclosure, the term “nitro” as used by itself or as part of another group refers to —NO₂.

In the present disclosure, the term “cyano” as used by itself or as part of another group refers to —CN.

In the present disclosure, the term “hydroxy” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₁₂ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl is a CHO alkyl. In another embodiment, the alkyl is a C₁₋₆ alkyl. In another embodiment, the alkyl is a C₁₋₄ alkyl. In another embodiment, the alkyl is a straight chain C₁₋₁₀ alkyl. In another embodiment, the alkyl is a branched chain C₃₋₁₀ alkyl. In another embodiment, the alkyl is a straight chain C₁₋₆ alkyl. In another embodiment, the alkyl is a branched chain C₃₋₆ alkyl. In another embodiment, the alkyl is a straight chain C₁₋₄ alkyl. In another embodiment, the alkyl is a branched chain C₃₋₄ alkyl. In another embodiment, the alkyl is a straight or branched chain C₃₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and No-butyl.

In the present disclosure, the term “optionally substituted alkyl” as used by itself or as part of another group refers to an alkyl that is either unsubstituted or substituted with one, two, or three substituents independently selected from the group consisting of nitro, haloalkoxy, aryloxy, aralkyloxy, alkylthio, sulfonamido, alkylcarbonyl, arylcarbonyl, alkylsulfonyl, arylsulfonyl, carboxy, carboxyalkyl, and alkylcarbonyloxy. In one embodiment, the optionally substituted alkyl is substituted with two substituents. In another embodiment, the optionally substituted alkyl is substituted with one substituent. In another embodiment, the optionally substituted alkyl is unsubstituted. Non-limiting exemplary substituted alkyl groups include —CH₂CH₂NO₂, —CH₂SO₂CH₃, CH₂CH₂SO₂CH₂CH₂CH₂CO₂H, —CH₂SCH₃, —CH₂CH₂SO₂CH₃, —CH₂CH₂COPh, and —CH₂OC(═O)CH₃.

In the present disclosure, the term “cycloalkyl” as used by itself or as part of another group refers to unsubstituted saturated or partially unsaturated, e.g., containing one or two double bonds, cyclic aliphatic hydrocarbons containing one to three rings having from three to twelve carbon atoms, i.e., C₃₋₁₂ cycloalkyl, or the number of carbons designated. In one embodiment, the cycloalkyl has two rings. In another embodiment, the cycloalkyl has one ring. In another embodiment, the cycloalkyl is saturated. In another embodiment, the cycloalkyl is unsaturated. In another embodiment, the cycloalkyl is a C₃₋₈ cycloalkyl. In another embodiment, the cycloalkyl is a C₃₋₆ cycloalkyl. The term “cycloalkyl” is meant to include groups wherein a ring —CH₂— is replaced with a —C(═O)—. Non-limiting exemplary cycloalkyl groups include cyclopropyl, cyclobutyl, cyclopentyl, cyclohexyl, cycloheptyl, cyclooctyl, norbomyl, decalin, adamantyl, cyclohexenyl, cyclopentenyl, and cyclopentanone.

In the present disclosure, the term “optionally substituted cycloalkyl” as used by itself or as part of another group refers to a cycloalkyl that is either unsubstituted or substituted with one, two, or three substituents independently selected from the group consisting of halo, nitro, cyano, hydroxy, alkylcarbonyloxy, cycloalkylcarbonyloxy, amino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, (heterocyclo)alkyl, —OC(═O)-amino, —N(R^{19a})C(═O)—R^{19b}, and —N(R^{20a})SO₂—R^{20b}, wherein R^{19a} is selected from the group consisting of hydrogen and alkyl, R^{19b} is selected from the group consisting of amino, alkoxy, alkyl, e.g., C₁-C₆, alkyl, and optionally substituted aryl, R^{20a} is selected from the group consisting of hydrogen and alkyl, and R^{20b} is selected from the group consisting of amino, alkyl, and optionally substituted aryl. The term optionally substituted cycloalkyl includes cycloalkyl groups having a fused optionally substituted aryl, e.g., phenyl, or fused optionally substituted heteroaryl, e.g., pyridyl. An optionally substituted cycloalkyl having a fused optionally substituted aryl or fused optionally substituted heteroaryl group may be attached to the remainder of the molecule at any available carbon atom on the cycloalkyl ring. In one embodiment, the optionally substituted cycloalkyl is substituted with two substituents. In another embodiment, the optionally substituted cycloalkyl is substituted with one substituent. In another embodiment, the optionally substituted cycloalkyl is unsubstituted. Non-limiting exemplary substituted cycloalkyl groups include:

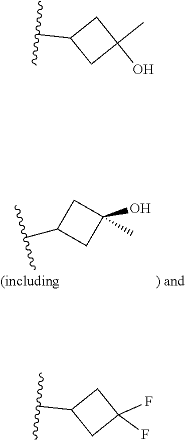

In the present disclosure, the term “cycloalkylenyl” as used herein by itself or part of another group refers to a divalent form of an optionally substituted cycloalkyl group. In one embodiment, the cycloalkylenyl is a 4-membered cycloalkylenyl. In another embodiment, the cycloalkylenyl is a 5-membered cycloalkylenyl. In another embodiment, the cycloalkylenyl is a 6-membered cycloalkylenyl. Non-limiting exemplary cycloalkylenyl groups include:

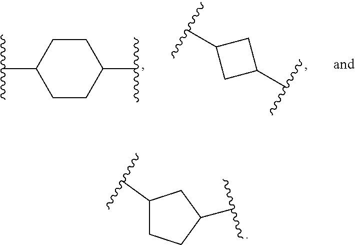 [SMILES: *C1CCC(*)CC1 ; *C1CC(*)C1 ; *C1CCC(*)C1]

In the present disclosure, the term “aryl” as used by itself or as part of another group refers to unsubstituted monocyclic or bicyclic aromatic ring systems having from six to fourteen carbon atoms, i.e., a C₆₋₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl.

In the present disclosure, the term “optionally substituted aryl” as used herein by itself or as part of another group refers to an aryl that is either unsubstituted or substituted with one to five substituents independently selected from the group consisting of halo, nitro, cyano, hydroxy, amino, —CO₂CH₂Ph, alkylamino, dialkylamino, optionally substituted alkyl, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, haloalkylsulfonyl cycloalkylsulfonyl, (cycloalkyl)alkylsulfonyl, arylsulfonyl, heteroarylsulfonyl, heterocyclosulfonyl, carboxy, carboxyalkyl, optionally substituted cycloalkyl, alkenyl, alkynyl, optionally substituted aryl, optionally substituted heteroaryl, optionally substituted heterocyclo, alkoxycarbonyl, alkoxyalkyl, (amino)alkyl, (carboxamido)alkyl, (heterocyclo)alkyl, —N(R^{19a})C(═O)—R^{19b}, and —N(R^{20a})SO₂—R^{20b}, wherein R^{19a}, R^{19b}, R^{20a}, and R^{20b} are as defined in connection with optionally substituted cycloalkyl.

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. In another embodiment, the optionally substituted phenyl is unsubstituted. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, 4-(pyridin-4-ylsulfonyl)phenyl The term optionally substituted aryl includes phenyl groups having a fused optionally substituted cycloalkyl or fused optionally substituted heterocyclo group. An optionally substituted phenyl having a fused optionally substituted cycloalkyl or fused optionally substituted heterocyclo group may be attached to the remainder of the molecule at any available carbon atom on the phenyl ring. Non-limiting examples include:

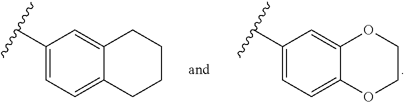 [SMILES: *c1ccc2c(c1)CCCC2 ; *c1ccc2c(c1)OCCO2]

Additional non-limiting examples of optionally substituted aryl include:

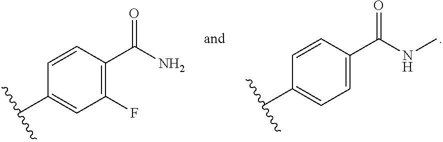 [SMILES: *c1ccc(C(N)=O)c(F)c1 ; *c1ccc(C(=O)NC)cc1]

In the present disclosure, the term “alkenyl” as used by itself or as part of another group refers to an alkyl containing one, two or three carbon-to-carbon double bonds. In one embodiment, the alkenyl has one carbon-to-carbon double bond. In another embodiment, the alkenyl is a C₂₋₆ alkenyl. In another embodiment, the alkenyl is a C₂₋₄ alkenyl. Non-limiting exemplary alkenyl groups include ethenyl, propenyl, isopropenyl, butenyl, sec-butenyl, pentenyl, and hexenyl.

In the present disclosure, the term “optionally substituted alkenyl” as used herein by itself or as part of another group refers to an alkenyl that is either unsubstituted or substituted with one, two or three substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, optionally substituted cycloalkyl, alkenyl, alkynyl, optionally substituted aryl, heteroaryl, and optionally substituted heterocyclo.

In the present disclosure, the term “alkynyl” as used by itself or as part of another group refers to an alkyl containing one to three carbon-to-carbon triple bonds. In one embodiment, the alkynyl has one carbon-to-carbon triple bond. In another embodiment, the alkynyl is a C₂₋₆ alkynyl. In another embodiment, the alkynyl is a C₂₋₄ alkynyl. Non-limiting exemplary alkynyl groups include ethynyl, propynyl, butynyl, 2-butynyl, pentynyl, and hexynyl groups.

In the present disclosure, the term “optionally substituted alkynyl” as used herein by itself or as part refers to an alkynyl that is either unsubstituted or substituted with one, two or three substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, cycloalkyl, alkenyl, alkynyl, optionally substituted aryl, optionally substituted heteroaryl, and heterocyclo.

In the present disclosure, the term “haloalkyl” as used by itself or as part of another group refers to an alkyl substituted by one or more fluorine, chlorine, bromine and/or iodine atoms. In one embodiment, the alkyl group is substituted by one, two, or three fluorine and/or chlorine atoms. In another embodiment, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “hydroxyalkyl” as used by itself or as part of another group refers to an alkyl substituted with one, two, or three hydroxy groups. In one embodiment, the hydroxyalkyl is a monohydroxyalkyl, i.e., a hydroxyalkyl substituted with one hydroxy group. In another embodiment, the hydroxyalkyl is a dihydroxyalkyl, i.e., a hydroxyalkyl substituted with two hydroxy groups. Non-limiting exemplary hydroxyalkyl groups include hydroxymethyl, hydroxyethyl, hydroxypropyl and hydroxybutyl groups, such as 1-hydroxyethyl, 2-hydroxy ethyl, 1,2-dihydroxy ethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

In the present disclosure, the term “heteroaralkyl” as used by itself or as part of another group refers to an alkyl substituted with one, two, or three optionally substituted heteroaryl groups. In one embodiment, the heteroaralkyl alkyl group is a C₁₋₄ alkyl substituted with one optionally substituted heteroaryl group. Non-limiting exemplary heteroaralkyl groups include:

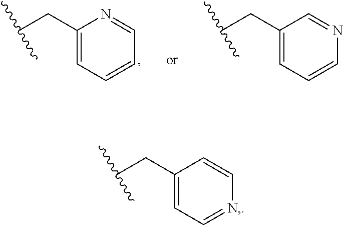 [SMILES: *Cc1ccccn1 ; *Cc1cccnc1 ; *Cc1ccncc1]

In the present disclosure, the term “(cycloalkyl)alkyl,” as used by itself or as part of another group refers to an alkyl substituted with an optionally substituted cycloalkyl. In one embodiment, the (cycloalkyl) alkyl, is a “(C₃₋₆ cycloalkyl)C₁₋₄ alkyl,” i.e., a C₁₋₄ alkyl substituted with an optionally substituted C₃₋₆ cycloalkyl. Non-limiting exemplary (cycloalkyl) alkyl groups include:

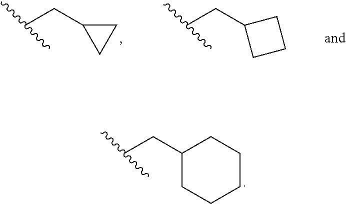 [SMILES: *CC1CC1 ; *CC1CCC1 ; *CC1CCCCC1]

In the present disclosure, the term “alkylsulfonyl” as used by itself or as part of another group refers to a sulfonyl, i.e., —SO₂—, substituted with an optionally substituted alkyl. A non-limiting exemplary alkylsulfonyl group is —SO₂CH₃.

In the present disclosure, the term “haloalkylsulfonyl” as used by itself or as part of another group refers to a sulfonyl, i.e., —SO₂—, substituted with a haloalkyl. A non-limiting exemplary alkylsulfonyl group is —SO₂CF₃.

In the present disclosure, the term “cycloalkylsulfonyl” as used by itself or as part of another group refers to a sulfonyl, i.e., —SO₂—, substituted with an optionally substituted cycloalkyl. Non-limiting exemplary alkylsulfonyl group include —SO₂— cyclopropyl and —SO₂-cyclopenyl.

In the present disclosure, the term “(cycloalkyl)alkylsulfonyl” as used by itself or as part of another group refers to a sulfonyl, i.e., —SO₂—, substituted with a (cycloalkyl)alkyl. Non-limiting exemplary (cycloalkyl)alkylsulfonyl groups include:

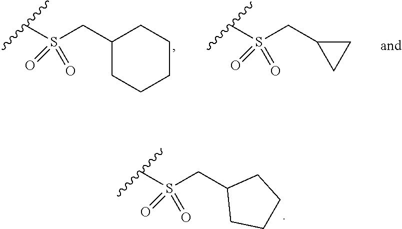 [SMILES: *S(=O)(=O)CC1CCCCC1 ; *S(=O)(=O)CC1CC1 ; *S(=O)(=O)CC1CCCC1]

In the present disclosure, the term “arylsulfonyl” as used by itself or as part of another group refers to a sulfonyl, i.e., —SO₂—, substituted with an optionally substituted aryl. A non-limiting exemplary arylsulfonyl group is —SO₂Ph.

In the present disclosure, the term “heteroarylsulfonyl” as used by itself or as part of another group refers to a sulfonyl, i.e., —SO₂—, substituted with an optionally substituted heteroaryl group. Non-limiting exemplary heteroarylsulfonyl groups include:

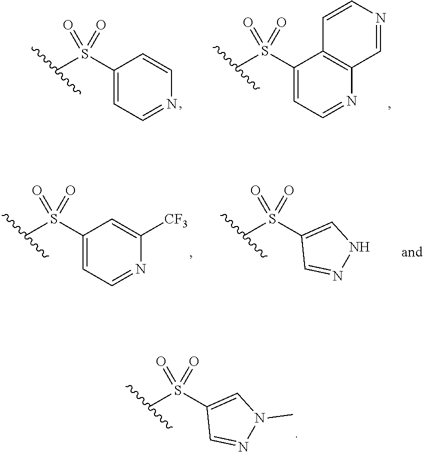

In the present disclosure, the term “heterocyclosulfonyl” as used by itself or as part of another group refers to a sulfonyl, i.e., —SO₂—, substituted with an optionally substituted heterocyclo group. A non-limiting exemplary heterocyclosulfonyl group is:

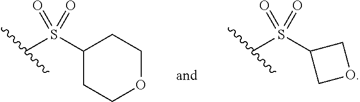 [SMILES: *S(=O)(=O)C1CCOCC1 ; "and" ; *S(=O)(=O)C1COC1]

In the present disclosure, the term “sulfonamido” as used by itself or as part of another group refers to a radical of the formula —SO₂NR^{21a}R^{21b}, wherein R^{21a} and R^{21b} are each independently selected from the group consisting of hydrogen, optionally substituted alkyl, and optionally substituted aryl, or R^{21a} and R^{21b} taken together with the nitrogen to which they are attached from a 3- to 8-membered heterocyclo group. Non-limiting exemplary sulfonamido groups include —SO₂NH₂, —SO₂N(H)CH₃, —SO₂N(CH₃)₂, and —SO₂N(H)Ph.

In the present disclosure, the term “alkoxy” as used by itself or as part of another group refers to an optionally substituted alkyl, optionally substituted cycloalkyl, optionally substituted alkenyl, or optionally substituted alkynyl attached to a terminal oxygen atom. In one embodiment, the alkoxy is an optionally substituted alkyl attached to a terminal oxygen atom. In one embodiment, the alkoxy group is a C₁₋₆ alkyl attached to a terminal oxygen atom. In another embodiment, the alkoxy group is a C₁₋₄ alkyl attached to a terminal oxygen atom. Non-limiting exemplary alkoxy groups include methoxy, ethoxy, tert-butoxy, and —OCH₂SO₂CH₃.

In the present disclosure, the term “alkylthio” as used by itself or as part of another group refers to an optionally substituted alkyl attached to a terminal sulfur atom. In one embodiment, the alkylthio group is a C₁₋₄ alkylthio group. Non-limiting exemplary alkylthio groups include —SCH₃ and —SCH₂CH₃.

In the present disclosure, the term “alkoxyalkyl” as used by itself or as part of another group refers to an optionally alkyl substituted with an alkoxy group. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

In the present disclosure, the term “haloalkoxy” as used by itself or as part of another group refers to a haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryloxy” as used by itself or as part of another group refers to an optionally substituted aryl attached to a terminal oxygen atom. A non-limiting exemplary aryloxy group is PhO—.

In the present disclosure, the term “aralkyloxy” as used by itself or as part of another group refers to an aralkyl attached to a terminal oxygen atom. Non-limiting exemplary aralkyloxy groups include PhCH₂O— and PhCH₂CH₂O—.

In the present disclosure, the term “heteroaryl” refers to unsubstituted monocyclic and bicyclic aromatic ring systems having 5 to 14 ring atoms, i.e., a 5- to 14-membered heteroaryl, wherein at least one carbon atom of one of the rings is replaced with a heteroatom independently selected from the group consisting of oxygen, nitrogen and sulfur. In one embodiment, the heteroaryl contains 1, 2, 3, or 4 heteroatoms independently selected from the group consisting of oxygen, nitrogen and sulfur. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl is a 5- or 6-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3/7-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is selected from the group consisting of thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl), isoxazolyl (e.g., isoxazol-3-yl, isoxazol-4-yl, and isoxazol-5-yl), and indazolyl (e.g., 1H-indazol-3-yl). The term “heteroaryl” is also meant to include possible N-oxides. A non-limiting exemplary N-oxide is pyridyl N-oxide.

In one embodiment, the heteroaryl is a 5- or 6-membered heteroaryl. In one embodiment, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Non-limiting exemplary 5-membered heteroaryl groups include thienyl, furyl, pyrrolyl, oxazolyl, pyrazolyl, imidazolyl, thiazolyl, isothiazolyl, and isoxazolyl. In another embodiment, the heteroaryl is a 6-membered heteroaryl, e.g., the heteroaryl is a monocyclic aromatic ring system having 6 ring atoms wherein at least one carbon atom of the ring is replaced with a nitrogen atom. Non-limiting exemplary 6-membered heteroaryl groups include pyridyl, pyrazinyl, pyrimidinyl, and pyridazinyl.

In the present disclosure, the term “optionally substituted heteroaryl” as used by itself or as part of another group refers to a heteroaryl that is either unsubstituted or substituted with one two, three, or four substituents, independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, haloalkylsulfonyl cycloalkylsulfonyl, (cycloalkyl)alkylsulfonyl, arylsulfonyl, heteroarylsulfonyl, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, (heterocyclo)alkyl, —N(R^{19a})C(═O)—R^{19b}, and —N(R^{20a})SO₂—R^{20b}, wherein R^{19a}, R^{19b}, R^{20a}, and R^{20b} are as defined in connection with optionally substituted cycloalkyl. In one embodiment, the optionally substituted heteroaryl has one substituent. In another embodiment, the optionally substituted heteroaryl is unsubstituted. Any available carbon or nitrogen atom can be substituted. The term optionally substituted heteroaryl includes heteroaryl groups having a fused optionally substituted cycloalkyl or fused optionally substituted heterocyclo group. An optionally substituted heteroaryl having a fused optionally substituted cycloalkyl or fused optionally substituted heterocyclo group may be attached to the remainder of the molecule at any available carbon atom on the heteroaryl ring.

In the present disclosure, the term “heteroarylenyl” as used herein by itself or part of another group refers to a divalent form of an optionally substituted heteroaryl group. In one embodiment, the heteroarylenyl is a 5-membered heteroaryl enyl. Non-limiting examples of a 5-membered heteroarylenyl include:

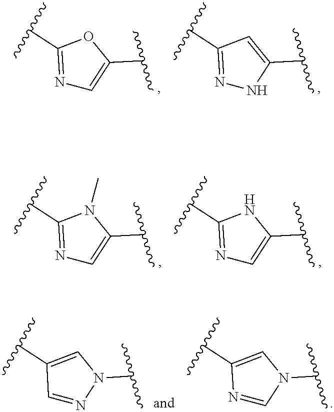

Additional non-limiting examples of a 5-membered heteroarylenyl include:

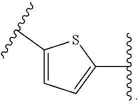 [SMILES: *c1ccc(*)s1]

In another embodiment, the heteroarylenyl is a 6-membered heteroarylenyl. Non-limiting examples of a 6-membered heteroarylenyl include:

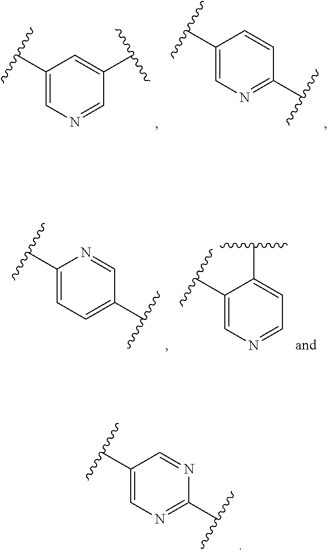 [SMILES: *c1cncc(*)c1 ; *c1ccc(*)nc1 ; *c1ccc(*)nc1 ; *c1ccncc1* ; *c1cnc(*)nc1]

In the present disclosure, the term “heterocyclo” as used by itself or as part of another group refers to unsubstituted saturated and partially unsaturated, e.g., containing one or two double bonds, cyclic groups containing one, two, or three rings having from three to fourteen ring members, i.e., a 3- to 14-membered heterocyclo, wherein at least one carbon atom of one of the rings is replaced with a heteroatom. Each heteroatom is independently selected from the group consisting of oxygen, sulfur, including sulfoxide and sulfone, and/or nitrogen atoms, which can be oxidized or quaternized. The term “heterocyclo” includes groups wherein a ring —CH₂— is replaced with a —C(═O)—, for example, cyclic ureido groups such as 2-imidazolidinone and cyclic amide groups such as β-lactam, γ-lactam, δ-lactam, ε-lactam, and piperazin-2-one. The term “heterocyclo” also includes groups having fused optionally substituted aryl groups, e.g., indolinyl or chroman-4-yl. In one embodiment, the heterocyclo group is a C₄₋₆ heterocyclo, i.e., a 4-, 5- or 6-membered cyclic group, containing one ring and one or two oxygen and/or nitrogen atoms. In one embodiment, the heterocyclo group is a C₄₋₆ heterocyclo containing one ring and one nitrogen atom. The heterocyclo can be optionally linked to the rest of the molecule through any available carbon or nitrogen atom. Non-limiting exemplary heterocyclo groups include azetidinyl, dioxanyl, tetrahydropyranyl, 2-oxopyrrolidin-3-yl, piperazin-2-one, piperazine-2,6-dione, 2-imidazolidinone, piperidinyl, morpholinyl, piperazinyl, pyrrolidinyl, and indolinyl. Additional non-limiting examples of heterocyclo groups include oxetanyl and tetrahydrofuranyl.

In the present disclosure, the term “optionally substituted heterocyclo” as used herein by itself or part of another group refers to a heterocyclo that is either unsubstituted or substituted with one, two, three, or four substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, cycloalkylcarbonyl, alkoxycarbonyl, CF₃C(═O)—, arylcarbonyl, alkyl sulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, (heterocyclo)alkyl, hydroxyalkylcarbonyl, and —N(R^{19a})C(═O)—R^{19b}, and —N(R^{20a})SO₂—R^{20b}, wherein R^{19a}, R^{19b}, R^{20a}, and R^{20b} are as defined in connection with optionally substituted cycloalkyl. Substitution may occur on any available carbon or nitrogen atom, or both. Non-limiting exemplary substituted heterocyclo groups include:

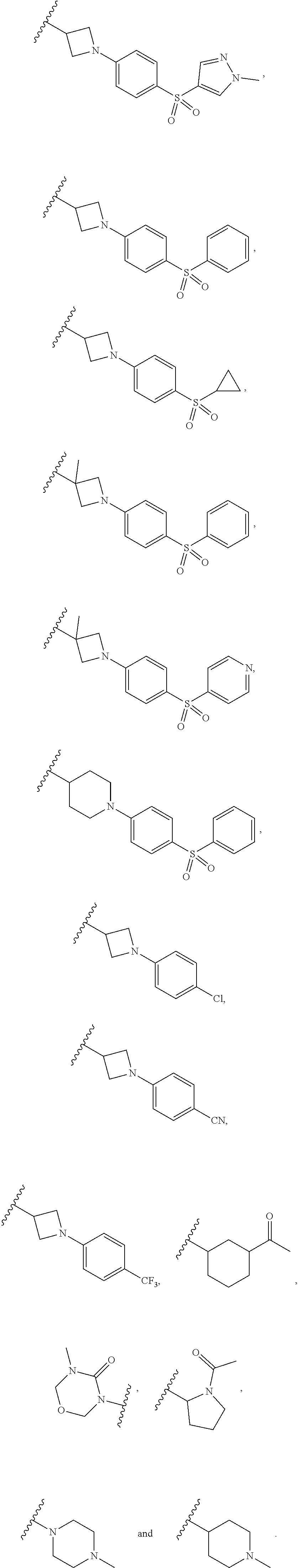

Additional non-limiting exemplary substituted heterocyclo groups include:

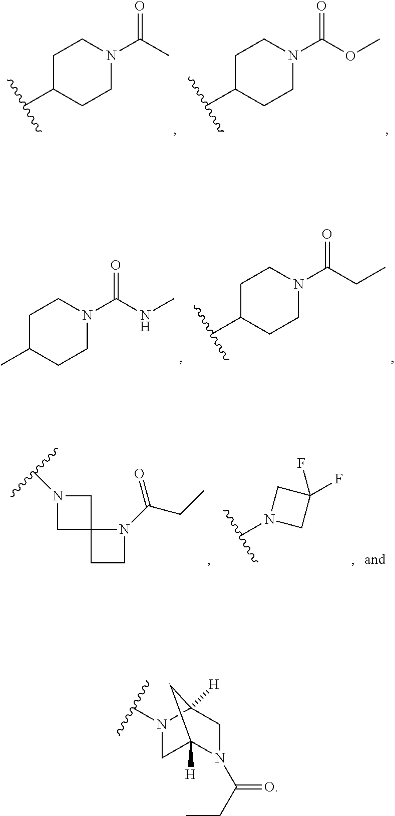

In the present disclosure, the term “amino” as used by itself or as part of another group refers to a radical of the formula —NR^{22a}R^{22b}, wherein R^{22a} and R^{22b} are each independently selected from the group consisting of hydrogen, alkyl, aralkyl, hydroxyalkyl, optionally substituted cycloalkyl, optionally substituted aryl, optionally substituted heterocyclo, and optionally substituted heteroaryl, or R^{22a} and R^{22b} are taken together to form a 3- to 8-membered optionally substituted heterocyclo. Non-limiting exemplary amino groups include —NH₂ and —N(H)(CH₃).

In the present disclosure, the term “(amino)alkyl” as used by itself or as part of another group refers to an alkyl substituted with an amino. Non-limiting exemplary (amino)alkyl groups include —CH₂CH₂NH₂, and —CH₂CH₂N(H)CH₃, —CH₂CH₂N(CH₃)₂, and —CH₂N(H)-cyclopropyl. Additional non-limiting exemplary (amino)alkyl groups include —CH₂N(CH₃)₂.

In the present disclosure, the term “carboxamido” as used by itself or as part of another group refers to a radical of formula —C(═O)NR^{23a}R^{23b}, wherein R^{23a} and R^{23b} are each independently selected from the group consisting of hydrogen, optionally substituted alkyl, hydroxyalkyl, optionally substituted cycloalkyl, optionally substituted aryl, optionally substituted heterocyclo, and optionally substituted heteroaryl, or R^{23a} and R^{23b} taken together with the nitrogen to which they are attached form a 3- to 8-membered optionally substituted heterocyclo group. In one embodiment, R^{23a} and R^{23b} are each independently hydrogen or optionally substituted alkyl. In one embodiment, R^{23a} and R^{23b} are taken together to taken together with the nitrogen to which they are attached form a 3- to 8-membered optionally substituted heterocyclo group. Non-limiting exemplary carboxamido groups include —CONH₂, —CON(H)CH₃, —CON(CH₃)₂, —CON(H)Ph,

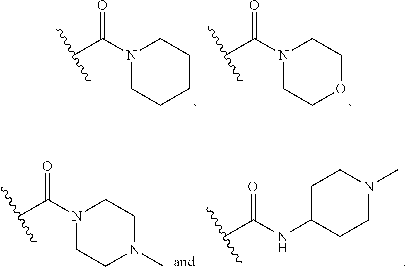

Additional non-limiting exemplary carboxamido groups include:

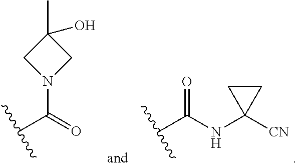

In the present disclosure, the term “alkylcarbonyl” as used by itself or as part of another group refers to a carbonyl group, i.e., —C(═O)—, substituted with an alkyl. Non-limiting exemplary alkylcarbonyl groups include —C(═O)CH₃ and —C(═O)CH₂CH₂CH₂CH₃.

In the present disclosure, the term “hydroxyalkylcarbonyl” as used by itself or as part of another group refers to a carbonyl group, i.e., —C(═O)—, substituted with an hydroxyalkyl. Non-limiting exemplary alkylcarbonyl groups include —C(═O)C(CH₃)₂OH and —C(═O)CH₂CH₂CH₂OH.

In the present disclosure, the term “cycloalkylcarbonyl” as used by itself or as part of another group refers to a carbonyl group, i.e., —C(═O)—, substituted with a cycloalkyl. A non-limiting exemplary cycloalkylcarbonyl group is —C(═O)-cyclopropyl.

In the present disclosure, the term “arylcarbonyl” as used by itself or as part of another group refers to a carbonyl group, i.e., —C(═O)—, substituted with an optionally substituted aryl. A non-limiting exemplary arylcarbonyl group is —COPh.

In the present disclosure, the term “alkoxycarbonyl” as used by itself or as part of another group refers to a carbonyl group, i.e., —C(═O)—, substituted with an alkoxy. In one embodiment, the alkoxy is a C₁₋₄ alkoxy. Non-limiting exemplary alkoxycarbonyl groups include —C(═O)OMe, —C(═O)OEt, and —C(═O)OtBu.

In the present disclosure, the term “(alkoxycarbonyl)alkyl” as used by itself or as part of another group refers to an alkyl substituted by an alkoxycarbonyl group. Non-limiting exemplary (alkoxycarbonyl)alkyl groups include —CH₂C(═O)OMe, —CH₂C(═O)OEt, and —CH₂C(═O)OtBu.

In the present disclosure, the term “carboxy” as used by itself or as part of another group refers to a radical of the formula —CO₂H.

In the present disclosure, the term “carboxyalkyl” as used by itself or as part of another group refers to an alkyl substituted with a —CO₂H. A non-limiting exemplary carboxyalkyl group is —CH₂CO₂H.

In the present disclosure, the term “aralkyl” as used by itself or as part of another group refers to an alkyl substituted with one, two, or three optionally substituted aryl groups. In one embodiment, aralkyl is a C₁₋₄ alkyl substituted with one optionally substituted C₅ or C₆ aryl group. In another embodiment, the aralkyl is a C₁ alkyl substituted with one optionally substituted aryl group. In another embodiment, the aralkyl is a C₂ alkyl substituted with one optionally substituted aryl group. In another embodiment, the aralkyl is a C₃ alkyl substituted with one optionally substituted aryl group. In one embodiment, the aralkyl is a C₁ or C₂ alkyl substituted with one optionally substituted phenyl group. Non-limiting exemplary aralkyl groups include benzyl, phenethyl, —CHPh₂, —CH(CH₃)Ph, —CH₂(4-F-Ph), —CH₂(4-Me-Ph), —CH-₂(4-CF₃-Ph), and —CH(4-F-Ph)₂.

In the present disclosure, the term “(heterocyclo)alkyl” as used by itself or part of another group refers to an alkyl substituted with an optionally substituted heterocyclo group. In one embodiment, the (heterocyclo)alkyl is a C₁₋₄ alkyl substituted with one optionally substituted heterocyclo group. Non-limiting exemplary (heterocyclo)alkyl groups include:

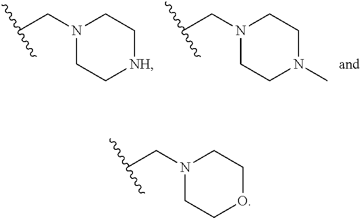

Additional non-limiting exemplary (heterocyclo)alkyl groups include:

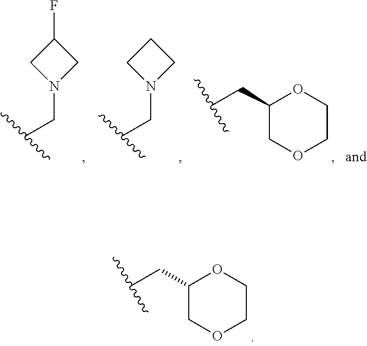 [SMILES: *CN1CC(F)C1 ; *CN1CCC1 ; *C[C@@H]1COCCO1 ; *C[C@H]1COCCO1]

In the present disclosure, the term “(heteroaryl)alkyl” as used by itself or part of another group refers to an alkyl substituted with an optionally substituted heteroaryl group. In one embodiment, the (heteroaryl)alkyl is a C₁₋₄ alkyl substituted with one optionally substituted heteroaryl group. In another embodiment, the (heteroaryl)alkyl is a C₁ alkyl substituted with one optionally substituted heteroaryl group Non-limiting exemplary (heteroaryl)alkyl groups include:

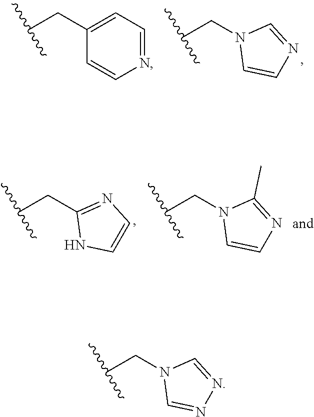

In the present disclosure, the term “(carboxamido)alkyl” as used by itself or as part of another group refers to an alkyl substituted with one or two carboxamido groups. In one embodiment, the (carboxamido)alkyl is a C₁₋₄ alkyl substituted with one carboxamido group, i.e., a (carboxamido)C₁₋₄ alkyl. In another embodiment, the (carboxamido)alkyl is a C₁₋₄ alkyl substituted with two carboxamido groups. Non-limiting exemplary (carboxamido)alkyl groups include —CH₂CONH₂, —C(H)CH₃—CONH₂, and —CH₂CON(H)CH₃.

In the present disclosure, the term “(aryloxy)alkyl” as used by itself or as part of another group refers to an alkyl substituted with an aryloxy group. In one embodiment, the “(aryloxy)alkyl” is a C₁₋₄ alkyl substituted with an aryloxy. In one embodiment, the “(aryloxy)alkyl” is a C₂₋₄ alkyl substituted with an aryloxy. Non-limiting exemplary (aryloxy)alkyl groups include —CH₂CH₂OPh and —CH₂CH₂CH₂OPh.

In the present disclosure, the term “alkylcarbonyloxy” as used by itself or as part of another group refers to an oxy, e.g., —O—, substituted with an alkylcarbonyl group. Non-limiting exemplary “alkylcarbonyloxy” groups include —OC(═O)CH₂CH₃, —OC(═O)CH₃, i.e., acetoxy, —OC(═O)CH₂CH₂CH₃, and —OC(═O)CH(CH₃)₂.

In the present disclosure, the term “cycloalkylcarbonyloxy” as used by itself or as part of another group refers to an oxy, e.g., —O—, substituted with an cycloalkylcarbonyl group. Non-limiting exemplary “cycloalkylcarbonyloxy” groups include —OC(═O)-cyclopropyl and —OC(═O)-cyclopenyl.

The term “menin inhibitor” or “inhibitor of menin” as used herein refers to a compound that disrupts, e.g., inhibits, the menin-MLL fusion protein interaction.

The term “a disease or condition wherein inhibition of menin provides a benefit” pertains to a disease or condition in which menin and/or the interaction of menin with a menin-interacting protein is important or necessary, e.g., for the onset, progress, or expression of that disease or condition, or a disease or a condition which is known to be treated by a menin inhibitor. Examples of such conditions include, but are not limited to, a cancer, a chronic autoimmune disease, an inflammatory disease, a proliferative disease, sepsis, and a viral infection. One of ordinary skill in the art is readily able to determine whether a compound treats a disease or condition mediated by menin for any particular cell type, for example, by assays which conveniently can be used to assess the activity of particular compounds.

The term “second therapeutic agent” refers to a therapeutic agent different from a Compound of the Disclosure and that is known to treat the disease or condition of interest. For example when a cancer is the disease or condition of interest, the second therapeutic agent can be a known chemotherapeutic drug, like taxol, or radiation, for example.

The term “disease” or “condition” denotes disturbances and/or anomalies that as a rule are regarded as being pathological conditions or functions, and that can manifest themselves in the form of particular signs, symptoms, and/or malfunctions. As demonstrated below, Compounds of the Disclosure are menin inhibitors and can be used in treating diseases and conditions wherein menin inhibition provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a Compound of the Disclosure to an individual in need of such treatment.

Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; reduce menin interactions in the target cells; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to an individual in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), to an individual in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at about 1 minute to about 24 hours apart.

The use of the terms “a”, “an”, “the”, and similar referents in the context of this disclosure (especially in the context of the claims) are to be construed to cover both the singular and the plural, unless otherwise indicated. Recitation of ranges of values herein are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number ±10%. Thus, “about 10” means 9 to 11.

Compounds of the Disclosure have asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem (55:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry. In one embodiment, ee is determined by chiral HPLC.

### V. Synthesis of Compounds of the Disclosure

Compounds of the Disclosure can be prepared by methods described in the Examples and by related methods known in the art.

For example, compounds of Formula I, wherein E is E-2, can be prepared by the general method shown in Scheme 1. A nucleophilic substitution reaction between phenol 1-A and electrophile 1-B (LG=leaving group) in the presence of a base (e.g., K₂CO₃) affords the Compound of Formula I (1-C).

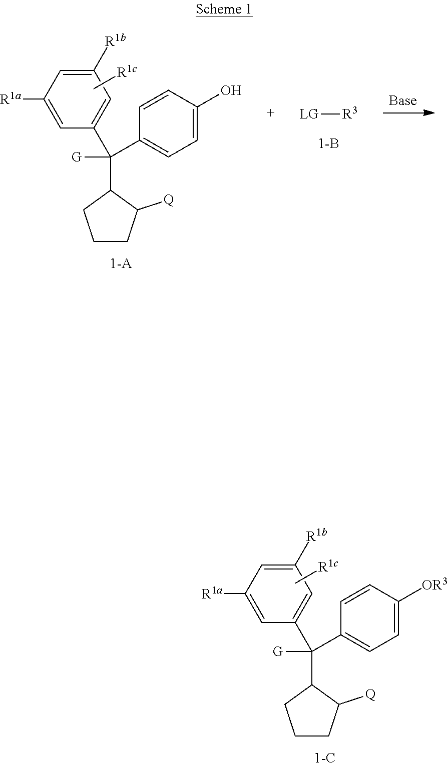

Compounds of Formula I, wherein E is E-3, and B is B2, can be prepared by the general method shown in Scheme 2. A nucleophilic aromatic substitution reaction between azetidine 2-A and aryl fluoride 2-B in the presence of a base (e.g., K₂CO₃) affords the Compound of Formula I (2-C).

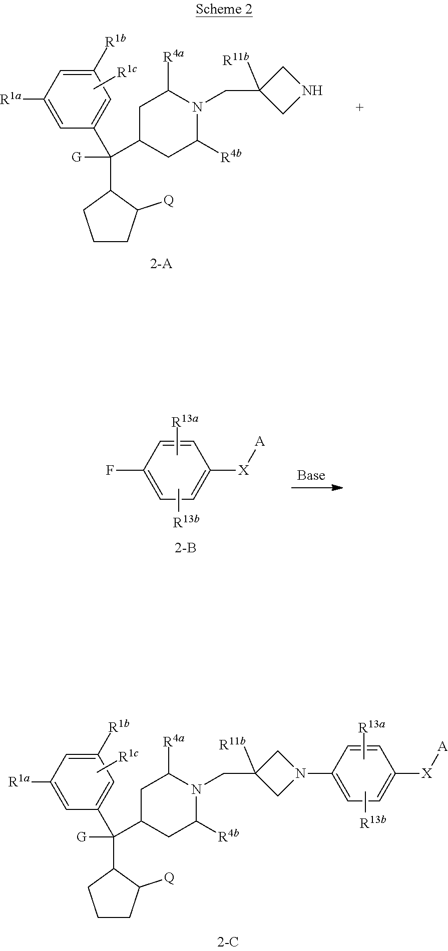

Alternatively, compounds of Formula I, wherein E is E-3, and B is B2, can be prepared by the general method shown in Scheme 3. A nucleophilic substitution reaction between piperidine 3-A and electrophile 3-B (LG=leaving group) in the presence of a base (e.g., K₂CO₃) affords the Compound of Formula I (3-C).

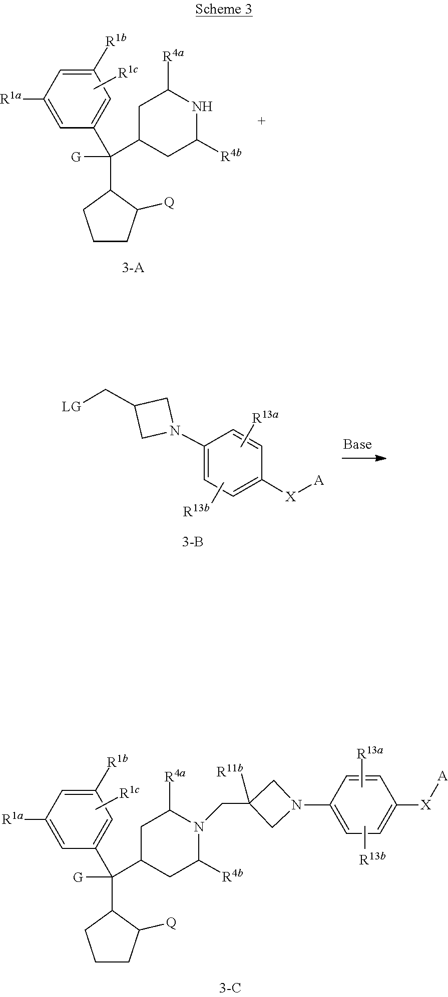

### VI. Additional Embodiments

In additional embodiments, the disclosure relates to:

1. A compound of Formula I:

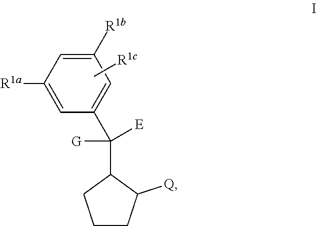

wherein:

Q is selected from the group consisting of —N(H)C(═O)OR, —N(R)C(═O)OR, —N(H)C(═O)R, —N(H)C(O)NR₂,

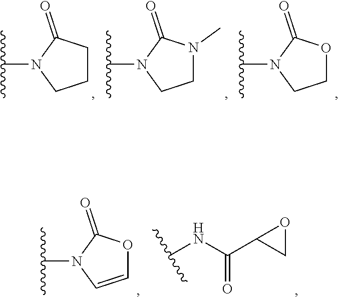

### —OR, and —OC(═O)R;

each R is independently C₁-C₄ alkyl or C₁-C₄ haloalkyl;

G is selected from the group consisting of:

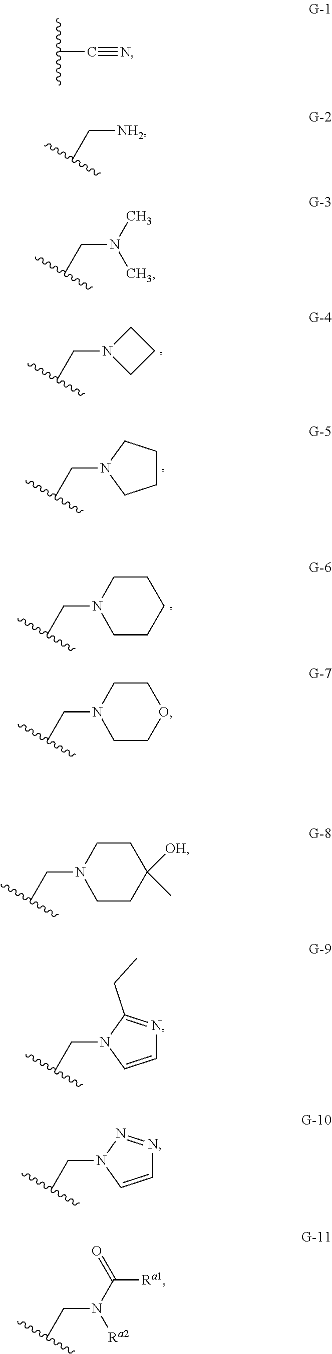

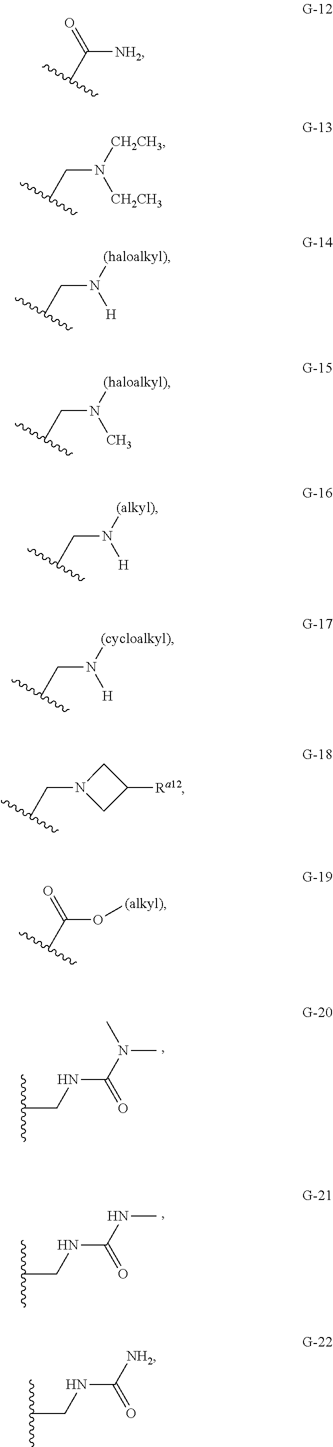

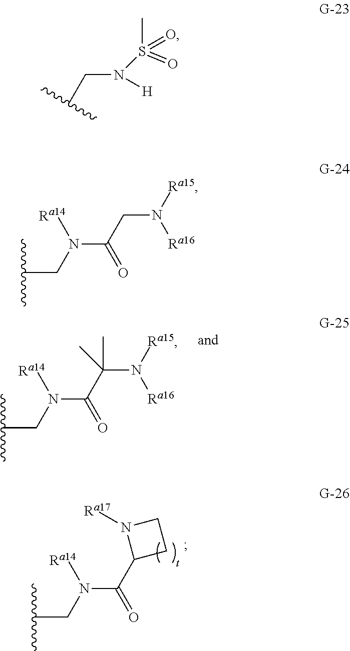

R^{a1} is selected from the group consisting of C₁-C₄ alkyl and C₁-C₄ alkoxy;

R^{a2} is selected from the group consisting of hydrogen and C₁-C₄ alkyl; or

R^{a1} and R^{a2} taken together with the atoms to which they are attached form an optionally substituted 5- or 6-membered heterocyclo;

R^{a12} is CN, C(O)OR^{a13}, C(O)N(R^{a13})₂, C₁-C₄ alkyl, OH, C₁-C₄ alkoxy, or F;

each R^{a13} is independently C₁-C₄ alkyl;

R^{a14} is H or C₁-C₄ alkyl;

R^{a15} and R^{a16} are each independently H or C₁-C₄ alkyl, or R^{a14} and R^{a15} together with the nitrogen atom to which they are attached form an optionally substituted 4- to 6-membered heterocyclo;

R^{a17} is H or C₁-C₄ alkyl;

t is 1, 2, or 3;

R^{1a}, R^{1b}, and R^{1c} are each independently selected from the group consisting of hydrogen and halo;

E is selected from the group consisting of:

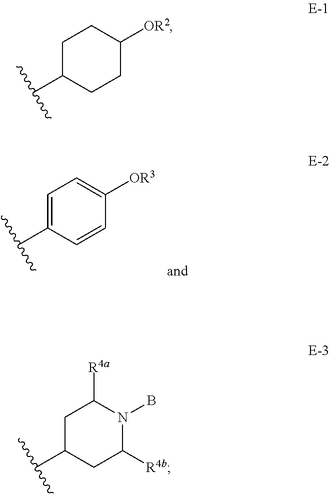

R² is selected from the group consisting of C₁-C₆ alkyl and —(CR^{5a}R^{5b})_{P}OR^{6a};

R³ is selected from the group consisting of hydrogen, —(CR^{5a}R^{5b})_{P}OR^{6b}, —CH₂C≡CR⁷, and

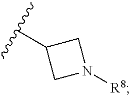

each R^{5a} and R^{5b} is independently selected from the group consisting of hydrogen and C₁-C₄ alkyl;

p is 2, 3, or 4;

R^{6a} is optionally substituted phenyl;

R^{6b} is selected from the group consisting of C₁-C₆ alkyl and optionally substituted phenyl;

R⁷ is optionally substituted phenyl;

R⁸ is optionally substituted phenyl;

R^{4a} and R^{4b} are independently selected from the group consisting of hydrogen, halo, and C₁-C₄ alkyl;

B is selected from the group consisting of C₁-C₆ alkyl, aralkyl, —C(═O)R⁹, —(CR^{5c}R^{5d})ₘOR¹⁰,

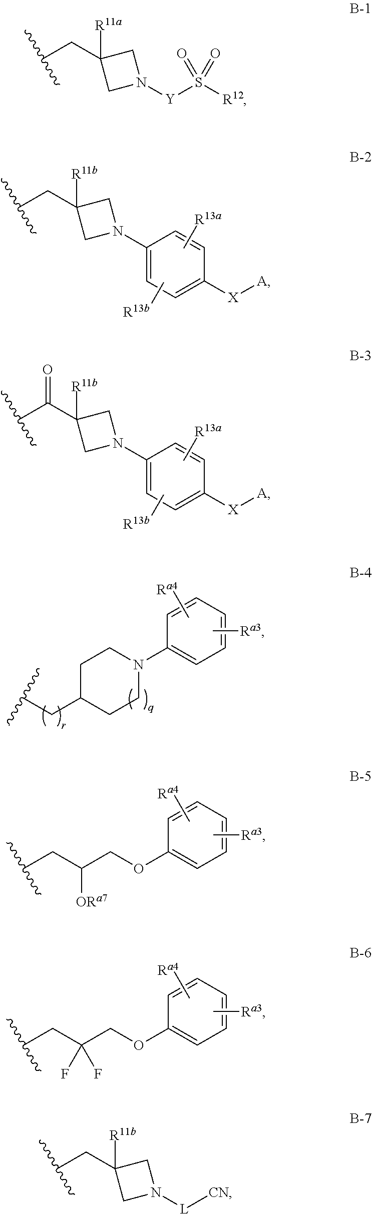

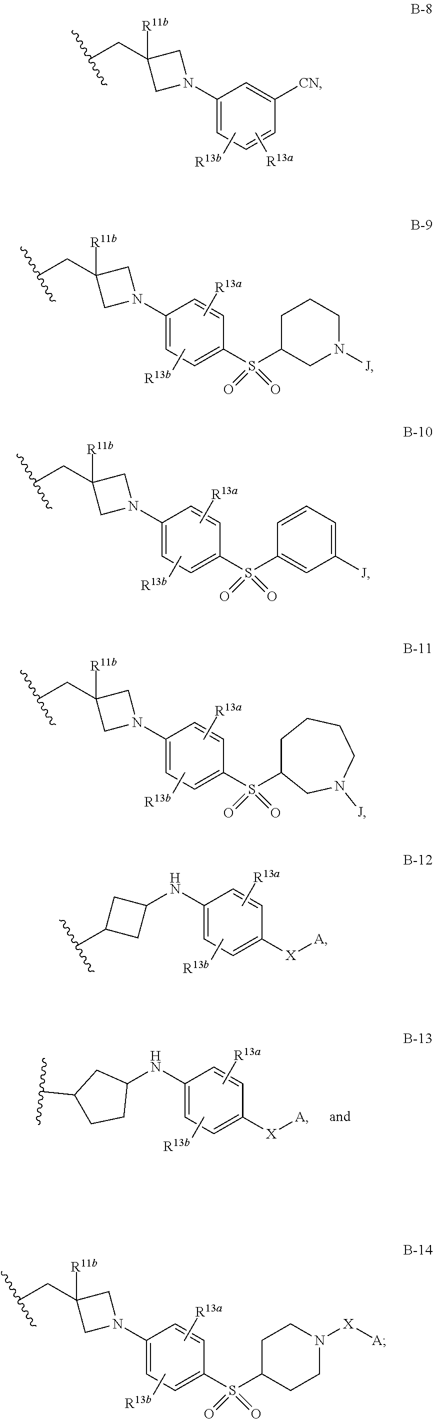

R⁹ is selected from the group consisting of C₁-C₆, alkyl, aralkyl, heteroaralkyl, and

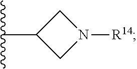

R¹⁴ is optionally substituted phenyl;

each R^{5c} and R^{5d} is independently selected from the group consisting of hydrogen and C₁-C₄ alkyl;

m is 2, 3, or 4;

R¹⁰ is optionally substituted phenyl;

R^{11a} is selected from the group consisting of hydrogen, halo, and C₁-C₄ alkyl;

Y is —(CR^{5e}R^{5f})ₒ;

each R^{5e} and R^{5f} is independently selected from the group consisting of hydrogen and C₁-C₄ alkyl;

o is 2, 3, or 4;

R¹² is optionally substituted phenyl;

R^{11b} is selected from the group consisting of hydrogen, halo, C₁-C₄ alkyl, and R^{a6};

R^{13a} and R^{13b} are independently selected from the group consisting of hydrogen, halo, C₁-C₄ alkyl, and R^{a5};

R^{a3} is selected from the group consisting of cyano, alkylsulfonyl, haloalkylsulfonyl, cycloalkylsulfonyl, arylsulfonyl, heteroarylsulfonyl, heterocyclosulfonyl, and carboxamido;

R^{a4} is selected from the group consisting of hydrogen, halo, C₁-C₄ alkyl, C₁-C₄ alkoxy, and C₁-C₄ haloalkyl;

R^{a5} is selected from the group consisting of (heterocyclo)alkyl, (heteroaryl)alkyl, (amino)alkyl, carboxamido, hydroxyalkyl, heteroaryloxy, heteroaralkyloxy, C₁-C₄ alkoxy, —OR^{a8}, and —CH₂NR^{a9}C(═O)R^{a10};

R^{a6} is selected from the group consisting of hydroxy, C₁-C₄ alkoxy, C₁-C₄ haloalkyl, C₁-C₄ hydroxyalkyl, alkoxyalkyl, carboxy, alkoxy carbonyl, and carboxamido;

R^{a7} is selected from the group consisting of hydrogen and C₁-C₄ alkyl;

R^{a8} is selected from the group consisting of heteroaryl, heteroaralkyl, alkoxyalkyl, and (heterocyclo)alkyl;

R^{a9} is selected from the group consisting of hydrogen and C₁-C₄ alkyl;

R^{a10} is C₁-C₄ alkyl;

r is 0 or 1;

q is 0, 1, 2, or 3;

L is selected from the group consisting of C₃-C₈ cycloalkylenyl, optionally substituted 5-membered heteroaryl enyl, and optionally substituted 6-membered heteroaryl enyl;

(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b},

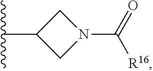

### and R^{a11}; or

(2) X is absent; and A is selected from the group consisting of cyano, C(═O)OH, C₁-C₄ alkyl, C₁-C₄ alkoxy, and C₁-C₄ haloalkyl;

J is carboxamido or C(O)CH₂CN;

R^{a11} is selected from the group consisting of hydroxyalkyl and (heterocyclo)alkyl;

R^{15a} and R^{15b} are independently selected from the group consisting of hydrogen, optionally substituted C₁-C₆ alkyl, optionally substituted C₃-C₁₂ cycloalkyl, optionally substituted 4- to 14-membered heterocyclo, optionally substituted C₆-C₁₀ aryl, and optionally substituted 5- to 14-membered heteroaryl; and

R¹⁶ is selected from the group consisting of (amino)alkyl and (heterocyclo)alkyl,

or a pharmaceutically acceptable salt or solvate thereof.

2. The compound of embodiment 1, wherein:

Q is selected from the group consisting of —N(H)C(═O)OR, —OR, and —OC(═O)R;

R is a C₁-C₄ alkyl;

G is selected from the group consisting of:

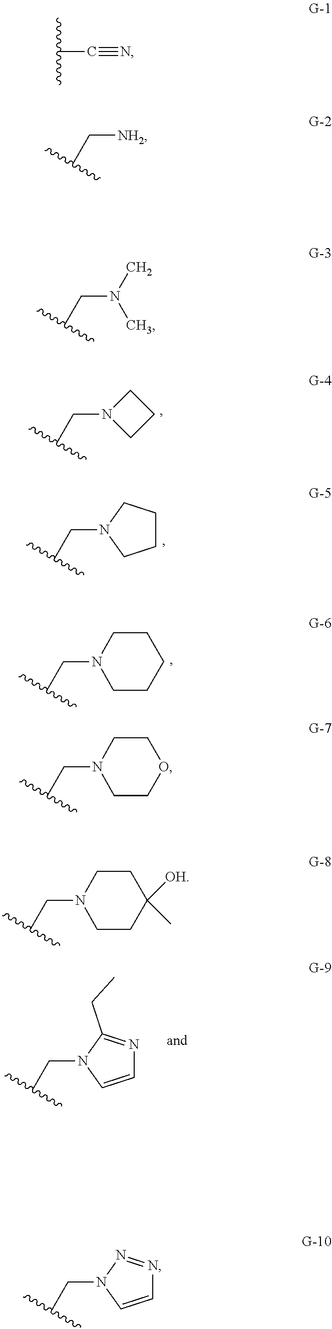

B is selected from the group consisting of C₁-C₆ alkyl, aralkyl, —C(═O)R⁹, —(CR^{5c}R^{5d})ₘOR¹⁰,

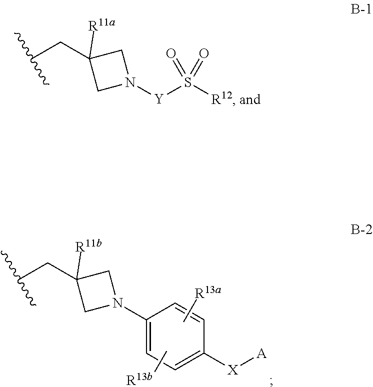

R^{11b} is selected from the group consisting of hydrogen, halo, and C₁-C₄ alkyl;

R^{13a} and R^{13b} are independently selected from the group consisting of hydrogen, halo, and C₁-C₄ alkyl; and

(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b}, and R¹⁶;

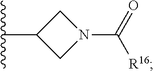

or

(2) X is absent; and A is selected from the group consisting of cyano and —C(═O)OH;

or a pharmaceutically acceptable salt or solvate thereof.

3. The compound of embodiment 1 or 2 of Formula II:

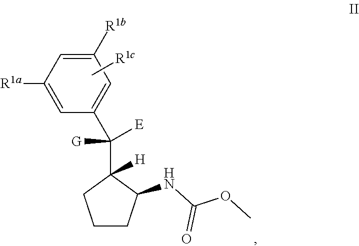

or a pharmaceutically acceptable salt or solvate thereof.

4. The compound of embodiment 1, 2 or 3, wherein E is E-1, or a pharmaceutically acceptable salt or solvate thereof.

5. The compound of embodiment 4, wherein R² is —(CH₂)_{P}OR^{6a}, or a pharmaceutically acceptable salt or solvate thereof.

6. The compound of embodiment 1, 2 or 3, wherein E is E-2, or a pharmaceutically acceptable salt or solvate thereof.

7. The compound of embodiment 6, wherein R³ is —(CH₂)_{P}OR^{6b}, or a pharmaceutically acceptable salt or solvate thereof.

8. The compound of embodiment 6, wherein R³ is —CH₂C≡CR⁷, or a pharmaceutically acceptable salt or solvate thereof.

9. The compound of embodiment 6, wherein R³ is

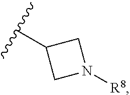

or a pharmaceutically acceptable salt or solvate thereof.

10. The compound of embodiment 1, 2 or 3, wherein E is E-3, or a pharmaceutically acceptable salt or solvate thereof.

11. The compound of any one of embodiments 1, 2, 3, and 10, wherein B is C₁-C₆ alkyl, or a pharmaceutically acceptable salt or solvate thereof.

12. The compound of any one of embodiments 1, 2, 3, and 10, wherein B is aralkyl, or a pharmaceutically acceptable salt or solvate thereof.

13. The compound of any one of embodiments 1, 2, 3, and 10, wherein B is —C(═O)R⁹, or a pharmaceutically acceptable salt or solvate thereof.

14. The compound of embodiment 13, wherein R⁹ is selected from the group consisting of aralkyl, heteroaralkyl, and

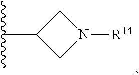

or a pharmaceutically acceptable salt or solvate thereof.

15. The compound of embodiment 14, wherein R⁹ is

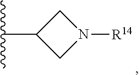

or a pharmaceutically acceptable salt or solvate thereof.

16. The compound of any one of embodiments 1, 2, 3, and 10, wherein B is —(CH₂)ₘOR¹⁰ or a pharmaceutically acceptable salt or solvate thereof.

17. The compound of embodiment 16, wherein m is 1, 2 or 3, or a pharmaceutically acceptable salt or solvate thereof.

18. The compound of any one of embodiments 1, 2, 3, and 10, wherein B is B-1, or a pharmaceutically acceptable salt or solvate thereof.

19. The compound of embodiment 18, wherein Y is —(CH₂)ₒ—, or a pharmaceutically acceptable salt or solvate thereof.

20. The compound of any one of embodiments 1, 2, 3, and 10, wherein B is B-2, or a pharmaceutically acceptable salt or solvate thereof.

21. The compound of embodiment 20, wherein X is —S(═O)₂, or a pharmaceutically acceptable salt or solvate thereof.

22. The compound of any one of embodiments 1-21, wherein G is —CN, or a pharmaceutically acceptable salt or solvate thereof.

23. The compound of any one of embodiments 1-21, wherein G is —CH₂NH₂, or a pharmaceutically acceptable salt or solvate thereof.

24. The compound of any one of embodiments 1-20, wherein G is —CH₂N(CH₃)₂, or a pharmaceutically acceptable salt or solvate thereof.

25. The compound of any one of embodiments 1-21, wherein G is

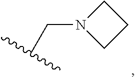 [SMILES: *CN1CCC1 ; ","]

or a pharmaceutically acceptable salt or solvate thereof.

26. The compound of any one of embodiments 1-21, wherein G is

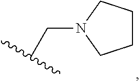 [SMILES: *CN1CCCC1 ; ","]

or a pharmaceutically acceptable salt or solvate thereof.

27. The compound of any one of embodiments 1-21, wherein G is

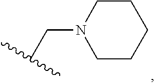 [SMILES: *CN1CCCCC1 ; ","]

or a pharmaceutically acceptable salt or solvate thereof.

28. The compound of any one of embodiments 1-21, wherein G is

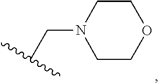 [SMILES: *CN1CCOCC1 ; ","]

or a pharmaceutically acceptable salt or solvate thereof.

29. The compound of any one of embodiments 1-21, wherein G is

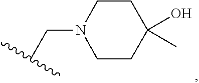 [SMILES: *CN1CCC(C)(O)CC1 ; ","]

or a pharmaceutically acceptable salt or solvate thereof.

30. The compound of any one of embodiments 1-21, wherein G is

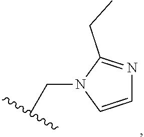 [SMILES: *Cn1ccnc1CC ; ","]

or a pharmaceutically acceptable salt or solvate thereof.

31. The compound of any one of embodiments 1-21, wherein G is

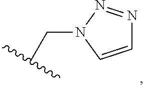 [SMILES: *Cn1ccnn1 ; ","]

or a pharmaceutically acceptable salt or solvate thereof.

32. The compound of embodiment 1 or 2 of Formula III:

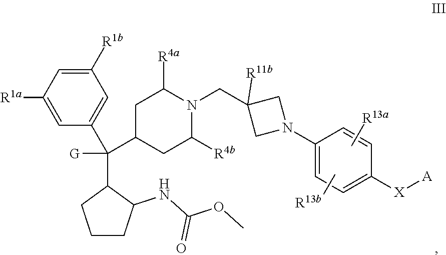

wherein:

G is selected from the group consisting of:

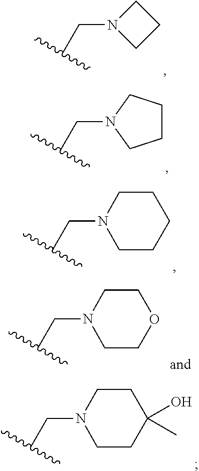

or a pharmaceutically acceptable salt or solvate thereof.

33. The compound of embodiment 32, wherein G is

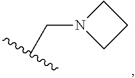 [SMILES: *CN1CCC1 ; ","]

or a pharmaceutically acceptable salt or solvate thereof.

34. The compound of embodiment 33 of Formula IV:

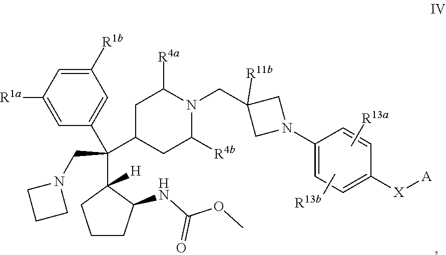

or a pharmaceutically acceptable salt or solvate thereof.

35. The compound of any one of embodiments 32-34, wherein R^{4a} and R^{4b} are hydrogen, or a pharmaceutically acceptable salt or solvate thereof.

36. The compound of any one of embodiments 32-35, wherein R^{11b} is selected from the group consisting of hydrogen and fluoro, or a pharmaceutically acceptable salt or solvate thereof.

37. The compound of any one of embodiments 32-36, wherein R^{13a} and R^{13b} are independently selected from the group consisting of hydrogen and fluoro, or a pharmaceutically acceptable salt or solvate thereof.

38. The compound of any one of embodiments 32-37, wherein X is —C(═O)—, or a pharmaceutically acceptable salt or solvate thereof.

39. The compound of any one of embodiments 32-37, wherein X is —S(═O)₂—, or a pharmaceutically acceptable salt or solvate thereof.

40. The compound of any one of embodiments 32-39, wherein A is optionally substituted C₃-C₁₂ cycloalkyl, or a pharmaceutically acceptable salt or solvate thereof.

41. The compound of embodiment 40, wherein A is selected from the group consisting of:

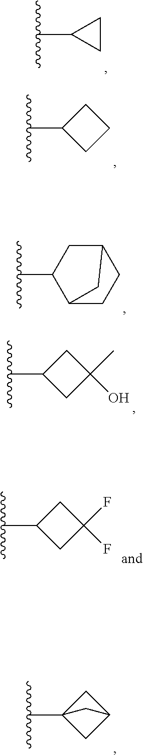

or a pharmaceutically acceptable salt or solvate thereof.

42. The compound of any one of embodiment 32-39, wherein A is optionally substituted 4- to 14-membered heterocyclo, or a pharmaceutically acceptable salt or solvate thereof.

43. The compound of embodiment 42, wherein A is selected from the group consisting of:

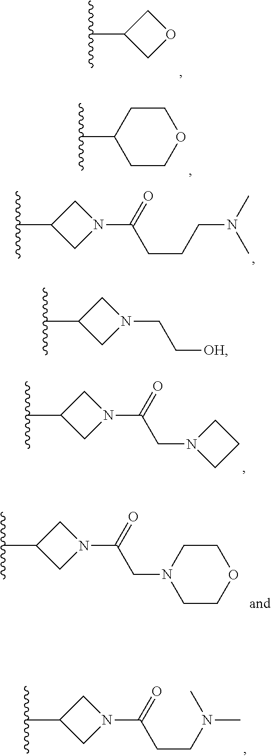

or a pharmaceutically acceptable salt or solvate thereof.

44. The compound of any one of embodiments 32-39, wherein A is optionally substituted phenyl, or a pharmaceutically acceptable salt or solvate thereof.

45. The compound of any one of embodiments 32-39, wherein A is optionally substituted 5- or 6-membered heteroaryl, or a pharmaceutically acceptable salt or solvate thereof.

46. The compound of embodiment 45, wherein A is selected from the group consisting of:

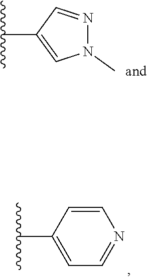

or a pharmaceutically acceptable salt or solvate thereof.

47. The compound of any one of embodiments 32-39, wherein A is —NR^{15a}R^{15b}, or a pharmaceutically acceptable salt or solvate thereof.

48. The compound of embodiment 47, wherein R^{15a} and R^{15b} are independently selected from the group consisting of hydrogen and optionally substituted C₁-C₆ alkyl, or a pharmaceutically acceptable salt or solvate thereof.

49. The compound of any one of embodiments 32-39, wherein A is

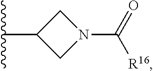

or a pharmaceutically acceptable salt or solvate thereof.

50. The compound of embodiment 49, wherein R¹⁶ is selected from the group consisting of —CH₂CH₂CH₂N(CH₃)₂, —CH₂CH₂N(CH₃)₂, and

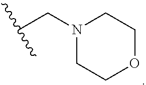 [SMILES: *CN1CCOCC1 ; "."]

51. The compound of any one of embodiments 32-37, wherein X is absent and A is cyano, or a pharmaceutically acceptable salt or solvate thereof.

52. The compound of any one of embodiments 1-52, wherein R^{1a} and R^{1b} are independently selected from the group consisting of hydrogen and fluoro, or a pharmaceutically acceptable salt or solvate thereof.

53. The compound of any one of embodiments 1-52, wherein R^{1c} is hydrogen, or a pharmaceutically acceptable salt or solvate thereof.

54. The compound of embodiment 2 selected from the group consisting of the compounds of Table 1.1, or a pharmaceutically acceptable salt thereof.

55. The compound of embodiment 54, selected from the group consisting of:
methyl ((1S,2R)-2-((S)-2-(azetidin-1-yl)-1-(1-((1-(4-(cyclopropylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-(cyclopropylsulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyanophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-((1-methyl-1H-pyrazol-4-yl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-(cyclobutylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-2,6-difluorophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-(oxetan-3-ylsulfonyl)phenyl)azetidin-3-yl)methyl)piperidin-4-yl)ethyl)cyclopentyl)carbamate;
methyl ((1S,2R)-2-((S)-2-(azetidin-1-yl)-1-(3-fluorophenyl)-1-(1-((1-(4-((tetrahydro-2H-pyran-4-yl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-(cyclopropylsulfonyl)phenyl)azetidin-3-yl)methyl)piperidin-4-yl)-1-(3,5-difluorophenyl)ethyl)cyclopentyl)carbamate;
methyl ((1S,2R)-2-((R)-2-(azetidin-1-yl)-1-(1-((1-(4-(cyclopropylsulfonyl)phenyl)azetidin-3-yl)methyl)piperidin-4-yl)-1-(3,5-difluorophenyl)ethyl)cyclopentyl)carbamate;
methyl ((1S,2R)-2-((1S)-2-(azetidin-1-yl)-1-(1-((1-(4-(bicyclo[2.2.1]heptan-2-ylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyanophenyl)-3-fluoroazetidin-3-yl)methyl)piperidin-4-yl)-1-(3,5-difluorophenyl)ethyl)cyclopentyl)carbamate;
methyl ((1S,2R)-2-((S)-2-(azetidin-1-yl)-1-(1-((1-(4-(cyclopropylsulfonyl)phenyl)-3-fluoroazetidin-3-yl)methyl)piperidin-4-yl)-1-(3,5-difluorophenyl)ethyl)cyclopentyl)carbamate;
methyl ((1S,2R)-2-((S)-2-(azetidin-1-yl)-1-(1-((1-(4-cyano-2,6-difluorophenyl)-3-fluoroazetidin-3-yl)methyl)piperidin-4-yl)-1-(3,5-difluorophenyl)ethyl)cyclopentyl)carbamate;
methyl ((1S,2R)-2-((S)-2-(azetidin-1-yl)-1-(1-((1-(4-cyanophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-2,6-difluorophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-((3-hydroxy-3-methylcyclobutyl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-((3,3-difluorocyclobutyl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-((1-(4-(dimethylamino)butanoyl)azetidin-3-yl)sulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-((3,3-difluorocyclobutyl)sulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-(bicyclo[1.1.1]pentan-1-ylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-(bicyclo[1.1.1]pentan-1-yl sulfonyl)phenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-((3-hydroxy-3-methylcyclobutyl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-((1-(2-hydroxyethyl)azetidin-3-yl)sulfonyl)phenyl)azetidin-3-yl)methyl)piperidin-4-yl)-1-(3-fluorophenyl)ethyl)cyclopentyl)carbamate;
4-((4-(3-((4-((S)-2-(azetidin-1-yl)-1-(3-fluorophenyl)-1-((1R,2S)-2-((methoxycarbonyl)amino)cyclopentyl)ethyl)piperidin-1-yl)methyl)azetidin-1-yl)phenyl)sulfonyl)benzoic acid;
methyl ((1S,2R)-2-((S)-2-(azetidin-1-yl)-1-((1r,4S)-4-(2-(4-(cyclopropylsulfonyl)phenoxy)ethoxy)cyclohexyl)-1-(3-fluorophenyl)ethyl)cyclopentyl)carbamate;
methyl ((1S,2R)-2-((R)-2-(azetidin-1-yl)-1-((1r,4R)-4-(2-(4-(cyclopropylsulfonyl)phenoxy)ethoxy)cyclohexyl)-1-(3-fluorophenyl)ethyl)cyclopentyl)carbamate;
methyl ((1S,2R)-2-((S)-2-(azetidin-1-yl)-1-(1-((1-(4-cyano-2-fluorophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-3-fluorophenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-2-fluorophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((1-(4-cyano-3-fluorophenyl)-3-fluoroazetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-(pyridin-4-ylsulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(1-((3-fluoro-1-(4-((1-methyl-1H-pyrazol-4-yl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-(methylcarbamoyl)phenyl)azetidin-3-yl)methyl)piperidin-4-yl)ethyl)cyclopentyl)carbamate;
methyl ((1S,2R)-2-((S)-2-(azetidin-1-yl)-1-(3-fluorophenyl)-1-(1-((1-(4-((4-(methylcarbamoyl)phenyl)sulfonyl)phenyl)azetidin-3-yl)methyl)piperidin-4-yl)ethyl)cyclopentyl)carbamate;
methyl ((1S,2R)-2-((S)-2-(azetidin-1-yl)-1-(1-((1-(4-((1-(2-(azetidin-1-yl)acetyl)azetidin-3-yl)sulfonyl)phenyl)azetidin-3-yl)methyl)piperidin-4-yl)-1-(3-fluorophenyl)ethyl)cyclopentyl)carbamate;
methyl ((1S,2R)-2-((S)-2-(azetidin-1-yl)-1-(3-fluorophenyl)-1-(1-((1-(4-((1-(2-morpholinoacetyl)azetidin-3-yl)sulfonyl)phenyl)azetidin-3-yl)methyl)piperidin-4-yl)ethyl)cyclopentyl)carbamate; and
methyl ((1S,2R)-2-((S)-2-(azetidin-1-yl)-1-(1-((1-(4-((1-(3-(dimethylamino)propanoyl)azetidin-3-yl)sulfonyl)phenyl)azetidin-3-yl)methyl)piperidin-4-yl)-1-(3-fluorophenyl)ethyl)cyclopentyl)carbamate,
   or a pharmaceutically acceptable salt or solvate thereof.

56. A pharmaceutical composition comprising the compound of any one of embodiments 2-55, or a pharmaceutically acceptable salt thereof, and a pharmaceutically acceptable carrier.

57. A method of treating a subject in need thereof, the method comprising administering to the subject a therapeutically effective amount of the compound of any one of embodiments 2-55, or a pharmaceutically acceptable salt or solvate thereof, wherein the subject has cancer.

58. The method of embodiment 57, wherein the cancer is any one or more of the cancers of Table 2.

59. The method of embodiment 58, wherein the cancer is a hematological cancer.

60. The method of embodiment 59, wherein the hematological cancer is any one or more of the cancers of Table 3.

61. The method of any one of embodiments 57-60 further comprising administering a therapeutically effective amount of a second therapeutic agent useful in the treatment of cancer.

62. The pharmaceutical composition of embodiment 56 for use in treating cancer.

63. The pharmaceutical composition of embodiment 62, wherein the cancer is any one or more of the cancers of Table 2.

64. The pharmaceutical composition of embodiment 63, wherein the cancer is a hematological cancer.

65. The pharmaceutical composition of embodiment 64, wherein the hematological cancer is any one or more of the cancers of Table 3.

66. A compound of any one of embodiments 1-55, or a pharmaceutically acceptable salt or solvate thereof, for use in treatment of cancer.

67. The compound for use of embodiment 66, wherein the cancer is any one or more of the cancers of Table 2.

68. The compound for use of embodiment 67, wherein the cancer is a hematological cancer.

69. The compound for use of embodiment 68, wherein the hematological cancer is any one or more of the cancers of Table 3.

70. Use of a compound of any one of embodiments 2-55, or a pharmaceutically acceptable salt or solvate thereof, for the manufacture of a medicament for treatment of cancer.

71. The use of embodiment 60, wherein the cancer is any one or more of the cancers of Table 2.

72. The use of embodiment 71, wherein the cancer is a hematological cancer.

73. The use of embodiment 72, wherein the hematological cancer is any one or more of the cancers of Table 3.

74. A kit comprising the compound of any one of embodiments 2-55, or a pharmaceutically acceptable salt or solvate thereof, and instructions for administering the compound, or a pharmaceutically acceptable salt or solvate thereof, to a subject having cancer.

75. The kit of embodiment 74, wherein the cancer is any one or more of the cancers of Table 2.

76. The kit of embodiment 75, wherein the cancer is a hematological cancer.

77. The kit of embodiment 76, wherein the hematological cancer is any one or more of the cancers of Table 3.

78. The kit of any one of embodiments 74-77 further comprising one or more additional therapeutic agents.

79. The compound of embodiment 1, wherein:

Q is selected from the group consisting of —N(H)C(═O)OR and —N(H)C(═O)R;

R is a C₁-C₄ alkyl;

G is selected from the group consisting of:

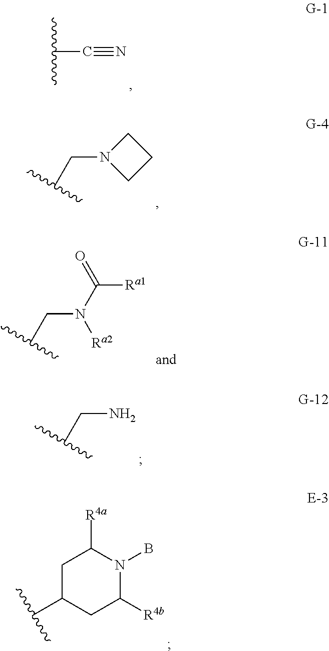

E is:

R^{4a} and R^{4b} are independently selected from the group consisting of hydrogen, halo, and C₁-C₄ alkyl;

B is selected from the group consisting of:

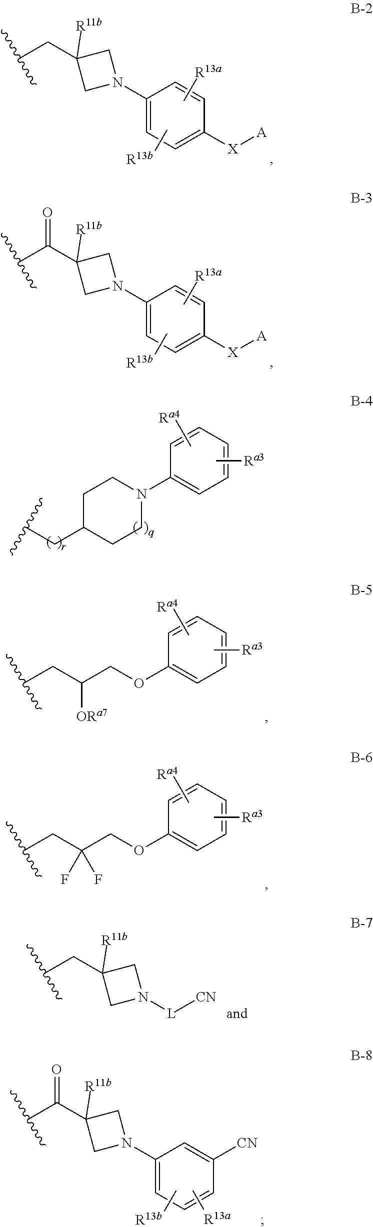

and

R^{11b} is selected from the group consisting of hydrogen, halo, C₁-C₄ alkyl, and R^{a6};

R^{a5} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10};

R^{a6} is selected from the group consisting of hydroxy, C₁-C₄ haloalkyl, alkoxyalkyl, carboxy, alkoxycarbonyl, and carboxamido;

(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b},

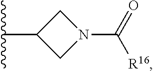

### and R^{a11}; or

(2) X is absent; and A is selected from the group consisting of cyano and —C(═O)OH;

or a pharmaceutically acceptable salt or solvate thereof.

80. The compound of embodiment 1 or 79 of Formula II:

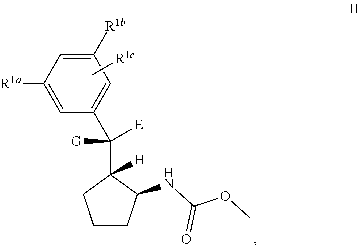

or a pharmaceutically acceptable salt or solvate thereof.

81. The compound of embodiment 1, 79 or 80, wherein B is B-2, or a pharmaceutically acceptable salt or solvate thereof.

82. The compound of embodiment 1, 79 or 80, wherein B is B-3, or a pharmaceutically acceptable salt or solvate thereof.

83. The compound of embodiment 82, wherein X is —S(═O)₂, or a pharmaceutically acceptable salt or solvate thereof.

84. The compound of embodiment 82, wherein X is absent and A is cyano, or a pharmaceutically acceptable salt or solvate thereof.

85. The compound of any one of embodiments 82-84, wherein R^{11b} is selected from the group consisting of hydrogen or fluoro, or a pharmaceutically acceptable salt or solvate thereof.

86. The compound of any one of embodiments 82-84, wherein R^{11b} is selected from the group consisting of C₁-C₄ haloalkyl, alkoxyalkyl, carboxy, alkoxycarbonyl, and carboxamido, or a pharmaceutically acceptable salt or solvate thereof.

87. The compound of any one of embodiments 82-84, wherein R^{13a} is hydrogen, or a pharmaceutically acceptable salt or solvate thereof.

88. The compound of any one of embodiments 82-87, wherein R^{13a} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10}, or a pharmaceutically acceptable salt or solvate thereof.

89. The compound of any one of embodiments 82, 83, and 85-88, wherein: A is selected from the group consisting of phenyl substituted with 1 or 2 substituents independently selected from the group consisting of halo and carboxamido, and 5- or 6-membered heteroaryl substituted with 1 or 2 substituents independently selected from the group consisting of halo and carboxamido.

90. The compound of any one of embodiments 82, 83, and 85-88, wherein: A is selected from the group consisting of unsubstituted C₃-C₆ cycloalkyl, C₃-C₆ cycloalkyl substituted with 1 or 2 substituents independently selected from the group consisting of halo, hydroxy, C₁-C₄ alkyl, and C₁-C₄ haloalkyl, unsubstituted 4- to 6-membered heterocyclo, and 4- to 6-membered heterocyclo substituted with 1 or 2 substituents independently selected from the group consisting of halo, hydroxy, C₁-C₄ alkyl, and C₁-C₄ haloalkyl, alkylcarbonyl, hydroxyalkylcarbonyl, and alkoxycarbonyl.

91. The compound of embodiment 1, 79 or 80, wherein B is B-4, or a pharmaceutically acceptable salt or solvate thereof.

92. The compound of embodiment 91, wherein r is 0, or a pharmaceutically acceptable salt or solvate thereof.

93. The compound of embodiment 91, wherein r is 1, or a pharmaceutically acceptable salt or solvate thereof.

94. The compound of any one of embodiments 91-93, wherein q is 0 or 1, or a pharmaceutically acceptable salt or solvate thereof.

95. The compound of embodiment 1, 79 or 80, wherein B is B-5, or a pharmaceutically acceptable salt or solvate thereof.

96. The compound of embodiment 1, 79 or 80, wherein B is B-6, or a pharmaceutically acceptable salt or solvate thereof.

97. The compound of embodiment 1, 79 or 80, wherein B is B-7, or a pharmaceutically acceptable salt or solvate thereof.

98. The compound of embodiment 97, wherein L is C₃-C₈ cycloalkyl, or a pharmaceutically acceptable salt or solvate thereof.

99. The compound of embodiment 97, wherein L is 5-membered heteroaryl, or a pharmaceutically acceptable salt or solvate thereof.

100. The compound of embodiment 1, 79 or 80, wherein B is B-8, or a pharmaceutically acceptable salt or solvate thereof.

101. The compound of any one of embodiments 82-90 and 100, wherein R^{11b} is selected from the group consisting of hydrogen or fluoro, or a pharmaceutically acceptable salt or solvate thereof.

102. The compound of any one of embodiments 1 and 79-101, wherein G is G-1, or a pharmaceutically acceptable salt or solvate thereof.

103. The compound of any one of embodiments 1 and 79-101, wherein G is G-4, or a pharmaceutically acceptable salt or solvate thereof.

104. The compound of any one of embodiments 1 and 79-101, wherein G is G-11, or a pharmaceutically acceptable salt or solvate thereof.

105. The compound of embodiment 104, wherein R^{a1} selected from the group consisting of methyl and methoxy, or a pharmaceutically acceptable salt or solvate thereof.

106. The compound of embodiment 104 or 105, wherein R^{a2} is hydrogen, or a pharmaceutically acceptable salt or solvate thereof.

107. The compound of embodiment 1 or 79 of Formula III:

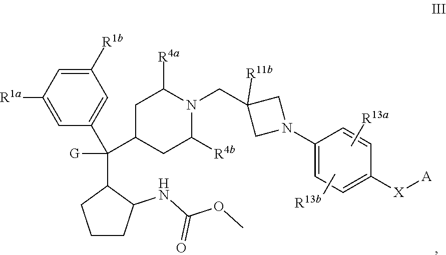

wherein G is selected from the group consisting of G-4 and G-11, or a pharmaceutically acceptable salt or solvate thereof.

108. The compound of embodiment 107, wherein R^{13b} is selected from the group consisting of hydrogen, or a pharmaceutically acceptable salt or solvate thereof.

109. The compound of embodiment 108 of Formula V:

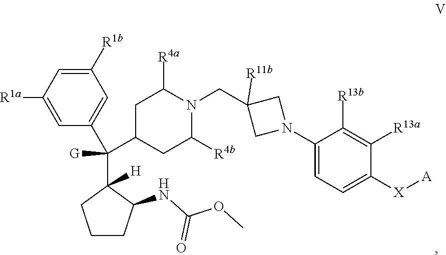

or a pharmaceutically acceptable salt or solvate thereof.

110. The compound of embodiment 109, wherein X is —S(═O)₂—, or a pharmaceutically acceptable salt or solvate thereof.

111. The compound of embodiment 108 of Formula VI:

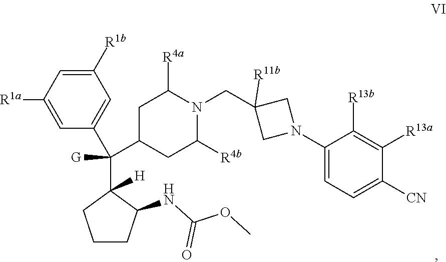

or a pharmaceutically acceptable salt or solvate thereof.

112. The compound of any one of embodiments 108-111, wherein G is G-4, or a pharmaceutically acceptable salt or solvate thereof.

113. The compound of any one of embodiments 108-111, wherein G is G-11, or a pharmaceutically acceptable salt or solvate thereof.

114. The compound of embodiment 113, wherein G is —CH₂N(H)C(═O)CH₃, or a pharmaceutically acceptable salt or solvate thereof.

115. The compound of any one of embodiments 1 and 79-114, wherein R^{4a} and R^{4b} are hydrogen, or a pharmaceutically acceptable salt or solvate thereof.

116. The compound of any one of embodiments 1 and 79-81 and 107-115, wherein R^{11b} is selected from the group consisting of hydrogen or fluoro, or a pharmaceutically acceptable salt or solvate thereof.

117. The compound of any one of embodiments 1, 79-81 and 107-115, wherein R^{11b} is selected from the group consisting of C₁-C₄ haloalkyl, alkoxyalkyl, carboxy, alkoxycarbonyl, and carboxamido, or a pharmaceutically acceptable salt or solvate thereof.

118. The compound of embodiment 117, where R^{11b} is selected from the group consisting of —C(═O)OH, —C(═O)OCH₃, —C(═O)N(H)CH₃, —CH₂F, and —CH₂OCH₃, or a pharmaceutically acceptable salt or solvate thereof.

119. The compound of any one of embodiments 1, 79-81 and 107-118, wherein R^{13a} selected from the group consisting of hydrogen and fluoro, or a pharmaceutically acceptable salt or solvate thereof.

120. The compound of any one of embodiments 1, 79-81 and 107-119, wherein R^{13b} selected from the group consisting of hydrogen and fluoro, or a pharmaceutically acceptable salt or solvate thereof.

121. The compound of any one of embodiments 1, 79-81, 107-118 and 120, wherein R^{13a} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10}, or a pharmaceutically acceptable salt or solvate thereof.

122. The compound of embodiment 120, wherein R^{13a} is selected from the group consisting of:

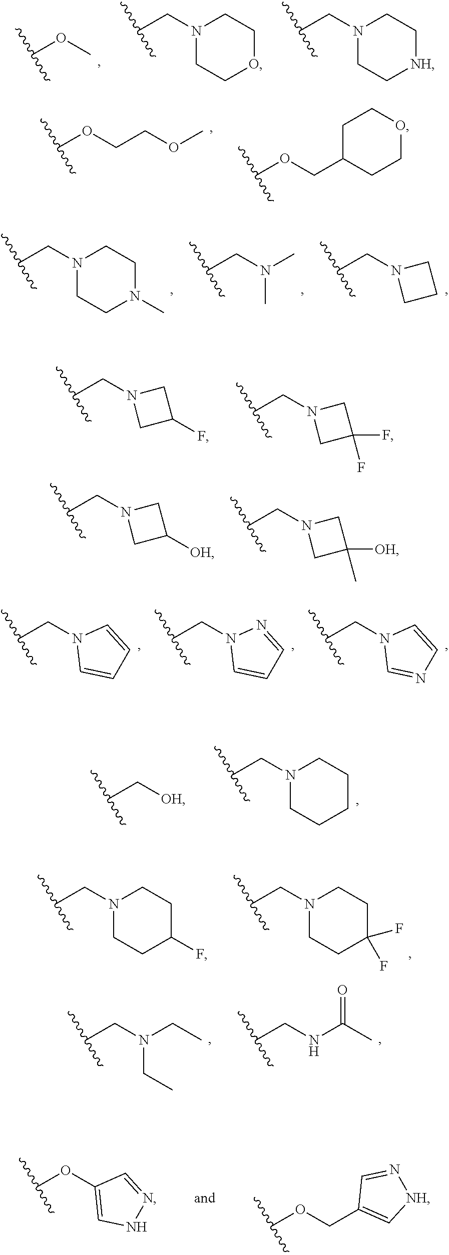

or a pharmaceutically acceptable salt or solvate thereof.

123. The compound of any one of embodiments 1, 79-81, 107-110 and 112-122, wherein:

A is selected from the group consisting of unsubstituted phenyl, phenyl substituted with 1 or 2 substituents independently selected from the group consisting of halo, carboxamido, and —N(H)C(═O)R^{19b}, and 5- or 6-membered heteroaryl substituted with 1 or 2 substituents independently selected from the group consisting of halo and carboxamido; and

R^{19b} is C₁-C₆ alkyl, or a pharmaceutically acceptable salt or solvate thereof.

124. The compound of embodiment 123, wherein A is phenyl substituted with 1 or 2 substituents independently selected from the group consisting of fluoro,

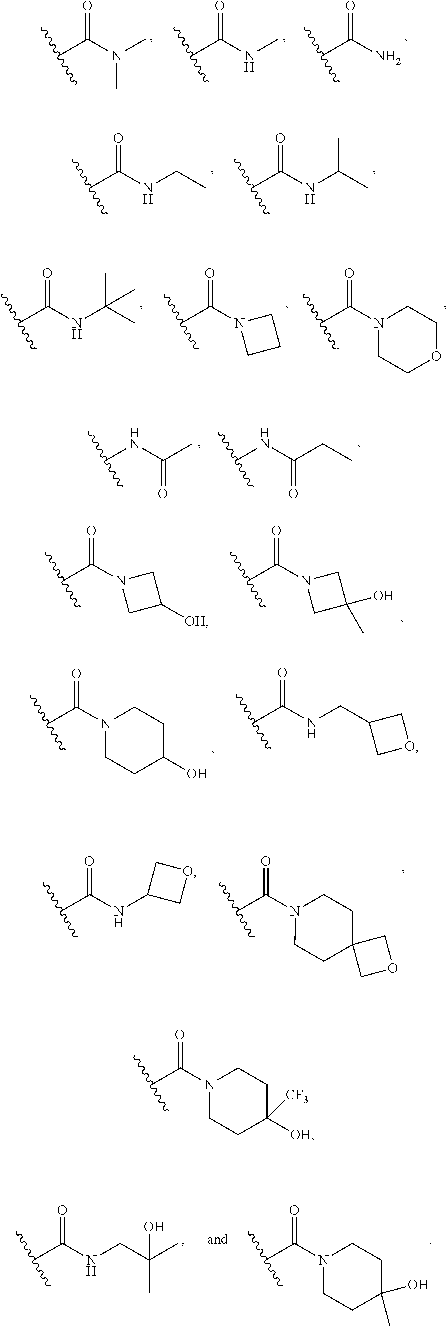

or a pharmaceutically acceptable salt or solvate thereof.

125. The compound of embodiment 123, wherein A is 6-membered heteroaryl substituted with 1 or 2 substituents independently selected from the group consisting of fluoro,

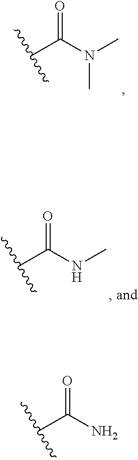

or a pharmaceutically acceptable salt or solvate thereof.

126. The compound of any one of embodiments 1, 79-81, 107-110 and 112-125, wherein:

A is selected from the group consisting of unsubstituted C₃-C₆ cycloalkyl, C₃-C₆ cycloalkyl substituted with 1 or 2 substituents independently selected from the group consisting of halo, hydroxy, C₁-C₄ alkyl, and C₁-C₄ haloalkyl, unsubstituted 4- to 6-membered heterocyclo, and 4- to 6-membered heterocyclo substituted with 1 or 2 substituents independently selected from the group consisting of halo, hydroxy, C₁-C₄ alkyl, and C₁-C₄ haloalkyl, alkylcarbonyl, hydroxyalkylcarbonyl, and alkoxycarbonyl, or a pharmaceutically acceptable salt or solvate thereof.

127. The compound of embodiment 126, wherein A is selected from the group consisting of:

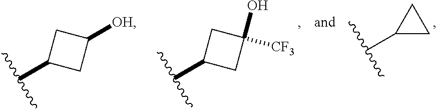

or a pharmaceutically acceptable salt or solvate thereof.

128. The compound of embodiment 126, wherein A is selected from the group consisting of

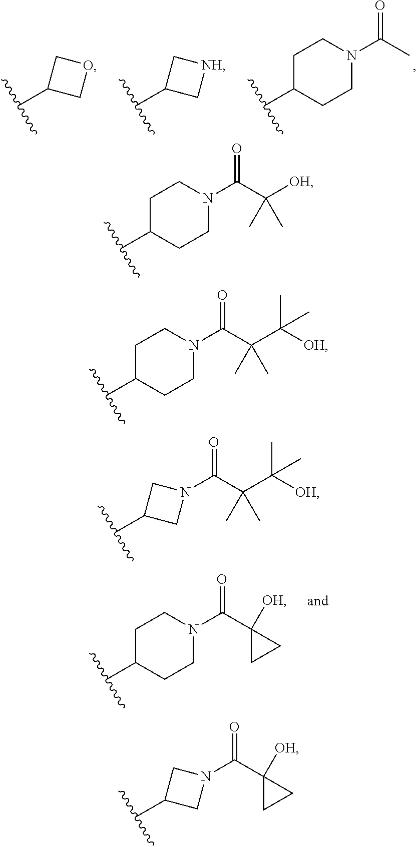

or a pharmaceutically acceptable salt or solvate thereof.

129. The compound of any one of embodiments 1, 79-128, wherein R^{1a} and R^{1b} are independently selected from the group consisting of hydrogen and fluoro, or a pharmaceutically acceptable salt or solvate thereof.

130. The compound of embodiment 129, wherein R^{1a} is fluoro, or a pharmaceutically acceptable salt or solvate thereof.

131. The compound of embodiment 129, wherein R^{1a} and R^{1b} are fluoro, or a pharmaceutically acceptable salt or solvate thereof.

132. The compound of any one of embodiments 1 and 79-106, wherein R^{1c} is hydrogen, or a pharmaceutically acceptable salt or solvate thereof.

133. The compound of embodiment 79 selected from the group consisting of the compounds of Table 1.2, or a pharmaceutically acceptable salt thereof.

134. A pharmaceutical composition comprising the compound of any one of embodiments 79-133, or a pharmaceutically acceptable salt thereof, and a pharmaceutically acceptable carrier.

135. A method of treating a subject in need thereof, the method comprising administering to the subject a therapeutically effective amount of the compound of any one of embodiments 79-133, or a pharmaceutically acceptable salt or solvate thereof, wherein the subject has cancer.

136. The method of embodiment 135, wherein the cancer is any one or more of the cancers of Table 2.

137. The method of embodiment 136, wherein the cancer is a hematological cancer.

138. The method of embodiment 137, wherein the hematological cancer is any one or more of the cancers of Table 3.

139. The method of any one of embodiments 135-138 further comprising administering a therapeutically effective amount of a second therapeutic agent useful in the treatment of cancer.

140. The pharmaceutical composition of embodiment 134 for use in treating cancer.

141. The pharmaceutical composition of embodiment 140, wherein the cancer is any one or more of the cancers of Table 2.

142. The pharmaceutical composition of embodiment 141, wherein the cancer is a hematological cancer.

143. The pharmaceutical composition of embodiment 142, wherein the hematological cancer is any one or more of the cancers of Table 3.

144. A compound of any one of embodiments 79-133, or a pharmaceutically acceptable salt or solvate thereof, for use in treatment of cancer.

145. The compound for use of embodiment 144, wherein the cancer is any one or more of the cancers of Table 2.

146. The compound for use of embodiment 145, wherein the cancer is a hematological cancer.

147. The compound for use of embodiment 146, wherein the hematological cancer is any one or more of the cancers of Table 3.

148. Use of a compound of any one of embodiments 79-133, or a pharmaceutically acceptable salt or solvate thereof, for the manufacture of a medicament for treatment of cancer.

149. The use of embodiment 138, wherein the cancer is any one or more of the cancers of Table 2.

150. The use of embodiment 149, wherein the cancer is a hematological cancer.

151. The use of embodiment 150, wherein the hematological cancer is any one or more of the cancers of Table 3.

152. A kit comprising the compound of any one of embodiments 79-133, or a pharmaceutically acceptable salt or solvate thereof, and instructions for administering the compound, or a pharmaceutically acceptable salt or solvate thereof, to a subject having cancer.

153. The kit of embodiment 152, wherein the cancer is any one or more of the cancers of Table 2.

154. The kit of embodiment 153, wherein the cancer is a hematological cancer.

155. The kit of embodiment 154, wherein the hematological cancer is any one or more of the cancers of Table 3.

156. The kit of any one of embodiments 152-155 further comprising one or more additional therapeutic agents.

157. The compound of embodiment 1, wherein Q is selected from the group consisting of —OR and —OC(═O)R, or a pharmaceutically acceptable salt or solvate thereof.

158. The compound of embodiment 1, wherein Q is —N(H)C(═O)R, or a pharmaceutically acceptable salt or solvate thereof.

159. The compound of embodiment 1, wherein Q is —N(H)C(═O)OR, or a pharmaceutically acceptable salt or solvate thereof.

160. The compound of embodiment 1, wherein Q is selected from the group consisting of —N(R)C(═O)OR, —N(H)C(O)NR₂,

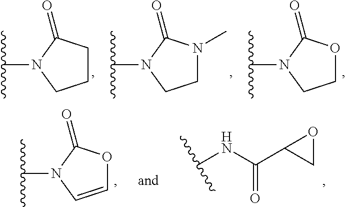

or a pharmaceutically acceptable salt or solvate thereof.

161. The compound of embodiment 1, wherein Q is selected from the group consisting of —N(R)C(═O)OR, —N(H)C(═O)R, —N(H)C(O)NR₂,

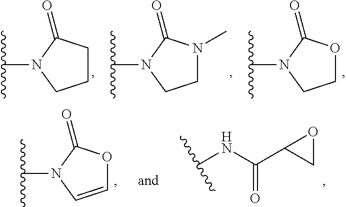

—OR, and —OC(═O)R, or a pharmaceutically acceptable salt or solvate thereof.

162. The compound of any one of embodiments 1 and 157-161, wherein G is selected from the group consisting of G-2, G-3, G-5, G-6, G-7, G-8, G-9, and G-10, or a pharmaceutically acceptable salt or solvate thereof.

163. The compound of any one of embodiments 1 and 157-161, wherein G is selected from the group consisting of G-11 and G-12, or a pharmaceutically acceptable salt or solvate thereof.

164. The compound of any one of embodiments 1 and 157-161, wherein G is selected from the group consisting of G-1 and G-4, or a pharmaceutically acceptable salt or solvate thereof.

165. The compound of any one of embodiments 1 and 157-161, wherein G is selected from the group consisting of G-13, G-14, G-15, G-16, G-17, G-18, G-19, G-20, G-21, G-22, G-23, G-24, G-25, and G-26, or a pharmaceutically acceptable salt or solvate thereof.

166. The compound of any one of embodiments 1 and 157-161, wherein G is selected from the group consisting of G-2, G-3, G-4, G-5, G-6, G-7, G-8, G-10, G-11, G-12, G-13, G-14, G-15, G-16, G-17, G-18, G-19, G-20, G-21, G-22, G-23, G-24, G-25, and G-26, or a pharmaceutically acceptable salt or solvate thereof.

167. The compound of any one of embodiments 1 and 157-161, wherein G is G-4 or is selected from the group consisting of G-2, G-3, G-5, G-6, G-7, G-8, and G-10, or a pharmaceutically acceptable salt or solvate thereof.

168. The compound of any one of embodiments 1 and 157-167, wherein E is selected from the group consisting of E-1 and E-2, or a pharmaceutically acceptable salt or solvate thereof.

169. The compound of any one of embodiments 1 and 157-167, wherein E is E-3, or a pharmaceutically acceptable salt or solvate thereof.

170. The compound of embodiment 169, wherein R^{4a} and R^{4b} are independently selected from the group consisting of halo and C₁-C₄ alkyl, or R, or a pharmaceutically acceptable salt or solvate thereof.

171. The compound of embodiment 169, wherein R^{4a} and R^{4b} are hydrogen, or a pharmaceutically acceptable salt or solvate thereof.

172. The compound of any one of embodiments 1 and 157-171, wherein B is selected from the group consisting of C₁-C₆ alkyl, aralkyl, —C(═O)R⁹, —(CR^{5c}R^{5d})ₘOR¹⁰ and B-1, or a pharmaceutically acceptable salt or solvate thereof.

173. The compound of any one of embodiments 1 and 157-171, wherein B is selected from the group consisting of B-3, B-4, B-5, B-6, B-7, and B-8, or a pharmaceutically acceptable salt or solvate thereof.

174. The compound of any one of embodiments 1 and 157-171, wherein B is B-2, or a pharmaceutically acceptable salt or solvate thereof.

175. The compound of embodiment 174, wherein R^{13a} and R^{13b} are independently selected from the group consisting of halo, C₁-C₄ alkyl, and R^{a5}, or a pharmaceutically acceptable salt or solvate thereof.

176. The compound of any one of embodiments 1 and 157-171, wherein B is selected from the group consisting of B-9, B-10, B-11, B-12, B-13, and B-14, or a pharmaceutically acceptable salt or solvate thereof.

177. The compound of any one of embodiments 1 and 157-171, wherein B is selected from the group consisting of C₁-C₆ alkyl, —C(═O)R⁹, —(CR^{5c}R^{5d})ₘOR¹⁰, B-1, B-3, B-4, B-5, B-6, B-7, B-8, B-9, B-10, B-11, B-12, B-13, and B-14, or a pharmaceutically acceptable salt or solvate thereof.

178. The compound of any one of embodiments 1 and 157-171, wherein B is selected from the group consisting of C₁-C₆ alkyl, —C(═O)R⁹, —(CR^{5c}R^{5d})ₘOR¹⁰, B-1, B-2, B-3, B-4, B-5, B-6, B-7, B-8, B-9, B-10, B-11, B-12, B-13, and B-14, or a pharmaceutically acceptable salt or solvate thereof.

179. The compound of any one of embodiments 1 and 157-178, wherein R^{a5} is carboxamido, or a pharmaceutically acceptable salt or solvate thereof.

180. The compound of any one of embodiments 1 and 157-179, wherein R^{a6} is selected from the group consisting of C₁-C₄ alkoxy and C₁-C₄ hydroxyalkyl, or a pharmaceutically acceptable salt or solvate thereof.

181. The compound of any one of embodiments 1 and 157-180, wherein X is selected from the group consisting of —S(═O)₂— and —C(═O)—; and A is R^{a11}, or a pharmaceutically acceptable salt or solvate thereof.

182. The compound of any one of embodiments 1 and 157-180, wherein:

(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b}, and

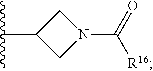

or

(2) X is absent; and A is selected from the group consisting of cyano and —C(═O)OH;

or a pharmaceutically acceptable salt or solvate thereof.

183. The compound of any one of embodiments 1 and 157-180, wherein:

(1) X is —C(═O)—; and A is selected from the group consisting of optionally substituted C₁-C₆ alkyl, optionally substituted C₃-C₁₂ cycloalkyl, optionally substituted 4-to 14-membered heterocyclo, optionally substituted C₆-C₁₀ aryl, optionally substituted 5-to 14-membered heteroaryl, —NR^{15a}R^{15b},

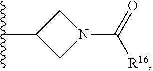

### and R^{a11}; or

(2) X is absent; and A is selected from the group consisting of C(═O)OH, C₁-C₄ alkyl, C₁-C₄ alkoxy, and C₁-C₄ haloalkyl;

or a pharmaceutically acceptable salt or solvate thereof.

184. The compound of any one of embodiments 1 and 157-180, wherein:

(1) X is selected from the group consisting of —S(═O)₂— and —C(═O)—; and A is selected from the group consisting of optionally substituted C₁-C₆ alkyl, 4-membered heterocyclo, optionally substituted 5- to 14-membered heterocyclo, optionally substituted C₆-C₁₀ aryl, optionally substituted 5- to 14-membered heteroaryl, —NR^{15a}R^{15b},

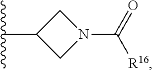

### and R^{a11}; or

(2) X is absent; and A is selected from the group consisting of cyano, C(═O)OH, C₁-C₄ alkyl, C₁-C₄ alkoxy, and C₁-C₄ haloalkyl;

or a pharmaceutically acceptable salt or solvate thereof.

185. The compound of any one of embodiments 1 and 157-180, wherein X is absent; and A is selected from the group consisting of C₁-C₄ alkyl, C₁-C₄ alkoxy, and C₁-C₄ haloalkyl, or a pharmaceutically acceptable salt or solvate thereof.

186. The compound of any one of embodiments 1 and 157-180, wherein X is absent; and A is C₁-C₄ haloalkyl, or a pharmaceutically acceptable salt or solvate thereof.

187. The compound of embodiment 1 selected from the group consisting of the compounds of Table 1.3, or a pharmaceutically acceptable salt thereof.

188. The compound of any one of embodiments 2-21, 32, and 35-53, wherein G is selected from the group consisting of G-2, G-3, G-4, G-5, G-6, G-7, G-8, and G-10, or a pharmaceutically acceptable salt or solvate thereof.

189. The compound of any one of embodiments 79-101 and 115-132, wherein G is selected from the group consisting of G-4, G-11, and G-12, or a pharmaceutically acceptable salt or solvate thereof.

190. A pharmaceutical composition comprising the compound of any one of embodiments 1 and 157-189, or a pharmaceutically acceptable salt thereof, and a pharmaceutically acceptable carrier.

191. A method of treating a subject in need thereof, the method comprising administering to the subject a therapeutically effective amount of the compound of any one of embodiments 1 and 157-189, or a pharmaceutically acceptable salt or solvate thereof, wherein the subject has cancer.

192. The method of embodiment 191, wherein the cancer is any one or more of the cancers of Table 2.

193. The method of embodiment 192, wherein the cancer is a hematological cancer.

194. The method of embodiment 193, wherein the hematological cancer is any one or more of the cancers of Table 3.

195. The method of any one of embodiments 191-194 further comprising administering a therapeutically effective amount of a second therapeutic agent useful in the treatment of cancer.

196. The pharmaceutical composition of embodiment 190 for use in treating cancer.

197. The pharmaceutical composition of embodiment 196, wherein the cancer is any one or more of the cancers of Table 2.

198. The pharmaceutical composition of embodiment 197, wherein the cancer is a hematological cancer.

199. The pharmaceutical composition of embodiment 198, wherein the hematological cancer is any one or more of the cancers of Table 3.

200. A compound of any one of embodiments 1 and 157-189, or a pharmaceutically acceptable salt or solvate thereof, for use in treatment of cancer.

201. The compound for use of embodiment 200, wherein the cancer is any one or more of the cancers of Table 2.

202. The compound for use of embodiment 201, wherein the cancer is a hematological cancer.

203. The compound for use of embodiment 202, wherein the hematological cancer is any one or more of the cancers of Table 3.

204. Use of a compound of any one of embodiments 1 and 157-189, or a pharmaceutically acceptable salt or solvate thereof, for the manufacture of a medicament for treatment of cancer.

205. The use of embodiment 204, wherein the cancer is any one or more of the cancers of Table 2.

206. The use of embodiment 205, wherein the cancer is a hematological cancer.

207. The use of embodiment 206, wherein the hematological cancer is any one or more of the cancers of Table 3.

208. A kit comprising the compound of any one of embodiments 1 and 157-189, or a pharmaceutically acceptable salt or solvate thereof, and instructions for administering the compound, or a pharmaceutically acceptable salt or solvate thereof, to a subject having cancer.

209. The kit of embodiment 208, wherein the cancer is any one or more of the cancers of Table 2.

210. The kit of embodiment 209, wherein the cancer is a hematological cancer.

211. The kit of embodiment 210, wherein the hematological cancer is any one or more of the cancers of Table 3.

212. The kit of any one of embodiments 208-211 further comprising one or more additional therapeutic agents.

### EXAMPLES

### Synthesis of Intermediates

### Synthesis of methyl ((1S,2R)-2-((S)-2-(azetidin-1-yl)-1-(3-fluorophenyl)-1-(1-((3-methoxyazetidin-3-yl)methyl)piperidin-4-yl)ethyl)cyclopentyl)carbamate (S16)

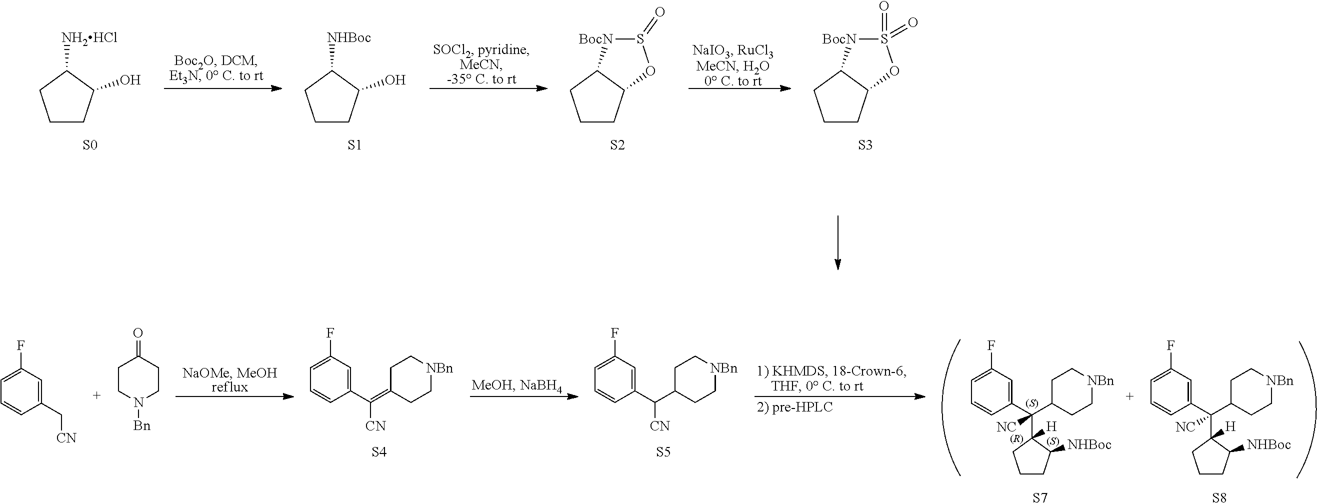

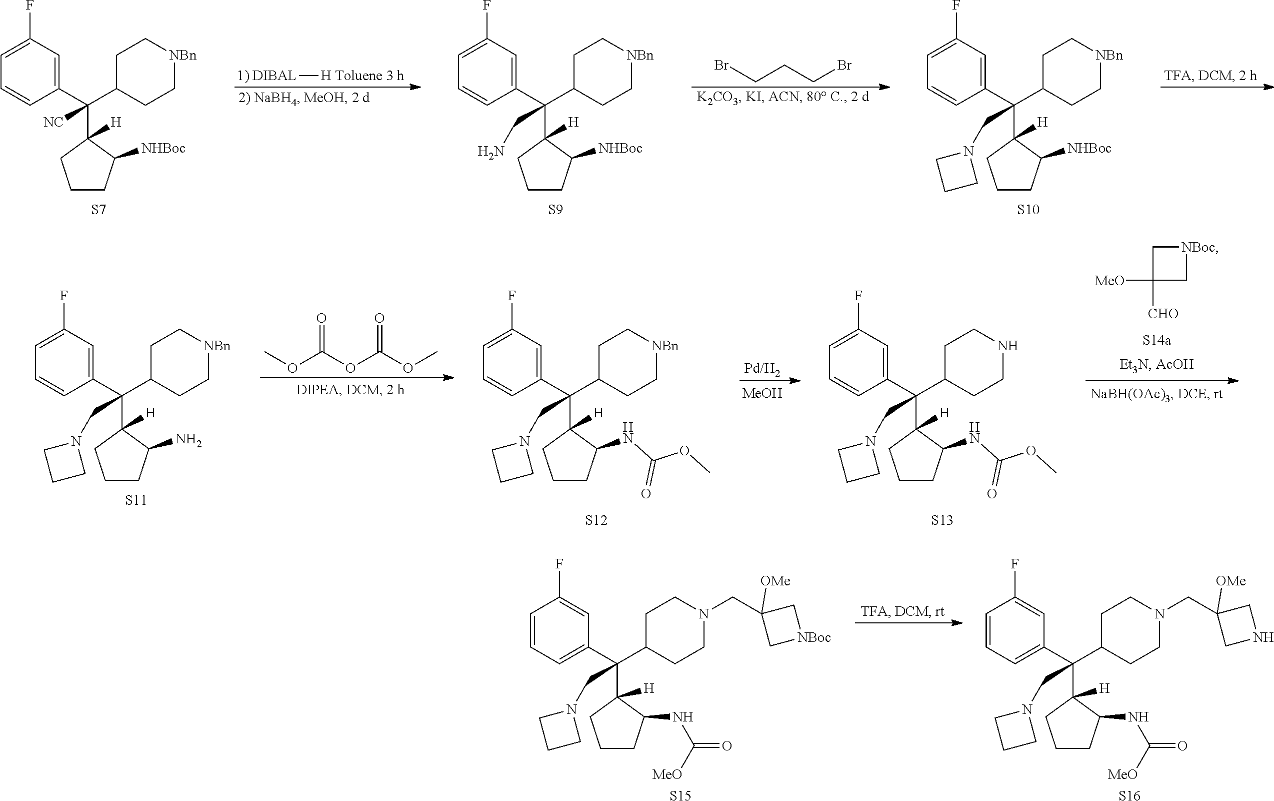

tert-Butyl ((1S,2R)-2-hydroxycyclopentyl)carbamate (S1): To a solution of (1R,2S)-2-aminocyclopentanol hydrochloride S0 (11 g, 79.9 mmol) and B0C₂O (20.9 g, 95.9 mmol) in dichloromethane (200 mL) was added dropwise Et₃N (20.9 mL, 119.9 mmol) at 0° C. The reaction mixture was allowed to warm to room temperature. After stirring overnight, the reaction mixture was washed with saturated brine and the water phase was extracted with dichloromethane twice. The combined organic solvent was dried over Na₂SO₄, filtered, and concentrated under vacuum. The residue was purified by flash column chromatography to give the intermediate S1 as oil (15.5 g, 96%). ¹H NMR (400 MHz, CDCl₃) δ 4.85 (s, 1H), 4.16 (s, 1H), 3.80 (s, 1H), 2.02-1.95 (m, 1H), 1.93-1.87 (m, 1H), 1.86-1.77 (m, 2H), 1.70-1.65 (m, 1H), 1.59-1.51 (m, 2H), 1.45 (s, 9H).

tert-Butyl (3aS,6R)-tetrahydrocyclopenta[d][1,2,3]oxathiazole-3(3aH)-carboxylate 2-oxide (S2): To a solution of thionyl chloride (7 mL, 96.3 mmol) in dry acetonitrile (150 mL) was added a solution of the intermediate S1 (15.5 g, 77.0 mmol) in acetonitrile (150 mL) at −35° C. Then, pyridine (18.7 mL, 231 mmol) was added dropwise and the reaction mixture was allowed to slowly warm to room temperature. After stirring overnight, the reaction mixture was concentrated, and water and ethyl acetate were added. The organic layer was separated and the aqueous layer was extracted three times with ethyl acetate. The combined organic solvent was dried over Na₂SO₄, filtered and concentrated. The residue was purified by column chromatography to produce the intermediate S2 as oil (18.8 g, 98%). 1H NMR (400 MHz, CDCl3) δ 5.74 (t, J=4.6 Hz, 1H), 4.46 (s, 1H), 2.14-2.09 (m, 1H), 1.90-1.68 (m, 5H), 1.52 (s, 9H).

tert-Butyl (3aS,6R)-tetrahydrocyclopenta[d][1,2,3]oxathiazole-3(3aH)-carboxylate 2,2-dioxide (S3): To a solution of the intermediate S2 (18.8 g, 76 mmol) in acetonitrile (100 mL) and H₂O (100 mL) was added NaIO₄ (24.4 g, 114 mmol) in portions, followed by addition of RuCl₃.H₂O (315 mg, 1.5 mmol) at 0° C. The reaction was stirred at room temperature for 2 hours. Then, the aqueous layer was extracted with diethyl ether three time. The combined organic solvent was dried over Na₂SO₄, filtered and concentrated. The residue was purified by column chromatography to produce the title compound S3 as a white solid (19 g, 95%). ¹H NMR (400 MHz, CDCl₃) δ 5.18-5.15 (m, 1H), 4.56-4.53 (m, 1H), 2.23-2.18 (m, 1H), 2.06-1.95 (m, 3H), 1.87-1.77 (m, 2H), 1.55 (s, 9H). ESI-MS calculated for C₁₀H₁₇NO₅S [M+Na]⁺=286.07, found: 286.10.

2-(1-Benzylpiperidin-4-yl)-2-(3-fluorophenyl)acetonitrile (S5): Sodium methoxide (12 mL, 55.52 mmol of 25% wt in methanol) was added to a solution of 2-(3-fluorophenyl)acetonitrile (5 g, 37.01 mmol) in MeOH (50 mL) and stirred briefly. To this solution was added 1-benzylpiperidin-4-one (7.01 g, 37.01 mmol) and reaction was refluxed. After overnight, the solvent was removed, water and EtOAc were added and separated. The aqueous layer was extracted two more times with EtOAc, dried over Na₂SO₄, filtered and concentrated to give S4 that was used without further purification.

Crude S4 (37.01 mmol) was redissolved in MeOH (50 mL) and NaBH₄ (4.2 g, 111.03 mmol) was slowly added. After overnight, the reaction was checked by TLC (if the reaction is not complete more NaBH₄ was added). After complete conversion of S4 to S5, 8 mL of water was added and the reaction was concentrated then more H₂O and EtOAc were added and separated. The aqueous layer was extracted three times with EtOAc, dried over Na₂SO₄, filtered, concentrated, and purified by column chromatography (DCM/EtOAc gradient) to produce S5 as an oil. ¹H NMR (400 MHz, MeOD) δ 7.44-7.38 (m, 1H), 7.32-7.28 (m, 4H), 7.27-7.22 (m, 1H), 7.18-7.16 (m, 1H), 7.13-7.05 (m, 2H), 3.98 (d, J=7.1 Hz, 1H), 3.48 (s, 2H), 2.96-2.87 (m, 2H), 2.00-1.92 (m, 2H), 1.87-1.80 (m, 1H), 1.79-1.72 (m, 1H), 1.59-1.52 (m, 1H), 1.50-1.39 (m, 2H); ESI-MS calculated for C₂₀H₂₁FN₂ [M+H]⁺=309.17, found: 309.16.

tert-Butyl ((1S,2R)-2-((S)-(1-benzylpiperidin-4-yl)(cyano)(3-fluorophenyl)methyl)cyclopentyl)carbamate (S7) and tert-butyl ((1S,2R)-2-((R)-(1-benzylpiperidin-4-yl)(cyano)(3-fluorophenyl)methyl)cyclopentyl)carbamate (S8): Compound S5 (2.18 g, 7.07 mmol), 18-Crown-6 (5.61 g, 21.21 mmol), and compound S3 (5.58 g, 21.21 mmol) were added to a dry round-bottom flask. Then, the flask was covered with a kimwipe and dried in a desiccator under vacuum for 1-2 days. After the drying step, the flask was removed from the desiccator and quickly capped with a septum. The system was vacuumed and protected under nitrogen atmosphere. The contents in the flask were then dissolved completely with 60 mL of freshly distilled THF. The solution was then briefly vacuumed then put under nitrogen atmosphere (This purging was repeated two more times). The reaction was cooled to 0° C., KHMDS (0.5M in toluene, 42.4 mL, 21.21 mmol) was added dropwise and then the reaction was allowed to warm to room temperature and stirred overnight. After overnight, a solution of concentrated H₂SCE (0.6 mL, 11.31 mmol) in H₂O (10 mL) was added (Note: PH of solution should be <7) and the solution was vigorously stirred overnight. Then, the reaction mixture was slowly quenched and basified with saturated NaHCO₃, extracted with ethyl acetate three times. The combined organic solvent was dried over Na₂SO₄, filtered and concentrated. The residue was purified by column chromatograph to give the mixture of diastereomers in a ratio of 3:2 as a yellow solid (2.5 g, 73%). Then, the diastereomers were separated by reverse phase preparative HPLC to give the enantiopure title compounds S7 (1.2 g, 36%) and S8 (0.8 g, 24%) as salts of trifluoroacetic acid, respectively. The enantiopure compound S7 can also be isolated by recrystallization in a solution of hexane and dichloromethane with a ratio of 4:1. Data for S7: ³H NMR (400 MHz, MeOD) δ 7.44-7.39 (m, 1H), 7.35 (d, J=7.9 Hz, 1H), 7.31-7.22 (m, 6H), 7.11-7.06 (m, 1H), 3.82-3.77 (m, 1H), 3.46 (s, 2H), 2.91 (t, J=12.5 Hz, 2H), 2.81-2.76 (m, 1H), 2.07-1.93 (m, 5H), 1.80-1.72 (m, 1H), 1.62-1.46 (m, 5H), 1.33 (s, 9H), 1.27-1.17 (m, 2H); ESI-MS calculated for C₃₀H₃₈FN₃O₂ [M+H]⁺=492.29, found: 492.36. [α]_{D}²⁰=+23.1, (c 1.17×10⁻³ g/mL, MeOH); t_{R} (UPLC)=4.46 min. Data for S8: ³H NMR (400 MHz, MeOD) δ 7.50-7.43 (m, 6H), 7.27 (d, J=7.3 Hz, 1H), 7.20 (d, J=9.9 Hz, 1H), 7.14 (t, J=8.3 Hz, 1H), 4.24 (s, 2H), 4.02-3.98 (m, 1H), 3.54-3.45 (m, 2H), 3.08 (t, J=11.4 Hz, 2H), 2.88-2.83 (m, 2H), 2.59 (t, J=11.8 Hz, 1H), 2.25 (d, J=14.0 Hz, 1H), 1.99-1.87 (m, 2H), 1.79-1.74 (m, 1H), 1.67-1.57 (m, 3H), 1.46 (s, 9H), 1.43-1.37 (m, 2H), 1.33-1.18 (m, 1H); ESI-MS calculated for C₃₀H₃₈FN₃O₂ [M+H]⁺=492.29, found: 492.36. [α]_{D}²⁰=+9.4, (c 1.07×10⁻³ g/mL, MeOH); t_{R} (UPLC)=4.63 min. The absolute stereochemistry of S7 and S8 was determined by single crystal x-ray analysis of S7. See S. Xu et al., “Design of the First-in-Class, Highly Potent Irreversible Inhibitor Targeting the Menin-MLL Protein-Protein Interaction,” 57 Angew. Chem. Int. Ed. 1601-05 (2018).

Synthesis of S9: S7 (3 g, 6.1 mmol) was added to a dry RB-flask then covered with a kimwipe and put in a desiccator that was put under vacuum for 1-2 days. After the vacuuming step, the flask was removed from the desiccator and quickly capped with a septum and the system was vacuumed under N2 atmosphere. The anhydrous toluene (30 ml, Sigma catalog no. 244511) was added to the flask, then was cooled to 0° C. in the ice-bath. Diisobutylaluminiumhydride (25% in toluene, 16.4 mL, 24.4 mmol, 4 eq) was injected into the reaction mixture with syringe slowly at 0° C. with stirring. Then the ice-bath was removed, the reaction was monitored using UPLC-Mass (about 4 h). After the mass (492) of S7 disappeared, 20 ml of NaOH (1M) solution was added slowly into the reaction mixture at 0° C. to quench the reaction. After stirring for 5 min, the ice-bath was removed and additional 20 ml saturated brine was added. Then about 50 mL EA was added, the gel will form. The gel was filtered with celite, and was washed with EA, combine the solvent. The solution was extracted with EA, DCM twice respectively. The organic solvent was dried with Na₂SO₄, filtered, and concentrated under rotatory vacuum. Then DCM (50 ml) was added, and concentrated again (repeat twice to remove EA completely).

Then the residue was redissolved in MeOH (100 mL), NaBH₄ (461 mg, 12.2 mmol, 4 eq) was added slowly at 0° C., the reaction mixture was stirred at room temperature, and the reaction was monitored using UPLC-Mass (about 2 days). NaBH₄ (1 eq) was added every 12 hour if there is still imine intermediate (mass: 495). After the imine intermediate disappear, the reaction mixture was concentrated, and diluted with water. The solution was extracted with EA, DCM twice respectively. The organic solvent was dried with Na₂SO₄, filter, and concentrated under rotatory vacuum to give crude product S9 (mass: 496) without further purification. ¹H NMR (400 MHz, MeOD) δ 7.41-7.35 (m, 1H), 7.33-7.23 (m, 6H), 7.18 (d, J=11.6 Hz, 1H), 6.99-6.95 (m, 1H), 4.07-4.02 (m, 1H), 3.52-3.44 (m, 2H), 3.24 (d, J=14.4 Hz, 1H), 3.09 (d, J=14.4 Hz, 1H), 2.98 (d, J=11.2 Hz, 1H), 2.91 (d, J=10.8 Hz, 1H), 2.35-2.29 (m, 1H), 2.12-2.04 (m, 2H), 2.01-1.94 (m, 2H), 1.77-1.69 (m, 1H), 1.61-1.58 (m, 1H), 1.54-1.47 (m, 2H), 1.44 (s, 9H), 1.41-1.29 (m, 3H), 1.22-1.14 (m, 2H); ESI-MS calculated for C₃OH₄₂FN₃O₂ [M+H]⁺=496.33, found: 496.48.

Synthesis of S10: To a solution of the intermediate S9 (3 g, 6.05 mmol) in acetonitrile (150 mL) was added 1,3-dibromopropane (1.47 g, 0.74 ml, 7.26 mmol, 1.2 eq), K₂CO₃ (2.51 g, 18 mmol, 3 eq) and KI (100 mg, 0.6 mmol, 0.1 eq). The mixture was stirred at 80° C. for 1˜2 days. Then, the mixture was filtered with celite to remove the most of K₂CO₃ solid. The mixture was concentrated, and dissolved in the water, extracted with ethyl acetate and DCM twice respectively, dried over Na₂SO₄, and the solvent was evaporated under vacuum to give crude product S10 without further purification. ¹H NMR (400 MHz, MeOD) δ 7.47-7.40 (m, 6H), 7.16-7.03 (m, 3H), 4.52-4.46 (m, 2H), 4.38-4.31 (m, 1H), 4.19-4.10 (m, 2H), 4.19 (s, 2H), 3.70-3.66 (m, 1H), 3.44-3.40 (m, 3H), 3.01-2.90 (m, 2H), 2.79-2.73 (m, 1H), 2.56-2.46 (m, 1H), 2.42-2.36 (m, 1H), 2.05-1.93 (m, 4H), 1.82-1.73 (m, 2H), 1.68-1.57 (m, 3H), 1.37-1.29 (m, 1H), 1.22 (s, 9H), 1.06-0.98 (m, 1H). ¹H NMR (400 MHz, MeOD) δ; ESI-MS calculated for C₃₃H₄₆FN₃O₂ [M+H]⁺=536.36, found: 536.44.

Synthesis of S11: Compound S10 (2.55 g, 4.76 mmol) was dissolved in dichloromethane (5 mL) and trifluoroacetic acid (10 mL) was added slowly at 0° C. After stirring for 2 h at room temperature, the reaction mixture was concentrated under vacuum, and redissolved in 100 mL of DCM. Amberlyst® A21 (3 g) (resin, Sigma catalog no. 216410) was added and stirred for 30 min to neutralized the TFA. Then, the resin was filtered, and the organic solvent was concentrated to give the crude product S11 without further purification. ESI-MS calculated for C₂₈H₃₈FN₃ [M+H]⁺=436.30, found: 436.32.

Synthesis of S12: S11 (2.07 g, 4.75 mmol) was dissolved in dry dichloromethane (50 mL). Then, DIPEA (3.31 mL, 19 mmol) and dimethyl dicarbonate (764 mg, 5.7 mmol, 1.2 eq) were added at 0° C. After stirring for 2 h at room temperature, the reaction mixture was concentrated under vacuum. The residue was purified by reverse phase preparative HPLC to give the title compound as a salt of trifluoroacetic acid. ¹H NMR (400 MHz, MeOD) δ 7.48-7.40 (m, 6H), 7.14-7.10 (m, 2H), 7.02 (d, J=7.6 Hz, 1H), 4.52-4.47 (m, 2H), 4.38-4.31 (m, 2H), 4.21 (s, 2H), 4.11 (d, J=15.6 Hz, 1H), 3.76 (d, J=15.6 Hz, 1H), 3.46-3.41 (m, 3H), 3.29 (s, 3H), 3.02-2.90 (m, 2H), 2.77-2.71 (m, 1H), 2.55-2.48 (m, 1H), 2.46-2.40 (m, 1H), 2.05-2.02 (m, 2H), 1.99-1.95 (m, 2H), 1.88-1.82 (m, 1H), 1.77-1.73 (m, 1H), 1.69-1.61 (m, 3H), 1.43-1.34 (m, 1H), 1.07-0.97 (m, 1H); ESI-MS calculated for C₃₀H₄₀FN₃O₂ [M+H]⁺=494.31, found: 494.45.

Synthesis of S13: To a solution of the salt of trifluoroacetic acid S12 (1.6 g, 3.24 mmol) in methanol (50 mL) was added 10% Pd/C (344 mg, 0.1 eq, Sigma catalog no. 205699) under N2 atmosphere. Then, the flask was degassed three times with stirring. Then the mixture was stirred for 2 h at room temperature under hydrogen atmosphere (normal pressure). After the Pd/C catalyst was filtered off, the solvent was removed by rotary evaporation to give the title compound. ¹H NMR (400 MHz, MeOD) δ 7.48-7.43 (m, 1H), 7.16-7.06 (m, 3H), 4.51-4.45 (m, 2H), 4.38-4.27 (m, 2H), 4.10 (d, J=15.6 Hz, 1H), 3.77 (d, J=15.2 Hz, 1H), 3.55-3.52 (m, 1H), 3.40-3.33 (m, 2H), 3.31 (s, 3H), 3.01-2.89 (m, 2H), 2.78-2.72 (m, 1H), 2.58-2.48 (m, 1H), 2.46-2.39 (m, 1H), 2.05-1.93 (m, 5H), 1.78-1.70 (m, 1H), 1.68-1.54 (m, 3H), 1.39-1.30 (m, 1H), 1.08-1.02 (m, 1H); ESI-MS calculated for C₂₃H₃₄FN₃O₂ [M+H]⁺=404.26, found: 404.42.

Synthesis of S15: To a solution of S13 (1.40 g, 3.48 mmol) in DCE (30 mL) was added Et₃N (1.2 mL, 8.70 mmol), AcOH (0.8 mL, 13.9 mmol) and S14a (748 mg, 3.48 mmol) subsequently. After 3 h, NaBH(OAc)₃ (2.21 g, 10.4 mmol) was added. The mixture was stirred overnight, quenched with water and concentrated under vacuum. The residue was purified by reverse phase preparative HPLC to give the title compound S15 as a salt of trifluoroacetic acid. ¹H NMR (400 MHz, Methanol-d4) δ 7.37 (td, J=8.4, 6.2 Hz, 1H), 7.10-7.01 (m, 2H), 6.97 (d, J=8.0 Hz, 1H), 4.40 (m, 1H), 4.32-4.15 (m, 1H), 4.14-3.93 (m, 4H), 3.86-3.68 (m, 4H), 3.64 (d, J=8.0 Hz, 2H), 3.38 (m, 4H), 3.28-3.14 (m, 6H), 2.99 (tp, J=23.5, 11.8, 11.2 Hz, 2H), 2.70 (q, J=9.1 Hz, 1H), 2.46 (dq, J=11.5, 9.2 Hz, 1H), 2.34 (m, 1H), 2.00-1.83 (m, 4H), 1.78 (d, J=5.8 Hz, 1H), 1.70 (dt, J=8.8, 4.4 Hz, 1H), 1.64-1.47 (m, 3H), 1.36 (m, 11H). ESI-MS calculated for C₃₃H₅₂FN₄O₅ [M+H]⁺=603.39, found: 603.13.

Synthesis of S16: Compound S15 (2.20 g, 3.48 mmol) was dissolved in DCM (50 mL), then trifluoroacetic acid (5.0 mL, 73.1 mmol) was added. After stirring for 2 hrs at rt, the reaction mixture was evaporated to give the crude title product S16 without further purification.

### Synthesis of tert-butyl 3-formyl-3-methoxyazetidine-1-carboxylate (S14a)

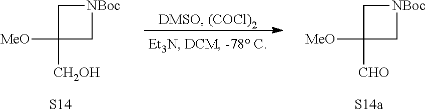

Synthesis of S14a: To a solution of DMSO (0.78 mL, 11.0 mmol) in DCM (30 mL) was added (COCl)2 (2.8 mL, 2M in DCM) under an argon atmosphere at −78° C. After 0.5 h, S14 (800 mg, 3.68 mmol) was added and the mixture was stirred at −78° C. for 2 h. Et₃N (3.1 mL, 22.0 mmol) was then added and the mixture was stirred for another 0.5 h before it was quenched with saturated NH₄C₁ (aq). The solution was extracted with DCM 3 times. The combined organic solvent was washed with brine and dried with Na₂SO₄, filtered, and concentrated under rotatory vacuum to give crude product S14a without further purification.

### Synthesis of tert-butyl 3-ethoxy-3-(((methylsulfonyl)oxy)methyl)azetidine-1-carboxylate (A4)

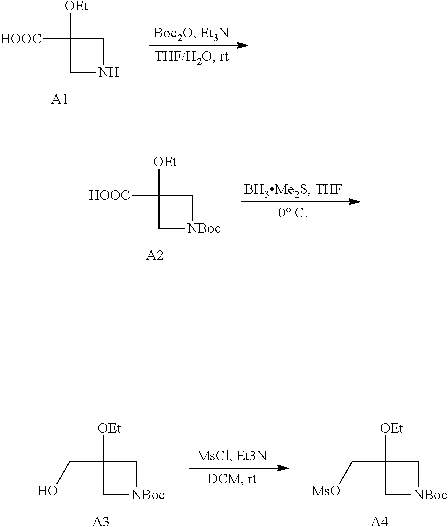

Synthesis of 1-(tert-butoxycarbonyl)-3-ethoxyazetidine-3-carboxylic acid (A2): A1 (1.00 g, 5.51 mmol) was dissolved in THF/H₂O (10 mL/10 mL). Then, Et₃N (1.70 mL, 12.1 mmol) and Di-tert-butyl dicarbonate (1.44 g, 6.61 mmol) were added. After stirring for 12 h at room temperature, 1M Hydrochloric acid (aq) was added. The mixture was extracted with ethyl acetate three times and dried over Na₂SO₄. The solvent was evaporated under vacuum to give crude product A2 without further purification.

Synthesis of tert-butyl 3-ethoxy-3-(hydroxymethyl)azetidine-1-carboxylate (A3): To a solution of A2 in THF (50 mL) was added dropwise BH₃.Me₂S (5.5 mL, 11.0 mmol) at 0° C. After stirring for 4 h, Methanol was added to quench the reaction. The organic solvent was evaporated under vacuum to give crude product A3 without further purification.

Synthesis of tert-butyl 3-ethoxy-3-(((methylsulfonyl)oxy)methyl)azetidine-1-carboxylate (A4): A3 was dissolved in dichloromethane (50 mL). Then, Et₃N (3.1 mL, 22.0 mmol) and Methanesulfonyl chloride (0.46 mL, 6.1 mmol) were added at 0° C. After stirring for 2 h at room temperature, the reaction mixture was concentrated under vacuum. The residue was purified by flash column chromatography to give A4 (533 mg) as colorless oil. ¹H NMR (400 MHz, Chloroform-7) δ 4.39 (s, 2H), 3.95 (d, J=9.5 Hz, 2H), 3.84-3.77 (m, 2H), 3.52 (q, J=7.0 Hz, 2H), 3.07 (s, 3H), 1.44 (s, 9H), 1.23 (t, 7=7.0 Hz, 3H).

### Synthesis of methyl ((1S,2R)-2-((S)-1-(1-benzylpiperidin-4-yl)-1-(3-fluorophenyl)-2-(3-hydroxyazetidin-1-yl)ethyl)cyclopentyl)carbamate (D-4), methyl ((1S,2R)-2-((S)-1-(1-benzylpiperidin-4-yl)-1-(3-fluorophenyl)-2-(3-methoxyazetidin-1-yl)ethyl)cyclopentyl)carbamate (D-6), and ((1S,2R)-2-((S)-1-(1-benzylpiperidin-4-yl)-2-(3-fluoroazetidin-1-yl)-1-(3-fluorophenyl)ethyl)cyclopentyl)carbamate (D-7)

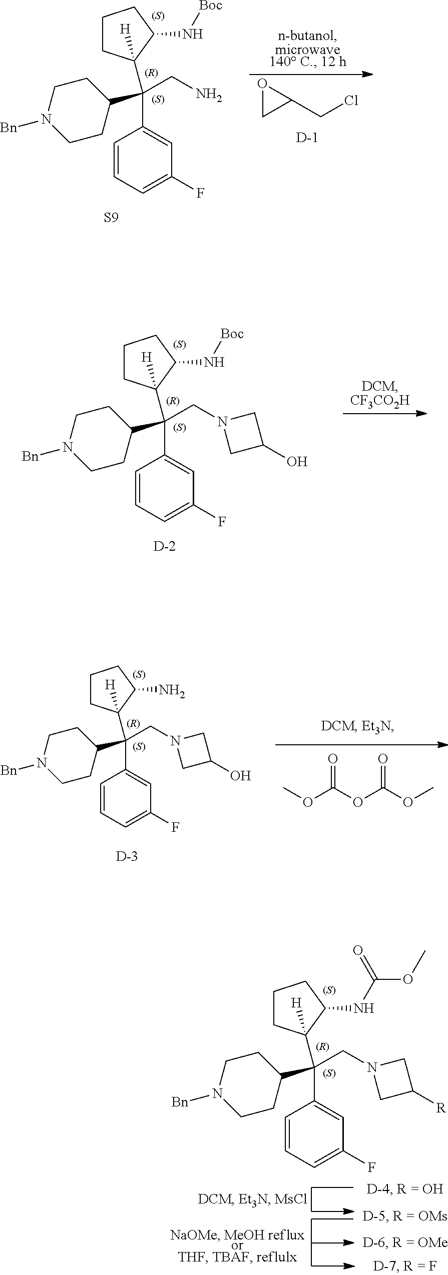

1-((S)-2-((1R,2S)-2-aminocyclopentyl)-2-(1-benzylpiperidin-4-yl)-2-(3-fluorophenyl)ethyl)azetidin-3-ol (D-3): In a microwave reaction tube, compound S9 (400 mg, 0.808 mmol) and epoxide D-1 (63 uL, 0.808 mmol) were dissolved in n-butanol (8 mL) and the reaction was microwaved at 140° C. for 20 hours. After cooling, the reaction was diluted with MeOH/H₂O (1:1), acidified with trifluoroacetic acid and purified by prep-HPLC to produce D-2. ESI-MS calculated for C₃₃H₄₇FN₃O₃ [M+H]⁺=552.35, found: 552.51. Compound D-2 was dissolved in DCM (1 mL) then CF₃CO₂H (3 mL) was added. After 5 minutes the reaction was complete and the solvent was removed by rotovap to produce D-3 (271 mg). ESI-MS calculated for C₂₈H₃₉FN₃O [M+H]⁺=452.30, found: 452.49.

methyl ((1S,2R)-2-((S)-1-(1-benzylpiperidin-4-yl)-1-(3-fluorophenyl)-2-(3-hydroxyazetidin-1-yl)ethyl)cyclopentyl)carbamate (D-4): At 0° C., dimethyl dicarbonate (97 mg, 0.720 mmol) was added to a solution of D-3 (271 mg, 0.600 mmol) and Et₃N (333 uL, 2.4 mmol) in DCM (11 mL). After 2 hours, the reaction was concentrated and purified by prep-HPLC to produce D-4 (205 mg). ESI-MS calculated for C₃₀H₄₁FN₃O₃ [M+H]⁺=510.31, found: 510.49.

methyl ((1S,2R)-2-((S)-1-(1-benzylpiperidin-4-yl)-1-(3-fluorophenyl)-2-(3-methoxyazetidin-1-yl)ethyl)cyclopentyl)carbamate (D-6): At 0° C., methanesulfonyl chloride (23 uL, 0.294 mmol) was added to a solution of D-4 (30 mg, 0.059 mmol) and Et₃N (33 uL, 0.235 mmol) in DCM (2 mL) then the reaction was allowed to warm to room temperature. After 3 hours, the reaction was quenched with saturated NaHCO₃ (2 mL), stirred for 10 minutes then the biphasic mixture was extracted 3 times with DCM. The combined organic layers were dried over sodium sulfate, filtered and concentrate to produce crude D-5. Sodium methoxide (1 mL, 1.0M in methanol) was added to a solution of crude D-5 in methanol (1 mL) and the reaction refluxed. After 1 hour the reaction was cooled, solvent removed and purified by prep-HPLC to produce D-6 (22 mg). ESI-MS calculated for C₃₁H₄₃FN₃O₃ [M+H]⁺=524.32, found: 524.50.

((1S,2R)-2-((S)-1-(1-benzylpiperidin-4-yl)-2-(3-fluoroazetidin-1-yl)-1-(3-fluorophenyl)ethyl)cyclopentyl)carbamate (D-7): Tetrabutylammonium fluoride (0.5 mL, 1.0M in THF) was added to a solution of crude D-5 in THF (2 mL) and the reaction refluxed. After 1 hour the reaction was cooled, then the solvent was removed and the crude purified by prep-HPLC to produce D-7 (18 mg). ESI-MS calculated for C₃₀H₄₀F₂N₃O₂ [M+H]⁺=512.30, found: 512.49.

### Synthesis of 4-fluoro-2-((3-fluoroazetidin-1-yl)methyl)benzonitrile (L7)

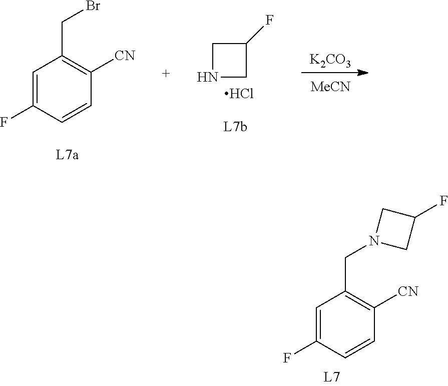

To a solution of 2-(bromomethyl)-4-fluorobenzonitrile L7a (500 mg, 2.3 mmoL, 1.0 eq) and 3-fluoroazetidine hydrochloride L7b (312 mg, 2.8 mmoL, 1.2 eq) in 10 mL of acetonitrile was added potassium carbonate (646 mg, 4.6 mmoL, 2 eq). After stirring for 2 h at room temperature, the reaction mixture was evaporated and purified using normal phase column (Hexane/Ethyl Acetate, 5/1) to give the intermediate 4-fluoro-2-((3-fluoroazetidin-1-yl)methyl)benzonitrile L7. ESI-MS [M+H]+=209.24.

### Example 1

### Synthesis of Methyl ((1S,2R)-2-(cyano(4-(2-(4-cyanophenoxy)ethoxy)phenyl)(3-fluorophenyl)methyl)cyclopentyl)carbamate (Cpd. No. 6)

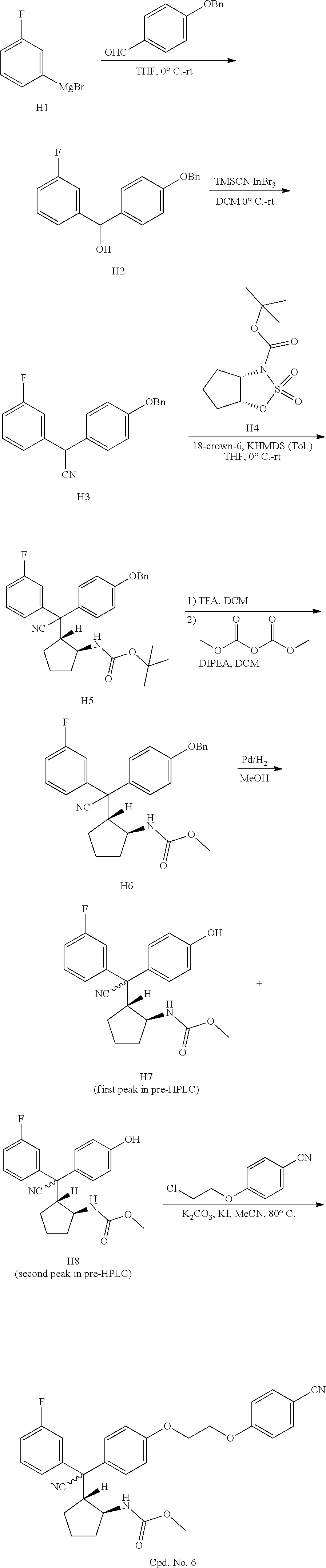

### Synthesis of (4-(benzyloxy)phenyl)(3-fluorophenyl)methanol (H2)

To a solution of 4-(benzyloxy)benzaldehyde (4 g, 18.85 mmol) in THF (50 mL) was added slowly (3-fluorophenyl)magnesium bromide (22.62 mL, 22.62 mmoL, 1M) at 0° C. under nitrogen atmosphere. Then, the reaction mixture was warmed to room temperature slowly and stirred overnight. After the completion of the reaction, the reaction mixture was quenched with saturated aqueous NH₄Cl, concentrated, extracted with ethyl acetate three times, washed with brine, dried over Na₂SO₄, and the solvent was evaporated under vacuum. The residue was purified by flash column chromatography to give the title compound (4.8 g, 83%).

### Synthesis of 2-(4-(benzyloxy)phenyl)-2-(3-fluorophenyl)acetonitrile (H3)

To a suspension of trimethylsilyl cyanide (1.62 mL, 12.97 mmoL), InBr₃ (230 mg, 0.648 mmol) in dichloromethane (13 mL) was added dropwise a solution of the intermediate H2 (2 g, 6.5 mmoL) at 0° C. under nitrogen atmosphere. Then, the reaction mixture was warmed to room temperature slowly and stirred for 1 h. After the completion of reaction, the reaction mixture was concentrated, extracted with ethyl acetate three times, washed with brine, dried over Na₂SO₄, and the solvent was evaporated under vacuum. The residue was purified by flash column chromatography to give the title compound (1.5 g, 73%).

### Synthesis of tert-butyl ((1S,2R)-2-((4-(benzyloxy)phenyl)(cyano)(3-fluorophenyl)methyl)cyclopentyl)carbamate (H5)

Compound H₃ (0.5 g, 1.58 mmol), 18-crown-6 (1.25 g, 4.73 mmol), and tert-butyl (3aS,6aR)-tetrahydrocyclopenta[d][1,2,3]oxathiazole-3(3aH)-carboxylate 2,2-dioxide (H₄) (1.24 g, 4.73 mmol) were added to a dry round-bottom flask. Then, the flask was covered with a kimwipe and dried in a desiccator under vacuum for 1-2 days. After the drying step, the flask was removed from the desiccator and quickly capped with a septum. The system was vacuumed and protected under nitrogen atmosphere. The contents in the flask were then dissolved completely with 20 mL of freshly distilled THF. The solution was then briefly vacuumed then put under nitrogen atmosphere (This purging was repeated two more times). The reaction was cooled to 0° C., KHMDS (0.5M in toluene, 9.45 mL, 4.73 mmol) was added dropwise and then the reaction was allowed to warm to room temperature and stirred overnight. After stirring overnight, a solution of concentrated H₂SO₄ (0.125 mL, 2.36 mmol) in H₂O (5 mL) was added (Note: PH of solution should be <7) and the solution was vigorously stirred overnight. Then, the reaction mixture was slowly quenched and basified with saturated NaHCO₃, extracted with ethyl acetate three times. The combined organic solvent was dried over Na₂SO₄, filtered and concentrated. The residue was purified by column chromatography to give the mixture of diastereomers (0.72 g, 91%).

### Synthesis of methyl ((1S,2R)-2-((4-(benzyloxy)phenyl)(cyano)(3-fluorophenyl)methyl)cyclopentyl)carbamate (H6)

Compound H5 (0.718 g, 1.43 mmoL) was dissolved in dichloromethane (5 mL) and trifluoroacetic acid (5 mL) was added at 0° C. After stirring for 20 min at room temperature, the reaction mixture was concentrated under vacuum, basified with saturated NaHCO₃, and extracted with dichloromethane three times. The combined organic layers were dried over Na₂SO₄, filtered and concentrated under vacuum. The resulting residue was redissolved in dry dichloromethane (10 mL). Then, Et₃N (0.45 mL, 3.25 mmol) and dimethyl dicarbonate (261 mg, 1.95 mmol) were added at 0° C. After stirring for 2 h at room temperature, the reaction mixture was concentrated under vacuum. The residue was purified by column chromatography to give the title compound (0.54 g, 73%).

### Synthesis of methyl ((1S,2R)-2-(cyano(3-fluorophenyl)(4-hydroxyphenyl)methyl)cyclopentyl)carbamate (H7 and H8)

To a solution of the salt of trifluoroacetic acid H6 (0.54 g, 1.18 mmol) in methanol (20 mL) was added 10% Pd/C (126 mg). The mixture was stirred for 4 h at room temperature under hydrogen atmosphere (normal pressure). After the Pd/C catalyst was filtered off, the solvent was removed by rotary evaporation to give the crude diastereomers. Then, the diastereomers were separated by reverse phase preparative HPLC to give the enantiopure title compounds H7 (130 mg, 23%, first peak in pre-HPLC) and H8 (190 mg, 33%, second peak in pre-HPLC) as salts of trifluoroacetic acid, respectively.

### Synthesis of methyl ((1S,2R)-2-(cyano(4-(2-(4-cyanophenoxy)ethoxy)phenyl)(3-fluorophenyl)methyl)cyclopentyl)carbamate (Cpd. No. 6)

To a solution of the intermediate H7 (10 mg, 0.027 mmol) in acetonitrile (1 mL) was added 4-(2-chloroethoxy)benzonitrile (6 mg, 0.032 mmol), K₂CO₃ (7.5 mg, 0.054 mmol) and KI (0.45 mg, 0.027 mmol). The mixture was stirred at 80° C. overnight. Then, the mixture was extracted with dichloromethane, washed with brine, dried over Na₂SO₄, and the solvent was evaporated under vacuum. The residue was purified by reverse phase preparative HPLC to give the trifluoroacetic acid salt of Cpd. No. 6 (5 mg, 36%).

### Example 2

### Synthesis of methyl ((1S,2R)-2-((S)-2-(azetidin-1-yl)-1-(1-((1-(4-(cyclopropylsulfonyl)phenyl)-3-fluoroazetidin-3-yl)methyl)piperidin-4-yl)-1-(3-fluorophenyl)ethyl)cyclopentyl)carbamate (Cpd. No. 41)

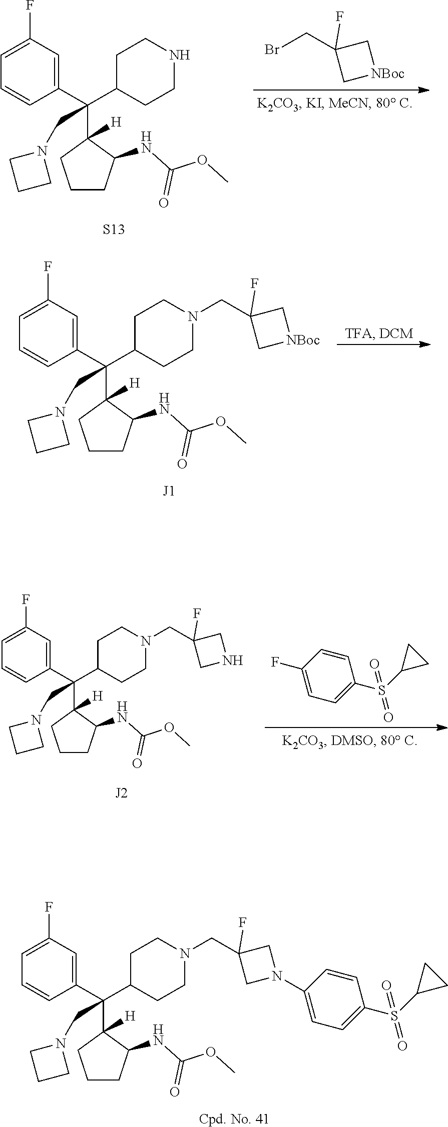

### Synthesis of tert-butyl 3-((4-((S)-2-(azetidin-1-yl)-1-(3-fluorophenyl)-1-((1R,2S)-2-((methoxycarbonyl)amino)cyclopentyl)ethyl)piperidin-1-yl)methyl)-3-fluoroazetidine-1-carboxylate (J1)

To a solution of the intermediate S13 (160 mg, 0.397 mmol) in acetonitrile (5 mL) was tert-butyl 3-(bromomethyl)-3-fluoroazetidine-1-carboxylate (117 mg, 0.436 mmol), K₂CO₃ (110 mg, 0.793 mmol) and KI (6.6 mg, 0.04 mmol). The mixture was stirred at 80° C. overnight. Then, the mixture was extracted with dichloromethane, washed with brine, dried over Na₂SO₄, and the solvent was evaporated under vacuum. The residue was purified by flash column chromatography to give the title compound (200 mg, 85%).

### Synthesis of tert-butyl 3-((4-((S)-2-(azetidin-1-yl)-1-(3-fluorophenyl)-1-((1R,2S)-2-((methoxycarbonyl)amino)cyclopentyl)ethyl)piperidin-1-yl)methyl)-3-fluoroazetidine-1-carboxylate (J2)

Compound J1 (200 mg, 0.34 mmoL) was dissolved in dichloromethane (5 mL) and trifluoroacetic acid (5 mL) was added at 0° C. After stirring for 20 min at room temperature, the reaction mixture was concentrated under vacuum, basified with saturated NaHCO₃, extracted with dichloromethane three times. The combined organic layers were dried over Na₂SO₄, filtered and concentrated under vacuum to give the title compound (120 mg, 72%).

### Synthesis of methyl ((1S,2R)-2-((S)-2-(azetidin-1-yl)-1-(1-((1-(4-(cyclopropylsulfonyl)phenyl)-3-fluoroazetidin-3-yl)methyl)piperidin-4-yl)-1-(3-fluorophenyl)ethyl)cyclopentyl)carbamate (Cpd. No. 41)

To a solution of the intermediate J2 (20 mg, 0.040 mmol) in DMSO (1 mL) was 1-(cyclopropylsulfonyl)-4-fluorobenzene (10 mg, 0.049 mmol), and K₂CO₃ (17 mg, 0.122 mmol). The mixture was stirred at 80° C. overnight. Then, the mixture was extracted with dichloromethane, washed with brine, dried over Na₂SO₄, and the solvent was evaporated under vacuum. The residue was purified by reverse phase preparative HPLC to give the trifluoroacetic acid salt of Cpd. No. 41 (15 mg, 47%).

### Example 3

### Synthesis of methyl ((1S,2R)-2-((S)-2-(azetidin-1-yl)-1-(1-((1-(4-cyanophenyl)azetidin-3-yl)methyl)piperidin-4-yl)-1-(3-fluorophenyl)ethyl)cyclopentyl)carbamate (Cpd. No. 42)

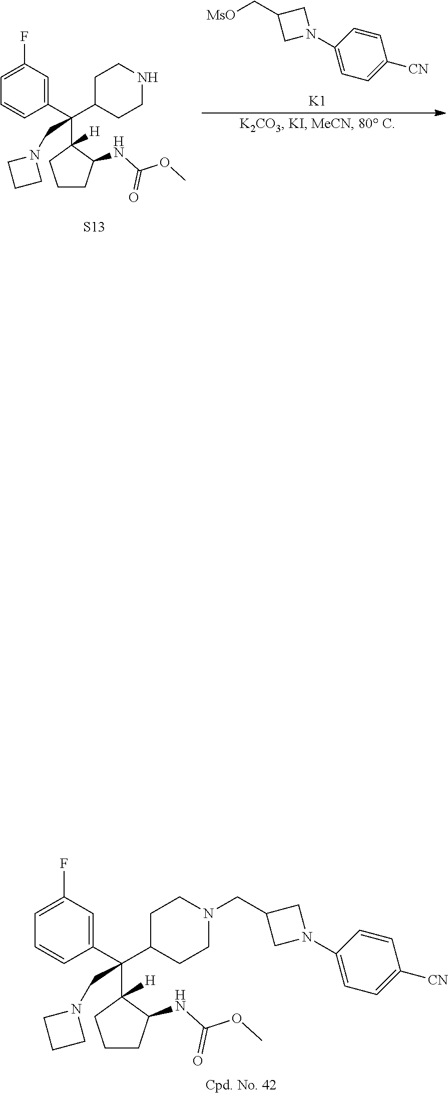

To a solution of the intermediate S13 (300 mg, 0.743 mmol) in acetonitrile (10 mL) was added (1-(4-cyanophenyl)azetidin-3-yl)methyl methanesulfonate (K1) (238 mg, 0.892 mmol), K₂CO₃ (206 mg, 1.49 mmol) and KI (12 mg, 0.074 mmol). The mixture was stirred at 80° C. overnight. Then, the mixture was extracted with dichloromethane, washed with brine, dried over Na₂SO₄, and the solvent was evaporated under vacuum. The residue was purified by reverse phase preparative HPLC to give the trifluoroacetic acid salt of Cpd. No. 42 (350 mg, 69%). ¹H NMR (400 MHz, MeOD) δ 7.48-7.43 (m, 2H), 7.15-7.12 (m, 2H), 7.05 (d, J=7.6 Hz, 1H), 6.46-6.43 (m, 2H), 4.53-4.47 (m, 2H), 4.39-4.32 (m, 2H), 4.12 (t, J=8.0 Hz, 3H), 3.78 (d, J=16.0 Hz, 1H), 3.76-3.68 (m, 2H), 3.55-3.48 (m, 3H), 3.39 (d, J=7.2 Hz, 2H), 3.31 (s, 3H), 3.25-3.17 (m, 1H), 3.03-2.91 (m, 2H), 2.81-2.74 (m, 1H), 2.56-2.49 (m, 1H), 2.47-2.39 (m, 1H), 2.08-1.87 (m, 5H), 1.78-1.76 (m 1H), 1.70-1.62 (m, 3H), 1.51-1.41 (m, 1H), 1.17-1.06 (m, 1H). ¹³C NMR (100 MHz, MeOD) δ 165.16, 162.72, 162.43, 162.26, 162.08, 161.90, 159.82, 154.82, 139.80, 134.39, 131.25, 131.17, 125.57, 121.22, 119.28, 119.18, 117.15, 116.92, 116.39, 116.29, 115.75, 115.54, 111.97, 99.58, 62.00, 60.88, 60.32, 56.15, 56.10, 54.80, 53.98, 52.95, 51.05, 41.27, 33.73, 26.88, 26.64, 26.37, 25.94, 21.25, 17.04.

### Example 4

### Synthesis of methyl ((1S,2R)-2-((S)-2-acetamido-1-(1-((1-(4-cyano-3-((3-fluoroazetidin-1-yl)methyl)phenyl)azetidin-3-yl)methyl)piperidin-4-yl)-1-(3-fluorophenyl)ethyl)cyclopentyl)carbamate (Cpd. No. 176)

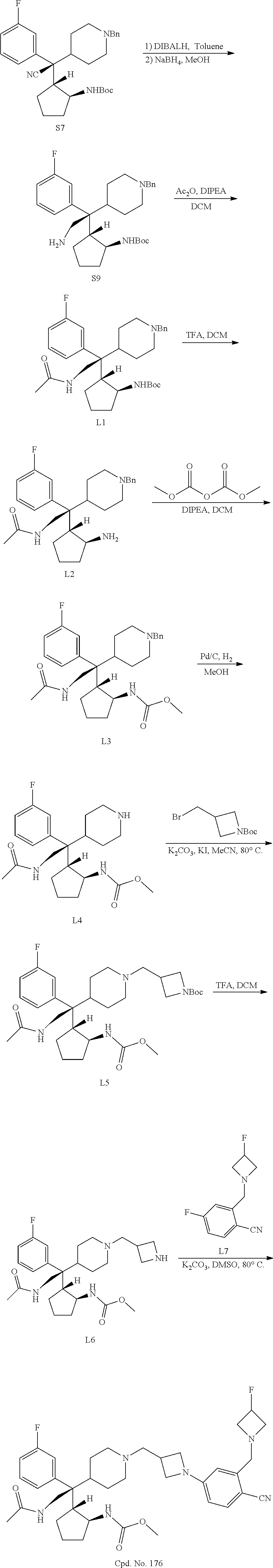

### Synthesis of tert-butyl ((1S,2R)-2-((S)-2-amino-1-(1-benzylpiperidin-4-yl)-1-(3-fluorophenyl)ethyl)cyclopentyl)carbamate (S9)

Intermediate S7 (3 g, 6.1 mmol) was added to a dry round bottom flask then covered with a kimwipe and put in a desiccator that was put under vacuum for 1-2 days. After the vacuuming step, the flask was removed from the desiccator and quickly capped with a septum and the system was vacuumed under N2 atmosphere. Anhydrous toluene (30 ml) was added to the flask, and it was cooled to 0° C. in the ice-bath. Diisobutylaluminiumhydride (25% in toluene, 16.4 mL, 24.4 mmol) was injected into the reaction mixture with syringe slowly at 0° C. with stirring. Then the ice-bath was removed, the reaction was monitored using UPLC-Mass (about 4 h). After the intermediate S7 was consumed, 20 ml of NaOH (1M) solution was added slowly into the reaction mixture at 0° C. to quench the reaction. After stirring for 5 min, the ice-bath was removed and additional 20 ml saturated brine was added. Then about 50 mL ethyl acetate was added, the solid in solution was filtered with celite, and was washed with ethyl acetate (EA). The solution was extracted with EA and DCM twice, respectively. The combined organic solvent was dried with Na₂SO₄, filtered, and concentrated under rotatory vacuum. Then the residue was redissolved in MeOH (100 mL), and NaBH₄ (461 mg, 12.2 mmol) was added slowly at 0° C. The reaction mixture was stirred at room temperature for 2 days. Then, the reaction mixture was concentrated, and diluted with water. The solution was extracted with EA and DCM twice, respectively. The combined organic solvent was dried with Na₂SO₄, filtered, and concentrated under rotatory vacuum to give crude title product S9 (2.8 g, 93%) without further purification. ¹H NMR (400 MHz, MeOD) δ 7.41-7.35 (m, 1H), 7.33-7.23 (m, 6H), 7.18 (d, J=11.6 Hz, 1H), 6.99-6.95 (m, 1H), 4.07-4.02 (m, 1H), 3.52-3.44 (m, 2H), 3.24 (d, J=14.4 Hz, 1H), 3.09 (d, J=14.4 Hz, 1H), 2.98 (d, 7=11.2 Hz, 1H), 2.91 (d, J=10.8 Hz, 1H), 2.35-2.29 (m, 1H), 2.12-2.04 (m, 2H), 2.01-1.94 (m, 2H), 1.77-1.69 (m, 1H), 1.61-1.58 (m, 1H), 1.54-1.47 (m, 2H), 1.44 (s, 9H), 1.41-1.29 (m, 3H), 1.22-1.14 (m, 2H); ESI-MS calculated for C₃₀H₄₂FN₃O₂ [M+H]⁺=496.33, found: 496.48.

### Synthesis of tert-butyl ((1S,2R)-2-((S)-2-acetamido-1-(1-benzylpiperidin-4-yl)-1-(3-fluorophenyl)ethyl)cyclopentyl)carbamate (L1)

Compound S9 (0.5 g, 1.01 mmol) was dissolved in dry dichloromethane (50 mL). Then, DIPEA (0.35 mL, 2.02 mmol) and acetic anhydride (0.11 mL, 1.21 mmol) were added at 0° C. After stirring for 2 h at room temperature, the reaction mixture was concentrated under vacuum. The residue was purified by flash chromatography column to give the title product (0.41 g, 76%). ESI-MS calculated for C₃₂H₄₄FN₃O₃ [M+H]⁺=538.34, found: 538.19.

### Synthesis of N—((S)-2-((1R,2S)-2-aminocyclopentyl)-2-(1-benzylpiperidin-4-yl)-2-(3-fluorophenyl)ethyl)acetamide (L2)

Intermediate L1 (410 mg, 0.762 mmol) was dissolved in dichloromethane (5 mL) and trifluoroacetic acid (5 mL) was added at 0° C. After stirring for 2 h at room temperature, the reaction mixture was concentrated under vacuum to give the trifluoroacetic acid salt of L2 (400 mg, 95%) without further purification. ESI-MS calculated for C₂₇H₃₆FN₃O [M+H]⁺=438.28, found: 438.50.

### Synthesis of methyl ((1S,2R)-2-((S)-2-acetamido-1-(1-benzylpiperidin-4-yl)-1-(3-fluorophenyl)ethyl)cyclopentyl)carbamate (L3)

Trifluoroacetic acid salt L2 (400 mg, 0.725 mmol) was dissolved in dry dichloromethane (50 mL). Then, DIPEA (0.25 mL, 1.45 mmol) and dimethyl dicarbonate (149 mg, 1.09 mmol) were added at 0° C. After stirring for 2 h at room temperature, the reaction mixture was concentrated under vacuum. The residue was purified by reverse phase preparative HPLC to give the title product (350 mg, 79%) as a salt of trifluoroacetic acid. ESI-MS calculated for C₂₉H₃₈FN₃O₃ [M+H]⁺=496.29, found: 496.44.

### Synthesis of methyl ((1S,2R)-2-((S)-2-acetamido-1-(3-fluorophenyl)-1-(piperidin-4-yl)ethyl)cyclopentyl)carbamate (L4)

To a solution of the salt of trifluoroacetic acid L3 (350 mg, 0.57 mmol) in methanol (50 mL) was added 10% Pd/C (61 mg, 10% wt.) under N2 atmosphere. Then, the flask was degassed three times with stirring. Then the mixture was stirred for 1 h at room temperature under hydrogen atmosphere (normal pressure). After the Pd/C catalyst was filtered off, the solvent was removed by rotary evaporation to give the title product (200 mg, 86%). ESI-MS calculated for C₂₂H₃₂FN₃O₃ [M+H]⁺=406.24, found: 406.47.

### Synthesis of methyl ((1S,2R)-2-((S)-2-acetamido-1-(1-(azetidin-3-ylmethyl)piperidin-4-yl)-1-(3-fluorophenyl)ethyl)cyclopentyl)carbamate (L6)

To a solution of the intermediate L4 (200 mg, 0.493 mmol) in acetonitrile (1 mL) was added compound tert-butyl 3-(bromomethyl)azetidine-1-carboxylate (148 mg, 0.592 mmol), K₂CO₃ (136 mg, 0.986 mmol) and KI (8 mg, 0.049 mmol). The mixture was stirred at 80° C. overnight. Then, the mixture was extracted with dichloromethane, washed with brine, dried over Na₂SO₄, and the solvent was evaporated under vacuum to obtain the crude intermediate L5, which was dissolved in dichloromethane (5 mL) and trifluoroacetic acid (5 mL) was added at 0° C. After stirring for 2 h at room temperature, the reaction mixture was concentrated under vacuum and was purified by reverse phase preparative HPLC to give the trifluoroacetic acid salt of L6 (210 mg, 72%). ESI-MS calculated for C₂₆H₃₉FN₄O₃ [M+H]⁺=475.30, found: 475.50.

### Synthesis of methyl ((1S,2R)-2-((S)-2-acetamido-1-(1-((1-(4-cyano-3-((3-fluoroazetidin-1-yl)methyl)phenyl)azetidin-3-yl)methyl)piperidin-4-yl)-1-(3-fluorophenyl)ethyl)cyclopentyl)carbamate (Cpd. No. 176)

To a solution of the intermediate L6 (40 mg, 0.068 mmol) in DMSO (1 mL) was added compound L7 (17 mg, 0.082 mmol), and K₂CO₃ (19 mg, 0.136 mmol). The mixture was stirred at 80° C. overnight and purified by reverse phase preparative HPLC to give the trifluoroacetic acid salt of Cpd. No. 176 (30 mg, 57%). ESI-MS calculated for Chemical Formula: C₃₇H₄₈F₂N₆O₃ [M+H]⁺=663.38, found: 663.53.

### Example 5

### Synthesis of 4-(3-((4-((S)-cyano(3-fluorophenyl)((1R,2S)-2-(2-oxooxazolidin-3-yl)cyclopentyl)methyl)piperidin-1-yl)methyl)azetidin-1-yl)benzonitrile (391)

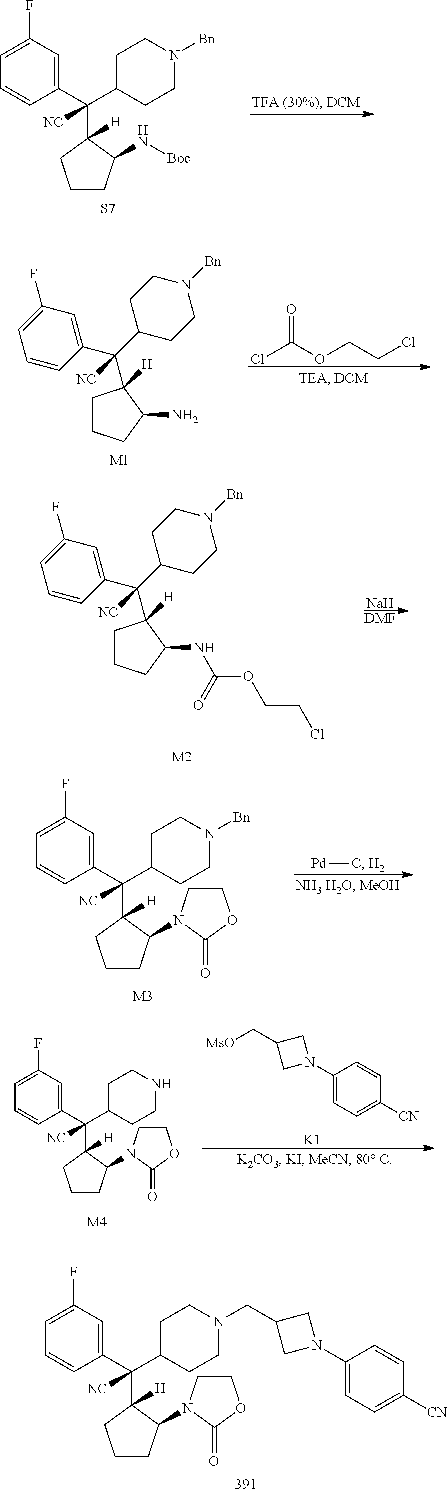

Intermediate S7 was treated with trifluoroacetic acid (TFA) to afford cyclopropyl amine intermediate M1, which was treated with chloroethyl chloroformate to afford intermediate M2. Intermediate M2 was treated with sodium hydride to afford oxazolidinone M3. Removal of the benzyl protecting group of M3 by hydrogenation in the presence of Pd/C afforded piperidine M4, which underwent nucleophilic substitution with mesylate K1 to afford compound 391. ¹H NMR (400 MHz, MeOD) δ 7.47-7.40 (m, 3H), 7.35 (d, J=ID Hz, 1H), 7.27 (d, J=10.5 Hz, 1H), 7.12 (td, J=8.3, 1.8 Hz, 1H), 6.44-6.36 (m, 2H), 4.12-4.01 (m, 4H), 3.94 (q, J=8.6 Hz, 1H), 3.60-3.48 (m, 3H), 3.28-3.21 (m, 1H), 3.15-3.09 (m, 1H), 2.96 (dd, J=12.2, 8.5 Hz, 3H), 2.61 (d, J=7.2 Hz, 2H), 2.18-1.99 (m, 4H), 1.90-1.64 (m, 7H), 1.46-1.36 (m, 1H), 1.16-1.05 (m, 1H).

### Example 6

### Synthesis of (R)—N-((1S,2R)-2-((S)-cyano(1-((1-(4-cyanophenyl)azetidin-3-yl)methyl)piperidin-4-yl)(3-fluorophenyl)methyl)cyclopentyl)oxirane-2-carboxamide and (S)—N-((1S,2R)-2-((S)-cyano(1-((1-(4-cyanophenyl)azetidin-3-yl)methyl)piperidin-4-yl)(3-fluorophenyl)methyl)cyclopentyl)oxirane-2-carboxamide (392 and 393)

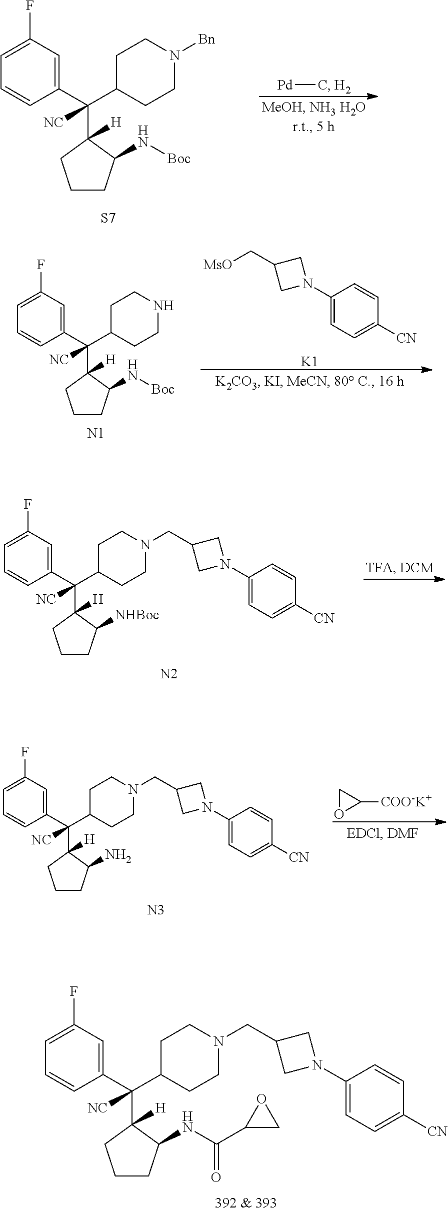

Removal of the benzyl protecting group of intermediate S7 by hydrogenation in the presence of Pd/C afforded piperidine N1, which underwent nucleophilic substitution with mesylate K1 to afford intermediate N2. Removal of the Boc protecting group from N2 by treatment with TFA afforded with N3 which was coupled with racemic potassium oxirane-2-carboxylate to afford a mixture of diastereomers 392 and 393. Separation of the diastereomers by supercritical fluid chromatography (SFC; Waters Thar 80 preparative SFC; ChiralPak IA, 250×21.2 mm I.D., 5 μM; Mobile Phase A: CO₂; Mobile Phase B: isopropyl alcohol+0.1% ammonium hydroxide; Gradient: B 40%; Flow rate: 55 mL/min; Back pressure: 100 bar; Column temperature: 35° C.; Wavelength: 285 nm; Cycle time: 6.1 min; Eluted time: 1.2 h) afforded the title compounds. The relative stereochemistry of the oxirane group of each isomer was not determined.

Compound 392 (first eluting isomer): ¹H NMR (400 MHz, MeOD) δ 7.47-7.39 (m, 3H), 7.33 (d, J=8.0 Hz, 1H), 7.26-7.19 (m, 1H), 7.12 (td, 7=8.3, 2.1 Hz, 1H), 6.41 (d, J=8.8 Hz, 2H), 4.13 (dd, J=12.5, 7.1 Hz, 1H), 4.04 (t, J=7.8 Hz, 2H), 3.57 (dd, J=7.9, 5.7 Hz, 2H), 3.15 (dd, J=4.4, 2.5 Hz, 1H), 3.00-2.89 (m, 4H), 2.84 (dd, J=6.0, 4.4 Hz, 1H), 2.62 (d, J=7.2 Hz, 2H), 2.52 (dd, J=6.1, 2.4 Hz, 1H), 2.17-1.97 (m, 4H), 1.97-1.89 (m, 1H), 1.87-1.79 (m, 1H), 1.72-1.49 (m, 5H), 1.34 (ddd, J=24.4, 12.3, 3.5 Hz, 1H), 1.16 (qd, J=12.5, 3.8 Hz, 1H).

Compound 393 (second eluting isomer): ¹H NMR (400 MHz, MeOD) δ 7.45-7.38 (m, 3H), 7.32 (d, J=8.0 Hz, 1H), 7.26-7.20 (m, 1H), 7.10 (td, J=8.3, 1.9 Hz, 1H), 6.44-6.37 (m, 2H), 4.18 (dd, J=14.2, 7.7 Hz, 1H), 4.04 (td, J=7.9, 1.8 Hz, 2H), 3.57 (dd, J=7.9, 5.7 Hz, 2H), 3.03 (dd, J=4.4, 2.4 Hz, 1H), 2.99-2.87 (m, 4H), 2.81 (dd, J=6.1, 4.4 Hz, 1H), 2.61 (d, J=7.2 Hz, 2H), 2.55 (dd, 7=6.1, 2.4 Hz, 1H), 2.17-1.92 (m, 5H), 1.83 (dt, J=13.5, 7.7 Hz, 1H), 1.76-1.55 (m, 4H), 1.49 (dt, J=12.4, 6.4 Hz, 1H), 1.42-1.32 (m, 1H), 1.16 (ddd, J=24.8, 14.1, 3.6 Hz, 1H).

### Example 7

### Synthesis of 4-(3-((4-((S)-cyano(3-fluorophenyl)((1R,2S)-2-(2-oxooxazol-3(2H)-yl)cyclopentyl)methyl)piperidin-1-yl)methyl)azetidin-1-yl)benzonitrile (394)

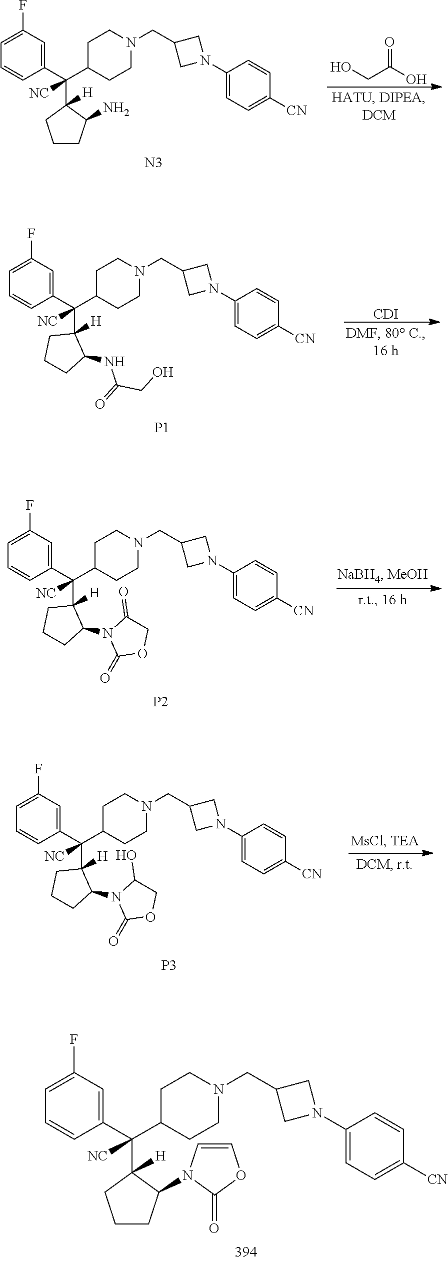

Intermediate N3 was coupled with glycolic acid to afford P1, which was then treated with carbonyn diimidazole (CDI) to afford oxazolidinedione P2. P2 was treated with sodium borohydride to afford hydroxyl oxazolidinone P3, which was treated with mesyl chloride to afford compound 394. ¹H NMR (400 MHz, MeOD) δ 7.45-7.40 (m, 2H), 7.38-7.31 (m, 1H), 7.28-7.17 (m, 1H), 7.12 (d, J=10.8 Hz, 1H), 7.04 (td, J=8.3, 1.8 Hz, 1H), 6.83 (dd, 7=11.8, 2.0 Hz, 2H), 6.41 (d, 7=8.8 Hz, 2H), 4.22 (dd, 7=15.8, 8.4 Hz, 1H), 4.03 (td, 7=7.9, 1.1 Hz, 2H), 3.56 (dd, 7=7.9, 5.7 Hz, 2H), 3.22 (q, J=8.6 Hz, 1H), 2.93 (t, J=11.5 Hz, 3H), 2.61 (d, J=7.2 Hz, 2H), 2.30-2.20 (m, 1H), 2.04 (dd, J=19.2, 7.8 Hz, 4H), 1.91-1.70 (m, 6H), 1.41-1.31 (m, 1H), 1.06 (qd, J=12.3, 3.3 Hz, 1H).

### Example 8

### Synthesis of methyl ((1S,2R)-2-(2-(azetidin-1-yl)-1-(1-((1-(4-(cyclopropylsulfonyl)phenyl)-3-ethoxyazetidin-3-yl)methyl)piperidin-4-yl)-1-(3-fluorophenyl)ethyl)cyclopentyl)carbamate (377)

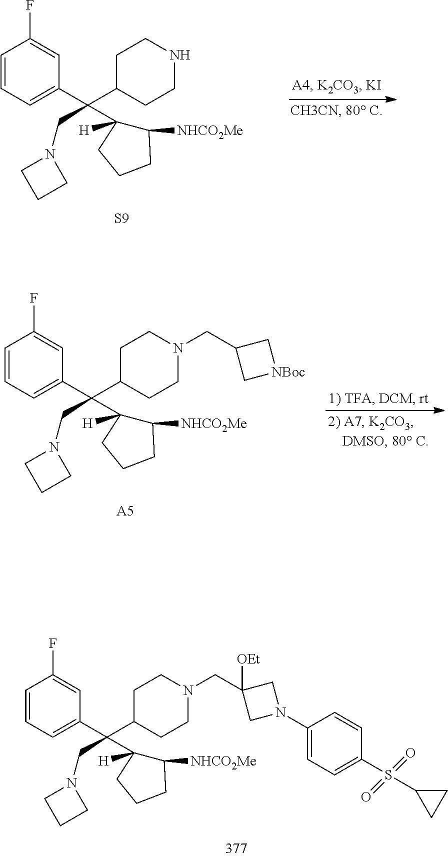

Synthesis of tert-butyl 3-((4-(2-(azetidin-1-yl)-1-(3-fluorophenyl)-1-((1R,2S)-2-((methoxycarbonyl)amino)cyclopentyl)ethyl)piperidin-1-yl)methyl)azetidine-1-carboxylate (A6): To a solution of the intermediate S9 (179 mg, 0.444 mmol) in acetonitrile (3 mL) was added A4 (206 mg, 0.666 mmol), K₂CO₃ (245 mg, 1.78 mmol) and KI (7 mg, 0.044 mmol). The mixture was stirred at 80° C. overnight. Then, the mixture purified by reverse phase preparative HPLC to give A5 (163 mg).

Synthesis of methyl ((1S,2R)-2-(2-(azetidin-1-yl)-1-(1-((1-(4-(cyclopropylsulfonyl)phenyl)-3-ethoxyazetidin-3-yl)methyl)piperidin-4-yl)-1-(3-fluorophenyl)ethyl)cyclopentyl)carbamate (377): A5 (32 mg, 0.052 mmoL) was dissolved in dichloromethane (1.5 mL) and trifluoroacetic acid (0.15 mL) was added. After stirring for 60 min at room temperature, the reaction mixture was concentrated under vacuum. The residue was then dissolved in DMSO (1 mL). 1-(cyclopropylsulfonyl)-4-fluorobenzene (A7) (21 mg, 0.062 mmol), and K₂CO₃ (29 mg, 0.21 mmol). The mixture was stirred at 80° C. overnight. Then, the mixture was purified by reverse phase preparative HPLC to give the trifluoroacetic acid salt of 377 (16 mg). ESI-MS calculated for C₃₈H₅₄FN₄O₅S [M+H]⁺=697.38, found: 697.44.

### Example 9

### Synthesis of methyl 2-(1-((1-(4-(cyclopropylsulfonyl)phenyl)azetidin-3-yl)methyl)piperidin-4-yl)-2-(3-fluorophenyl)-2-((1R,2S)-2-((methoxycarbonyl)amino)cyclopentyl)acetate (379)

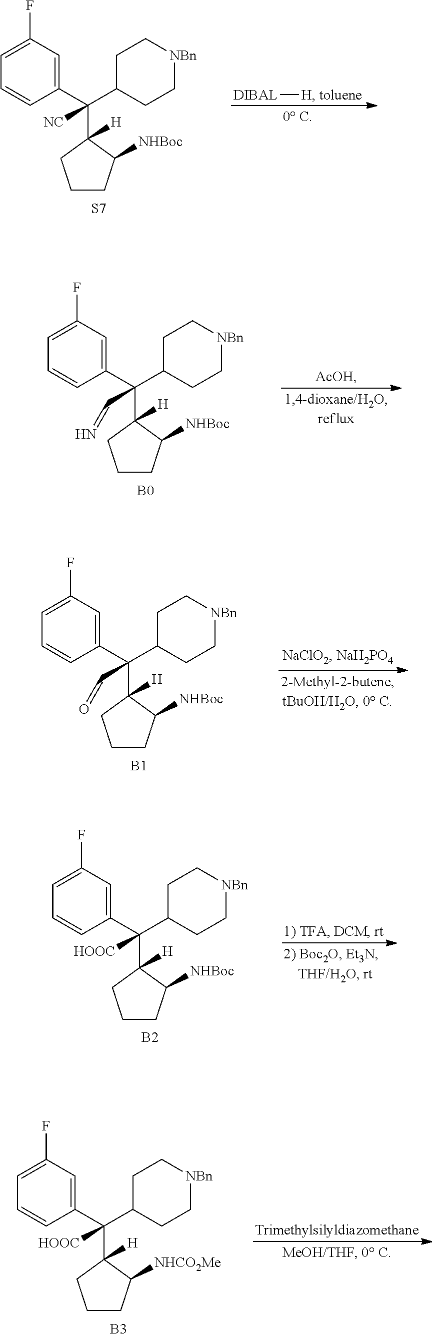

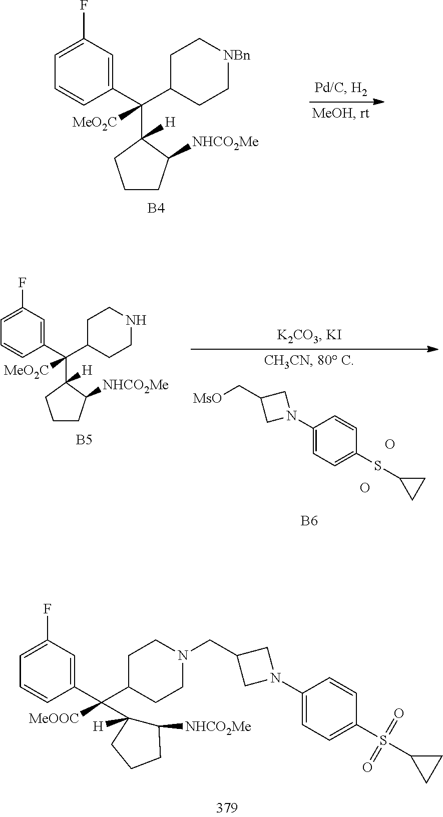

Synthesis of tert-butyl ((1S,2R)-2-((S)-1-(1-benzylpiperidin-4-yl)-1-(3-fluorophenyl)-2-iminoethyl)cyclopentyl)carbamate (B0): S7 (2 g, 4.1 mmol) an anhydrous toluene (40 ml) was added to the flask, then was cooled to 0° C. in the ice-bath. Diisobutylaluminiumhydride (25% in toluene, 10.8 mL, 16.3 mmol) was injected into the reaction mixture with syringe slowly at 0° C. with stirring. Then the ice-bath was removed, the reaction was monitored using UPLC-Mass (about 4 h). After the mass (492) of S7 disappeared, 20 ml of NaOH (1M) solution was added slowly into the reaction mixture at 0° C. to quench the reaction. After stirring for 5 min, the ice-bath was removed and additional 20 ml saturated brine was added. Then about 50 mL EA was added, the gel will form. The gel was filtered with celite, and was washed with EA, combine the solvent. The solution was extracted with EA, DCM twice. The organic solvent was dried with Na₂SO₄, filtered, and concentrated under rotatory vacuum to give crude product B0 without further purification.

Synthesis of tert-butyl ((1S,2R)-2-((S)-1-(1-benzylpiperidin-4-yl)-1-(3-fluorophenyl)-2-oxoethyl)cyclopentyl)carbamate (B1): B0 (obtained last step) was dissolved in 1,4-dioxane (30 mL). H₂O and acetic acid (5 mL) were added and the mixture was heated under reflux overnight. Saturated NaHCO₃ solution was than added to the mixture carefully then the solution was extracted with three times. The organic solvent was dried with Na₂SO₄, filter, and concentrated under rotatory vacuum to give crude product B1 (1.86 g) without further purification. ESI-MS calculated for C₃OH₄₀FN₂O₃ [M+H]⁺=495.30, found: 495.51.

Synthesis of (S)-2-(1-benzylpiperidin-4-yl)-2-((1R,2S)-2-((tert-butoxycarbonyl)amino)cyclopentyl)-2-(3-fluorophenyl)acetic acid (B2): B1 (200 mg, 0.41 mmol) was dissolved in tert-Butanol (5 mL), NaH₂PO₄ (146 mg, 1.2 mmol) and 2-Methyl-2-butene (0.24 mL, 2.2 mmol) were added. Sodium chlorite (69 mg, 0.61 mmol) was added under 0° C. After stirring for 4 h, the mixture was acidified with TFA and was purified by reverse phase preparative HPLC to give B2 (204 mg) as white solid. ESI-MS calculated for C₃₀H₄₀FN₂O₄ [M+H]⁺=511.30, found: 511.56.

Synthesis of (S)-2-(1-benzylpiperidin-4-yl)-2-(3-fluorophenyl)-2-((1R,2S)-2-((methoxycarbonyl)amino)cyclopentyl)acetic acid (B3): B2 (204 mg, 0.41 mmol) was dissolved in dichloromethane (3 mL) and trifluoroacetic acid (0.6 mL) was added. After stirring for 60 min at room temperature, the reaction mixture was concentrated under vacuum. The residue was dissolved in THF/H₂O (1.5 mL/1.5 mL). Then, Et₃N (0.14 mL, 1.0 mmol) and Dimethyl dicarbonate (81 mg, 0.61 mmol) were added. After stirring for 12 h at room temperature, 1M Hydrochloric acid (aq) was added. The mixture was purified by reverse phase preparative HPLC to give B3 (163 mg) as white solid. ESI-MS calculated for C₂₇H₃₄FN₂CU [M+H]⁺=469.25, found: 469.41.

Synthesis of methyl (S)-2-(1-benzylpiperidin-4-yl)-2-(3-fluorophenyl)-2-((1R,2S)-2-((methoxycarbonyl)amino)cyclopentyl)acetate (B4): B3 (125 mg, 0.267 mmol) was dissolved in MeOH/THF (1.5 mL/1.5 mL). Trimethylsilyldiazomethane was then added under 0° C. After 1 h, the reaction was quenched with acetic acid. The solvent was evaporated under vacuum to give crude product B4 (120 mg) without further purification. ESI-MS calculated for C₂₈H₃₆FN₂O₄ [M+H]⁺=483.27, found: 483.35.

Synthesis of methyl (S)-2-(3-fluorophenyl)-2-((1R,2S)-2-((methoxycarbonyl)amino)cyclopentyl)-2-(piperidin-4-yl)acetate (B5): To a solution of B4 (72 mg, 0.15 mmol) in methanol (2 mL) was added 10% Pd/C (20 mg). The mixture was stirred for 4 h at room temperature under hydrogen atmosphere (normal pressure). After the Pd/C catalyst was filtered off, the solvent was removed by rotary evaporation to give the crude product B5 (45 mg). ESI-MS calculated for C₂₁H₃₀FN₂O₄ [M+H]⁺=393.22, found: 393.36.

Synthesis of methyl 2-(1-((1-(4-(cyclopropylsulfonyl)phenyl)azetidin-3-yl)methyl)piperidin-4-yl)-2-(3-fluorophenyl)-2-((1R,2S)-2-((methoxycarbonyl)amino)cyclopentyl)acetate (379): To a solution of the intermediate B5 (45 mg, 0.091 mmol) in acetonitrile (1 mL) was added (1-(4-(cyclopropylsulfonyl)phenyl)azetidin-3-yl)methyl methanesulfonate (B6) (62 mg, 0.18 mmol), K₂CO₃ (82 mg, 0.60 mmol) and KI (1 mg, 0.005 mmol). The mixture was stirred at 80° C. overnight. Then, the mixture purified by reverse phase preparative HPLC to give 379 (30 mg). ¹H NMR (400 MHz, Methanol-r/r) δ 7.65 (d, J=8.8 Hz, 2H), 7.35 (q, J=7.9 Hz, 1H), 7.04 (t, J=9.5 Hz, 3H), 6.52 (d, J=8.6 Hz, 2H), 4.17 (t, J=7.9 Hz, 2H), 4.04 (s, 1H), 3.80 (s, 3H), 3.73 (t, J=6.9 Hz, 2H), 3.59 (s, 3H), 3.53 (d, J=14.7 Hz, 1H), 3.42 (d, J=7.1 Hz, 2H), 3.24-3.01 (m, 1H), 2.94 (t, J=12.1 Hz, 2H), 2.55 (tt, J=7.9, 4.8 Hz, 2H), 2.28-2.14 (m, 1H), 2.02 (d, 7=13.1 Hz, 2H), 1.64-1.36 (m, 5H), 1.36-1.20 (m, 4H), 1.13 (dt, J=6.6, 3.1 Hz, 2H), 0.99 (ddd, J=7.8, 5.7, 1.9 Hz, 2H). ESI-MS calculated for C₃₄H₄₅FN₃O₆S [M+H]⁺=642.30, found: 697.39.

### Example 10

### Synthesis of 1-((1S,2R)-2-((S)-2-(azetidin-1-yl)-1-(1-((1-(4-(cyclopropylsulfonyl)phenyl)azetidin-3-yl)methyl)piperidin-4-yl)-1-(3-fluorophenyl)ethyl)cyclopentyl)-3-methylimidazolidin-2-one (350)

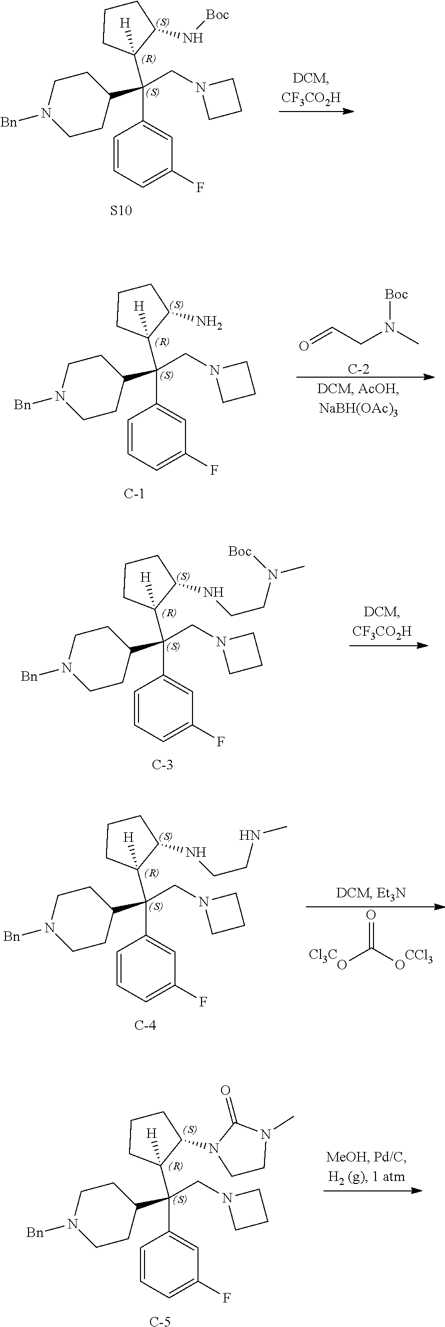

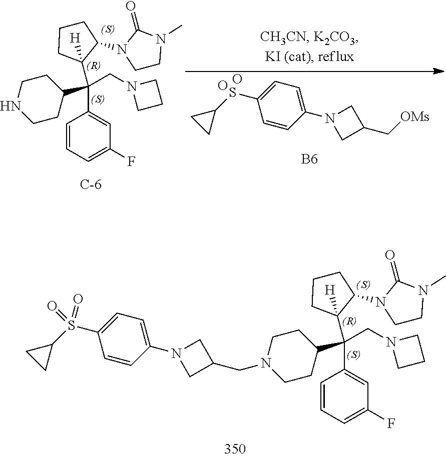

N1-((1S,2R)-2-((S)-2-(azetidin-1-yl)-1-(1-benzylpiperidin-4-yl)-1-(3-fluorophenyl)ethyl)cyclopentyl)-N2-methylethane-1,2-diamine (C-4): Compound S10 (102 mg, 0.191 mmol) was dissolved in DCM (1 mL) then CF₃CO₂H (4 mL) was added. After 5 minutes the reaction was complete and the solvent was removed by rotary evaporation to produce crude C-1 that was used without purification. Aldehyde C-2 (66 mg, 0.381 mmol) and crude C-1 were dissolved in DCM (3 mL) with catalytic AcOH and stirred. After 10 minutes, NaBH(OAc)₃ (162 mg, 0.764 mmol) was added and the reaction was stirred. After overnight the reaction was quenched with methanol then the solvent was removed and the crude was purified by prep HPLC to produce C-3. C-3 was treated with trifluoroacetic acid (2 mL) for 5 minutes then concentrated to produce C-4 (84 mg). ESI-MS calculated for C31H46FN [M+H]+=493.36, found: 493.51.

1-((1S,2R)-2-((S)-2-(azetidin-1-yl)-1-(1-benzylpiperidin-4-yl)-1-(3-fluorophenyl)ethyl)cyclopentyl)-3-methylimidazolidin-2-one (C-5): At 0° C., triphosgene (30 mg, 0.102 mmol) was added to a solution of C-4 (84 mg, 0.170 mmol), and Et₃N (118 uL, 0.85 mmol) in DCM (6 mL). After 1 hour the reaction was quenched with methanol, concentrated and purified by prep-HPLC to produce C-5 (23 mg). ESI-MS calculated for C32H44FN₄O [M+H]+=519.34, found: 519.49.

1-((1S,2R)-2-((S)-2-(azetidin-1-yl)-1-(1-((1-(4-(cyclopropylsulfonyl)phenyl)azetidin-3-yl)methyl)piperidin-4-yl)-1-(3-fluorophenyl)ethyl)cyclopentyl)-3-methylimidazolidin-2-one (350): Compound C-5 (23 mg, 0.044 mmol) was dissolved with 2 mL of methanol and the solution was purged twice by vacuuming briefly followed by adding nitrogen atmosphere. Pd/C (50 mg) was quickly added then the reaction was vacuumed and put under H₂ atmosphere for 2 hours. After the Pd/C catalyst was filtered off through celite, the solvent was removed by rotary evaporation to give the crude product C-6. To a solution of the crude C-6 in acetonitrile (2 mL) was added B6 (20 mg, 0.058 mmol), K₂CO₃ (18 mg, 0.133 mmol), KI (1 mg, 0.005 mmol) and the mixture was stirred at reflux. After overnight the mixture was cooled to room temperature, filtered, concentrated, purified by prep HPLC, and lyophilized to give 350 (15 mg). ESI-MS calculated for C38H53FN5O3S [M+H]⁺=678.38, found: 678.51.

methyl ((1S,2R)-2-((S)-1-(1-((1-(4-(cyclopropylsulfonyl)phenyl)azetidin-3-yl)methyl)piperidin-4-yl)-1-(3-fluorophenyl)-2-(3-hydroxyazetidin-1-yl)ethyl)cyclopentyl)carbamate (351). Starting with D-4 (20 mg, 0.039 mmol), 351 was prepared according to the procedure described for 350. ESI-MS calculated for C₃₆H₅₀FN₄O₅S [M+H]⁺=669.34, found: 669.49.

((1S,2R)-2-((S)-1-(1-((1-(4-(cyclopropylsulfonyl)phenyl)azetidin-3-yl)methyl)piperidin-4-yl)-1-(3-fluorophenyl)-2-(3-methoxyazetidin-1-yl)ethyl)cyclopentyl)carbamate (352): Starting with D-6 (22 mg, 0.042 mmol), 352 was prepare according to the procedure described for 350. ESI-MS calculated for C₃₇H₅₂FN₄O₅S [M+H]⁺=683.36, found: 683.45.

((1S,2R)-2-((S)-1-(1-((1-(4-(cyclopropylsulfonyl)phenyl)azetidin-3-yl)methyl)piperidin-4-yl)-2-(3-fluoroazetidin-1-yl)-1-(3-fluorophenyl)ethyl)cyclopentyl)carbamate (353): Starting with D-7 (18 mg, 0.035 mmol), 353 was prepare according to the procedure described for 350. ESI-MS calculated for C₃₆H₄₈F₂N₄O₄S [M+H]⁺=671.34, found: 671.48.

### Example 11

### Synthesis of methyl ((1S,2R)-2-((S)-1-(1-((1-(4-(cyclopropylsulfonyl)phenyl)azetidin-3-yl)methyl)piperidin-4-yl)-2-(ethylamino)-1-(3-fluorophenyl)ethyl)cyclopentyl)carbamate (355)

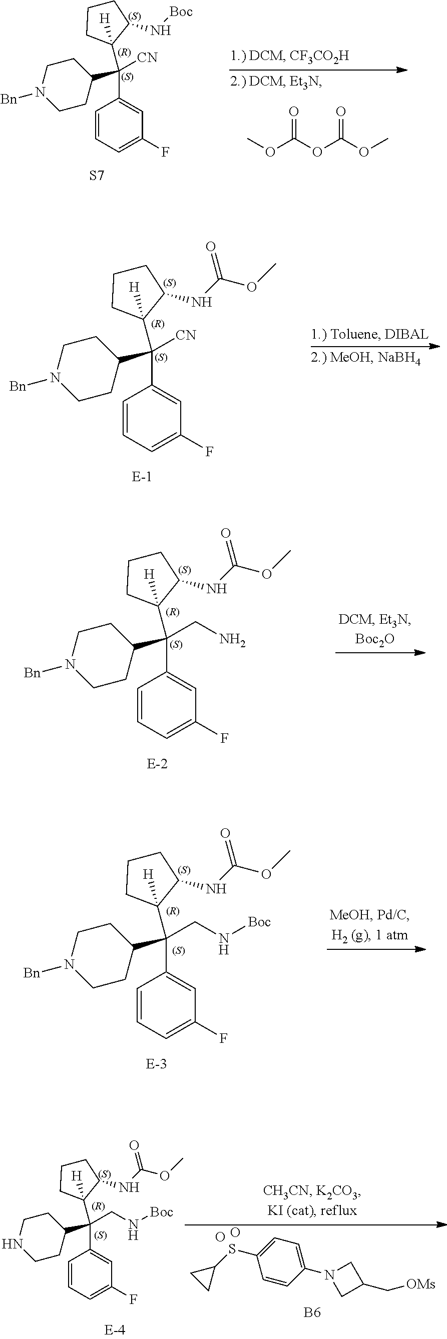

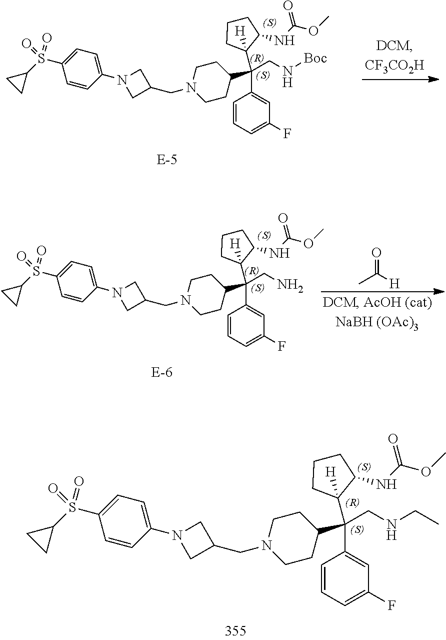

methyl ((1S,2R)-2-((S)-(1-benzylpiperidin-4-yl)(cyano)(3-fluorophenyl)methyl)cyclopentyl)carbamate (E-1): Compound S7 (1.0 g, 2.04 mmol) was dissolved in DCM (2 mL) then CF₃C₀₂H (6 mL) was added. After 15 minutes the reaction was complete and the solvent was removed by rotary evaporation to produce that was used without purification. At 0° C., dimethyl dicarbonate (410 mg, 3.05 mmol) was added to a solution of crude S7-deprotected and Et₃N (1.13 mL, 8.16 mmol) in DCM (30 mL). After 2 hours, the reaction was concentrated and purified by column chromatography to produce E-1 (770 mg). ESI-MS calculated for C₂₇H₃₃FN₃O₂ [M+H]⁺=450.25, found: 450.45.

methyl ((1S,2R)-2-((S)-2-amino-1-(1-benzylpiperidin-4-yl)-1-(3-fluorophenyl)ethyl)cyclopentyl)carbamate (E-2): At 0° C., diisobutylaluminium hydride (3.90 mL, 6.86 mmol) was added to a solution of E-1 (770 mg, 1.715 mmol) in toluene (17 mL). After 1 hour at 0° C. the reaction was allowed to warm to room temperature for 15 minutes then the reaction was slowly quenched with 2M NaOH. The quenched reaction was diluted with ethyl acetate, brine, and extracted 3 times. The combined organic layers were dried over sodium sulfate, filtered through celite, concentrated, and vacuumed to remove the residual solvent. This crude product was re-dissolved in methanol then treated with NaBH₄ (130 mg, 3.43 mmol). After overnight the reaction was quenched with 2M NaOH, diluted with ethyl acetate, and brine, then extracted 3 times. The combined organic layers were dried over sodium sulfate, filtered, concentrated, and vacuumed to remove the residual solvent to produce crude E-2 (775 mg). ESI-MS calculated for C₂₇H₃₇FN₃O₂ [M+H]⁺=454.28, found: 454.41.

methyl ((1S,2R)-2-((S)-1-(1-benzylpiperidin-4-yl)-2-((tert-butoxycarbonyl)amino)-1-(3-fluorophenyl)ethyl)cyclopentyl)carbamate (E-3): At 0° C., di-tert-butyl dicarbonate (316 mg, 1.66 mmol) was added to a solution of E-2 (500 mg, 1.10 mmol) and Et₃N (459 uL, 3.30 mmol) in DCM (15 mL). After 2 hours, the reaction was concentrated and purified by column chromatography to produce E-3 (485 mg). ESI-MS calculated for C₃₂H₄₅FN₃O [M+H]⁺=554.33, found: 554.51.

methyl ((1S,2R)-2-((S)-2-((tert-butoxycarbonyl)amino)-1-(1-((1-(4-(cyclopropylsulfonyl)phenyl)azetidin-3-yl)methyl)piperidin-4-yl)-1-(3-fluorophenyl)ethyl)cyclopentyl)carbamate (E-5): Starting with E-3 (485 mg), E-5 was prepare according to the procedure described for 350. ESI-MS calculated for C₃₈H₅₄FN₄O₆S [M+H]⁺=713.37, found: 713.53.

methyl ((1S,2R)-2-((S)-2-amino-1-(1-((1-(4-(cyclopropylsulfonyl)phenyl)azetidin-3-yl)methyl)piperidin-4-yl)-1-(3-fluorophenyl)ethyl)cyclopentyl)carbamate (E-6): Compound E-5 (356 mg) was dissolved in DCM (2 mL) then CF₃CO₂H (6 mL) was added. After 15 minutes the reaction was complete and the solvent was removed by rotary evaporation. The residue was re-dissolved in 0.5 mL acetonitrile and 4 mL H₂O, frozen and lyophilized to produce E-6 (320 mg). ESI-MS calculated for C₃₃H₄₆FN₄O₄S [M+H]⁺=613.31, found: 613.49.

methyl ((1S,2R)-2-((S)-1-(1-((1-(4-(cyclopropylsulfonyl)phenyl)azetidin-3-yl)methyl)piperidin-4-yl)-2-(ethylamino)-1-(3-fluorophenyl)ethyl)cyclopentyl)carbamate (355): Acetaldehyde (7.1 uL, 0.126 mmol) and E-6 (25 mg, 0.041 mmol) were dissolved in DCM (1 mL) with catalytic AcOH and stirred. After 10 minutes, NaBH(OAc)₃ (36 mg, 0.168 mmol) was added and the reaction was stirred. After overnight the reaction was quenched with methanol then the solvent was removed and the crude was purified by prep HPLC to produce 355. ESI-MS calculated for C₃₅H₅₀FN₄O₄S [M+H]⁺=641.35, found: 641.45.

### Example 12

### Synthesis of methyl ((1S,2R)-2-((1S)-2-(azetidin-1-yl)-1-(1-((3S)-3-((4-(cyclopropylsulfonyl)phenyl)amino)cyclopentyl)piperidin-4-yl)-1-(3-fluorophenyl)ethyl)cyclopentyl)carbamate (385)

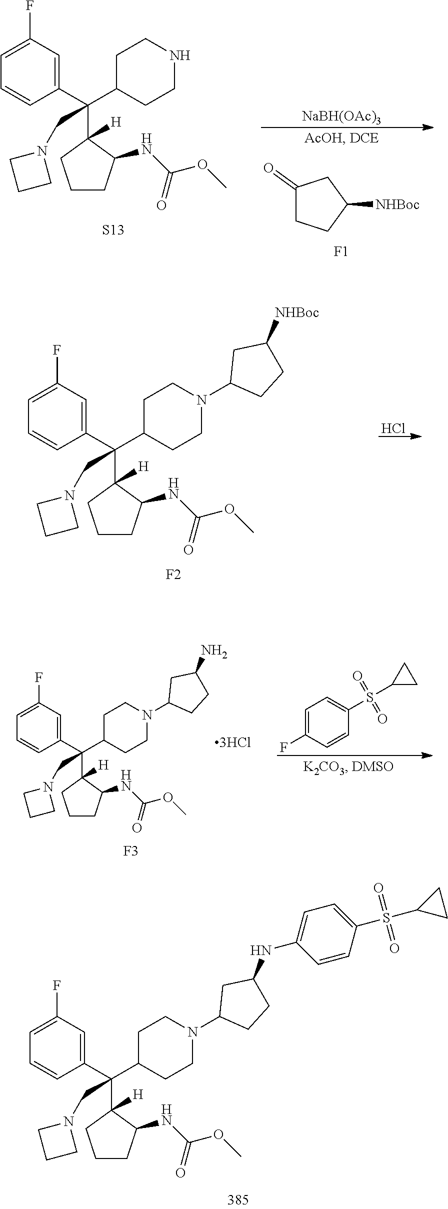

Methyl ((1S,2R)-2-((1S)-2-(azetidin-1-yl)-1-(1-((3S)-3-((tert-butoxycarbonyl)amino)cyclopentyl)piperidin-4-yl)-1-(3-fluorophenyl)ethyl)cyclopentyl)carbamate (F2): To a solution of methyl ((1S,2R)-2-((S)-2-(azetidin-1-yl)-1-(3-fluorophenyl)-1-(piperidin-4-yl)ethyl)cyclopentyl)carbamate S13 (50 mg, 0.12 mmol, 1 eq) and tert-butyl (S)-(3-oxocyclopentyl)carbamate F1 (37 mg, 0.19 mmol, 1.5 eq) in 1,2-dichloroethane (5 mL), at room temperature was added acetic acid (11 mg, 0.19 mmoL, 1.5 eq) and sodium triacetoxyborohyride (40 mg, 0.19 mmoL, 1.5 eq) subsequently. After stirring for 6 h at rt, the reaction mixture was evaporated and the residue was purified by reverse phase preparative HPLC to give F2. ESI-MS [M+H]⁺=587.53.

methyl ((1S,2R)-2-((1S)-2-(azetidin-1-yl)-1-(1-((3S)-3-((4-(cyclopropylsulfonyl)phenyl)amino)cyclopentyl)piperidin-4-yl)-1-(3-fluorophenyl)ethyl)cyclopentyl)carbamate (385): F2 (11 mg, 0.02 mmoL, 1 eq) was added to a solution of 4.0 M hydrogen chloride in dioxane (2 mL) at room temperature. After 0.5 h, the solvent was evaporated to give crude F3 which was used for next step without further purification. F3 was dissolved in 1 mL of DMSO, then 1-(cyclopropylsulfonyl)-4-fluorobenzene (7.5 mg, 0.04 mmoL, 2 eq) and potassium carbonate (11 mg, 0.07 mmoL, 4 eq) were added in the solution. The resulting mixture was stirred and heated at 120° C. for 2 h and then purified by reverse phase preparative HPLC to give the title compound 385. ESI-MS [M+H]⁺=667.82.

### Example 13

### Synthesis of methyl ((1S,2R)-2-((S)-1-(1-((1-(4-(cyclopropylsulfonyl)phenyl)azetidin-3-yl)methyl)piperidin-4-yl)-2-(2-(dimethylamino)acetamido)-1-(3-fluorophenyl)ethyl)cyclopentyl)carbamate (395), methyl ((1S,2R)-2-((S)-1-(1-((1-(4-(cyclopropylsulfonyl)phenyl)azetidin-3-yl)methyl)piperidin-4-yl)-1-(3-fluorophenyl)-2-(2-morpholinoacetamido)ethyl)cyclopentyl)carbamate (396), and methyl ((1S,2R)-2-((S)-2-(2-(azetidin-1-yl)acetamido)-1-(1-((1-(4-(cyclopropylsulfonyl)phenyl)azetidin-3-yl)methyl)piperidin-4-yl)-1-(3-fluorophenyl)ethyl)cyclopentyl)carbamate (397)

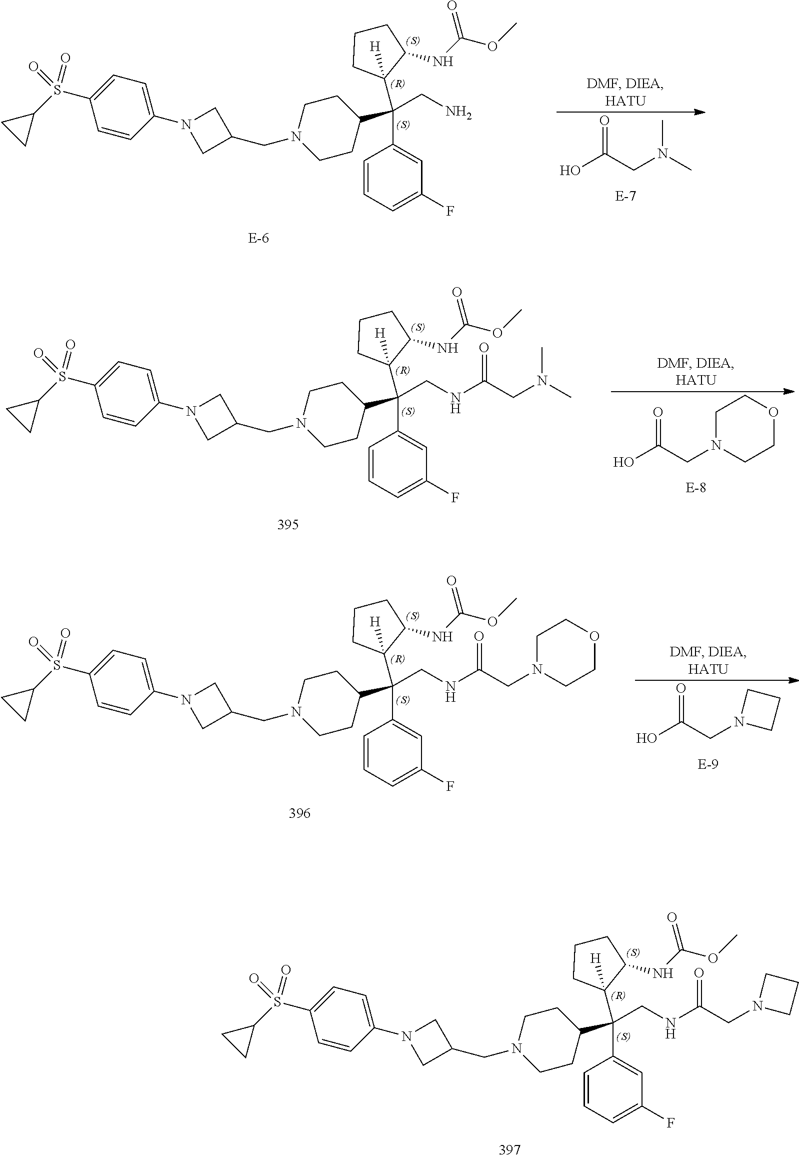

methyl ((1S,2R)-2-((S)-1-(1-((1-(4-(cyclopropylsulfonyl)phenyl)azetidin-3-yl)methyl)piperidin-4-yl)-2-(2-(dimethylamino)acetamido)-1-(3-fluorophenyl)ethyl)cyclopentyl)carbamate (395): HATU (19 mg, 0.0489 mmol) was added to a solution of E-6 (20 mg, 0.033 mmol), E-7 (5 mg, 0.0489 mmol), and DIEA (22 uL, 0.134 mmol) in DMF (0.5 mL). After 5 minutes the reaction was determined to be complete by UPLC so it was purified by prep-HPLC to produce 395 (14 mg). ESI-MS calculated for C₃₇H₅₃FN₅O₅S [M+H]⁺=698.37, found: 698.62.

methyl ((1S,2R)-2-((S)-1-(1-((1-(4-(cyclopropylsulfonyl)phenyl)azetidin-3-yl)methyl)piperidin-4-yl)-1-(3-fluorophenyl)-2-(2-morpholinoacetamido)ethyl)cyclopentyl)carbamate (396): Starting with 2-morpholinoacetic acid (E-8) in place of E-7, 396 was prepared according to the procedure described for the synthesis of 395. ESI-MS calculated for C₃₉H₅₅FN₅O₆S [M+H]⁺=740.38, found: 740.57.

methyl ((1S,2R)-2-((S)-2-(2-(azetidin-1-yl)acetamido)-1-(1-((1-(4-(cyclopropylsulfonyl)phenyl)azetidin-3-yl)methyl)piperidin-4-yl)-1-(3-fluorophenyl)ethyl)cyclopentyl)carbamate (397): Starting with 2-(azetidin-1-yl)acetic acid (E-9) in place of E-7, 397 was prepared according to the procedure described for the synthesis of 395. ESI-MS calculated for C₃₈H₅₃FN₅O₅S [M+H]⁺=710.37, found: 710.66.

### Example 14

### Synthesis of methyl ((1S,2R)-2-((S)-2-(2-amino-2-methylpropanamido)-1-(1-((1-(4-(cyclopropylsulfonyl)phenyl)azetidin-3-yl)methyl)piperidin-4-yl)-1-(3-fluorophenyl)ethyl)cyclopentyl)carbamate (398)

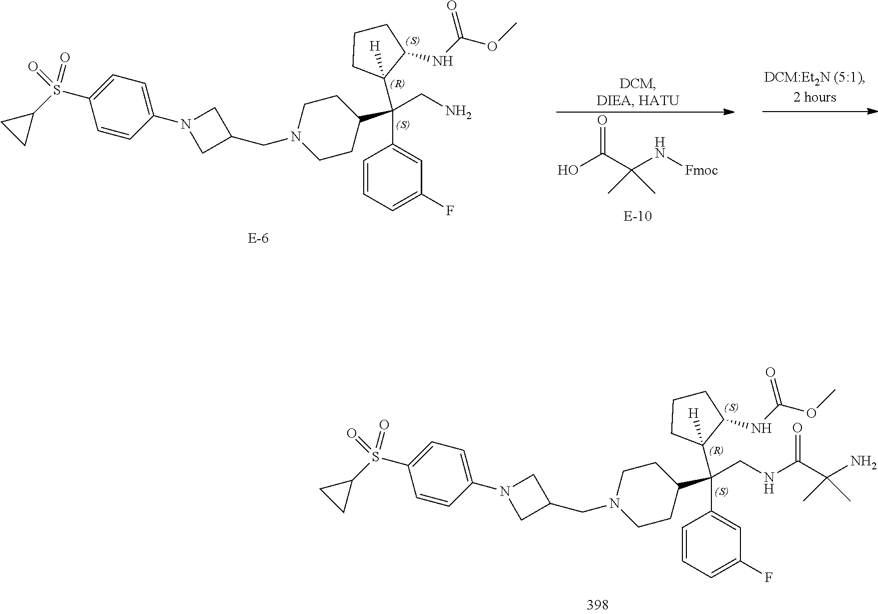

HATU (19 mg, 0.0489 mmol) was added to a solution of E-6 (20 mg, 0.033 mmol), E-10 (16 mg, 0.0489 mmol), and DIEA (22 uL, 0.134 mmol) in DCM (1.5 mL). After 5 minutes the reaction was determined to be complete by UPLC and the solvent was removed by rotary evaporation. The crude was redissolved in a solution of DCM/Et₂N (5:1) and stirred at room temperature. After 2 hours, the reaction was determined to be complete by UPLC so the solvent was removed and the crude was purified by prep-HPLC to produce 398 (8 mg). ESI-MS calculated for C₃₇H₅₃FN₅O₅S [M+H]⁺=698.37, found: 698.68.

### Example 15

### Synthesis of methyl ((1S,2R)-2-((S)-2-((R)-azetidine-2-carboxamido)-1-(1-((1-(4-(cyclopropylsulfonyl)phenyl)azetidin-3-yl)methyl)piperidin-4-yl)-1-(3-fluorophenyl)ethyl)cyclopentyl)carbamate (399) and methyl ((1S,2R)-2-((S)-2-((S)-azetidine-2-carboxamido)-1-(1-((1-(4-(cyclopropylsulfonyl)phenyl)azetidin-3-yl)methyl)piperidin-4-yl)-1-(3-fluorophenyl)ethyl)cyclopentyl)carbamate (400)

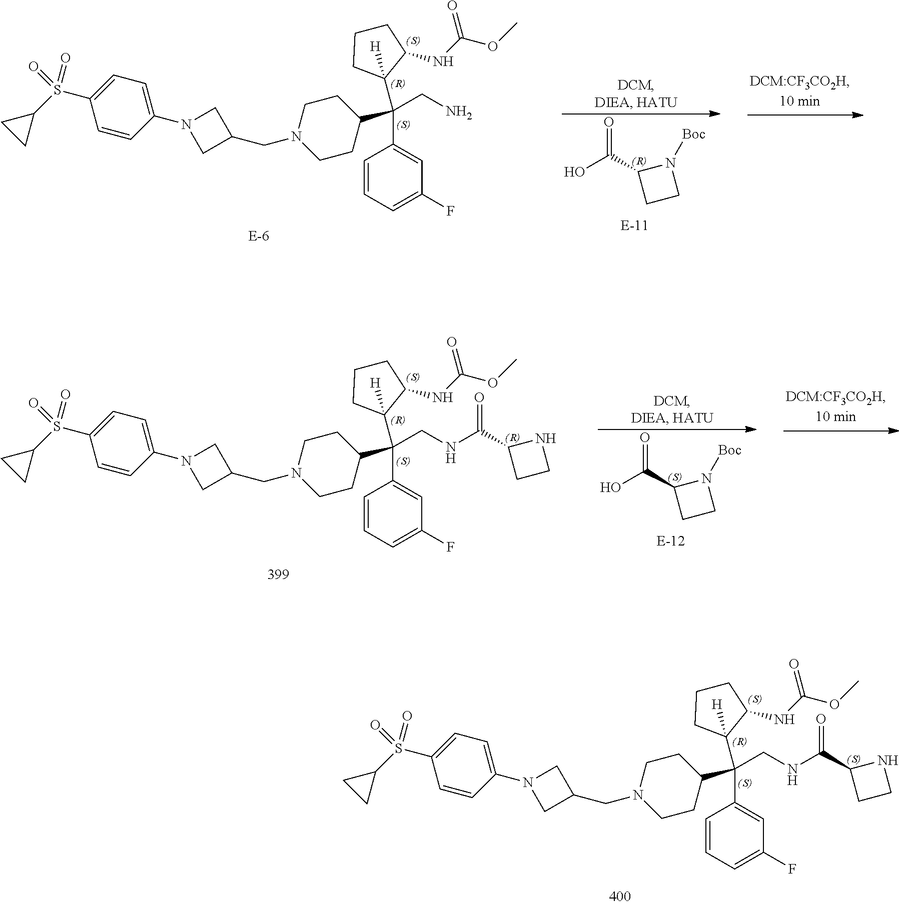

methyl ((1S,2R)-2-((S)-2-((R)-azetidine-2-carboxamido)-1-(1-((1-(4-(cyclopropylsulfonyl)phenyl)azetidin-3-yl)methyl)piperidin-4-yl)-1-(3-fluorophenyl)ethyl)cyclopentyl)carbamate (399): HATU (19 mg, 0.0489 mmol) was added to a solution of E-6 (20 mg, 0.033 mmol), E-11 (10 mg, 0.0489 mmol), and DIEA (22 uL, 0.134 mmol) in DCM (1.5 mL). After 5 minutes the reaction was determined to be complete by UPLC and the solvent was removed by rotary evaporation. The crude was redissolved in a solution of DCM/CF₃C₀₂H (1 mL:2 mL) and stirred at room temperature. After 10 minutes the reaction was determined to be complete by UPLC so the solvent was removed and the crude was purified by prep-HPLC to produce 399 (14 mg). ESI-MS calculated for C₃₇H₅₁FN₅O₅S [M+H]⁺=696.35, found: 696.62.

methyl ((1S,2R)-2-((S)-2-((S)-azetidine-2-carboxamido)-1-(1-((1-(4-(cyclopropylsulfonyl)phenyl)azetidin-3-yl)methyl)piperidin-4-yl)-1-(3-fluorophenyl)ethyl)cyclopentyl)carbamate (400): Starting with (E-12) in place of E-11, 400 was prepared according to the procedure described for the synthesis of 399. ESI-MS calculated for C₃₇H₅₁FN₅O₅S [M+H]⁺=696.35, found: 696.63.

### Example 16

### Synthesis and Characterization of Compounds of the Disclosure

Other Compounds of the Disclosure can be prepared using methods described in Schemes 1-3 and in the preceding EXAMPLES and related methods, see, e.g., WO 2017/192543. The MS (ESI) data for representative Compounds of the Disclosure prepared by these methods are provided in Tables 1.1, 1.2, and 1.3.

¹H NMR and/or ¹³C NMR data for additional Compounds of the Disclosure is provided in the following table:

| Cpd No. | ¹H NMR and/or ¹³C NMR Data |
|---|---|
| 1 | ¹H NMR (400 MHz, Methanol-d₄) δ 8.07-8.00 (m, 2H), |
| | 7.54 (q, J = 7.5 Hz, 1H), 7.50-7.38 (m, 4H), 7.32 (d, J = |
| | 2.2 Hz, 1H), 7.20 (t, J = 7.6 Hz, 1H), 6.95 (d, J = 2.3 Hz, |
| | 1H), 4.83 (d, J = 15.8 Hz, 1H), 4.57 (d, J = 15.5 Hz, 1H), |
| | 4.38 (t, J = 9.4 Hz, 2H), 4.17-4.04 (m, 2H), 4.02-3.92 |
| | (m, 1H), 3.78-3.55 (m, 8H), 3.50-3.34 (m, 4H), 3.10 (q, |
| | J = 8.1, 7.5 Hz, 2H), 2.97 (t, J = 12.3 Hz, 1H), 2.83 (t, J = |
| | 12.1 Hz, 1H), 2.76-2.57 (m, 2H), 2.29 (d, J = 13.8 Hz, |
| | 1H), 2.03-1.85 (m, 2H), 1.68-1.46 (m, 2H), 1.46-1.09 |
| | (m, 8H), 0.99 (dd, J = 23.7, 11.4 Hz, 1H). |
| 47 | ¹H NMR (400 MHz, Methanol-d₄) δ 7.67 (d, J = 8.9 Hz, |
| | 2H), 7.51-7.40 (m, 1H), 7.14 (dd, J = 8.4, 6.3 Hz, 2H), |
| | 7.05 (d, J = 8.0 Hz, 1H), 6.51 (d, J = 8.9 Hz, 2H), 4.79 |
| | (dd, J = 7.0, 1.6 Hz, 4H), 4.58 (tt, J = 7.9, 6.3 Hz, 1H), |
| | 4.54-4.44 (m, 2H), 4.41-4.26 (m, 2H), 4.19-4.09 (m, |
| | 3H), 3.78 (d, J = 15.6 Hz, 1H), 3.75-3.70 (m, 2H), 3.60- |
| | 3.44 (m, 3H), 3.39 (d, J = 7.1 Hz, 2H), 3.21 (p, J = 7.1 Hz, |
| | 1H), 2.97 (dt, J = 25.0, 12.4 Hz, 2H), 2.77 (q, J = 9.2, 8.5 |
| | Hz, 1H), 2.61-2.34 (m, 2H), 2.15-1.82 (m, 5H), 1.82- |
| | 1.72 (m, 1H), 1.72-1.55 (m, 3H), 1.54-1.37 (m, 1H), |
| | 1.20-0.98 (m, 1H). |
| 72 | ¹H NMR (400 MHz, Methanol-d₄) δ 7.49-7.41 (m, 1H), |
| | 7.36-7.28 (m, 2H), 7.13 (dd, J = 8.5, 6.5 Hz, 2H), 7.05 |
| | (d, J = 8.0 Hz, 1H), 6.52 (t, J = 8.8 Hz, 1H), 4.58-4.42 |
| | (m, 2H), 4.41-4.31 (m, 2H), 4.26 (td, J = 8.2, 2.4 Hz, |
| | 3H), 4.12 (d, J = 15.6 Hz, 1H), 3.89-3.81 (m, 2H), 3.78 |
| | (d, J = 15.6 Hz, 1H), 3.57-3.43 (m, 3H), 3.39 (d, J = 7.1 |
| | Hz, 2H), 3.19 (p, J = 7.0 Hz, 1H), 2.97 (dt, J = 25.3, 12.4 |
| | Hz, 2H), 2.76 (q, J = 9.3, 8.5 Hz, 1H), 2.59-2.34 (m, |
| | 2H), 2.14-1.82 (m, 6H), 1.82-1.54 (m, 5H), 1.46 (q, J = |
| | 12.9 Hz, 1H), 1.22-0.95 (m, 1H). |
| 73 | ¹H NMR (400 MHz, Methanol-d₄) δ 7.50-7.38 (m, 2H), |
| | 7.14 (dd, J = 8.5, 6.3 Hz, 2H), 7.05 (d, J = 8.0 Hz, 1H), |
| | 6.29-6.19 (m, 2H), 4.50 (d, J = 9.2 Hz, 2H), 4.42-4.24 |
| | (m, 2H), 4.20-4.09 (m, 3H), 3.85-3.69 (m, 3H), 3.59- |
| | 3.43 (m, 3H), 3.39 (d, J = 7.1 Hz, 2H), 3.21 (dt, J = 12.9, |
| | 6.3 Hz, 1H), 2.97 (dt, J = 24.7, 12.4 Hz, 2H), 2.77 (q, J = |
| | 9.1 Hz, 1H), 2.62-2.36 (m, 2H), 2.13-1.55 (m, 10H), |
| | 1.46 (q, J = 11.6, 10.5 Hz, 1H), 1.23-0.96 (m, 1H). |
| 77 | ¹H NMR (400 MHz, Methanol-d₄) δ 8.12 (s, 1H), 7.76 (d, |
| | J = 8.8 Hz, 2H), 7.72 (d, J = 0.7 Hz, 1H), 7.47 (q, J = 7.9 |
| | Hz, 1H), 7.20-7.10 (m, 2H), 7.04 (d, J = 8.0 Hz, 1H), |
| | 6.56 (d, J = 8.9 Hz, 2H), 4.60-4.47 (m, 2H), 4.42-4.30 |
| | (m, 2H), 4.24-4.11 (m, 5H), 3.91-3.86 (m, 3H), 3.82- |
| | 3.67 (m, 3H), 3.56 (t, J = 13.0 Hz, 2H), 3.47-3.38 (m, |
| | 1H), 3.28 (s, 3H), 3.19-3.02 (m, 2H), 2.83-2.73 (m, |
| | 1H), 2.56-2.41 (m, 2H), 2.12-1.97 (m, 4H), 1.87-1.74 |
| | (m, 2H), 1.74-1.59 (m, 3H), 1.50 (t, J = 12.7 Hz, 1H), |
| | 1.13-1.01 (m, 1H). |
| 79 | ¹H NMR (400 MHz, Methanol-d₄) δ 7.67 (d, J = 8.7 Hz, |
| | 2H), 7.46 (q, J = 7.9 Hz, 1H), 7.15 (t, J = 8.2 Hz, 2H), |
| | 7.03 (d, J = 7.9 Hz, 1H), 6.43 (d, J = 8.8 Hz, 2H), 4.62- |
| | 4.48 (m, 2H), 4.37 (q, J = 9.0 Hz, 2H), 4.17 (d, J = 15.8 |
| | Hz, 1H), 4.09 (t, J = 7.8 Hz, 2H), 3.79 (d, J = 15.7 Hz, |
| | 1H), 3.69-3.59 (m, 2H), 3.50 (t, J = 14.1 Hz, 2H), 3.37 |
| | (d, J = 7.1 Hz, 3H), 3.26 (s, 3H), 3.20-3.07 (m, 1H), 2.98 |
| | (dt, J = 23.9, 12.8 Hz, 2H), 2.87 (s, 3H), 2.80 (dd, J = |
| | 18.1, 8.9 Hz, 1H), 2.60-2.40 (m, 2H), 2.15-1.95 (m, |
| | 4H), 1.87-1.58 (m, 5H), 1.41 (q, J = 13.1 Hz, 1H), 1.04- |
| | 0.85 (m, 1H). |
| 93 | ¹H NMR (400 MHz, MeOD) δ 7.79-7.40 (m, 3H), 7.29- |
| | 6.69 (m, 4H), 6.54 (d, J = 8.9 Hz, 1H), 4.80-4.00 (m, |
| | 6H), 3.89-3.36 (m, 7H), 3.30-2.16 (m, 13H), 2.10- |
| | 0.59 (m, 14H). |
| 95 | ¹H NMR (400 MHz, MeOD) δ 8.12-7.89 (m, 4H), 7.77- |
| | 7.39 (m, 3H), 7.27-6.66 (m, 4H), 6.48 (d, J = 8.9 Hz, |
| | 1H), 4.64-3.95 (m, 6H), 3.88-3.36 (m, 7H), 3.26-2.36 |
| | (m, 7H), 2.24-0.79 (m, 14H). |
| 98 | ¹H NMR (400 MHz, CD₃OD) δ 7.99-7.94 (m, 4H), 7.78 |
| | (d, J = 8.4 Hz, 2H), 7.49-7.43 (m, 1H), 7.17-7.13 (m, 2H), |
| | 7.05-7.03 (m, 1H), 6.57 (d, J = 8.8 Hz, 2H), 4.53-4.50 (m, |
| | 2H), 4.354.33 (m, 2H), 4.20-4.11 (m, 5H), 3.79-3.62 (m, |
| | 3H), 3.51-3.31 (m, 6H), 3.13-3.03 (m, 2H), 2.78-2.76 (m, |
| | 1H), 2.48 (br, 2H), 2.05-1.98 (m, 4H), 1.82-1.76 (m, 2H), |
| | 1.64-1.62 (m, 3H), 1.48-1.45 (m, 1H). |
| 100 | ¹H NMR (400 MHz, MeOD) δ 8.38 (t, J = 1.6 Hz, 1H), |
| | 8.14-7.97 (m, 2H), 7.82-7.31 (m, 4H), 7.24-6.91 (m, |
| | 3H), 6.72-6.48 (m, 2H), 4.68-4.04 (m, 6H), 3.62 (ddd, J = |
| | 53.1, 41.4, 29.8 Hz, 7H), 3.24-2.32 (m, 7H), 2.27-0.54 |
| | (m, 14H). |
| 101 | ¹H NMR (400 MHz, MeOD) δ 8.58-7.87 (m, 4H), 7.82- |
| | 7.39 (m, 4H), 7.28-6.98 (m, 3H), 6.48 (d, J = 8.9 Hz, |
| | 1H), 4.73-3.95 (m, 6H), 3.81-3.37 (m, 7H), 3.23-2.30 |
| | (m, 10H), 2.22-0.64 (m, 14H). |
| 102 | ¹H NMR (400 MHz, Methanol-d₄) δ 8.07-7.86 (m, 4H), |
| | 7.59 (dd, J = 8.6, 2.1 Hz, 1H), 7.52 (dd, J = 12.1, 2.0 Hz, |
| | 1H), 7.45 (q, J = 7.7 Hz, 1H), 7.13 (q, J = 6.9, 5.7 Hz, |
| | 2H), 7.02 (d, J = 7.9 Hz, 1H), 6.55 (t, J = 8.6 Hz, 1H), |
| | 4.52 (s, 2H), 4.35 (d, J = 9.2 Hz, 1H), 4.30-4.19 (m, 2H), |
| | 4.14 (d, J = 15.7 Hz, 1H), 3.88-3.70 (m, 3H), 3.57-3.39 |
| | (m, 2H), 3.39-3.32 (m, 2H), 3.27 (m, 4H), 3.22-3.04 |
| | (m, 1H), 2.94 (dt, J = 24.8, 12.3 Hz, 2H), 2.77 (d, J = 9.3 |
| | Hz, 1H), 2.67-2.35 (m, 2H), 2.13-1.90 (m, 4H), 1.79 (s, |
| | 2H), 1.65 (d, J = 12.5 Hz, 3H), 1.49-1.22 (m, 2H), 0.95 |
| | (m, 1H). |
| 103 | ¹H NMR (400 MHz, CD₃OD) δ 7.98-7.91 (m, 4H), 7.58 |
| | (dd, J = 8.4, 2.0 Hz, 1H), 7.51 (dd, J = 12.0, 2.0 Hz, 1H), |
| | 7.48-7.42 (m, 1H), 7.15-7.11 (m, 2H), 7.05-7.03 (m, 1H), |
| | 6.54 (t, J = 8.4 Hz, 1H), 4.51-4.50 (m, 2H), 4.36-4.23 (m, |
| | 4H), 4.14-4.10 (m, 1H), 3.83-3.75 (m, 3H), 3.49-3.30 (m, |
| | 8H), 3.18-3.13 (m, 1H)7 3.01-2.88 (m, 5H), 2.77-2.75 (m, |
| | 1H), 2.54-2.43 (m, 2H), 2.06-1.86 (m, 5H), 1.78-1.62 (m, |
| | 5H), 1.45-1.42 (m, 1H). |
| 106 | ¹H NMR (400 MHz, MeOD) δ 8.05-7.96 (m, 4H), 7.62 |
| | (ddd, J = 13.8, 10.2, 2.0 Hz, 2H), 7.47 (dd, J = 14.9, 8.2 |
| | Hz, 1H), 7.15 (dd, J = 8.3, 6.5 Hz, 2H), 7.07 (d, J = 7.3 |
| | Hz, 1H), 6.65 (t, J = 8.6 Hz, 1H), 4.52-4.46 (m, 2H), 4.41- |
| | 4.09 (m, 7H), 3.95-3.39 (m, 7H), 3.23-3.04 (m, 2H), |
| | 2.82-2.75 (d, J = 8.7 Hz, 1H), 2.59-2.37 (m, 2H), 2.17- |
| | 1.00 (m, 13H). |
| 107 | ¹H NMR (400 MHz, CD₃OD) δ 7.96-7.90 (m, 4H), 7.79- |
| | 7.76 (m, 2H), 7.49-7.43 (m, 1H), 7.17-7.13 (m, 2H), 7.05- |
| | 7.03 (m, 1H), 6.58-6.55 (m, 2H), 4.51-4.50 (m, 2H), 4.35- |
| | 4.33 (m, 2H), 4.21-4.10 (m, 5H), 3.79-3.67 (m, 3H), 3.57- |
| | 3.51 (m, 2H), 3.44-3.31 (m, 4H), 3.14-3.05 (m, 2H), 2.78- |
| | 2.76 (m, 1H), 2.48 (br, 2H), 2.07-1.98 (m, 4H), 1.85-1.78 |
| | (m, 2H), 1.64-1.62 (m, 3H), 1.50-1.47 (n, 1H), 1.21 (t, J = |
| | 7.6 Hz, 1H). |
| 115 | ¹H NMR (400 MHz, Methanol-d₄) δ 9.06 (dd, J = 2.3, 0.8 |
| | Hz, 1H), 8.39 (dd, J = 8.3, 2.3 Hz, 1H), 8.21 (dd, J = 8.2, |
| | 0.8 Hz, 1H), 7.88-7.79 (m, 2H), 7.45 (q, J = 7.8 Hz, 1H), |
| | 7.14 (t, J = 8.1 Hz, 2H), 7.04 (d, J = 8.0 Hz, 1H), 6.63- |
| | 6.53 (m, 2H), 4.51 (d, J = 10.3 Hz, 2H), 4.34 (d, J = 9.8 |
| | Hz, 2H), 4.28-4.04 (m, 5H), 3.83-3.67 (m, 3H), 3.55 (t, |
| | J = 12.2 Hz, 2H), 3.51-3.33 (m, 2H), 3.21-3.01 (m, |
| | 2H), 2.77 (d, J = 9.5 Hz, 1H), 2.48 (d, J = 30.1 Hz, 2H), |
| | 2.02 (dd, J = 24.1, 11.8 Hz, 5H), 1.81 (m, 2H), 1.71-1.41 |
| | (m, 4H), 1.11 (s, 1H). |
| 121 | ¹H NMR (400 MHz, Methanol-d₄) δ 9.09 (dd, J = 2.3, 0.8 |
| | Hz, 1H), 8.43 (dd, J = 8.3, 2.3 Hz, 1H), 8.23 (dd, J = 8.3, |
| | 0.8 Hz, 1H), 7.73-7.58 (m, 2H), 7.46 (q, J = 7.6 Hz, 1H), |
| | 7.19-7.08 (m, 2H), 7.04 (d, J = 7.8 Hz, 1H), 6.67 (t, J = |
| | 8.5 Hz, 1H), 4.53 (s, 2H), 4.33 (d, J = 20.5 Hz, 6H), 4.14 |
| | (d, J = 15.7 Hz, 1H), 3.82-3.65 (m, 3H), 3.55 (t, J = 12.2 |
| | Hz, 2H), 3.50-3.37 (m, 1H), 3.29-3.26 (m, 3H), 3.20- |
| | 3.02 (m, 2H), 2.84-2.70 (m, 1H), 2.47 (s, 2H), 2.15- |
| | 1.92 (m, 4H), 1.80 (s, 2H), 1.66 (d, J = 12.6 Hz, 3H), 1.49 |
| | (d, J = 12.9 Hz, 1H), 1.31 (d, J = 17.6 Hz, 1H), 1.09 (s, |
| | 1H). |
| 158 | ¹H NMR (400 MHz, Methanol-d₄) δ 7.67 (d, J = 8.6 Hz, |
| | 1H), 7.47(m, 1H), 7.19-7.11 (m, 2H), 7.07 (d, J = 7.9 Hz, |
| | 1H), 6.80 (d, J = 2.3 Hz, 1H), 6.66 (dd, J = 8.6, 2.3 Hz, |
| | 1H), 4.51 (s, 2H), 4.43 (s, 2H), 4.41-4.20 (m, 6H), 4.15 |
| | (d, J = 15.6 Hz, 1H), 3.86-3.71 (m, 3H), 3.59 (t, J = 11.8 |
| | Hz, 2H), 3.53-3.41 (m, 1H), 3.25-3.07 (m, 2H), 2.94 (s, |
| | 6H), 2.85-2.72 (m, 1H), 2.61-2.34 (m, 2H), 2.13-1.93 |
| | (m, 4H), 1.78 (s, 1H), 1.72-1.48 (m, 5H), 1.21 (s, 1H). |
| 159 | ¹H NMR (400 MHz, Methanol-d₄) δ 8.00-7.90 (m, 2H), |
| | 7.81-7.72 (m, 2H), 7.58-7.51 (m, 2H), 7.45 (td, J = 8.2, |
| | 6.3 Hz, 1H), 7.14 (td, J = 8.3, 1.5 Hz, 2H), 7.04 (d, J = 8.0 |
| | Hz, 1H), 6.60-6.50 (m, 2H), 4.50 (d, J = 10.2 Hz, 2H), |
| | 4.34 (d, J = 9.5 Hz, 1H), 4.25-4.04 (m, 5H), 3.84-3.64 |
| | (m, 6H), 3.64-3.37 (m, 5H), 3.29 (s, 4H), 3.23-3.01 (m, |
| | 2H), 2.77 (d, J = 9.6 Hz, 1H), 2.61-2.32 (m, 2H), 2.02 |
| | (dd, J = 23.9, 11.6 Hz, 5H), 1.91-1.71 (m, 2H), 1.71- |
| | 1.35 (m, 8H), 1.13 (s, 1H). |
| 199 | ¹H NMR (400 MHz, Methanol-d₄) δ 7.69 (d, J = 8.9 Hz, |
| | 2H), 7.46 (q, J = 7.6 Hz, 1H), 7.18-7.09 (m, 4H), 7.04 (d, |
| | J = 7.9 Hz, 1H), 4.59-4.28 (m, 6H), 4.13 (d, J = 15.6 Hz, |
| | 1H), 3.77 (d, J = 15.6 Hz, 1H), 3.66 (t, J = 15.5 Hz, 2H), |
| | 3.59-3.49 (m, 2H), 3.47-3.40 (m, 1H), 3.06-2.90 (m, |
| | 2H), 2.82-2.69 (m, 2H), 2.59-2.40 (m, 3H), 2.06-1.90 |
| | (m, 4H), 1.85-1.71 (m, 2H), 1.71-1.54 (m, 4H), 1.41 (d, |
| | J = 12.7 Hz, 1H), 1.06 (s, 1H). |
| 205 | ¹H NMR (400 MHz, Methanol-d₄) δ 7.70 (d, J = 9.0 Hz, |
| | 2H), 7.46 (q, J = 7.6 Hz, 1H), 7.15 (t, J = 7.8 Hz, 2H), |
| | 7.08 (d, J = 8.9 Hz, 2H), 7.03 (s, 1H), 4.62-4.46 (m, 2H), |
| | 4.36 (dd, J = 18.8, 9.2 Hz, 2H), 4.12 (d, J = 14.1 Hz, 2H), |
| | 3.77 (d, J = 15.8 Hz, 1H), 3.55 (t, J = 11.5 Hz, 2H), 3.28 |
| | (s, 3H), 3.04 (dd, J = 24.1, 12.1 Hz, 3H), 2.93 (t, J = 12.9 |
| | Hz, 3H), 2.84-2.73 (m, 2H), 2.62-2.39 (m, 4H), 2.16- |
| | 1.95 (m, 6H), 1.86-1.58 (m, 7H), 1.48-1.38 (m, 1H), |
| | 1.21-1.12 (m, 2H), 1.04-0.96 (m, 2H). |
| 210 | ¹H NMR (400 MHz, Methanol-d₄) δ 8.02-7.88 (m, 4H), |
| | 7.72 (d, J = 8.8 Hz, 2H), 7.46 (q, J = 7.7 Hz, 1H), 7.19- |
| | 7.10 (m, 2H), 7.05 (d, J = 8.0 Hz, 1H), 6.62 (d, J = 8.9 Hz, |
| | 2H), 4.59-4.45 (m, 2H), 4.42-4.27 (m, 2H), 4.14 (d, J = |
| | 15.6 Hz, 1H), 3.79 (d, J = 15.9 Hz, 1H), 3.66 (d, J = 12.8 |
| | Hz, 1H), 3.59 (t, J = 8.6 Hz, 2H), 3.52-3.39 (m, 3H), |
| | 3.19 (d, J = 6.9 Hz, 2H), 3.10-2.93 (m, 3H), 2.91 (s, 3H), |
| | 2.83-2.70 (m, 2H), 2.62-2.36 (m, 3H), 2.32-2.19 (m, |
| | 2H), 2.14-1.92 (m, 5H), 1.91-1.73 (m, 3H), 1.71-1.59 |
| | (m, 3H), 1.53-1.42 (m, 1H), 1.16-1.03 (m, 1H). |
| 251 | ¹H NMR (400 MHz, CD₃OD) δ 7.43 (d, J = 8.4 Hz, 1H), |
| | 7.39-7.33 (m, 2H), 7.26-7.19 (m, 2H), 6.99 (td, J = 8.4, 2.4 |
| | Hz, 1H), 6.43 (d, J = 2.0 Hz, 1H), 6.35 (dd, J = 8.4, 2.0 |
| | Hz, 1H), 5.23-5.03 (m, 1H), 4.11-4.06 (m, 3H), 3.81-3.73 |
| | (m, 4H), 3.70-3.59 (in, 7H), 3.51 (s, 3H), 3.37-3.28 (m, |
| | 2H), 3.02-2.92 (m, 3H), 2.71-2.65 (m, 2H), 2.51-2.45 (m, |
| | 1H), 2.05-1.80 (m, 5H)7 1.67-1.64 (m, 1H), 1.54-1.30 (m, |
| | 7H). |
| 254 | ¹H NMR (400 MHz, Methanol-d₄) δ 7.87 (d, J = 8.7 Hz, |
| | 1H), 7.40 (q, J = 7.7 Hz, 1H), 7.36-7.17 (m, 2H), 7.04 (t, |
| | J = 8.2 Hz, 1H), 6.75-6.58 (m, 2H), 5.41 (d, J = 62.7 Hz, |
| | 1H), 4.71 (s, 2H), 4.67-4.36 (m, 4H), 4.25 (td, J = 8.2, |
| | 3.4 Hz, 2H), 4.11 (d, J = 6.9 Hz, 1H), 3.81 (dd, J = 14.5, |
| | 9.1 Hz, 4H), 3.68 (s, 3H), 3.60-3.39 (m, 7H), 3.13 (s, |
| | 3H), 3.00 (t, J = 12.2 Hz, 1H), 2.87 (t, J = 12.3 Hz, 1H), |
| | 2.52 (d, J = 8.1 Hz, 1H), 2.34-2.16 (m, 2H), 1.94 (dt, J = |
| | 27.9, 12.5 Hz, 2H), 1.71 (q, J = 13.2 Hz, 1H), 1.61-1.20 |
| | (m, 7H). |
| 264 | ¹H NMR (400 MHz, Methanol-d₄) δ 7.45 (d, J = 8.5 Hz, |
| | 1H), 7.41-7.22 (m, 3H), 6.95 (t, J = 8.0 Hz, 1H), 6.45 (d, |
| | J = 2.3 Hz, 1H), 6.38 (dd, J = 8.6, 2.3 Hz, 1H), 5.14 (dp, |
| | J = 57.4, 5.0 Hz, 1H), 4.14 (s, 2H), 4.00 (s, 1H), 3.77 (s, |
| | 2H), 3.74-3.60 (m, 4H), 3.55 (s, 2H), 3.40-3.33 (m, |
| | 1H), 3.18-2.80 (m, 8H), 2.54 (q, J = 35.0, 34.4 Hz, 7H), |
| | 2.08 (s, 2H), 1.83 (s, 3H), 1.56 (d, J = 27.7 Hz, 8H), 1.32 |
| | (d, J = 26.0 Hz, 3H), 1.17 (s, 3H). |
| 265 | ¹H NMR (400 MHz, CD₃OD) δ 7.40-7.33 (m, 2H), 7.25- |
| | 7.17 (m, 2H), 7.01 (td, J = 8.4, 2.4 Hz, 1H), 6,47 (t, J = 8.4 |
| | Hz, 1H), 5.18-4.98 (m, 1H), 4.25-4.20 (m, 2H), 4.10-4.04 |
| | (m, 1H), 3.89-3.86 (m, 1H), 3.81-3.64 (m, 7H), 3.44 (s, |
| | 3H), 3.39-3.30 (m, 2H), 3.00-2.90 (m, 3H), 2.64-2.58 (m, |
| | 3H), 2.02-1.94 (m, 5H), 1.92-1.73 (m, 4H), 1.65-1.55 (m, |
| | 2H), 1.52-1.30 (m, 5H). |
| 266 | ¹H NMR (400 MHz, CD₃OD) δ 7.37-7.32 (m, 1H), 7.26- |
| | 7.15 (m, 3H), 7.01-6.96 (m, 1H), 6.47 (d, J = 8.8 Hz, 1H), |
| | 5.21-5.03 (m, 1H), 4.22-4.17 (m, 2H), 4.08-4.02 (m, 1H), |
| | 3.87-3.83 (m, 1H), 3.77-3.60 (m, 7H), 3.42 (s, 3H), 3.35- |
| | 3.26 (m, 2H), 2.98-2.88 (m, 3H), 2.60-2.56 (m, 3H), 2.00- |
| | 1.92 (m, 5H), 1.88-1.71 (m, 4H), 1.63-1.52 (m, 2H), 1.49- |
| | 1.28 (m, 5H). |
| 271 | ¹H NMR (400 MHz, Methanol-d₄) δ 7.44 (d, J = 8.5 Hz, |
| | 1H), 7.36 (q, J = 7.6 Hz, 1H), 7.23 (dd, J = 19.2, 10.0 Hz, |
| | 2H), 7.04-6.94 (m, 1H), 6.48 (d, J = 2.3 Hz, 1H), 6.37 |
| | (dd, J = 8.5, 2.4 Hz, 1H), 4.08 (td, J = 7.9, 3.0 Hz, 3H), |
| | 3.76 (d, J = 3.4 Hz, 2H), 3.65 (s, 3H), 3.64-3.57 (m, 2H), |
| | 3.52 (d, J = 13.2 Hz, 4H), 3.05-2.86 (m, 3H), 2.64 (d, J = |
| | 7.0 Hz, 2H), 2.47 (q, J = 8.6 Hz, 1H), 2.27 (s, 6H), 2.09 |
| | 1.77 (m, 5H), 1.65 (d, J = 13.2 Hz, 1H), 1.58-1.13 (m, |
| | 8H). |
| 272 | ¹H NMR (400 MHz, MeOD) δ 7.42-7.37 (m, 2H), 7.25 |
| | (dd, J = 17.4, 9.8 Hz, 2H), 7.03 (dd, J = 9.1, 7.0 Hz, 1H), |
| | 6.53-6.49 (m, 1H), 4.63 (s, 3H), 4.27 (d, J = 8.1 Hz, 2H), |
| | 4.11 (d, J = 6.4 Hz, 1H), 3.89-3.76 (m, 3H), 3.71-3.61 |
| | (m, 4H), 3.52 (s, 2H), 3.30-2.76 (m, 6H), 2.51-2.49 (m, |
| | 2H), 2.33 (s, 6H), 2.06-1.75 (m, 4H), 1.62-1.25 (m, 7H). |
| 273 | ¹H NMR (400 MHz, Methanol-d₄) δ 7.38 (td, J = 8.0, 6.4 |
| | Hz, 1H), 7.32-7.15 (m, 3H), 7.00 (td, J = 8.3, 2.4 Hz, |
| | 1H), 6.57 (d, J = 8.8 Hz, 1H), 4.30-4.19 (m, 2H), 4.10 (q, |
| | J = 6.9 Hz, 1H), 3.88-3.71 (m, 4H), 3.66 (s, 3H), 3.56 (s, |
| | 2H), 3.51 (s, 3H), 3.21-2.98 (m, 3H), 2.90 (s, 2H), 2.48 |
| | (q, J = 8.2 Hz, 1H), 2.30 (s, 6H), 2.15-1.94 (m, 2H), 1.85 |
| | (d, J = 6.2 Hz, 1H), 1.74 (d, J = 13.5 Hz, 1H), 1.53 (q, J = |
| | 12.3, 11.1 Hz, 3H), 1.46-1.17 (m, 6H). |
| 274 | ¹H NMR (400 MHz, Methanol-d₄) δ 7.43-7.33 (m, 2H), |
| | 7.28-7.16 (m, 2H), 7.02 (td, J = 8.3, 2.4 Hz, 1H), 6.51 (t, |
| | J = 8.6 Hz, 1H), 4.32-4.22 (m, 2H), 4.07 (q, J = 7.3 Hz, |
| | 1H), 3.92-3.76 (m, 4H), 3.69 (s, 2H), 3.42 (s, 3H), 3.29- |
| | 3.23 (m, 1H), 3.21-3.02 (m, 3H), 2.72-2.47 (m, 3H), |
| | 2.35 (s, 6H), 2.11-2.00 (m, 2H), 1.98 (s, 3H), 1.94-1.83 |
| | (m, 2H), 1.68-1.26 (m, 7H), 1.25-1.08 (m, 1H). |
| 275 | ¹H NMR (400 MHz, Methanol-d₄) δ 7.37 (q, J = 7.9 Hz, |
| | 1H), 7.30 (d, J = 12.3 Hz, 1H), 7.26-7.14 (m, 2H), 7.02 |
| | (td, J = 8.3, 2.4 Hz, 1H), 6.57 (d, J = 8.8 Hz, 1H), 4.32- |
| | 4.20 (m, 2H), 4.06 (q, J = 7.3 Hz, 1H), 3.93-3.73 (m, |
| | 4H), 3.59 (s, 2H), 3.42 (s, 3H), 3.25-2.82 (m, 6H), 2.57 |
| | (q, J = 8.6 Hz, 1H), 2.32 (s, 6H), 2.06-2.00 (m, 1H), 1.98 |
| | (s, 3H), 1.93-1.80 (m, 2H), 1.68-1.26 (m, 8H), 1.22- |
| | 1.06 (m, 1H). |
| 278 | ¹H NMR (400 MHz, Methanol-d₄) δ 7.54-7.49 (m, 1H), |
| | 7.44 (m, 1H), 7.13 (d, J = 8.7 Hz, 3H), 7.05 (d, J = 7.9 Hz, |
| | 1H), 6.66 (t, J = 8.7 Hz, 1H), 4.49 (dd, J = 10.2, 2.1 Hz, |
| | 4H), 4.42-4.24 (m, 4H), 4.12 (d, J = 15.6 Hz, 1H), 3.94 |
| | (t, J = 7.4 Hz, 2H), 3.78 (d, J = 15.4 Hz, 2H), 3.60-3.46 |
| | (m, 3H), 3.40 (d, J = 7.1 Hz, 2H), 2.97 (s, 5H), 2.82-2.71 |
| | (m, 2H), 2.60-2.34 (m, 3H), 2.02 (dd, J = 24.2, 12.4 Hz, |
| | 7H), 1.83-1.71 (m, 2H), 1.71-1.54 (m, 4H), 1.53-1.36 m, |
| | 2H), 1.10 (s, 2H). |
| 283 | ¹H NMR (400 MHz, Methanol-d₄) δ 7.55 (d, J = 12.1 Hz, |
| | 1H), 7.46 (q, J = 8.0 Hz, 1H), 7.15 (t, J = 8.2 Hz, 2H), |
| | 7.05 (d, J = 8.0 Hz, 1H), 6.86 (d, J = 8.4 Hz, 1H), 4.52 (s, |
| | 2H), 4.39 (d, J = 18.8 Hz, 8H), 4.14 (d, J = 15.6 Hz, 1H), |
| | 3.77 (dd, J = 18.0, 10.3 Hz, 4H), 3.56 (t, J = 12.2 Hz, |
| | 2H), 3.45 (s, 1H), 3.20-3.02 (m, 2H), 2.93 (s, 6H), |
| | 2.84-2.72 (m, 2H), 2.59-2.37 (m, 2H), 2.11-1.93 (m, 5H), |
| | 1.91-1.73 (m, 3H), 1.66 (d, J = 13.3 Hz, 3H), 1.51 (d, J = |
| | 13.2 Hz, 1H), 1.13 (s, 1H). |
| 287 | ¹H NMR (400 MHz, Methanol-d₄) δ 7.52-7.45 (m, 1H), |
| | 7.36 (d, J = 7.1 Hz, 2H), 7.15-6.95 (m, 2H), 6.76 (d, J = |
| | 8.3 Hz, 1H), 4.57-4.40 (m, 1H), 4.37 (d, J = 13.6 Hz, |
| | 2H), 3.95 (s, 4H), 3.88-3.38 (m, 11H), 3.17-2.93 (m, |
| | 6H), 2.93 (s, 4H), 2.50 (s, 3H), 2.28-1.78 (m, 7H), 1.45 |
| | (d, J = 113.2 Hz, 7H). |
| 290 | ¹H NMR (400 MHz, Methanol-d₄) δ 7.57 (d, J = 8.6 Hz, |
| | 1H), 7.54-7.42 (m, 1H), 7.17 (t, J = 8.0 Hz, 2H), 7.07 |
| | (d, J = 8.0 Hz, 1H), 6.52 (dd, J = 8.6, 2.4 Hz, 1H), 6.40 |
| | (d, J = 2.3 Hz, 1H), 4.54 (s, 2H), 4.38 (d, J = 10.4 Hz, |
| | 2H), 4.17 (q, J = 8.1 Hz, 3H), 3.86-3.71 (m, 4H), 3.64- |
| | 3.45 (m, 2H), 3.41 (d, J = 7.1 Hz, 3H), 3.13 (s, 5H), 3.09- |
| | 2.87 (m, 5H), 2.87-2.71 (m, 1H), 2.51 (d, J = 30.8 Hz, |
| | 2H), 2.05 (dd, J = 29.0, 12.4 Hz, 5H), 1.84 (d, J = 28.6 |
| | Hz, 2H), 1.67 (d, J = 13.5 Hz, 4H), 1.55-1.36 (m, 1H). |
| 304 | ¹H NMR (400 MHz, CD₃OD) δ 8.06 (s, 1H), 7.78 (s, |
| | 1H), 7.51-7.36 (m, 4H), 7.10-7.05 (m, 1H), 6.66 (t, J = |
| | 8.4 Hz, 1H), 527 (s, 2H), 449 (s, 2H), 4.37-4.32 (m, |
| | 2H), 4.26-4.24 (m, 1H), 3.97-3.92 (m, 2H), 3.54-3.40 (m, |
| | 7H), 3.28-3.23 (m, 1H), 3.02-2.91 (m, 9H), 2.86-2.80 (m, |
| | 1H), 2.52-2.46 (m, 1H), 2.20 (d, J = 14.0 Hz, 1H), 1.96- |
| | 1.80 (m, 2H) 1.58-1.12 (m, 6H). |
| 336 | ¹H NMR (400 MHz, MeOD) δ 7.65 (d, J = 8.8 Hz, 2H), |
| | 7.47-7.41 (m, 1H), 7.34 (d, J = 7.6 Hz, 1H), 7.25 (d, J = |
| | 10.8 Hz, 1H), 7.16-7.12 (m, 1H), 6.49 (d, J = 8.8 Hz, 2H), |
| | 4.15-4.11 (m, 2H), 3.89-3.84 (m 1H), 3.73-3.70 (m, 2H), |
| | 3.56 (t, J = 11 Hz, 2H), 3.43 (s, 3H), 3.42-3.34 (m, 2H), |
| | 3.23-3.17 (m, 1H), 3.07-3.04 (m, 2H), 2.88-2.82 (m, 1H), |
| | 2.59-2.53 (m, 1H), 2.46 (t, J = 12.0 Hz, 1H), 2.25-2.21 (m, |
| | 1H), 2.16-2.14 (m, 1H), 2.00-1.96 (m, 1H), 1.85-1.78 (m, |
| | 1H), 1.74-1.53 (m, 5H), 1.45-1.36 (m, 1H), 1.16-1.12 (m, |
| | 2H), 1.02-0.96 (m, 2H); ¹³C NMR (100 MHz, MeOD) δ |
| | 166.0, 163.6, 163.3, 162.9, 162.5, 162.2, 158.9, 156.4, |
| | 138.0, 137.9, 132.3, 132.2, 131.1, 129.6, 126.5, 122.8, |
| | 122.5, 119.9, 117.7, 117.4, 117.3, 117.1, 114.1, 112.4, |
| | 61.8, 57.7, 57.1, 56.7, 54.4, 53.1, 41.9, 35.9, 35.0, 30.9, |
| | 28.4, 27.6, 26.5, 24.7, 6.9. |
| 339 | ¹H NMR (400 MHz, Methanol-d₄) δ 7.65 (d, J = 8.8 Hz, |
| | 2H), 7.51-7.39 (m, 1H), 7.18-6.95 (m, 3H), 6.50 (d, J = |
| | 8.9 Hz, 2H), 4.70-4.51 (m, 1H), 4.48-4.32 (m, 1H), |
| | 4.32-4.18 (m, 1H), 4.13 (t, J = 7.8 Hz, 4H), 3.99 (t, J = |
| | 9.5 Hz, 1H), 3.88-3.67 (m, 3H), 3.57-3.44 (m, 3H), |
| | 3.43-3.34 (m, 2H), 3.26-3.15 (m, 2H), 3.06-2.86 (m, |
| | 3H), 2.83-2.69 (m, 1H), 2.56 (tt, J = 7.9, 4.8 Hz, 1H), |
| | 2.15-1.91 (m, 5H), 1.91-1.53 (m, 6H), 1.44 (q, J = 12.5 |
| | Hz, 1H), 1.29 (dd, J = 17.5, 6.9 Hz, 3H), 1.17-1.11 (m, |
| | 2H), 1.02-0.96 (m, 2H). |
| 390 | ¹H NMR (400 MHz, MeOD) δ 7.74-7.63 (m, 2H), 7.46 |
| | (dd, J = 14.6, 8.1 Hz, 1H), 7.32-7.18 (m, 2H), 7.12-7.08 |
| | (m, 1H), 6.55 (d, J = 8.8 Hz, 2H), 6.14 (t, J = 55.5 Hz, |
| | 1H), 4.20 (dd, J = 8.0, 6.2 Hz, 2H), 3.99-3.92 (m, 1H), |
| | 3.79-3.73 (m, 2H), 3.62-3.44 (m, 9H), 3.29-3.17 (m, |
| | 2H), 3.03-2.86 (m, 2H), 2.61-2.55 (m, 2H), 2.36-1.83 |
| | (m, 6H), 1.67-1.27 (m, 8H), 1.20-1.11 (m, 2H), 1.04- |
| | 0.99 (qd, J = 5.4, 1.1 Hz, 2H). |

### Example 17

### Menin Binding Affinity

A fluorescence polarization (FP) competitive binding assay was used to determine the binding affinities of representative Compounds of the Disclosure. A FAM labeled fluorescent probe was designed and synthesized based on a MLL1 peptide (FAM-MM2). Equilibrium dissociation constant (KA) value of FAM-MM2 to menin protein was determined from protein saturation experiments by monitoring the total fluorescence polarization of mixtures composed with the fluorescent probe at a fixed concentration and the protein with increasing concentrations up to full saturation. Serial dilutions of the protein were mixed with FAM-MM2 to a final volume of 200 μl in the assay buffer (PBS with 0.02% Bovine γ-Globulin and 4% DMSO. 0.01% Triton X-100 was added right before assays). Final FAM-MM2 concentration was 2 nM. Plates were incubated at room temperature for 30 minutes with gentle shaking to assure equilibrium. FP values in millipolarization units (mP) were measured using the Infinite M-1000 plate reader (Tecan U.S., Research Triangle Park, NC) in Microfluor 1 96-well, black, v-bottom plates (Thermo Scientific, Waltham, Mass.) at an excitation wavelength of 485 nm and an emission wavelength of 530 nm. K_{d} value of FAM-MM2, which was calculated by fitting the sigmoidal dose-dependent FP increases as a function of protein concentrations using Graphpad Prism 6.0 software (Graphpad Software, San Diego, Calif.), was determined as 1.4 nM.

The IC₅₀ of representative Compounds of the Disclosure were determined in a competitive binding experiment. See Tables 4-6. Mixtures of 5 μl of the tested compounds in DMSO and 195 μl of preincubated protein/probe complex solution in the assay buffer were added into assay plates which were incubated at room temperature for 30 minutes with gentle shaking. Final concentration of the menin protein was 4 nM, and final probe concentration is 2 nM. Negative controls containing protein/probe complex only (equivalent to 0% inhibition), and positive controls containing only free probes (equivalent to 100% inhibition), were included in each assay plate. FP values were measured as described above. IC₅₀ values were determined by nonlinear regression fitting of the competition curves.

TABLE 4

| | Menin Binding |
|---|---|
| Cpd. No. | Affinity IC₅₀ (μM) |
| 1 | 0.051 |
| 2 | 0.020 |
| 3 | >10 |
| 4 | 0.304 |
| 5 | 0.006 |
| 6 | >10 |
| 7 | >10 |
| 10 | 3.836 |
| 11 | 0.030 |
| 12 | 0.042 |
| 13 | 0.904 |
| 14 | >10 |
| 15 | >10 |
| 16 | >10 |
| 17 | >10 |
| 18 | >10 |
| 19 | >10 |
| 20 | >10 |
| 21 | >10 |
| 22 | >10 |
| 23 | 0.608 |
| 24 | 1.578 |
| 25 | >10 |
| 26 | 0.002 |
| 27 | 1.689 |
| 28 | >10 |
| 29 | >10 |
| 30 | >10 |
| 31 | >10 |
| 32 | 5.647 |
| 33 | 0.002 |
| 34 | 0.003 |
| 35 | 0.003 |
| 36 | 0.002 |
| 37 | 0.002 |
| 38 | 0.038 |
| 39 | 0.003 |
| 40 | 0.004 |
| 41 | 0.003 |
| 42 | 0.003 |
| 43 | 0.001 |
| 44 | 0.001 |
| 45 | 0.001 |
| 46 | 0.002 |
| 47 | 0.002 |
| 48 | 0.002 |
| 49 | 0.002 |
| 50 | 2.201 |
| 51 | >10 |
| 52 | 0.003 |
| 53 | 0.004 |
| 54 | 0.006 |
| 55 | 0.003 |
| 56 | 0.003 |
| 57 | 0.002 |
| 58 | 0.003 |
| 59 | 0.002 |
| 60 | 0.002 |
| 61 | 0.002 |
| 62 | 0.003 |
| 63 | 0.003 |
| 64 | 0.003 |
| 65 | 0.002 |
| 66 | >10 |
| 67 | 1.285 |
| 68 | 0.002 |
| 69 | 0.002 |
| 70 | 2.509 |
| 71 | 2.728 |
| 72 | 0.002 |
| 73 | 0.001 |
| 74 | 0.004 |
| 75 | 0.005 |
| 76 | 0.002 |
| 77 | 0.002 |
| 78 | 0.009 |
| 79 | 0.005 |
| 80 | 0.002 |
| 81 | 0.002 |
| 82 | 0.001 |
| 83 | 0.001 |

TABLE 5

| | Menin Binding |
|---|---|
| Cpd No. | Affinity IC₅₀ (μM) |
| 84 | 0.12 |
| 85 | 0.004 |
| 86 | 0.006 |
| 87 | 0.003 |
| 88 | 0.002 |
| 89 | 0.002 |
| 90 | 0.018 |
| 91 | 0.006 |
| 92 | 0.002 |
| 93 | 0.002 |
| 94 | 0.006 |
| 95 | 0.002 |
| 96 | 0.038 |
| 97 | >10 |
| 98 | 0.002 |
| 99 | 0.017 |
| 100 | 0.001 |
| 101 | 0.001 |
| 102 | 0.001 |
| 103 | 0.001 |
| 104 | 0.002 |
| 105 | 0.002 |
| 106 | 0.001 |
| 107 | 0.002 |
| 108 | 0.002 |
| 109 | 0.002 |
| 110 | 0.002 |
| 111 | 0.001 |
| 112 | 0.002 |
| 113 | 0.002 |
| 114 | 0.002 |
| 115 | 0.001 |
| 116 | 0.001 |
| 117 | 0.002 |
| 118 | 0.001 |
| 119 | 0.002 |
| 120 | 0.002 |
| 121 | 0.001 |
| 122 | 0.002 |
| 123 | 0.001 |
| 124 | 0.002 |
| 125 | 0.002 |
| 126 | 0.002 |
| 127 | 0.001 |
| 128 | 0.002 |
| 129 | 0.002 |
| 130 | 0.002 |
| 131 | 0.002 |
| 132 | 0.002 |
| 133 | 0.002 |
| 134 | 0.003 |
| 135 | 0.002 |
| 136 | 0.003 |
| 137 | 0.003 |
| 138 | 0.001 |
| 139 | 0.001 |
| 140 | 0.002 |
| 141 | 0.001 |
| 142 | 0.011 |
| 143 | 0.002 |
| 144 | 0.003 |
| 145 | 0.002 |
| 146 | 0.050 |
| 147 | 0.002 |
| 148 | 0.004 |
| 149 | 0.002 |
| 150 | 0.078 |
| 151 | 0.034 |
| 152 | 0.004 |
| 153 | 0.008 |
| 154 | 0.004 |
| 155 | 0.003 |
| 156 | 0.001 |
| 157 | 0.001 |
| 158 | 0.002 |
| 159 | 0.002 |
| 160 | 0.003 |
| 161 | 0.004 |
| 162 | 0.003 |
| 163 | 0.003 |
| 164 | 0.002 |
| 165 | 0.005 |
| 166 | 0.002 |
| 167 | 0.008 |
| 168 | 0.004 |
| 169 | 0.001 |
| 170 | 0.007 |
| 171 | 0.003 |
| 172 | 0.003 |
| 173 | 0.007 |
| 174 | 0.007 |
| 175 | 0.004 |
| 176 | 0.004 |
| 177 | 0.006 |
| 178 | 0.005 |
| 179 | 0.001 |
| 180 | 0.002 |
| 181 | 0.001 |
| 182 | 0.002 |
| 183 | 0.002 |
| 184 | 0.004 |
| 185 | 0.003 |
| 186 | 0.003 |
| 187 | 0.003 |
| 188 | 0.003 |
| 189 | 0.017 |
| 190 | 0.002 |
| 191 | 0.001 |
| 192 | 0.001 |
| 193 | 0.001 |
| 194 | 0.002 |
| 195 | 0.046 |
| 196 | 0.002 |
| 197 | 0.097 |
| 198 | 0.008 |
| 199 | 0.083 |
| 200 | 0.006 |
| 201 | 0.032 |
| 202 | 0.444 |
| 203 | 0.002 |
| 204 | 0.004 |
| 205 | 0.021 |
| 206 | >10 |
| 207 | 0.005 |
| 208 | 0.005 |
| 209 | 0.002 |
| 210 | 0.003 |
| 211 | 0.004 |
| 212 | 0.002 |
| 213 | 0.003 |
| 214 | 0.003 |

TABLE 6

| | Menin Binding |
|---|---|
| Cpd. No. | Affinity IC₅₀ (μM) |
| 250 | 0.001 |
| 251 | 0.001 |
| 252 | 0.002 |
| 253 | 0.002 |
| 254 | 0.001 |
| 255 | 0.003 |
| 256 | 0.007 |
| 257 | 0.001 |
| 258 | 0.005 |
| 259 | 0.015 |
| 260 | 0.002 |
| 261 | 0.003 |
| 262 | 0.001 |
| 263 | 0.001 |
| 264 | 0.001 |
| 265 | 0.001 |
| 266 | 0.001 |
| 267 | 0.005 |
| 268 | 0.002 |
| 269 | 0.002 |
| 270 | 0.001 |
| 271 | 0.001 |
| 272 | 0.001 |
| 273 | 0.001 |
| 274 | 0.002 |
| 275 | 0.002 |
| 276 | 0.004 |
| 277 | 0.002 |
| 278 | 0.002 |
| 279 | 0.002 |
| 280 | 0.003 |
| 281 | 0.002 |
| 282 | 0.003 |
| 283 | 0.002 |
| 284 | 0.003 |
| 285 | 0.002 |
| 286 | 0.002 |
| 287 | 0.002 |
| 288 | 0.005 |
| 289 | 0.002 |
| 290 | 0.002 |
| 291 | 0.003 |
| 292 | 0.002 |
| 293 | 0.002 |
| 294 | 0.003 |
| 295 | 0.003 |
| 296 | 0.002 |
| 297 | 0.002 |
| 298 | 0.002 |
| 299 | 0.004 |
| 300 | 0.011 |
| 301 | 0.004 |
| 302 | 0.007 |
| 303 | 0.004 |
| 304 | 0.004 |
| 305 | 0.009 |
| 306 | 0.005 |
| 307 | 0.002 |
| 308 | 0.008 |
| 309 | 0.006 |
| 310 | 0.006 |
| 311 | 0.008 |
| 312 | 0.021 |
| 313 | 0.004 |
| 314 | 0.180 |
| 315 | 0.086 |
| 316 | 0.117 |
| 317 | 0.003 |
| 318 | 0.007 |
| 319 | 0.004 |
| 320 | 0.003 |
| 321 | 0.005 |
| 322 | 0.004 |
| 323 | 0.002 |
| 324 | 0.012 |
| 325 | 0.003 |
| 326 | 0.002 |
| 327 | 0.002 |
| 328 | 0.003 |
| 329 | 0.148 |
| 330 | 0.007 |
| 331 | 0.003 |
| 332 | 0.003 |
| 333 | 0.003 |
| 334 | 0.004 |
| 335 | 0.004 |
| 336 | 0.009 |
| 337 | 0.006 |
| 338 | 0.005 |
| 339 | 0.004 |
| 340 | 0.045 |
| 341 | 0.004 |
| 342 | 0.004 |
| 343 | 0.002 |
| 344 | 0.003 |
| 345 | 0.002 |
| 346 | 0.0017 |
| 347 | 0.0015 |
| 348 | 0.0026 |
| 349 | 0.0039 |
| 350 | 0.005 |
| 351 | 0.006 |
| 352 | 0.010 |
| 353 | 0.009 |
| 354 | 0.0033 |
| 355 | 0.0021 |
| 356 | 0.0023 |
| 357 | 0.0019 |
| 358 | 0.004 |
| 359 | 0.004 |
| 360 | 0.003 |
| 361 | 0.002 |
| 362 | 0.004 |
| 363 | 0.003 |
| 364 | 0.008 |
| 365 | 0.003 |
| 366 | 0.003 |
| 367 | 0.004 |
| 368 | 0.004 |
| 369 | 0.008 |
| 370 | 0.009 |
| 371 | 0.01 |
| 372 | 0.006 |
| 373 | 0.005 |
| 374 | 0.006 |
| 375 | 0.005 |
| 376 | 0.005 |
| 377 | 0.002 |
| 378 | 0.02 |
| 379 | 0.002 |
| 380 | 0.004 |
| 381 | 0.002 |
| 382 | 0.002 |
| 383 | >0.25 |
| 384 | 0.154 |
| 385 | 0.007 |
| 386 | 0.005 |
| 387 | 0.021 |
| 388 | 0.022 |
| 389 | 0.008 |
| 390 | 0.005 |
| 392 | 0.001 |
| 393 | 0.086 |
| 395 | 0.005 |
| 396 | 0.009 |
| 397 | 0.005 |
| 398 | 0.006 |
| 399 | 0.004 |
| 400 | 0.005 |

### Example 18

### Cell Growth Inhibition

The effect of representative Compounds of the Disclosure on cell viability was determined in a 4-day or 7-day proliferation assay. Cells were maintained in the appropriate culture medium with 10% FBS at 37° C. and an atmosphere of 5% CO₂.

Cells were seeded in 96-well flat bottom (Corning COSTAR, Corning, N.Y., cat #3595) at a density of 2,000-3,000 cells/well in 100 μl of culture medium. Compounds were serially diluted in the appropriate medium, and 100 μl of the diluted compounds were added to the appropriate wells of the cell plate. After the addition of compounds, the cells were incubated at 37° C. in an atmosphere of 5% CO₂ for 4 or 7 days. Cell viability was determined using the WST (2-(2-methoxy-4-nitrophenyl)-3-(4-nitrophenyl)-5-(2,4-disulfophenyl)-2H-tetrazolium, monosodium salt) Cell Counting-8 Kit (Dojindo Molecular Technologies, Inc., Rockville, Md.) according to the manufacturers' instructions.

WST-8 reagent was added to each well at a final concentration of 10% (v/v), and then the plates were incubated at 37° C. for 1-2 hours for color development. The absorbance was measured at 450 nm using a SPECTRAmax PLUS plate reader (Molecular Devices, Sunnyvale, Calif.). The readings were normalized to the DMSO-treated cells and the half maximal inhibitory concentration (IC₅₀) was calculated by nonlinear regression (four parameters sigmoid fitted with variable slope, least squares fit, and no constraint) analysis using the GraphPad Prism 5 software (GraphPad Software, La Jolla, Calif.). See Tables 7-9.

TABLE 7

| | Cell Growth Inhibition | |
|---|---|---|
| | IC₅₀ (nM) | |
| Cpd. No. | MV4; 11 | MOLM13 |
| 2 | <500 (4d) | |
| 3 | >1000 (4d) | |
| 4 | >1000 (4d) | >1000 |
| 5 | >1000 (4d) | >1000 (4d) |
| 26 | <50 (4d) | |
| 33 | <100 | <100 |
| 34 | <500 | <1000 |
| 35 | <1000 | >1000 |
| 36 | <50 | <100 |
| 37 | <50 | <50 |
| 38 | <1000 | |
| 39 | <500 | |
| 40 | <500 | |
| 41 | <500 | <500 |
| 42 | <50 | <100 |
| 43 | <50 | <100 |
| 44 | <50 | <100 |
| 45 | <50 | <500 |
| 46 | <100 | <500 |
| 47 | <10 | <50 |
| 48 | <50 | <500 |
| 49 | <50 | |
| 26 | <50 | |
| 52 | <500 | |
| 53 | <500 | |
| 54 | <500 | |
| 55 | <100 | <500 |
| 56 | <500 | |
| 57 | <50 | <100 |
| 58 | <10 | <100 |
| 59 | <500 | <1000 |
| 60 | <100 | <100 |
| 61 | <100 | <500 |
| 62 | <500 | >1000 |
| 63 | <100 | <1000 |
| 64 | <50 | <1000 |
| 65 | <10 | <500 |
| 68 | <10 | <500 |
| 69 | <100 | >1000 |
| 72 | <10 | <500 |
| 73 | <10 | <500 |
| 74 | <50 | <500 |
| 75 | <50 | <500 |
| 76 | <50 | <500 |
| 77 | <10 | <500 |
| 78 | <100 | >1000 |
| 79 | <50 | <500 |
| 80 | <10 | <50 |
| 81 | <50 | <500 |
| 82 | <10 | <500 |
| 83 | <10 | <500 |

TABLE 8

| | Cell Growth Inhibition | |
|---|---|---|
| | IC₅₀ (nM) | |
| Cpd No. | MV4; 11 | MOLM13 |
| 84 | >1000 | >1000 |
| 85 | <500 | <500 |
| 86 | <100 | <500 |
| 87 | <50 | <100 |
| 88 | <10 | <50 |
| 89 | <50 | <100 |
| 90 | <500 | <1000 |
| 91 | <500 | <500 |
| 92 | <50 | <500 |
| 93 | <10 | <50 |
| 94 | <500 | <1000 |
| 95 | <10 | <10 |
| 96 | <1000 | >1000 |
| 97 | >1000 (4d) | >1000 (4d) |
| 98 | <10 | <50 |
| 99 | <500 (4d) | >1000 (4d) |
| 100 | <10 | <10 |
| 101 | <10 | <50 |
| 102 | <10 | <50 |
| 103 | <10 | <10 |
| 104 | <50 | <50 |
| 105 | <50 | <500 |
| 106 | <10 | <100 |
| 107 | <10 | <50 |
| 108 | <50 | <50 |
| 109 | <50 | <100 |
| 110 | <50 | <50 |
| 111 | <50 | <50 |
| 112 | <50 | <100 |
| 113 | <50 | <100 |
| 114 | <50 | <50 |
| 115 | <10 | <50 |
| 116 | <50 | <50 |
| 117 | <50 | <50 |
| 118 | <10 | <50 |
| 119 | <50 | <50 |
| 120 | <50 | <100 |
| 121 | <10 | <50 |
| 122 | <50 | <50 |
| 123 | <50 | <50 |
| 124 | <50 | <50 |
| 125 | <50 | <50 |
| 126 | <100 | <500 |
| 127 | <50 | <50 |
| 128 | <50 | <100 |
| 129 | <100 | <100 |
| 130 | <50 | <50 |
| 131 | <50 | <50 |
| 132 | <100 | <500 |
| 133 | <50 | <500 |
| 134 | <500 | <1000 |
| 135 | <100 | <500 |
| 136 | <500 | >1000 |
| 137 | <50 | <500 |
| 138 | <50 | <100 |
| 139 | <50 | <500 |
| 140 | <50 | <100 |
| 141 | <50 | <100 |
| 142 | <500 | >1000 |
| 143 | <50 | <100 |
| 144 | <500 | >1000 |
| 145 | <500 | >1000 |
| 146 | >1000 | >1000 |
| 147 | <10 | <50 |
| 148 | <500 | <1000 |
| 149 | <100 | <500 |
| 150 | | <1000 |
| 151 | <500 | >1000 |
| 152 | <500 | <500 |
| 153 | <1000 | >1000 |
| 154 | <1000 | <500 |
| 155 | <1000 | <500 |
| 156 | <50 | <50 |
| 157 | <50 | <100 |
| 158 | <10 | <100 |
| 159 | <10 | <100 |
| 160 | <500 | <1000 |
| 161 | <100 | <500 |
| 162 | <50 | <50 |
| 163 | <50 | <500 |
| 164 | <10 | <100 |
| 165 | <500 | <1000 |
| 166 | <50 | <100 |
| 167 | <50 | <500 |
| 168 | <100 | <500 |
| 169 | <10 | <10 |
| 170 | <500 | <1000 |
| 171 | <500 | <1000 |
| 172 | <500 | <1000 |
| 173 | <50 | <500 |
| 174 | <500 | <1000 |
| 175 | <10 | <10 |
| 176 | <10 | <10 |
| 177 | <500 | <500 |
| 178 | <1000 | >1000 |
| 179 | <100 | <500 |
| 180 | <100 | <500 |
| 181 | <50 | <100 |
| 182 | <100 | <500 |
| 183 | <50 | <50 |
| 184 | <500 | <500 |
| 185 | <100 | <500 |
| 186 | <50 | <500 |
| 187 | <10 | <50 |
| 188 | <500 | <500 |
| 189 | <1000 | >1000 |
| 190 | <50 | <500 |
| 191 | <10 | <50 |
| 192 | <10 | <50 |
| 193 | <50 | <500 |
| 194 | <10 | <50 |
| 195 | >1000 | >1000 |
| 196 | <10 | <50 |
| 197 | >1000 | >1000 |
| 199 | >1000 | >1000 |
| 200 | <500 | <500 |
| 201 | <1000 | >1000 |
| 202 | >1000 | >1000 |
| 203 | <50 | <500 |
| 204 | <100 | <500 |
| 205 | <500 | >1000 |
| 207 | <500 | <500 |
| 208 | <500 | <1000 |
| 209 | <500 | <1000 |
| 210 | <500 | <1000 |
| 211 | <500 | >1000 |
| 212 | <100 | <500 |
| 213 | <500 | <1000 |
| 214 | <500 | <500 |

TABLE 9

| | Cell Growth Inhibition | |
|---|---|---|
| | IC₅₀ (nM) | |
| Cpd No. | MV4; 11 | MOLM13 |
| 250 | | <50 |
| 251 | <10 | <10 |
| 252 | <50 | <50 |
| 253 | <500 | <1000 |
| 254 | <10 | <50 |
| 255 | <100 | <500 |
| 256 | <500 | >1000 |
| 257 | <100 | <500 |
| 258 | <500 | >1000 |
| 259 | >1000 | >1000 |
| 260 | <500 | <500 |
| 261 | <50 | <500 |
| 262 | <50 | <50 |
| 263 | <50 | <50 |
| 264 | <10 | <10 |
| 265 | <10 | <10 |
| 266 | <10 | <10 |
| 267 | <500 | <500 |
| 268 | <100 | <500 |
| 269 | <50 | <50 |
| 270 | <500 | <500 |
| 271 | <10 | <10 |
| 272 | <10 | <10 |
| 273 | <10 | <10 |
| 274 | <10 | <10 |
| 275 | <10 | <10 |
| 276 | <50 | <100 |
| 277 | <10 | <50 |
| 278 | <10 | <10 |
| 279 | <10 | <10 |
| 280 | <50 | <100 |
| 281 | <10 | <50 |
| 282 | <100 | <500 |
| 283 | <10 | <50 |
| 284 | <10 | <50 |
| 285 | <10 | <10 |
| 286 | <50 | <50 |
| 287 | <10 | <10 |
| 288 | <100 | <500 |
| 289 | <50 | <50 |
| 290 | <10 | <50 |
| 291 | <50 | <50 |
| 292 | <50 | <50 |
| 293 | <50 | <50 |
| 294 | <50 | <100 |
| 295 | <50 | <50 |
| 296 | <50 | <50 |
| 297 | <10 | <10 |
| 298 | <50 | <50 |
| 299 | <50 | <50 |
| 300 | <100 | <500 |
| 301 | <500 | <100 |
| 302 | <500 | <500 |
| 303 | <500 | <1000 |
| 304 | <10 | <10 |
| 305 | <500 | <1000 |
| 306 | <50 | <500 |
| 307 | <50 | <500 |
| 308 | <500 | <1000 |
| 309 | <500 | <1000 |
| 310 | <500 | <1000 |
| 311 | <500 | <1000 |
| 312 | <1000 | <1000 |
| 313 | <100 | <500 |
| 316 | >1000 | >1000 |
| 317 | <50 | <500 |
| 318 | <100 | <500 |
| 319 | <50 | <100 |
| 320 | <50 | <100 |
| 321 | <500 | <500 |
| 322 | <50 | <100 |
| 323 | <500 | <500 |
| 324 | <500 | <1000 |
| 325 | <50 | <100 |
| 331 | <50 | <100 |
| 332 | <500 | <50 |
| 333 | <50 | <50 |
| 334 | <100 | <500 |
| 335 | <500 | <500 |
| 336 | <500 | <500 |
| 337 | <50 | <500 |
| 338 | <500 | <1000 |
| 339 | <10 | <50 |
| 344 | <500 | <500 |
| 345 | <100 | <500 |
| 346 | <50 | <50 |
| 347 | <50 | <50 |
| 348 | <500 | <1000 |
| 349 | <500 | <500 |
| 350 | <500 | <1000 |
| 351 | <100 | <100 |
| 352 | <100 | <1000 |
| 353 | <1000 | >1000 |
| 359 | <100 | |
| 360 | <100 | |
| 361 | <50 | <50 |
| 362 | <100 | |
| 363 | <50 | <50 |
| 365 | <50 | <50 |
| 366 | <100 | <100 |
| 367 | <100 | |
| 369 | <50 | |
| 370 | <100 | |
| 371 | <100 | |
| 372 | <50 | |
| 373 | <100 | |
| 374 | <50 | <100 |
| 375 | <50 | |
| 379 | <50 | |
| 389 | <500 | >1000 |
| 390 | >1000 | >1000 |
| 395 | <50 | <100 |
| 396 | <500 | >1000 |
| 397 | <10 | <50 |
| 398 | <100 | <500 |
| 399 | <50 | <100 |
| 400 | <50 | <500 |

### Example 19

### MV4-11 Xenograft Tumor Model

MV4;11 cells were grown in suspension and collected in log phase. A cell sample was mixed 1:1 with Trypan Blue (GIBCO™, Invitrogen Corp.) and counted on a hemocytometer to determine the number of live/dead cells. Cells were washed twice with IX PBS (GIBCO™, Invitrogen Corp.) and resuspended in an ice cold mixture of 1:1 PBS and Matrigel (BD Biosciences, Invitrogen Corp.) for a final Matrigel protein concentration of 5 mg/ml.

MV4;11 cells were inoculated into female C.B-17 SCID mice at 5×10⁶ cells in 0.1 ml with Matrigel. Cells were injected s.c. into the flank region of each mouse. The size of tumors growing in the mice was measured in two dimensions using calipers. Tumor volume (mm³)=(A×B²)/2 where A and B are the tumor length and width (in mm), respectively. For efficacy study, before treatment began, tumors were allowed to grow to 70-200 mm³ in volume. Mice with tumors within acceptable size range were randomized into treatment groups of 7 mice per group.

Cpd. No. 42 was given orally, in 100% PEG 200 vehicle, at a volume of 10 ul per gram of body weight. The Control group was given vehicle only. Cpd. No. 42 was dosed at three concentrations (25, 50, and 100 mg/kg) initially for five days with 2 days off, then daily thereafter. During treatment, tumor volume and body weight was measured two or three times per week. After the treatment was stopped, tumor volume and body weight was measured at least once per week. The tumor volume data is provided in FIG. 1.

All patents, patent applications and publications cited herein are fully incorporated by reference herein in their entirety.

What is claimed is:

1. A compound of Formula I: 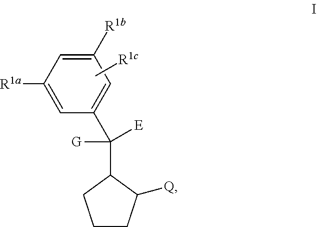
wherein:
Q is selected from the group consisting of —N(H)C(═O)OR, —N(R)C(═O)OR, —N(H)C(═O)R, —N(H)C(O)NR₂, 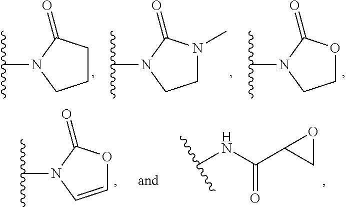
—OR, and —OC(═O)R;
each R is independently C₁-C₄ alkyl or C₁-C₄ haloalkyl;
G is selected from the group consisting of: 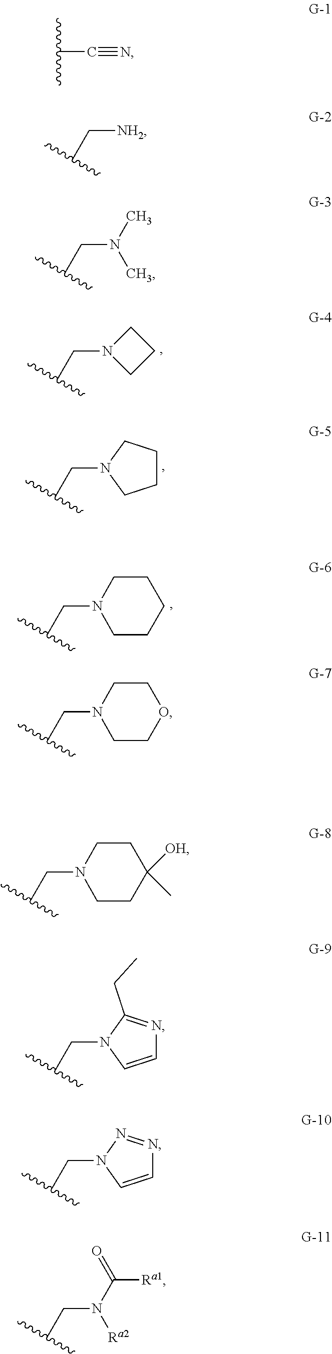 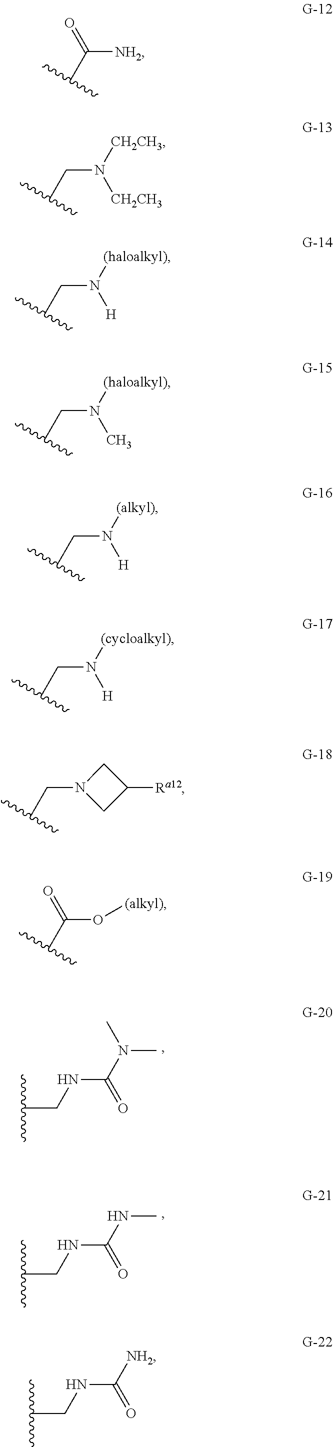 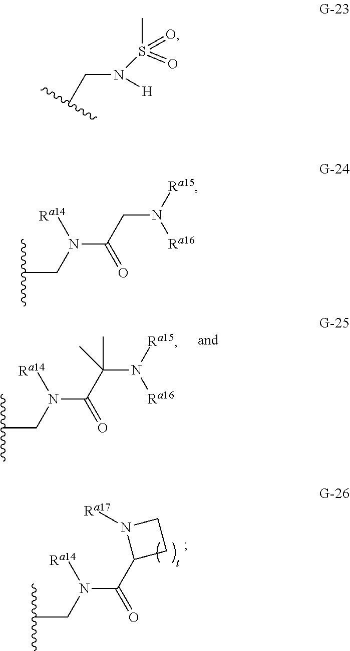
R^{a1} is selected from the group consisting of C₁-C₄ alkyl and C₁-C₄ alkoxy;
R^{a2} is selected from the group consisting of hydrogen and C₁-C₄ alkyl; or
R^{a1} and R^{a2} taken together with the atoms to which they are attached form an optionally substituted 5- or 6-membered heterocyclo;
R^{a12} is CN, C(O)OR^{a13}, C(O)N(R^{a13})₂, C₁-C₄ alkyl, OH, C₁-C₄ alkoxy, or F;
each R^{a13} is independently C₁-C₄ alkyl;
R^{a14} is H or C₁-C₄ alkyl;
R^{a15} and R^{a16} are each independently H or C₁-C₄ alkyl, or R^{a14} and R^{a15} together with the nitrogen atom to which they are attached form an optionally substituted 4- to 6-membered heterocyclo;
R^{a17} is H or C₁-C₄ alkyl;
t is 1, 2, or 3;
R^{1a}, R^{1b}, and R^{1c} are each independently selected from the group consisting of hydrogen and halo;
E is selected from the group consisting of: 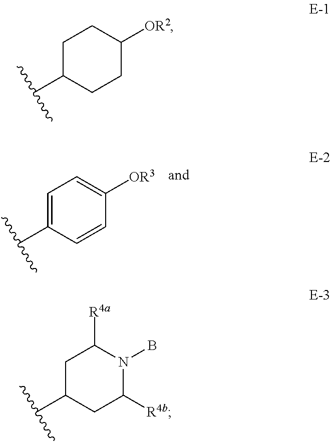
R² is selected from the group consisting of C₁-C₆ alkyl and —(CR^{5a}R^{5b})_{P}OR^{6a};
R³ is selected from the group consisting of hydrogen, —(CR^{5a}R^{5b})_{P}OR^{6b}, —CH₂C≡CR⁷, and 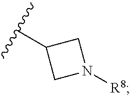
each R^{5a} and R^{5b} is independently selected from the group consisting of hydrogen and C₁-C₄ alkyl;
p is 2, 3, or 4;
R^{6a} is optionally substituted phenyl;
R^{6b} is selected from the group consisting of C₁-C₆, alkyl and optionally substituted phenyl;
R⁷ is optionally substituted phenyl;
R⁸ is optionally substituted phenyl;
R^{4a} and R^{4b} are independently selected from the group consisting of hydrogen, halo, and C₁-C₄ alkyl;
B is selected from the group consisting of C₁-C₆ alkyl, aralkyl, —C(═O)R⁹, —(CR^{5c}R^{5d})ₘOR¹⁰, 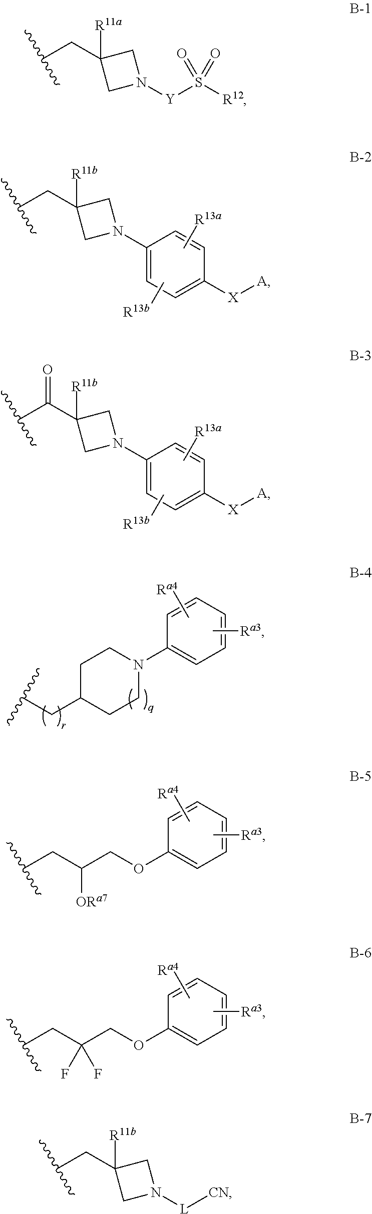 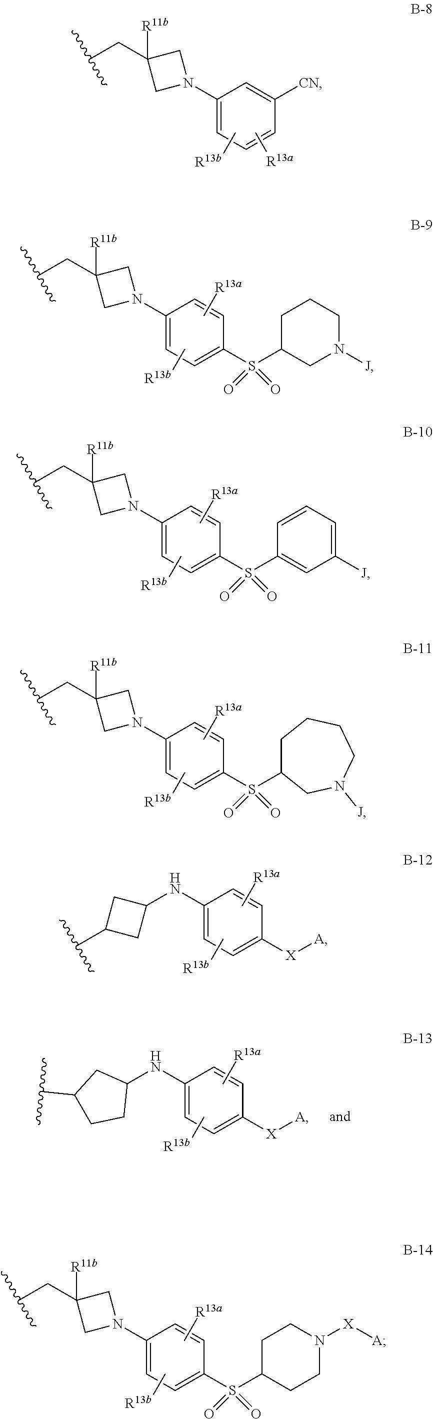
R⁹ is selected from the group consisting of C₁-C₆, alkyl, aralkyl, heteroaralkyl, and 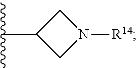
R¹⁴ is optionally substituted phenyl;
each R^{5c} and R^{5d} is independently selected from the group consisting of hydrogen and C₁-C₄ alkyl;
m is 2, 3, or 4;
R¹⁰ is optionally substituted phenyl;
R^{11a} is selected from the group consisting of hydrogen, halo, and C₁-C₄ alkyl;
Y is —(CR^{5e}R^{5f})ₒ;
each R^{5e} and R^{5f} is independently selected from the group consisting of hydrogen and C₁-C₄ alkyl;
o is 2, 3, or 4;
R¹² is optionally substituted phenyl;
R^{11b} is selected from the group consisting of hydrogen, halo, C₁-C₄ alkyl, and R^{a6};
R^{13a} and R^{13b} are independently selected from the group consisting of hydrogen, halo, C₁-C₄ alkyl, and R^{a5};
R^{a3} is selected from the group consisting of cyano, alkylsulfonyl, haloalkylsulfonyl, cycloalkylsulfonyl, arylsulfonyl, heteroarylsulfonyl, heterocyclosulfonyl, and carboxamido;
R^{a4} is selected from the group consisting of hydrogen, halo, C₁-C₄ alkyl, C₁-C₄ alkoxy, and C₁-C₄ haloalkyl;
R^{a5} is selected from the group consisting of (heterocyclo)alkyl, (heteroaryl)alkyl, (amino)alkyl, carboxamido, hydroxyalkyl, heteroaryloxy, heteroaralkyloxy, C₁-C₄ alkoxy, —OR^{a8}, and —CH₂NR^{a9}C(═O)R^{a10};
R^{a6} is selected from the group consisting of hydroxy, C₁-C₄ alkoxy, C₁-C₄ haloalkyl, C₁-C₄ hydroxyalkyl, alkoxyalkyl, carboxy, alkoxy carbonyl, and carboxamido;
R^{a7} is selected from the group consisting of hydrogen and C₁-C₄ alkyl;
R^{a8} is selected from the group consisting of heteroaryl, heteroaralkyl, alkoxyalkyl, and (heterocyclo)alkyl;
R^{a9} is selected from the group consisting of hydrogen and C₁-C₄ alkyl;
R^{a10} is C₁-C₄ alkyl;
r is 0 or 1;
q is 0, 1, 2, or 3;
L is selected from the group consisting of C₃-C₈ cycloalkylenyl, optionally substituted 5-membered heteroaryl enyl, and optionally substituted 6-membered heteroaryl enyl;
(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b}, 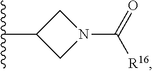
and R^{a11}; or
(2) X is absent; and A is selected from the group consisting of cyano, C(═O)OH, C₁-C₄ alkyl, C₁-C₄ alkoxy, and C₁-C₄ haloalkyl;
J is carboxamido or C(O)CH₂CN;
R^{a11} is selected from the group consisting of hydroxyalkyl and (heterocyclo)alkyl;
R^{15a} and R^{15b} are independently selected from the group consisting of hydrogen, optionally substituted C₁-C₆ alkyl, optionally substituted C₃-C₁₂ cycloalkyl, optionally substituted 4- to 14-membered heterocyclo, optionally substituted C₆-C₁₀ aryl, and optionally substituted 5- to 14-membered heteroaryl; and
R¹⁶ is selected from the group consisting of (amino)alkyl and (heterocyclo)alkyl,
or a pharmaceutically acceptable salt or solvate thereof.

2. The compound of claim 1, wherein:
Q is selected from the group consisting of —N(H)C(═O)OR, —OR, and —OC(═O)R;
R is a C₁-C₄ alkyl;
G is selected from the group consisting of: 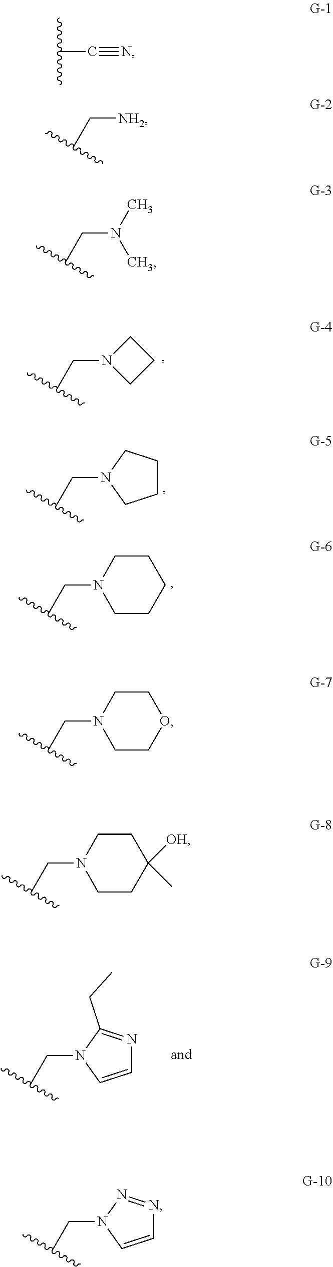
B is selected from the group consisting of C₁-C₆ alkyl, aralkyl, —C(═O)R⁹, —(CR^{5c}R^{5d})ₘOR¹⁰, 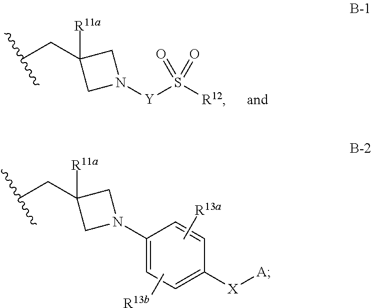
R^{11b} is selected from the group consisting of hydrogen, halo, and C₁-C₄ alkyl;
R^{13a} and R^{13b} are independently selected from the group consisting of hydrogen, halo, and C₁-C₄ alkyl; and
(1) X is selected from the group consisting of —S(═O)₂— and —C(═O)—; and A is selected from the group consisting of optionally substituted C₁-C₆ alkyl, optionally substituted C₁-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b}, and R¹⁶; 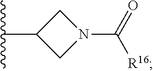
or
(2) X is absent; and A is selected from the group consisting of cyano and —C(═O)OH;
or a pharmaceutically acceptable salt or solvate thereof.

3. The compound of claim 1 or 2 of Formula II: 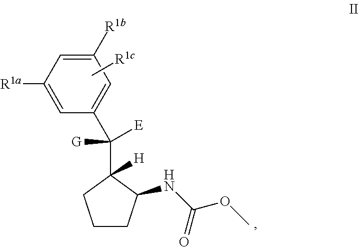
or a pharmaceutically acceptable salt or solvate thereof.

4. The compound of claim 1, 2 or 3, wherein E is E-1, or a pharmaceutically acceptable salt or solvate thereof.

5. The compound of claim 1, 2 or 3, wherein E is E-2, or a pharmaceutically acceptable salt or solvate thereof.

6. The compound of claim 1, 2 or 3, wherein E is E-3, or a pharmaceutically acceptable salt or solvate thereof.

7. The compound of any one of claims 1, 2, 3, and 6, wherein B is C₁-C₆ alkyl, or a pharmaceutically acceptable salt or solvate thereof.

8. The compound of any one of claims 1, 2, 3, and 6, wherein B is aralkyl, or a pharmaceutically acceptable salt or solvate thereof.

9. The compound of any one of claims 1, 2, 3, and 6, wherein B is —C(═O)R⁹, or a pharmaceutically acceptable salt or solvate thereof.

10. The compound of any one of claims 1, 2, 3, and 6, wherein B is —(CH₂)ₘOR¹⁰ or a pharmaceutically acceptable salt or solvate thereof.

11. The compound of any one of claims 1, 2, 3, and 6, wherein B is B-1, or a pharmaceutically acceptable salt or solvate thereof.

12. The compound of any one of claims 1, 2, 3, and 6, wherein B is B-2, or a pharmaceutically acceptable salt or solvate thereof.

13. The compound of any one of claims 1-12, wherein G is —CN, or a pharmaceutically acceptable salt or solvate thereof.

14. The compound of any one of claims 1-12, wherein G is 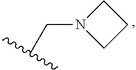 [SMILES: *CN1CCC1]
or a pharmaceutically acceptable salt or solvate thereof.

15. The compound of claim 1 or 2 of Formula III: 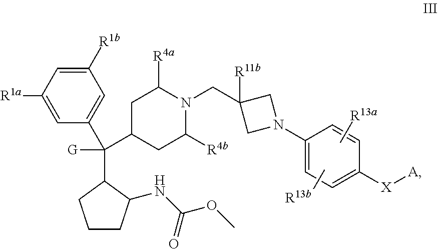
wherein:
G is selected from the group consisting of: 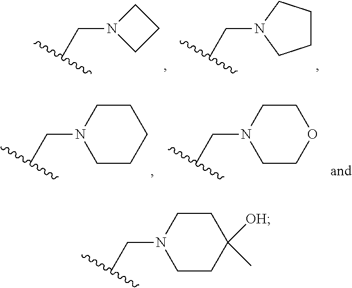
or a pharmaceutically acceptable salt or solvate thereof.

16. The compound of claim 15 of Formula IV: 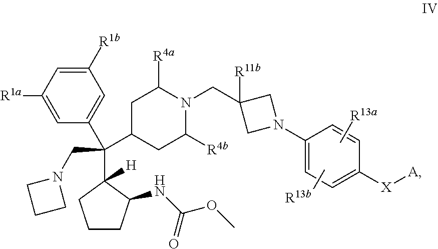
or a pharmaceutically acceptable salt or solvate thereof.

17. The compound of claim 15 or 16, wherein R^{4a} and R^{4b} are hydrogen, or a pharmaceutically acceptable salt or solvate thereof.

18. The compound of any one of claims 15-17, wherein R^{11b} is selected from the group consisting of hydrogen and fluoro, or a pharmaceutically acceptable salt or solvate thereof.

19. The compound of any one of claims 15-18, wherein R^{13a} and R^{13b} are independently selected from the group consisting of hydrogen and fluoro, or a pharmaceutically acceptable salt or solvate thereof.

20. The compound of any one of claims 15-19, wherein A is optionally substituted C₃-C₁₂ cycloalkyl, or a pharmaceutically acceptable salt or solvate thereof.

21. The compound of any one of claim 15-19, wherein A is optionally substituted 4- to 14-membered heterocyclo, or a pharmaceutically acceptable salt or solvate thereof.

22. The compound of any one of claims 15-19, wherein X is absent and A is cyano, or a pharmaceutically acceptable salt or solvate thereof.

23. The compound of claim 2 selected from the group consisting of the compounds of Table 1.1, or a pharmaceutically acceptable salt thereof.

24. The compound of claim 1, wherein:
Q is selected from the group consisting of —N(H)C(═O)OR and —N(H)C(═O)R;
R is a C₁-C₄ alkyl;
G is selected from the group consisting of: 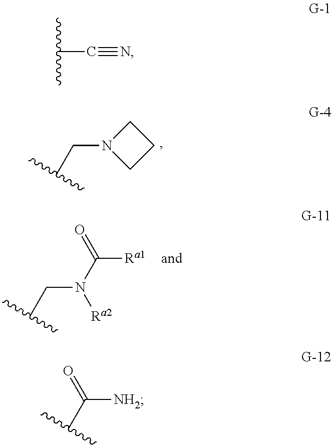
E is: 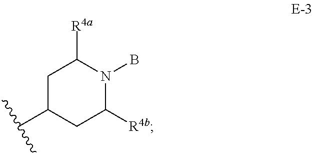
R^{4a} and R^{4b} are independently selected from the group consisting of hydrogen, halo, and C₁-C₄ alkyl;
B is selected from the group consisting of: 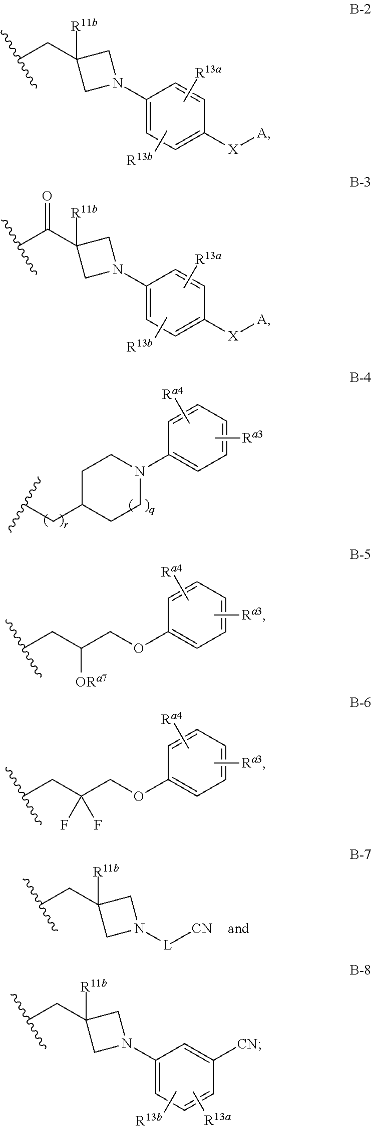
and
R^{11b} is selected from the group consisting of hydrogen, halo, C₁-C₄ alkyl, and R^{a6};
R^{a5} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10};
R^{a6} is selected from the group consisting of hydroxy, C₁-C₄ haloalkyl, alkoxyalkyl, carboxy, alkoxycarbonyl, and carboxamido;
(1) X is selected from the group consisting of —S(═O)₂— and —C(═O)—; and A is selected from the group consisting of optionally substituted C₁-C₆ alkyl, optionally substituted C₃-C₁₂ cycloalkyl, optionally substituted 4- to 14-membered heterocyclo, optionally substituted C₆-C₁₀ aryl, optionally substituted 5- to 14-membered heteroaryl, —NR^{15a}R^{15b}, 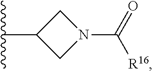
and R^{a11}; or
(2) X is absent; and A is selected from the group consisting of cyano and —C(═O)OH;
or a pharmaceutically acceptable salt or solvate thereof.

25. The compound of claim 1 or 14 of Formula II: 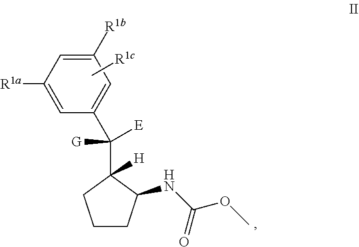
or a pharmaceutically acceptable salt or solvate thereof.

26. The compound of claim 1, 24 or 25, wherein B is B-2, or a pharmaceutically acceptable salt or solvate thereof.

27. The compound of claim 1, 24 or 25, wherein B is B-3, or a pharmaceutically acceptable salt or solvate thereof.

28. The compound of claim 27, wherein X is absent and A is cyano, or a pharmaceutically acceptable salt or solvate thereof.

29. The compound of claim 27, wherein:
A is selected from the group consisting of phenyl substituted with 1 or 2 substituents independently selected from the group consisting of halo and carboxamido, and 5- or 6-membered heteroaryl substituted with 1 or 2 substituents independently selected from the group consisting of halo and carboxamido.

30. The compound of claim 27, wherein:
A is selected from the group consisting of unsubstituted C₃-C₆ cycloalkyl, C₃-C₆ cycloalkyl substituted with 1 or 2 substituents independently selected from the group consisting of halo, hydroxy, C₁-C₄ alkyl, and C₁-C₄ haloalkyl, unsubstituted 4- to 6-membered heterocyclo, and 4- to 6-membered heterocyclo substituted with 1 or 2 substituents independently selected from the group consisting of halo, hydroxy, C₁-C₄ alkyl, and C₁-C₄ haloalkyl, alkylcarbonyl, hydroxyalkylcarbonyl, and alkoxycarbonyl.

31. The compound of claim 1, 24 or 25, wherein B is B-4, or a pharmaceutically acceptable salt or solvate thereof.

32. The compound of claim 1, 24 or 25, wherein B is B-5, or a pharmaceutically acceptable salt or solvate thereof.

33. The compound of claim 1, 24 or 25, wherein B is B-6, or a pharmaceutically acceptable salt or solvate thereof.

34. The compound of claim 1, 24 or 25, wherein B is B-7, or a pharmaceutically acceptable salt or solvate thereof.

35. The compound of claim 1, 24 or 25, wherein B is B-8, or a pharmaceutically acceptable salt or solvate thereof.

36. The compound of any one of claims 27-28 and 35, wherein R^{11b} is selected from the group consisting of hydrogen or fluoro, or a pharmaceutically acceptable salt or solvate thereof.

37. The compound of any one of claims 1 and 24-36, wherein G is G-1, or a pharmaceutically acceptable salt or solvate thereof.

38. The compound of any one of claims 1 and 24-36, wherein G is G-4, or a pharmaceutically acceptable salt or solvate thereof.

39. The compound of any one of claims 1 and 24-36, wherein G is G-11, or a pharmaceutically acceptable salt or solvate thereof.

40. The compound of claim 1 or 24 of Formula III: 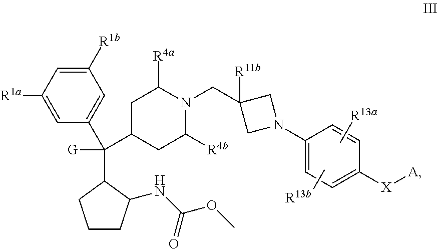
wherein G is selected from the group consisting of G-4 and G-11, or a pharmaceutically acceptable salt or solvate thereof.

41. The compound of claim 40, wherein R^{13b} is selected from the group consisting of hydrogen, or a pharmaceutically acceptable salt or solvate thereof.

42. The compound of claim 41 of Formula V: 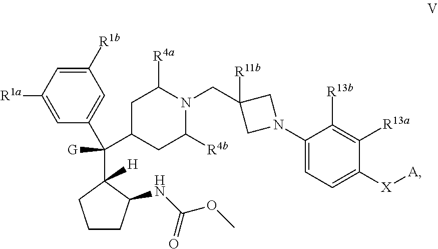
or a pharmaceutically acceptable salt or solvate thereof.

43. The compound of claim 41 of Formula VI: 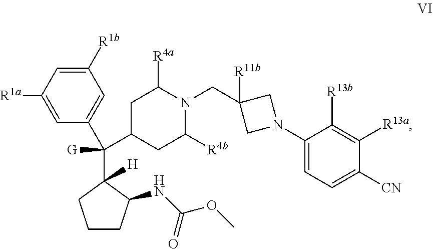
or a pharmaceutically acceptable salt or solvate thereof.

44. The compound of any one of claims 1 and 24-43, wherein R^{4a} and R^{4b} are hydrogen, or a pharmaceutically acceptable salt or solvate thereof.

45. The compound of any one of claims 1 and 24-26 and 40-44, wherein R^{11b} is selected from the group consisting of hydrogen or fluoro, or a pharmaceutically acceptable salt or solvate thereof.

46. The compound of any one of claims 1, 24-26 and 40-45, wherein R^{13a} selected from the group consisting of hydrogen and fluoro, or a pharmaceutically acceptable salt or solvate thereof.

47. The compound of any one of claims 1, 24-26 and 40-46, wherein R^{13b} selected from the group consisting of hydrogen and fluoro, or a pharmaceutically acceptable salt or solvate thereof.

48. The compound of any one of claims 1, 24-26, 40-45 and 47, wherein R^{13a} is selected from the group consisting of (heterocyclo)alkyl, (heteroaryl)alkyl, (amino)alkyl, hydroxyalkyl, heteroaryloxy, heteroaralkyloxy, C₁-C₄ alkoxy, —OR^{a8}, and —CH₂NR^{a9}C(═O)R^{a10}, or a pharmaceutically acceptable salt or solvate thereof.

49. The compound of any one of claims 1 and 24-48, wherein R^{1a} and R^{1b} are independently selected from the group consisting of hydrogen and fluoro, or a pharmaceutically acceptable salt or solvate thereof.

50. The compound of claim 24 selected from the group consisting of the compounds of Table 1.2, or a pharmaceutically acceptable salt thereof.

51. The compound of claim 1, wherein Q is —N(H)C(═O)OR, or a pharmaceutically acceptable salt or solvate thereof.

52. The compound of claim 1, wherein Q is selected from the group consisting of —N(R)C(═O)OR, —N(H)C(O)NR₂, 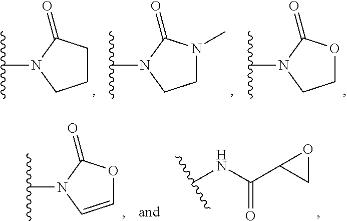
or a pharmaceutically acceptable salt or solvate thereof.

53. The compound of any one of claims 1 and 51-52, wherein G is selected from the group consisting of G-1 and G-4, or a pharmaceutically acceptable salt or solvate thereof.

54. The compound of any one of claims 1 and 51-52, wherein G is selected from the group consisting of G-13, G-14, G-15, G-16, G-17, G-18, G-19, G-20, G-21, G-22, and G-23, or a pharmaceutically acceptable salt or solvate thereof.

55. The compound of any one of claims 1 and 51-54, wherein E is E-3, or a pharmaceutically acceptable salt or solvate thereof.

56. The compound of any one of claims 1 and 51-55, wherein B is B-2, or a pharmaceutically acceptable salt or solvate thereof.

57. The compound of any one of claims 1 and 51-55, wherein B is selected from the group consisting of B-9, B-10, B-11, B-12, B-13, and B-14, or a pharmaceutically acceptable salt or solvate thereof.

58. The compound of any one of claims 1 and 51-57, wherein R^{a5} is carboxamido, or a pharmaceutically acceptable salt or solvate thereof.

59. The compound of any one of claims 1 and 51-58, wherein R^{a6} is selected from the group consisting of C₁-C₄ alkoxy and C₁-C₄ hydroxyalkyl, or a pharmaceutically acceptable salt or solvate thereof.

60. The compound of any one of claims 1 and 51-59, wherein X is absent; and A is C₁-C₄ haloalkyl, or a pharmaceutically acceptable salt or solvate thereof.

61. The compound of claim 1 selected from the group consisting of the compounds of Table 1.3, or a pharmaceutically acceptable salt thereof.

62. A pharmaceutical composition comprising the compound of any one of claims 1-61, or a pharmaceutically acceptable salt thereof, and a pharmaceutically acceptable carrier.

63. A method of treating a subject in need thereof, the method comprising administering to the subject a therapeutically effective amount of the compound of any one of claims 1-61, or a pharmaceutically acceptable salt or solvate thereof, wherein the subject has cancer.

64. The method of claim 63, wherein the cancer is any one or more of the cancers of Table 2.

65. The method of claim 64, wherein the cancer is a hematological cancer.

66. The method of claim 65, wherein the hematological cancer is any one or more of the cancers of Table 3.

67. The method of any one of claims 63-66 further comprising administering a therapeutically effective amount of a second therapeutic agent useful in the treatment of cancer.
